SEQUENCE LISTING (306 sequences)

SEQ ID NO: 1 - protein, 22 aa, synthetic construct
KMSPARPRTSCREACLQGWTEA

SEQ ID NO: 2 - protein, 24 aa, synthetic construct
RRKMSPARPRTSCREACLQGWTEA

SEQ ID NO: 3 - protein, 421 aa, synthetic construct
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDNCRRMMRTKMRMRRMRRTRRKMRRKMSPARPRTSCREACLQGWTEADYKDDDDK

SEQ ID NO: 4 - protein, 408 aa, synthetic construct
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDELDYKDDDDK

SEQ ID NO: 5 - protein, 434 aa, synthetic construct
MRGSHHHHHHGMASMTGGQQMGRDLYDDDDKDRWGSELEAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 6 - protein, 407 aa, synthetic construct
MGSHHHHHHGSSGEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRSKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 7-9 - skipped (3 entries)

SEQ ID NO: 10 - protein, 5 aa, synthetic construct
IYGMS

SEQ ID NO: 11 - protein, 19 aa, synthetic construct
IIYIEGITYYANWAKGRFT

SEQ ID NO: 12 - protein, 5 aa, synthetic construct
GVGRI

SEQ ID NO: 13 - protein, 112 aa, synthetic construct
QEQLKESGGRLVTPGTPLTLTCAVSGFSLDIYGMSWVRQAPGKGLEWIGIIYIEGITYYANWAKGRFTISKTSSTTLELKMTSLTTEDTAMYFCVAGVGRIWGPGTLVTVSS

SEQ ID NO: 14 - protein, 13 aa, synthetic construct
QASQSLYNNNNLA

SEQ ID NO: 15 - protein, 7 aa, synthetic construct
PASTLAS

SEQ ID NO: 16 - protein, 13 aa, synthetic construct
LGEFSCSSADCYA

SEQ ID NO: 17 - protein, 113 aa, synthetic construct
YVMMTQTPSSVSAAVGGTVTINCQASQSLYNNNNLAWFQQKPGQPPKRLIYPASTLASGVSSRFKGSGSGTQFTLTISDVQRDDAATYFCLGEFSCSSADCYAFGGGTEVVVK

SEQ ID NO: 18 - skipped

SEQ ID NO: 19 - protein, 19 aa, synthetic construct
VIGNTGTTYYASWAKGRFT

SEQ ID NO: 20 - protein, 12 aa, synthetic construct
GPPSYSNSVKNI

SEQ ID NO: 21 - protein, 118 aa, synthetic construct
QSVEESGGRLVTPGTPLTLTCTVSGIDLSNNAISWVRQAPGKGLEYIGVIGNTGTTYYASWAKGRFTISKTSSTTVDLKMTSLTASDTATYFCVRGPPSYSNSVKNIWGPGTLVTVSS

SEQ ID NO: 22 - protein, 13 aa, synthetic construct
QSSQSVYDKNWLS

SEQ ID NO: 23 - protein, 7 aa, synthetic construct
DASKLES

SEQ ID NO: 24 - protein, 10 aa, synthetic construct
AGGFTGNVYA

SEQ ID NO: 25 - protein, 110 aa, synthetic construct
DPMLTQTPSSVSAAVGGTVTIGCQSSQSVYDKNWLSWYQQKPGQPPSRLIYDASKLESGVPSRFSGSGSGTQFTLTIGGVQCDDAATYYCAGGFTGNVYAFGGGTKLEIK

SEQ ID NO: 26 - protein, 5 aa, synthetic construct
NNAVS

SEQ ID NO: 27 - protein, 19 aa, synthetic construct
VIGNTGSTYYANWAKGRFT

SEQ ID NO: 28 - protein, 14 aa, synthetic construct
VRGPPSYSNSVKNI

SEQ ID NO: 29 - protein, 119 aa, synthetic construct
QEQLKESGGRLVAPGTPLRLTCRVSGIDLSNNAVSWVRQAPGKGLEYIGVIGNTGSTYYANWAKGRFTISKTSSTTVDLKMTSLTADDTATYFCVRGPPSYSNSVKNIWGPGTLVTVSS

SEQ ID NO: 30 - protein, 110 aa, synthetic construct
DPMLTQTPSSVSAAVGGTVTIGCQSSQSVYDKNWLSWYQQKPGQPPSRLIYDASKLESGVPSRFSGSGSGTQFTLTISGVQCDDAATYYCAGGFTGNVYAFGGGTEVVVK

SEQ ID NO: 31 - protein, 19 aa, synthetic construct
VIGNTGDTYYASWAKGRFT

SEQ ID NO: 32 - protein, 119 aa, synthetic construct
QEQLEESGGRLVTPGTPLTLTCKVSGIDLSNNAISWVRQAPGKGLEYIGVIGNTGDTYYASWAKGRFTISKTSSTTVDLKMTSLTTEDTATYFCVRGPPSYSNSVKNIWGPGTLVTISS

SEQ ID NO: 33 - protein, 13 aa, synthetic construct
QSSQSVYNNNWLS

SEQ ID NO: 34 - protein, 10 aa, synthetic construct
AGGFTGNVYT

SEQ ID NO: 35 - protein, 110 aa, synthetic construct
DPMLTQTPSSVSAAVGGTVTINCQSSQSVYNNNWLSWLQQKPGQPPNRLIYDASKLESGVPSRFSGSGSGTQFTLTISGVQRDDAATYYCAGGFTGNVYTFGGGTEVVVK

SEQ ID NO: 36 - protein, 119 aa, synthetic construct
QEQLKESGGRLVAPGTPLRLTCTVSGIDLSNNAVSWVRQAPGKGLEYIGVIGNTGTTYYASWAKGRFTISKTTSTTVDLKMTSLTTDDTATYFCVRGPPSYSNSVKNIWGPGTLVTVSS

SEQ ID NO: 37 - protein, 10 aa, synthetic construct
AGGFTGNVYG

SEQ ID NO: 38 - protein, 110 aa, synthetic construct
DPVLTQTPSSVSAAVGGTVTIGCQSSQSVYDKNWLSWYQQKPGQPPSRLIYDASKLESGVPSRFSGSGSGTQFTLTISGVQCDDAATYYCAGGFTGNVYGFGGGTEVVVK

SEQ ID NO: 39 - protein, 19 aa, synthetic construct
VIGNTGTTYYANWAKGRFT

SEQ ID NO: 40 - protein, 12 aa, synthetic construct
GPPSYSNSVRNI

SEQ ID NO: 41 - protein, 118 aa, synthetic construct
QQLEESGGRLVAPGTPLRLTCTVSGIDLSNNAVSWVRQAPGKGLEYIGVIGNTGTTYYANWAKGRFTISKTSSTTVDLKMTSLTTDDTATYFCVRGPPSYSNSVRNIWGPGTLVTVSS

SEQ ID NO: 42 - protein, 13 aa, synthetic construct
QSSQSVYDNNWLS

SEQ ID NO: 43 - protein, 7 aa, synthetic construct
DASKLAS

SEQ ID NO: 44 - protein, 10 aa, synthetic construct
AGGYTGNVYS

SEQ ID NO: 45 - protein, 110 aa, synthetic construct
DPMLTQTPSSVSAAVGGTVTIGCQSSQSVYDNNWLSWYQQKPGQPPNRLIYDASKLASGVPSRFSGSGSGTQFTLTISGVKCDDAATYYCAGGYTGNVYSFGGGTMVEIK

SEQ ID NO: 46 - protein, 14 aa, synthetic construct
TVSGIDLSNNAISW

SEQ ID NO: 47 - protein, 118 aa, synthetic construct
QSVEESGGRLVTPGTPLTLTCTVSGIDLSNNAISWVRQAPGKGLEYIGVIGNTGDTYYASWAKGRFTISKTSSTTVDLKMTSLTTEDTATYFCVRGPPSYSNSVKNIWGPGTLVTISS

SEQ ID NO: 48 - protein, 110 aa, synthetic construct
DPMLTQTPSSVSAAVGGTVTIGCQSSQSVYDKNWLSWYQQKPGQPPSRLIYDASKLESGVPSRFSGSGSGTQFTLTISGVQCDDVATYYCAGGFTGNVYAFGGGTEVVVK

SEQ ID NO: 49 - protein, 13 aa, synthetic construct
TVSGIDLSNNAVS

SEQ ID NO: 50 - protein, 119 aa, synthetic construct
QEQLKESGGRLVAPGTPLRLTCTVSGIDLSNNAVSWVRQAPGKGLEYIGVIGNTGTTYYANWAKGRFTISKTSSTTVDLKMTSLTTDDTATYFCVRGPPSYSNSVKNIWGPGTLVTVSS

SEQ ID NO: 51 - protein, 13 aa, synthetic construct
QASQSVYNNNWLT

SEQ ID NO: 52 - protein, 10 aa, synthetic construct
AAGWTGNVYS

SEQ ID NO: 53 - protein, 110 aa, synthetic construct
DPMLTQTPSPVSAAVGGTVTINCQASQSVYNNNWLTWYQQKPGQPPNRLIYDASKLASGVPSRFSGSGFGTQFTLTISGVQCDDAATYYCAAGWTGNVYSFGGGTEVVVK

SEQ ID NO: 54 - protein, 118 aa, synthetic construct
QQLEESGGRLVTPGTPLTLTCTVSGIDLSNNAISWVRQAPGKGLEYIGVIGNTGTTYYASWAKGRFTISKTSSTTVDLKMTSLTASDTATYFCVRGPPSYSNSVKNIWGPGTLVTISS

SEQ ID NO: 55 - protein, 110 aa, synthetic construct
DVVMTQTPSSVSAAVGGTVTINCQSSQSVYNNNWLSWLQQKPGQPPNRLIYDASKLESGVPSRFSGSGSGTQFTLTISGVQCDDAATYYCAGGFTGNVYTFGGGTEVVVK

SEQ ID NO: 56 - protein, 5 aa, synthetic construct
NYYMS

SEQ ID NO: 57 - protein, 19 aa, synthetic construct
GISSRAGTWYASWAKGRFT

SEQ ID NO: 58 - protein, 4 aa, synthetic construct
GLKI

SEQ ID NO: 59 - protein, 110 aa, synthetic construct
QEQLKESGGRLVTPGTPLTLTCTASGFSLSNYYMSWVRQAPGKGLEWIGGISSRAGTWYASWAKGRFTISGTSTTMDLKITSPTTEDTATYFCARGLKIWGPGTLVTVSS

SEQ ID NO: 60 - protein, 12 aa, synthetic construct
ASQSVYDDNNLA

SEQ ID NO: 61 - protein, 13 aa, synthetic construct
QGEFSCTSADCVA

SEQ ID NO: 62 - protein, 113 aa, synthetic construct
DPVLTQTESPVSAAVGGTVTINCQASQSVYDDNNLAWYQQKPGQPPKRLIYPASTLASGVSSRFSGSGSGSQFTLTISDVQCDDAATYYCQGEFSCTSADCVAFGGGTEVVVK

SEQ ID NO: 63 - protein, 5 aa, synthetic construct
NNAMS

SEQ ID NO: 64 - protein, 119 aa, synthetic construct
QEQLEESGGRLVTPGTPLTLTCTVSGIDLSNNAMSWVRQAPGKGLEYIGVIGNTGTTYYANWAKGRFTISKTSSATVDLKMTSLTTDDTATYFCVRGPPSYSNSVKNIWGPGTLVTVSS

SEQ ID NO: 65 - protein, 110 aa, synthetic construct
DVMLTQTPSSVSAAVGGTVTINCQSSQSVYDKNWLSWLQQKPGQPPSRLIYDASKLESGVPSRFSGSGSGTQFTLTISGVQCDDAATYYCAGGFTGNVYTFGGGTKVVVK

SEQ ID NO: 66 - protein, 5 aa, synthetic construct
SYAMS

SEQ ID NO: 67 - protein, 16 aa, synthetic construct
VIDSGGTTYYATWAKS

SEQ ID NO: 68 - protein, 19 aa, synthetic construct
ESYAGYDGYGHPIPYGMDP

SEQ ID NO: 69 - protein, 124 aa, synthetic construct
QSVEESGGRLVTPGTPLTITCTVSGFSLSSYAMSWVRQAPGKGLEWIGVIDSGGTTYYATWAKSRFTISKTQTKVALKITSPTTEDTATYFCARESYAGYDGYGHPIPYGMDPWGPGTLVTVSS

SEQ ID NO: 70 - protein, 13 aa, synthetic construct
QASQSLYNNKNLA

SEQ ID NO: 71 - protein, 7 aa, synthetic construct
YASALAS

SEQ ID NO: 72 - protein, 13 aa, synthetic construct
QGEFNCNSADCNS

SEQ ID NO: 73 - protein, 113 aa, synthetic construct
DVVMTQTPSSVSAAVGGTVTINCQASQSLYNNKNLAWYQQKPGQPPKLLIYYASALASGVSSRFKGSGSGTQFTLTISGVQCDDAATYYCQGEFNCNSADCNSFGGGTEVVVK

SEQ ID NO: 74 - protein, 13 aa, synthetic construct
TVSGIDLSNNAIS

SEQ ID NO: 75 - protein, 118 aa, synthetic construct
QSLEESGGRLVTPGTPLTLTCTVSGIDLSNNAISWVRQAPGKGLEYIGVIGNTGDTYYASWAKGRFTISKTSSTTVDLKMTSLTTEDTATYFCVRGPPSYSNSVKNIWGPGTLVTISS

SEQ ID NO: 76 - protein, 110 aa, synthetic construct
DPMLTQTPSSVSAAVGGTVTIGCQSSQSVYDKNWLSWYQQKPGQPPSRLIYDASKLESGVPSRFSGSGSGTQFTLTISGVQCDDAATYYCAGGFTGNVYAFGGGTKLEIK

SEQ ID NO: 77 - protein, 119 aa, synthetic construct
QEQLKESGGRLVAPGTPLRLTCTVSGIDLSNNAVSWVRQAPGKGLEYIGVIGNTGTTYYANWAKGRFTISKTSSTTVDLKMTSLTTDDTATYFCVRGPPSYSNSVKNIWGPGTLVTISS

SEQ ID NO: 78 - protein, 110 aa, synthetic construct
DVVMTQTPSSVSAAVGGTVTIGCQSSQSVYDKNWLSWYQQKPGQPPSRLIYDASKLESGVPSRFSGSGSGTQFTLTISGVQCDDAATYYCAGGFTGNVYAFGGGTMVEIK

SEQ ID NO: 79 - protein, 117 aa, synthetic construct
QQLEESGGRLVTPGTPLTLTCTVSGIDLSNNAMSWVRQAPGKGLEYIGVIGNTGTTYYASWAKGRFTISKTSSTTVDLKMTSLTTEDTATYFCVRGPPSYSNSVKNIWGPGTLVTIS

SEQ ID NO: 80 - protein, 110 aa, synthetic construct
DPVLTQTPSSVSAAVGGTVTINCQSSQSVYNNNWLSWLQQKPGQPPNRLIYDASKLESGVPSRFSGSGSGTQFTLTISGVQCDDAATYYCAGGFTGNVYTFGGGTEVVVK

SEQ ID NO: 81 - protein, 5 aa, synthetic construct
NNAIS

SEQ ID NO: 82-99 - skipped (18 entries)

SEQ ID NO: 100 - protein, 225 aa, synthetic construct
YVMMTQTPSSVSAAVGGTVTINCQASQSLYNNNNLAWFQQKPGQPPKRLIYPASTLASGVSSRFKGSGSGTQFTLTISDVQSDDAATYFCLGEFSCSSADCYAFGGGTEVVVKQEQLKESGGRLVTPGTPLTLTCAVSGFSLDIYGMSWVRQAPGKGLEWIGIIYIEGITYYANWAKGRFTISKTSSTTLELKMTSLTTEDTAMYFCVAGVGRIWGPGTLVTVSS

SEQ ID NO: 101 - protein, 229 aa, synthetic construct
DPMLTQTPSSVSAAVGGTVTINCQSSQSVYNNNWLSWLQQKPGQPPNRLIYDASKLESGVPSRFSGSGSGTQFTLTISGVQSDDAATYYCAGGFTGNVYTFGGGTEVVVKQEQLEESGGRLVTPGTPLTLTCKVSGIDLSNNAISWVRQAPGKGLEYIGVIGNTGDTYYASWAKGRFTISKTSSTTVDLKMTSLTTEDTATYFCVRGPPSYSNSVKNIWGPGTLVTISS

SEQ ID NO: 102 - protein, 228 aa, synthetic construct
DPMLTQTPSSVSAAVGGTVTIGCQSSQSVYDNNWLSWYQQKPGQPPNRLIYDASKLASGVPSRFSGSGSGTQFTLTISGVKSDDAATYYCAGGYTGNVYSFGGGTMVEIKQQLEESGGRLVAPGTPLRLTCTVSGIDLSNNAVSWVRQAPGKGLEYIGVIGNTGTTYYANWAKGRFTISKTSSTTVDLKMTSLTTDDTATYFCVRGPPSYSNSVRNIWGPGTLVTVSS

SEQ ID NO: 103 - protein, 228 aa, synthetic construct
DPMLTQTPSSVSAAVGGTVTIGCQSSQSVYDKNWLSWYQQKPGQPPSRLIYDASKLESGVPSRFSGSGSGTQFTLTISGVQSDDVATYYCAGGFTGNVYAFGGGTEVVVKQSVEESGGRLVTPGTPLTLTCTVSGIDLSNNAISWVRQAPGKGLEYIGVIGNTGDTYYASWAKGRFTISKTSSTTVDLKMTSLTTEDTATYFCVRGPPSYSNSVKNIWGPGTLVTISS

SEQ ID NO: 104 - protein, 224 aa, synthetic construct
DPVLTQTESPVSAAVGGTVTINCQASQSVYDDNNLAWYQQKPGQPPKRLIYPASTLASGVSSRFSGSGSGSQFTLTISDVQSDDAATYYCQGEFSCTSADCVAFGGGTEVVVKQEQLKESGGRLVTPGTPLTLTCTASGFSLSNYYMSWVRQAPGKGLEWIGGISSRAGTWYASWAKGRFTISGTSSTTMDLKITSPTTEDTATYFCARGLKIWGPGTLVTVSS

SEQ ID NO: 105 - protein, 223 aa, synthetic construct
DPVLTQTESPVSAAVGGTVTINCQASQSVYDDNNLAWYQQKPGQPPKRLIYPASTLASGVSSRFSGSGSGSQFTLTISDVQSDDAATYYCQGEFSCTSADCVAFGGGTEVVVKQEQLKESGGRLVTPGTPLTLTCTASGFSLSNYYMSWVRQAPGKGLEWIGGISSRAGTWYASWAKGRFTISGTSTTMDLKITSPTTEDTATYFCARGLKIWGPGTLVTVSS

SEQ ID NO: 106 - protein, 238 aa, synthetic construct
DVVMTQTPSSVSAAVGGTVTINCQASQSLYNNKNLAWYQQKPGQPPKLLIYYASALASGVSSRFKGSGSGTQFTLTISGVQSDDAATYYCQGEFNCNSADCNSFGGGTEVVVKQSVEESGGRLVTPGTPLTITCTVSGFSLSSYAMSWVRQAPGKGLEWIGVIDSGGTTYYATWAKSRFTISKTSSTKVALKITSPTTEDTATYFCARESYAGYDGYGHPIPYGMDPWGPGTLVTVSS

SEQ ID NO: 107 - protein, 236 aa, synthetic construct
DVVMTQTPSSVSAAVGGTVTINCQASQSLYNNKNLAWYQQKPGQPPKLLIYYASALASGVSSRFKGSGSGTQFTLTISGVQSDDAATYYCQGEFNCNSADCNSFGGGTEVVVKQSVEESGGRLVTPGTPLTITCTVSGFSLSSYAMSWVRQAPGKGLEWIGVIDSGGTTYYATWAKSRFTISKTTKVALKITSPTTEDTATYFCARESYAGYDGYGHPIPYGMDPWGPGTLVTVSS

SEQ ID NO: 108-199 - skipped (92 entries)

SEQ ID NO: 200 - protein, 10 aa, synthetic construct
GIDLSNNAIS

SEQ ID NO: 201 - protein, 9 aa, synthetic construct
VIGNTGDTY

SEQ ID NO: 202 - protein, 12 aa, synthetic construct
GPPSYSSSVKNI

SEQ ID NO: 203 - protein, 120 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSS

SEQ ID NO: 204 - protein, 450 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 205 - protein, 110 aa, synthetic construct
DIQMTQSPSSVSASVGDRVTITCQSSQSVYNNNWLSWLQQKPGKAPKRLIYDASKLESGVPSRFSGSGSGTDFTLTISSVQPEDAATYYCAGGFTGNVYTFGGGTKVEIK

SEQ ID NO: 206 - protein, 217 aa, synthetic construct
DIQMTQSPSSVSASVGDRVTITCQSSQSVYNNNWLSWLQQKPGKAPKRLIYDASKLESGVPSRFSGSGSGTDFTLTISSVQPEDAATYYCAGGFTGNVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 1416 bp, synthetic construct
  source 1..1416: mol_type = other DNA
atggccaggaagtccgctctgctcgctctggcacttctgcttctgggatttggacctgcttgggctgaggttcagctgctggaatctggcggaggattggttcagcctggcggctctctgagactgtcttgtgctgtgtctggcatcgacctgtccaacaacgccatctcctgggtccgacaggctcctggcaaaggcctggaatatgtgggcgtgatcggcaacaccggcgacacctactacgccgattctgccaagggcagattcaccatctctcgggactcctccaagaccacactgtacctgcagatgaactccctgagagccgaggataccgccgtgtacttttgtgtgcggggacctcctagctactcctccagcgtgaagaacatctggggccagggcacactggtcaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 208 - DNA, 717 bp, synthetic construct
  source 1..717: mol_type = other DNA
atggccaggaagtccgctctgctcgctctggcacttctgcttctgggatttggacctgcttgggctgacatccagatgacccagtctccatcctccgtgtctgcctctgtgggcgacagagtgaccatcacctgtcagtccagccaatccgtgtacaacaacaactggctgtcctggctgcagcagaagcctggcaaggctcccaagagactgatctacgacgcctccaagctggaatccggcgtgccctccagattttccggctctggctctggcaccgactttaccctgaccatctcctccgtgcagcctgaggatgccgccacctattattgtgctggaggcttcaccggcaacgtgtacacatttggcggaggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 209 - skipped

SEQ ID NO: 210 - protein, 10 aa, synthetic construct
GFTFSRYNMN

SEQ ID NO: 211 - protein, 10 aa, synthetic construct
SISTSSNYIY

SEQ ID NO: 212 - protein, 7 aa, synthetic construct
GWGPFDY

SEQ ID NO: 213 - protein, 116 aa, synthetic construct
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGCGTLVTVSS

SEQ ID NO: 214 - protein, 11 aa, synthetic construct
RARQSIGTAIH

SEQ ID NO: 215 - protein, 7 aa, synthetic construct
YASESIS

SEQ ID NO: 216 - protein, 9 aa, synthetic construct
QQSGSWPYT

SEQ ID NO: 217 - protein, 107 aa, synthetic construct
DIQMTQSPSSLSASVGDRVTITCRARQSIGTAIHWYQQKPGCAPKLLIKYASESISGVPSRFSGSGSGTDFTLTISSLQPEDFATYYCQQSGSWPYTFGQGTKLEIK

SEQ ID NO: 218 - protein, 473 aa, synthetic construct
DIQMTQSPSSLSASVGDRVTITCRARQSIGTAIHWYQQKPGCAPKLLIKYASESISGVPSRFSGSGSGTDFTLTISSLQPEDFATYYCQQSGSWPYTFGQGTKLEIKGGGSGGSGGCPPCGGSGGEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - DNA, 1485 bp, synthetic construct
  source 1..1485: mol_type = other DNA
atggccaggaagtccgctctgctcgctctggcacttctgcttctgggatttggacctgcttgggctgacatccagatgacccagtctccatcctctctgtccgcctctgtgggcgacagagtgaccattacctgccgggccagacagtctatcggcaccgctatccactggtatcagcagaagcctggctgtgcccctaagctgctgattaagtacgcctccgagtccatctccggcgtgccctccagattttctggctctggatctggcaccgactttaccctgacaatctccagcctgcagcctgaggacttcgccacctactactgtcagcagtccggctcttggccttacacctttggtcagggcaccaagctggaaatcaagggcggaggttctggtggatctggcggatgtcctccttgtggtggaagcggcggagaggtgcagctggttgaatctggcggaggactggttaagcctggcggctctctgagactgtcttgtgctgcttctggcttcaccttcagccggtacaacatgaactgggtccgacaggctcctggcaaaggcctggaatgggtgtcctccatctccacctccagcaactacatctactacgccgactccgtgaagggcagattcaccttctccagagacaacgccaagaactccctggacctgcagatgtctggcctgagagctgaggacaccgctatctactactgcaccagaggctggggacccttcgattattggggctgtggaaccctggtcaccgtgtcatctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 220 - protein, 45 aa, synthetic construct
TRRMMRTKMRMRRMRRTRRKMRRKMSPARPRTSCREACLQGWTEA

SEQ ID NO: 221 - protein, 46 aa, synthetic construct
NCRRMMRTKMRMRRMRRTRRKMRRKMSPARPRTSCREACLQGWTEA

SEQ ID NO: 222 - protein, 47 aa, synthetic construct
QRTRRMMRTKMRMRRMRRTRRKMRRKMSPARPRTSCREACLQGWTEA

SEQ ID NO: 223 - protein, 50 aa, synthetic construct
RRRQRTRRMMRTKMRMRRMRRTRRKMRRKMSPARPRTSCREACLQGWTEA

SEQ ID NO: 224 - protein, 47 aa, synthetic construct
GRTRRMMRTKMRMRRMRRTRRKMRRKMSPARPRTSCREACLQGWTEA

SEQ ID NO: 225 - protein, 49 aa, synthetic construct
RRQRTRRMMRTKMRMRRMRRTRRKMRRKMSPARPRTSCREACLQGWTEA

SEQ ID NO: 226 - protein, 44 aa, synthetic construct
RRMMRTKMRMRRMRRTRRKMRRKMSPARPRTSCREACLQGWTEA

SEQ ID NO: 227-228 - skipped (2 entries)

SEQ ID NO: 229 - protein, 46 aa, synthetic construct
MCRRMMRTKMRMRRMRRTRRKMRRKMSPARPRTSCREACLQGWTEA

SEQ ID NO: 230 - protein, 50 aa, synthetic construct
DQRQRTRRMMRTKMRMRRMRRTRRKMRRKMSPARPRTSCREACLQGWTEA

SEQ ID NO: 231 - protein, 51 aa, synthetic construct
RRRRQRTRRMMRTKMRMRRMRRTRRKMRRKMSPARPRTSCREACLQGWTEA

SEQ ID NO: 232 - protein, 51 aa, synthetic construct
QRRRQRTRRMMRTKMRMRRMRRTRRKMRRKMSPARPRTSCREACLQGWTEA

SEQ ID NO: 233 - skipped

SEQ ID NO: 234 - protein, 50 aa, synthetic construct
RRRERTRRMMRTKMRMRRMRRTRRKMRRKMSPARPRTSCREACLQGWTEA

SEQ ID NO: 235 - protein, 50 aa, synthetic construct
QRRQRTRRMMRTKMRMRRMRRTRRKMRRKMSPARPRTSCREACLQGWTEA

SEQ ID NO: 236 - protein, 49 aa, synthetic construct
RRQWTRRMMRTKMRMRRMRRTRRKMRRKMSPARPRTSCREACLQGWTEA

SEQ ID NO: 237 - protein, 43 aa, synthetic construct
RMMRTKMRMRRMRRTRRKMRRKMSPARPRTSCREACLQGWTEA

SEQ ID NO: 238 - skipped

SEQ ID NO: 239 - protein, 50 aa, synthetic construct
GRRQRTRRMMRTKMRMRRMRRTRRKMRRKMSPARPRTSCREACLQGWTEA

SEQ ID NO: 240 - protein, 49 aa, synthetic construct
AFKRTRRMMRTKMRMRRMRRTRRKMRRKMSPARPRTSCREACLQGWTEA

SEQ ID NO: 241 - protein, 53 aa, synthetic construct
NAKRRRRQRTRRMMRTKMRMRRMRRTRRKMRRKMSPARPRTSCREACLQGWTE

SEQ ID NO: 242 - protein, 53 aa, synthetic construct
CVRRRRQRTRRMMRTKMRMRRMRRTRRKMRRKMSPARPRTSCREACLQGWTEA

SEQ ID NO: 243-245 - skipped (3 entries)

SEQ ID NO: 246 - protein, 53 aa, synthetic construct
CFAKRRRRQRTRRMMRTKMRMRRMRRTRRKMRRKMSPARPRTSCREACLQGWT

SEQ ID NO: 247 - skipped

SEQ ID NO: 248 - protein, 49 aa, synthetic construct
PPLCLRRMMRTKMRMRRMRRTRRKMRRKMSPARPRTSCREACLQGWTEA

SEQ ID NO: 249 - protein, 48 aa, synthetic construct
DHPCRRMMRTKMRMRRMRRTRRKMRRKMSPARPRTSCREACLQGWTEA

SEQ ID NO: 250 - protein, 47 aa, synthetic construct
GNCRRMMRTKMRMRRMRRTRRKMRRKMSPARPRTSCREACLQGWTEA

SEQ ID NO: 251 - protein, 45 aa, synthetic construct
CRRMMRTKMRMRRMRRTRRKMRRKMSPARPRTSCREACLQGWTEA

SEQ ID NO: 252 - skipped

SEQ ID NO: 253 - protein, 46 aa, synthetic construct
TCRRMMRTKMRMRRMRRTRRKMRRKMSPARPRTSCREACLQGWTEA

SEQ ID NO: 254 - protein, 46 aa, synthetic construct
ICRRMMRTKMRMRRMRRTRRKMRRKMSPARPRTSCREACLQGWTEA

SEQ ID NO: 255 - skipped

SEQ ID NO: 256 - protein, 8 aa, synthetic construct
QSVYNNNW

SEQ ID NO: 257-258 - skipped (2 entries)

SEQ ID NO: 259 - protein, 16 aa, synthetic construct
VIGNTGDTYYADSAKG

SEQ ID NO: 260 - protein, 7 aa, synthetic construct
GIDLSNN

SEQ ID NO: 261 - protein, 5 aa, synthetic construct
GNTGD

SEQ ID NO: 262 - protein, 8 aa, synthetic construct
GIDLSNNA

SEQ ID NO: 263 - protein, 7 aa, synthetic construct
IGNTGDT

SEQ ID NO: 264 - protein, 14 aa, synthetic construct
VRGPPSYSSSVKNI

SEQ ID NO: 265 - protein, 5 aa, synthetic construct
RYNMN

SEQ ID NO: 266 - protein, 17 aa, synthetic construct
SISTSSNYIYYADSVKG

SEQ ID NO: 267 - protein, 7 aa, synthetic construct
GFTFSRY

SEQ ID NO: 268 - protein, 6 aa, synthetic construct
STSSNY

SEQ ID NO: 269 - protein, 8 aa, synthetic construct
GFTFSRYN

SEQ ID NO: 270 - protein, 8 aa, synthetic construct
ISTSSNYI

SEQ ID NO: 271 - protein, 9 aa, synthetic construct
TRGWGPFDY

SEQ ID NO: 272 - protein, 6 aa, synthetic construct
QSIGTA

SEQ ID NO: 273 - protein, 10 aa, synthetic construct
GFSLSSYAMS

SEQ ID NO: 274 - protein, 9 aa, synthetic construct
VIDSGGTTY

SEQ ID NO: 275 - protein, 19 aa, synthetic construct
ESYAGYDYYGHPIPYGMEP

SEQ ID NO: 276 - protein, 7 aa, synthetic construct
GFSLSSY

SEQ ID NO: 277 - protein, 5 aa, synthetic construct
DSGGT

SEQ ID NO: 278 - protein, 16 aa, synthetic construct
VIDSGGTTYYAQKFQG

SEQ ID NO: 279 - protein, 8 aa, synthetic construct
GFSLSSYA

SEQ ID NO: 280 - protein, 7 aa, synthetic construct
IDSGGTT

SEQ ID NO: 281 - protein, 21 aa, synthetic construct
ARESYAGYDYYGHPIPYGMEP

SEQ ID NO: 282 - protein, 127 aa, synthetic construct
QVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGQGTLVTVSS

SEQ ID NO: 283 - protein, 8 aa, synthetic construct
QSLYNNKN

SEQ ID NO: 284 - protein, 113 aa, synthetic construct
DIQMTQSPSTLSASVGDRVTITCQASQSLYNNKNLAWYQQKPGKAPKLLIYYASALASGVPSRFSGSGSGTEFTLTISSLQPDDFATYYCQGEFNCNSADCNSFGGGTKVEIK

SEQ ID NO: 285 - protein, 116 aa, synthetic construct
EVQLVESGGGLVKPGGSLRLSCAASGFTFSRYNMNWVRQAPGCGLEWVSSISTSSNYIYYADSVKGRFTFSRDNAKNSLDLQMSGLRAEDTAIYYCTRGWGPFDYWGQGTLVTVSS

SEQ ID NO: 286 - protein, 113 aa, synthetic construct
DIQMTQSPSTLSASVGDRVTITCQASQSLYNNKNLAWYQQKPGCAPKLLIYYASALASGVPSRFSGSGSGTEFTLTISSLQPDDFATYYCQGEFNCNSADCNSFGGGTKVEIK

SEQ ID NO: 287 - protein, 127 aa, synthetic construct
QVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGCGTLVTVSS

SEQ ID NO: 288 - protein, 128 aa, synthetic construct
QVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGCGTLVTVSSH

SEQ ID NO: 289 - protein, 707 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSDIQMTQSPSSLSASVGDRVTITCRARQSIGTAIHWYQQKPGCAPKLLIKYASESISGVPSRFSGSGSGTDFTLTISSLQPEDFATYYCQQSGSWPYTFGQGTKLEIKGGGSGGSGGCPPCGGSGGEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - protein, 457 aa, synthetic construct
QVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 291 - protein, 220 aa, synthetic construct
DIQMTQSPSTLSASVGDRVTITCQASQSLYNNKNLAWYQQKPGKAPKLLIYYASALASGVPSRFSGSGSGTEFTLTISSLQPDDFATYYCQGEFNCNSADCNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - protein, 713 aa, synthetic construct
QVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDIQMTQSPSSLSASVGDRVTITCRARQSIGTAIHWYQQKPGCAPKLLIKYASESISGVPSRFSGSGSGTDFTLTISSLQPEDFATYYCQQSGSWPYTFGQGTKLEIKGGGSGGSGGCPPCGGSGGEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 718 aa, synthetic construct
DIQMTQSPSTLSASVGDRVTITCQASQSLYNNKNLAWYQQKPGCAPKLLIYYASALASGVPSRFSGSGSGTEFTLTISSLQPDDFATYYCQGEFNCNSADCNSFGGGTKVEIKGGGSGGSGGCPPCGGSGGQVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGCGTLVTVSSGGGGSGGGGSEVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 294 - protein, 723 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDIQMTQSPSTLSASVGDRVTITCQASQSLYNNKNLAWYQQKPGCAPKLLIYYASALASGVPSRFSGSGSGTEFTLTISSLQPDDFATYYCQGEFNCNSADCNSFGGGTKVEIKGGGSGGSGGCPPCGGSGGQVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 295 - protein, 719 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSDIQMTQSPSTLSASVGDRVTITCQASQSLYNNKNLAWYQQKPGCAPKLLIYYASALASGVPSRFSGSGSGTEFTLTISSLQPDDFATYYCQGEFNCNSADCNSFGGGTKVEIKGGGSGGSGGCPPCGGSGGQVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGCGTLVTVSSH

SEQ ID NO: 296 - protein, 683 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 297 - protein, 713 aa, synthetic construct
QVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVKPGGSLRLSCAASGFTFSRYNMNWVRQAPGCGLEWVSSISTSSNYIYYADSVKGRFTFSRDNAKNSLDLQMSGLRAEDTAIYYCTRGWGPFDYWGQGTLVTVSSGGGSGGSGGCPPCGGSGGDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 697 aa, synthetic construct
QVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGASVKVSCKASGFSLSSYAMSWVRQAPGQGLEWMGVIDSGGTTYYAQKFQGRVTMTRDTSTSTVYMELSSLRSEDTAVYYCARESYAGYDYYGHPIPYGMEPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 299 - DNA, 17 bp, Homo sapiens
  source 1..17: mol_type = genomic DNA
gcagcagagaaacaaat

SEQ ID NO: 300 - DNA, 15 bp, Homo sapiens
  source 1..15: mol_type = genomic DNA
aaggacgagctgtag

SEQ ID NO: 301 - protein, 5 aa, Homo sapiens
AAEKQ

SEQ ID NO: 302 - protein, 5 aa, Homo sapiens
QQRNK

SEQ ID NO: 303 - protein, 5 aa, Homo sapiens
SRETN

SEQ ID NO: 304 - protein, 4 aa, Homo sapiens
KDEL

SEQ ID NO: 305 - protein, 4 aa, Homo sapiens
RTSC

SEQ ID NO: 306 - protein, 4 aa, Homo sapiens
GRAV

(19) United States
(12) Patent Application Publication
(10) Pub. No.: US 2026/0125457 A1
(43) Pub. Date: May 7, 2026

(54) MOLECULES THAT BIND TO MUTANT CALRETICULIN AND USES THEREOF

(71) Applicant: Janssen Biotech, Inc., Horsham, PA (US)

(72) Inventors: Wan Cheung CHEUNG, Lexington, MA (US); Elisabeth Geyer PRINSLOW, Harleysville, PA (US); Lutgart JANSSEN, Herentals (BE); Anna KUCHNIO, Beerse (BE); Jinquan LUO, Malvern, PA (US); Ulrike PHILIPPAR, Beerse (BE); Steven C. POMERANTZ, Huntingdon Valley, PA (US); Elsie SAMAKAI, Warrington, PA (US); Stefan CONSTANTINESCU, Brussels (BE); Oleh ZAGRIJTSCHUK, Vienna (AT); Eva HUG, Munchen (DE)

(21) Appl. No.: 19/310,734

(22) Filed: Aug. 26, 2025

Related U.S. Application Data
(63) Continuation of application No. PCT/IB2024/057337, filed on Jul. 30, 2024.
(60) Provisional application No. 63/516,520, filed on Jul. 30, 2023.

(51) Int. Cl. C07K 16/18; A61K 39/00; A61P 35/02; C07K 16/28

(52) U.S. Cl. CPC C07K 16/18; A61P 35/02; C07K 16/2809; A61K 2039/505; C07K 2317/31; C07K 2317/565; C07K 2317/622; C07K 2317/73; C07K 2317/92

(57) ABSTRACT

The present disclosure relates to antibodies, and antigen-binding fragments thereof, that bind to mutant calreticulin. The present disclosure also relates to bispecific antibodies, and bispecific antigen-binding fragments thereof, that bind to mutant calreticulin and cluster of differentiation 3. Also provided are cells expressing the antibodies, polynucleotides and vectors expressing all of some of the antibodies, pharmaceutical compositions comprising the antibodies, and methods of inhibiting the growth or proliferation, or treating, myeloproliferative neoplasm.

### CROSS-REFERENCE TO RELATED APPLICATIONS

This application claims the benefit of U.S. Provisional Application 63/516,520 filed on Jul. 30, 2023, which is hereby incorporated by reference in its entirety.

### SEQUENCE LISTING

The instant application contains a Sequence Listing which has been submitted electronically in XML format and is hereby incorporated by reference in its entirety. Said XML copy, created on Jul. 26, 2024, is named “258199050102 (JBI6823WOPCT1) Sequence Listing.xml” and is 194,109 bytes in size.

### FIELD

The present disclosure relates to molecules that bind to mutant calreticulin, methods of treating or reducing the severity of myeloproliferative neoplasms in a subject.

### BACKGROUND

Myeloproliferative Neoplasms (MPNs) are clonal disorders of hematopoiesis arising in the hematopoietic stem cell (HSC) compartment that are characterized by excessive production of mature blood cells of the myeloid lineage. Transformation to secondary acute myeloid leukemia (sAML) represents a significant cause of death among MPN patients. Current treatment options for MPN patients are not curative and are limited to symptomatic treatment. Therefore, identification of novel therapeutic approaches with a disease-modifying effect for the treatment of MPNs and intercepting their progression to sAML is an unmet medical need.

Mutations in JAK2, thrombopoietin receptor (TPOR, also known as myeloproliferative leukemia protein or MPL), and calreticulin (CALR) are phenotypic drivers in the pathogenesis of MPN. CALR mutations (CALRmut) are the second most frequent in MPN. CALRmut are insertions or deletions resulting in a frameshift in the last exon of the gene, resulting in loss of the KDEL ER-retention motif and generation of a 36 amino acid positively charged C-terminal neoantigen. Due to loss of the KDEL motif, CALRmut is not confined to the ER. Specifically, a tetrameric complex formed between 2 CALRmut and 2 MPL proteins is shuttled through the Golgi apparatus and is presented on the cell membrane, leading to constitutive activation of downstream kinase JAK2. Cell surface presentation of CALRmut/MPL complex is required for constitutive activation of JAK2 and oncogenic transformation. In contrast to wild type cells, where MPL is only present on the cell surface with mature glycosylation, MPL in complex with CALRmut remains in an immature glycosylation status on the cell surface.

As CALRmut is expressed on the cell surface in MPNs, it can be considered as a target for immunotherapeutic treatment. There is a need for disease modifying treatments of MPN, which target CALRmut. Disclosed herein are antibodies, and antigen-binding fragments thereof, that bind to CALRmut, as well as bispecific antibodies, and antigen-binding fragments thereof, that bind to CALRmut and CD3.

### SUMMARY

Provided herein are antibodies, or antigen-binding fragments thereof, comprising: a heavy chain comprising a heavy chain complementarity determining region (CDR) 1 (CDR1) comprising the amino acid sequence of SEQ ID NO: 200, a heavy chain CDR2 comprising the amino acid sequence of SEQ ID NO: 201, and a heavy chain CDR3 comprising the amino acid sequence of SEQ ID NO: 202; and a light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34;
a heavy chain comprising a heavy chain complementarity determining region (CDR) 1 (CDR1) comprising the amino acid sequence of SEQ ID NO: 260, a heavy chain CDR2 comprising the amino acid sequence of SEQ ID NO: 261, and a heavy chain CDR3 comprising the amino acid sequence of SEQ ID NO: 202; and a light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34;
a heavy chain comprising a heavy chain complementarity determining region (CDR) 1 (CDR1) comprising the amino acid sequence of SEQ ID NO: 81, a heavy chain CDR2 comprising the amino acid sequence of SEQ ID NO: 259, and a heavy chain CDR3 comprising the amino acid sequence of SEQ ID NO: 202; and a light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; or
a heavy chain comprising a heavy chain complementarity determining region (CDR) 1 (CDR1) comprising the amino acid sequence of SEQ ID NO: 262, a heavy chain CDR2 comprising the amino acid sequence of SEQ ID NO: 263, and a heavy chain CDR3 comprising the amino acid sequence of SEQ ID NO: 264; and a light chain comprising a light chain CDR1 comprising the amino acid sequence of SEQ ID NO: 256, a light chain CDR2 comprising the amino acid sequence of DAS, and a light chain CDR3 comprising the amino acid sequence of SEQ ID NO: 34.

In some embodiments, the antibody, or antigen-binding fragment thereof, comprises a heavy chain variable region (VH) comprising an amino acid sequence that is at least 90%, 91%, 92%, 93%, 94%, 95%, 96%, 97%, 98%, or 99% identical the amino acid sequence of SEQ ID NO: 203. In some embodiments, the antibody, or antigen-binding fragment thereof, comprises a heavy chain variable region (VH) comprising the amino acid sequence of SEQ ID NO: 203. In some embodiments, the antibody, or antigen-binding fragment thereof, comprises a light chain variable region (VL) comprising an amino acid sequence that is at least 90%, 91%, 92%, 93%, 94%, 95%, 96%, 97%, 98%, or 99% identical the amino acid sequence of SEQ ID NO: 205. In some embodiments, the antibody, or antigen-binding fragment thereof, comprises a light chain variable region (VL) comprising the amino acid sequence of SEQ ID NO: 205. In some embodiments, the antibody, or antigen-binding fragment thereof, comprises a heavy chain variable region (VH) comprising the amino acid sequence of SEQ ID NO: 203, and a light chain variable region (VL) comprising the amino acid sequence of SEQ ID NO: 205.

In some embodiments, the antibody, or antigen-binding fragment thereof, is an IgG. In some embodiments, the antibody, or antigen-binding fragment thereof, comprises an IgG1 isotype Fc region.

In some embodiments, the antibody, or antigen-binding fragment thereof, further comprises L234A, L235A, and D265S substitutions in the Fc region. In some embodiments, the antibody, or antigen-binding fragment thereof, further comprises knob-into-hole (KiH) substitutions. In some embodiments, the antibody, or antigen-binding fragment thereof, further comprises H435R and Y436F substitutions in the Fc region.

In some embodiments, the heavy chain comprises an amino acid sequence that is at least 90%, 91%, 92%, 93%, 94%, 95%, 96%, 97%, 98%, or 99% identical the amino acid sequence of SEQ ID NO: 204. In some embodiments, the heavy chain comprises the amino acid sequence of SEQ ID NO: 204. In some embodiments, the light chain comprises an amino acid sequence that is at least 90%, 91%, 92%, 93%, 94%, 95%, 96%, 97%, 98%, or 99% identical the amino acid sequence of SEQ ID NO: 206. In some embodiments, the light chain comprises the amino acid sequence of SEQ ID NO: 206. In some embodiments, the heavy chain comprising the amino acid sequence of SEQ ID NO: 204 and the light chain comprising the amino acid sequence of SEQ ID NO: 206.

In some embodiments, the antibody, or antigen-binding fragment thereof, immunospecifically binds to mutant calreticulin (CALRmut). In some embodiments, the CALRmut comprises an amino acid sequence of SEQ ID NO: 1 or SEQ ID NO: 2.

In some embodiments, a cell expressing any antibody, or antigen-binding fragment thereof, disclosed herein is provided. In some embodiments, the cell is a hybridoma. In some embodiments, the antibody, or antigen-binding fragment thereof, is recombinantly produced in the cell.

In some embodiments, a synthetic polynucleotide encoding the heavy chain and light chain of any antibody, or antigen-binding fragment thereof, disclosed herein is provided. In some embodiments, a vector comprising any synthetic polynucleotide is also provided.

In some embodiments, a pharmaceutical composition comprising any antibody, or antigen-binding fragment thereof, disclosed herein and a pharmaceutically acceptable carrier is provided.

In some embodiments, a method for generating any antibody, or antigen-binding fragment thereof, disclosed herein is provided, the method comprising culturing any cell disclosed herein.

In some embodiments, a method of redirecting a T cell to a cell expressing a mutant calreticulin is provided, the method comprising administering to a subject in need thereof a therapeutically effective amount of any antibody, or antigen-binding fragment thereof, disclosed herein to redirect a T cell to the cell expressing a mutant calreticulin.

In some embodiments, a method of inhibiting the growth or proliferation of a myeloproliferative neoplasm (MPN) is provided, the method comprising administering to a subject in need thereof a therapeutically effective amount of any antibody, or antigen-binding fragment thereof, disclosed herein to inhibit the growth or proliferation of the MPN.

In some embodiments, a method of treating a MPN is provided, the method comprising administering to a subject in need thereof any antibody, or antigen-binding fragment thereof, disclosed here, to the subject for a time sufficient to treat the cancer.

In some embodiments, the MPN is characterized by the presence of a mutant calreticulin. In some embodiments, the MPN is selected from the group consisting of chronic myelogenous leukemia, polycythemia vera, primary myelofibrosis, secondary myelofibrosis, secondary acute myeloid leukemia, essential thrombocythemia, chronic neutrophilic leukemia, and chronic eosinophilic leukemia.

In some embodiments, soluble mutant calreticulin is present in the blood of the subject.

In some embodiments, a kit is provided comprising any antibody, or antigen-binding fragment thereof, disclosed herein and packaging for the same. In some embodiments, a kit is provided comprising any polynucleotide disclosed herein and packaging for the same. In some embodiments, a kit is provided comprising any antibody, or antigen-binding fragment thereof, disclosed herein, any polynucleotide disclosed herein, and packaging for the same.

Also provided herein are bispecific antibodies, or bispecific antigen-binding fragments thereof, comprising a heavy chain, a light chain, and a stapled single chain fragment variable (spFv) chain, wherein:
the heavy chain comprises a heavy chain complementarity determining region (CDR) 1 (CDR1) comprising the amino acid sequence of SEQ ID NO: 200, a heavy chain CDR2 comprising the amino acid sequence of SEQ ID NO: 201, and a heavy chain CDR3 comprising the amino acid sequence of SEQ ID NO: 202; the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; and the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 215, a heavy chain CDR1 comprising the amino acid sequence of SEQ ID NO: 210, a heavy chain CDR2 comprising the amino acid sequence of SEQ ID NO: 211, and a heavy chain CDR3 comprising the amino acid sequence of SEQ ID NO: 212;
the heavy chain comprising a heavy chain complementarity determining region (CDR) 1 (CDR1) comprising the amino acid sequence of SEQ ID NO: 260, a heavy chain CDR2 comprising the amino acid sequence of SEQ ID NO: 261, and a heavy chain CDR3 comprising the amino acid sequence of SEQ ID NO: 202; the light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; and the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 216, a heavy chain CDR1 comprising the amino acid sequence of SEQ ID NO: 267, a heavy chain CDR2 comprising the amino acid sequence of SEQ ID NO: 268, and a heavy chain CDR3 comprising the amino acid sequence of SEQ ID NO: 212;
the heavy chain comprising a heavy chain complementarity determining region (CDR) 1 (CDR1) comprising the amino acid sequence of SEQ ID NO: 81, a heavy chain CDR2 comprising the amino acid sequence of SEQ ID NO: 259, and a heavy chain CDR3 comprising the amino acid sequence of SEQ ID NO: 202; the light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; and the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 216, a heavy chain CDR1 comprising the amino acid sequence of SEQ ID NO: 265, a heavy chain CDR2 comprising the amino acid sequence of SEQ ID NO: 266, and a heavy chain CDR3 comprising the amino acid sequence of SEQ ID NO: 212; or
the heavy chain comprising a heavy chain complementarity determining region (CDR) 1 (CDR1) comprising the amino acid sequence of SEQ ID NO: 262, a heavy chain CDR2 comprising the amino acid sequence of SEQ ID NO: 263, and a heavy chain CDR3 comprising the amino acid sequence of SEQ ID NO: 264; the light chain comprising a light chain CDR1 comprising the amino acid sequence of SEQ ID NO: 256, a light chain CDR2 comprising the amino acid sequence of DAS, and a light chain CDR3 comprising the amino acid sequence of SEQ ID NO: 34; and the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 216, a heavy chain CDR1 comprising the amino acid sequence of SEQ ID NO: 269, a heavy chain CDR2 comprising the amino acid sequence of SEQ ID NO: 270, and a heavy chain CDR3 comprising the amino acid sequence of SEQ ID NO: 271;
wherein the heavy chain associates with the light chain to form an antigen binding site that immunospecifically binds to a mutant calreticulin (CALRmut); and
wherein the spFv chain immunospecifically binds to CD3.

In some embodiments, the bispecific antibody, or bispecific antigen-binding fragment thereof, comprises a heavy chain comprising a VH comprising an amino acid sequence that is at least 90%, 91%, 92%, 93%, 94%, 95%, 96%, 97%, 98%, or 99% identical the amino acid sequence of SEQ ID NO: 203. In some embodiments, the bispecific antibody, or bispecific antigen-binding fragment thereof, comprises a heavy chain comprising a VH comprising the amino acid sequence of SEQ ID NO: 203. In some embodiments, the bispecific antibody, or bispecific antigen-binding fragment thereof, comprises a light chain comprising a VL comprising an amino acid sequence that is at least 90%, 91%, 92%, 93%, 94%, 95%, 96%, 97%, 98%, or 99% identical the amino acid sequence of SEQ ID NO: 205. In some embodiments, the bispecific antibody, or bispecific antigen-binding fragment thereof, comprises a light chain comprising a VL comprising the amino acid sequence of SEQ ID NO: 205. In some embodiments, the antibody, or antigen-binding fragment thereof, of claim 31, comprises a heavy chain variable region (VH) comprising the amino acid sequence of SEQ ID NO: 203, and a light chain variable region (VL) comprising the amino acid sequence of SEQ ID NO: 205.

In some embodiments, the bispecific antibody, or bispecific antigen-binding fragment thereof, is an IgG. In some embodiments, the bispecific antibody, or bispecific antigen-binding fragment thereof, comprises an IgG1 isotype Fc region.

In some embodiments, the antibody, or antigen-binding fragment thereof, further comprises L234A, L235A, and D265S substitutions in the Fc region. In some embodiments, the antibody, or antigen-binding fragment thereof, further comprises knob-into-hole (KiH) substitutions. In some embodiments, the antibody, or antigen-binding fragment thereof, further comprises H435R and Y436F substitutions in the Fc region.

In some embodiments, the heavy chain comprises an amino acid sequence that is at least 90%, 91%, 92%, 93%, 94%, 95%, 96%, 97%, 98%, or 99% identical the amino acid sequence of SEQ ID NO: 204. In some embodiments, the heavy chain comprises the amino acid sequence of SEQ ID NO: 204. In some embodiments, light chain comprises an amino acid sequence that is at least 90%, 91%, 92%, 93%, 94%, 95%, 96%, 97%, 98%, or 99% identical the amino acid sequence of SEQ ID NO: 206. In some embodiments, light chain comprises the amino acid sequence of SEQ ID NO: 206. In some embodiments, the spFv chain comprises an amino acid sequence that is at least 90%, 91%, 92%, 93%, 94%, 95%, 96%, 97%, 98%, or 99% identical the amino acid sequence of SEQ ID NO: 218. In some embodiments, the spFv chain comprises the amino acid sequence of SEQ ID NO: 218. In some embodiments, the heavy chain comprises the amino acid sequence of SEQ ID NO: 204, the light chain comprises the amino acid sequence of SEQ ID NO: 206, and the spFv chain comprises the amino acid sequence of SEQ ID NO: 218, wherein the heavy chain associates with the light chain to form an antigen binding site that immunospecifically binds to a mutant calreticulin (CALRmut); and wherein the spFv chain immunospecifically binds to CD3. In some embodiments, the CALRmut comprises an amino acid sequence of SEQ ID NO: 1 or SEQ ID NO: 2.

In some embodiments, a cell expressing any bispecific antibody, or bispecific antigen-binding fragment thereof, disclosed herein is provided. In some embodiments, the cell is a hybridoma. In some embodiments, the bispecific antibody, or bispecific antigen-binding fragment thereof, is recombinantly produced in the cell.

In some embodiments, at least one synthetic polynucleotide encoding the heavy chain, the light chain, and/or the spFv chain of any bispecific antibody, or bispecific antigen-binding fragment thereof, disclosed herein is provided. In some embodiments, a vector comprising any synthetic polynucleotide is also provided.

In some embodiments, a pharmaceutical composition comprising any bispecific antibody, or bispecific antigen-binding fragment thereof, disclosed herein and a pharmaceutically acceptable carrier is provided.

In some embodiments, a method for generating any bispecific antibody, or bispecific antigen-binding fragment thereof, disclosed herein is provided, the method comprising culturing any cell disclosed herein.

In some embodiments, a method of redirecting a T cell to a cell expressing a mutant calreticulin is provided, the method comprising administering to a subject in need thereof a therapeutically effective amount of any bispecific antibody, or bispecific antigen-binding fragment thereof, disclosed herein to redirect a T cell to the cell expressing a mutant calreticulin.

In some embodiments, a method of inhibiting the growth or proliferation of a myeloproliferative neoplasm (MPN) is provided, the method comprising administering to a subject in need thereof a therapeutically effective amount of any bispecific antibody, or bispecific antigen-binding fragment thereof, disclosed herein to inhibit the growth or proliferation of the MPN.

In some embodiments, a method of treating a MPN is provided, the method comprising administering to a subject in need thereof any bispecific antibody, or bispecific antigen-binding fragment thereof, disclosed here, to the subject for a time sufficient to treat the cancer.

In some embodiments, the MPN is characterized by the presence of a mutant calreticulin. In some embodiments, the MPN is selected from the group consisting of chronic myelogenous leukemia, polycythemia vera, primary myelofibrosis, secondary myelofibrosis, secondary acute myeloid leukemia, essential thrombocythemia, chronic neutrophilic leukemia, and chronic eosinophilic leukemia.

In some embodiments, soluble mutant calreticulin is present in the blood of the subject.

In some embodiments, a kit is provided comprising any bispecific antibody, or bispecific antigen-binding fragment thereof, disclosed herein and packaging for the same. In some embodiments, a kit is provided comprising any polynucleotide disclosed herein and packaging for the same. In some embodiments, a kit is provided comprising any bispecific antibody, or bispecific antigen-binding fragment thereof, disclosed herein, any polynucleotide disclosed herein, and packaging for the same.

Also provided herein are several additional bispecific antibodies and bispecific antibody fragments that bind mutant calreticulin and CD3.

In some embodiments, a bispecific antibody, or a bispecific antigen-binding fragment thereof, is provided, comprising a Chain A and a Chain B,
wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein Chain B comprises a heavy chain and a light chain, wherein the heavy chain associates with the light chain to form a CALRmut antigen binding site,
wherein the CALRmut antigen binding site of Chain A and the CALRmut antigen binding site of Chain B immunospecifically binds to CALRmut,
wherein the spFv region immunospecifically binds to CD3,
wherein the Chain A CALRmut heavy binding fragment comprises a heavy complementarity determining region (CDR) 1 (CDR1) comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202;
wherein the Chain A spFv region comprises a light CDR1 comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain A CALRmut light binding fragment comprises a light CDR1 comprising the amino acid sequence of SEQ ID NO: 33, a CDR2 comprising the amino acid sequence of SEQ ID NO: 23, and a CDR3 comprising the amino acid sequence of SEQ ID NO: 34;
wherein the Chain B heavy chain comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202; and wherein the Chain B light chain comprises a light CDR1 comprising the amino acid sequence of SEQ ID NO: 33, a CDR2 comprising the amino acid sequence of SEQ ID NO: 23, and a CDR3 comprising the amino acid sequence of SEQ ID NO: 34.

In some embodiments, the Chain A CALRmut heavy binding fragment comprises a VH comprising an amino acid sequence of SEQ ID NO: 203, the Chain A spFv region comprises a VL comprising an amino acid sequence of SEQ ID NO: 217, and a VH comprising an amino acid sequence of SEQ ID NO: 213, the Chain A CALRmut light binding fragment comprises a VL comprising an amino acid sequence of SEQ ID NO: 205, the Chain B heavy chain comprises a VH comprising an amino acid sequence of SEQ ID NO: 203, and the Chain B light chain comprises a VL comprising an amino acid sequence of SEQ ID NO: 205. In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 289, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 206, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 204, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, a bispecific antibody, or a bispecific antigen-binding fragment thereof, is provided comprising a Chain A and a Chain B,
wherein Chain A comprises a stapled single chain fragment variable (spFv) region that immunospecifically binds to CD3,
wherein Chain B comprises a heavy chain and a light chain, wherein the heavy chain associates with the light chain to form a mutant calreticulin (CALRmut) antigen binding site that immunospecifically binds to CALRmut,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B heavy chain comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275; and
wherein the Chain B light chain comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.

In some embodiments, the Chain A spFv region comprises a VL comprising an amino acid sequence of SEQ ID NO: 217, and a VH comprising an amino acid sequence of SEQ ID NO: 213, the Chain B heavy chain comprises a VH comprising an amino acid sequence of SEQ ID NO: 282, and the Chain B light chain comprises a VL comprising an amino acid sequence of SEQ ID NO: 284. In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, a bispecific antibody, or a bispecific antigen-binding fragment thereof, comprising a Chain A and a Chain B,
wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein Chain B comprises a heavy chain and a light chain, wherein the heavy chain associates with the light chain to form a CALRmut antigen binding site,
wherein the CALRmut antigen binding site of Chain A and the CALRmut antigen binding site of Chain B immunospecifically binds to CALRmut,
wherein the Chain A spFv region immunospecifically binds to CD3,
wherein the Chain A CALRmut heavy binding fragment comprises a heavy complementarity determining region (CDR) 1 (CDR1) comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275;
wherein the Chain A spFv region comprises a light CDR1 comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain A CALRmut light binding fragment comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72;
wherein the Chain B heavy chain comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275; and
wherein the Chain B light chain comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.

In some embodiments, the Chain A CALRmut heavy binding fragment comprises a VH comprising an amino acid sequence of SEQ ID NO: 282, the Chain A spFv region comprises a VL comprising an amino acid sequence of SEQ ID NO: 217, and a VH comprising an amino acid sequence of SEQ ID NO: 213, the Chain A CALRmut light binding fragment comprises a VL comprising an amino acid sequence of SEQ ID NO: 284, the Chain B heavy chain comprises a VH comprising an amino acid sequence of SEQ ID NO: 282, and the Chain B light chain comprises a VL comprising an amino acid sequence of SEQ ID NO: 284.

In some embodiments, the Chain A CALRmut heavy binding fragment comprises a VH comprising an amino acid sequence of SEQ ID NO: 282, the Chain A spFv region comprises a VL comprising an amino acid sequence of SEQ ID NO: 217, and a VH comprising an amino acid sequence of SEQ ID NO: 285, the Chain A CALRmut light binding fragment comprises a VL comprising an amino acid sequence of SEQ ID NO: 284, the Chain B heavy chain comprises a VH comprising an amino acid sequence of SEQ ID NO: 282, and the Chain B light chain comprises a VL comprising an amino acid sequence of SEQ ID NO: 284.

In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 292, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 291, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 297, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 291, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a stapled single chain fragment variable (spFv) region and a mutant calreticulin (CALRmut) heavy binding fragment, and a light chain comprising a CALRmut light binding fragment, and wherein the CALRmut heavy binding fragment is associated with a CALRmut light binding fragment to form a CALRmut antigen binding site,
wherein the Chain B spFv region and the Chain B CALRmut antigen binding site immunospecifically bind to CALRmut,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202, and
wherein the Chain B CALRmut light binding fragment comprises a light CDR1 comprising the amino acid sequence of SEQ ID NO: 33, a light CDR2 comprising the amino acid sequence of SEQ ID NO: 23, and a heavy CDR3 comprising the amino acid sequence of SEQ ID NO: 34.

In some embodiments, the Chain A spFv region comprises a VL comprising an amino acid sequence of SEQ ID NO: 217, and a VH comprising an amino acid sequence of SEQ ID NO: 213, wherein the Chain B spFv region comprises a VL comprising an amino acid sequence of SEQ ID NO: 286, and a VH comprising an amino acid sequence of SEQ ID NO: 287, wherein the Chain B CALRmut heavy binding fragment comprises a VH comprising an amino acid sequence of SEQ ID NO: 203, wherein the Chain B CALRmut light binding fragment comprises a VL comprising an amino acid sequence of SEQ ID NO: 205. In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 293, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein the Chain B CALRmut antigen binding site and the Chain B spFv region immunospecifically bind to CALRmut,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain B spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275, and
wherein the Chain B CALRmut light binding fragment comprises a light CDR1 comprising the amino acid sequence of SEQ ID NO: 33, a light CDR2 comprising the amino acid sequence of SEQ ID NO: 23, and a light CDR3 comprising the amino acid sequence of SEQ ID NO: 34.

In some embodiments, the Chain A spFv region comprises a VL comprising an amino acid sequence of SEQ ID NO: 217, and a VH comprising an amino acid sequence of SEQ ID NO: 213, the Chain B CALRmut heavy binding fragment comprises a VH comprising an amino acid sequence of SEQ ID NO: 203, the Chain B spFv region comprises a VL comprising an amino acid sequence of SEQ ID NO: 286, and a VH comprising an amino acid sequence of SEQ ID NO: 287, the Chain B CALRmut light binding fragment comprises a VL comprising an amino acid sequence of SEQ ID NO: 205. In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 294, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, a bispecific antibody, or a bispecific antigen-binding fragment thereof, comprising a Chain A and a Chain B,
wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein the Chain A CALRmut antigen binding site and the Chain A spFv region immunospecifically bind to CALRmut,
wherein Chain B comprises a stapled single chain fragment variable (spFv) region that immunospecifically binds to CD3,
wherein the Chain A CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain A spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B CALRmut light binding fragment comprises a light CDR1 comprising the amino acid sequence of SEQ ID NO: 33, a light CDR2 comprising the amino acid sequence of SEQ ID NO: 23, and a light CDR3 comprising the amino acid sequence of SEQ ID NO: 34, and
wherein the Chain B spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212.

In some embodiments, the Chain A CALRmut heavy binding fragment comprises a VH comprising an amino acid sequence of SEQ ID NO: 203, the Chain A spFv region comprises a VL comprising an amino acid sequence of SEQ ID NO: 286, and a VH comprising an amino acid sequence of SEQ ID NO: 288, the Chain A CALRmut light binding fragment comprises a VL comprising an amino acid sequence of SEQ ID NO: 205, and the Chain B spFv region comprises a VL comprising an amino acid sequence of SEQ ID NO: 217, and a VH comprising an amino acid sequence of SEQ ID NO: 213. In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 295, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 206, and Chain B comprises an amino acid sequence that is at least 90%, 91%, 92%, 93%, 94%, 95%, 96%, 97%, 98%, 99%, or 100% identical to the amino acid sequence of SEQ ID NO: 218.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a first mutant calreticulin (CALRmut) heavy binding fragment and a second CALRmut heavy binding fragment, a first light chain comprising a first CALRmut light binding fragment, and a second light chain comprising a second CALRmut light binding fragment,
wherein the first CALRmut heavy binding fragment is associated with the first CALRmut light binding fragment to form a first CALRmut antigen binding site,
wherein the second CALRmut heavy binding fragment is associated with the second CALRmut light binding fragment to form a second CALRmut antigen binding site,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B first CALRmut heavy binding fragment comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B second CALRmut heavy binding fragment comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B first CALRmut light binding fragment comprises a light CDR1 comprising the amino acid sequence of SEQ ID NO: 70, a heavy CDR2 comprising the amino acid sequence of SEQ ID NO: 71, and a heavy CDR3 comprising the amino acid sequence of SEQ ID NO: 72, and
wherein the Chain B second CALRmut light binding fragment comprises a light CDR1 comprising the amino acid sequence of SEQ ID NO: 70, a heavy CDR2 comprising the amino acid sequence of SEQ ID NO: 71, and a heavy CDR3 comprising the amino acid sequence of SEQ ID NO: 72.

In some embodiments, the Chain A spFv region comprises a VL comprising an amino acid sequence of SEQ ID NO: 217, and a VH comprising an amino acid sequence of SEQ ID NO: 213, the Chain B first CALRmut heavy binding fragment comprises a VH comprising an amino acid sequence of SEQ ID NO: 282, the Chain B second CALRmut heavy binding fragment comprises a VH comprising an amino acid sequence of SEQ ID NO: 282, the Chain B first CALRmut light binding fragment comprises a VL comprising an amino acid sequence of SEQ ID NO: 284, and the Chain B second CALRmut light binding fragment comprises a VL comprising an amino acid sequence of SEQ ID NO: 284. In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 298, the Chain B first light chain comprises an amino acid sequence that is at least 90%, 91%, 92%, 93%, 94%, 95%, 96%, 97%, 98%, 99%, or 100% identical to the amino acid sequence of SEQ ID NO: 291, and the Chain B second light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a first mutant calreticulin (CALRmut) heavy binding fragment and a second CALRmut heavy binding fragment, a first light chain comprising a first CALRmut light binding fragment, and a second light chain comprising a second CALRmut light binding fragment,
wherein the first CALRmut heavy binding fragment is associated with the first CALRmut light binding fragment to form a first CALRmut antigen binding site,
wherein the second CALRmut heavy binding fragment is associated with the second CALRmut light binding fragment to form a second CALRmut antigen binding site,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B first CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain B second CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain B first CALRmut light binding fragment comprises a light CDR1 comprising the amino acid sequence of SEQ ID NO: 33, a heavy CDR2 comprising the amino acid sequence of SEQ ID NO: 23, and a heavy CDR3 comprising the amino acid sequence of SEQ ID NO: 34, and
wherein the Chain B second CALRmut light binding fragment comprises a light CDR1 comprising the amino acid sequence of SEQ ID NO: 33, a heavy CDR2 comprising the amino acid sequence of SEQ ID NO: 23, and a heavy CDR3 comprising the amino acid sequence of SEQ ID NO: 34.

In some embodiments, the Chain A spFv region comprises a VL comprising an amino acid sequence of SEQ ID NO: 217, and a VH comprising an amino acid sequence of SEQ ID NO: 213, the Chain B first CALRmut heavy binding fragment comprises a VH comprising an amino acid sequence of SEQ ID NO: 203, the Chain B second CALRmut heavy binding fragment comprises a VH comprising an amino acid sequence of SEQ ID NO: 203, the Chain B first CALRmut light binding fragment comprises a VL comprising an amino acid sequence of SEQ ID NO: 205, and the Chain B second CALRmut light binding fragment comprises a VL comprising an amino acid sequence of SEQ ID NO: 205. In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 296, the Chain B first light chain comprises an amino acid sequence that is at least 90%, 91%, 92%, 93%, 94%, 95%, 96%, 97%, 98%, 99%, or 100% identical to the amino acid sequence of SEQ ID NO: 206, and the Chain B second light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, wherein the CALRmut comprises an amino acid sequence of SEQ ID NO: 1 or SEQ ID NO:2.

In some embodiments, a cell expressing any bispecific antibody, or bispecific antigen-binding fragment thereof, disclosed herein is provided. In some embodiments, the cell is a hybridoma. In some embodiments, the bispecific antibody, or bispecific antigen-binding fragment thereof, is recombinantly produced in the cell.

In some embodiments, at least one synthetic polynucleotide encoding the Chain A and/or Chain B of any bispecific antibody, or bispecific antigen-binding fragment thereof, disclosed herein is provided. In some embodiments, a vector comprising any synthetic polynucleotide is also provided.

In some embodiments, a pharmaceutical composition comprising any bispecific antibody, or bispecific antigen-binding fragment thereof, disclosed herein and a pharmaceutically acceptable carrier is provided.

In some embodiments, a method for generating any bispecific antibody, or bispecific antigen-binding fragment thereof, disclosed herein is provided, the method comprising culturing any cell disclosed herein.

In some embodiments, a method of redirecting a T cell to a cell expressing a mutant calreticulin is provided, the method comprising administering to a subject in need thereof a therapeutically effective amount of any bispecific antibody, or bispecific antigen-binding fragment thereof, disclosed herein to redirect a T cell to the cell expressing a mutant calreticulin.

In some embodiments, a method of inhibiting the growth or proliferation of a myeloproliferative neoplasm (MPN) is provided, the method comprising administering to a subject in need thereof a therapeutically effective amount of any bispecific antibody, or bispecific antigen-binding fragment thereof, disclosed herein to inhibit the growth or proliferation of the MPN.

In some embodiments, a method of treating a MPN is provided, the method comprising administering to a subject in need thereof any bispecific antibody, or bispecific antigen-binding fragment thereof, disclosed here, to the subject for a time sufficient to treat the cancer.

In some embodiments, the MPN is characterized by the presence of a mutant calreticulin. In some embodiments, the MPN is selected from the group consisting of chronic myelogenous leukemia, polycythemia vera, primary myelofibrosis, secondary myelofibrosis, secondary acute myeloid leukemia, essential thrombocythemia, chronic neutrophilic leukemia, and chronic eosinophilic leukemia.

In some embodiments, soluble mutant calreticulin is present in the blood of the subject.

In some embodiments, a kit is provided comprising any bispecific antibody, or bispecific antigen-binding fragment thereof, disclosed herein and packaging for the same. In some embodiments, a kit is provided comprising any polynucleotide disclosed herein and packaging for the same. In some embodiments, a kit is provided comprising any bispecific antibody, or bispecific antigen-binding fragment thereof, disclosed herein, any polynucleotide disclosed herein, and packaging for the same.

### BRIEF DESCRIPTION OF THE DRAWINGS

FIG. 1 is a flow diagram showing method of Fab assembly and expression in TG1 cells.

FIG. 2A-2C depicts CALRmutxCD3 antibodies at a range of concentrations (1000, 100, 10, 0 ng/mL) binding to CALRmut-negative and -positive cell lines at 1 and 24 hours. FIG. 2A depicts OCI-M2 MPL CALRwt binding; FIG. 2B depicts OCI-M2 MPL CALRdel52 binding; and FIG. 2C depicts OCI-M2 MPL CALRins5 binding.

FIG. 3 depicts an anti-CALRmut x CD3 bispecific antibody.

FIGS. 4A-4F depict graphs of CALRmutxCD3 bispecific antibody C3CRB73 binding to cells CALRmut-negative or CALRmut-positive. Flow cytometry was used to evaluate C3CRB73 binding to CALRmut-negative and CALRmut-positive cancer cells in vitro. C3CRB73 and NullxCD3 control were tested on a panel of cell lines, engineered to express MPL and CALRmut, after 1 hour 37° C. incubation, with subsequent incubation with secondary AF647-labeled antibody. Geometric mean fluorescence intensity of AF647 was measured to determine antibody binding. FIG. 4A depicts ELF-153 MPL binding, FIG. 4B depicts ELF-153 MPL CALRdel52 binding, FIG. 4C depicts ELF-153 MPL CALRins5 binding, FIG. 4D depicts OCI-M2 MPL CALRwt binding, FIG. 4E depicts OCI-M2 MPL CALRdel52 binding, and FIG. 4F depicts OCI-M2 MPL CALRins5 binding. Abbreviations: CALR, calreticulin; CALRmut, mutant calreticulin; CD, cluster of differentiation; del, deletion; ins, insertion; MPL, thrombopoietin receptor; spFv, stapled single chain fragment variable; wt, wild type.

FIG. 5 depicts the CALRmutxCD3 bispecific antibody C3CRB73 binding to primary CD34⁺ cells isolated from CALRmut MPN patients or healthy donors. Flow cytometry was used to evaluate C3CRB73 and NullxNull binding at 100 nM to CD34+ cells from CALRmut MPN patients (n=11) or healthy donors (n=5) after 1 hour 37° C. incubation. Representative histograms representing binding to CD34⁺ cells from CALRmut and healthy donors are shown. Delta MFI (i.e., MFI of C3CRB73 binding after subtraction of MFI background of NulxNull binding) was used to report the data from all the donors tested. Abbreviations: CALRmut, mutant calreticulin; CD, cluster of differentiation; del, deletion; HD, healthy donor; MFI, mean fluorescence intensity; MPN, myeloproliferative neoplasm; pt, patient.

FIGS. 6A-6F depicts graphs of the effect of C3CRB73 on cytotoxicity in T cell redirection assays using purified T cells and cancer cells expressing CALRmut, conducted at 5:1 E:T ratio. T cells from n=6 to 10 healthy donors were tested in T cell redirection assays with the indicated antibodies and target cell lines. Cytotoxicity of cancer cells was measured after 72 hours. Pairs of isogenic cell lines were used either expressing CALRmut or negative for tumor antigen. The assay was conducted at an E:T ratio of 5:1; data were averaged (single point per condition per donor) and means±SEM are graphed. FIG. 6A depicts ELF-153 MPL cytotoxicity, FIG. 6B depicts ELF-153 MPL CALRdel52 cytotoxicity, FIG. 6C depicts ELF-153 MPL CALRins5 cytotoxicity, FIG. 6D depicts OCI-M2 MPL CALRwt cytotoxicity, FIG. 6E depicts OCI-M2 MPL CALRdel52 cytotoxicity, and FIG. 6F depicts OCI-M2 MPL CALRins5 cytotoxicity. Abbreviations: CALR, calreticulin; CALRmut, mutant calreticulin; CD, cluster of differentiation; del, deletion; E:T ratio, effector-to-target ratio; ins, insertion; MPL, thrombopoietin receptor; SEM, standard error of the mean; spFv, stapled single chain fragment variable; wt, wild type.

FIGS. 7A-7F depicts the effect of C3CRB73 on T-cell activation in T-cell redirection assays. T cells from n=6 to 10 healthy donors were tested in T-cell redirection assays with the indicated antibodies and target cell lines. The percent T-cell activation was determined after 72 hours. The assay was conducted at an E:T ratio of 5:1; data were averaged (single point per condition per donor) and means±SEM are graphed. FIG. 7A depicts ELF-153 MPL T-cell activation, FIG. 7B depicts ELF-153 MPL CALRdel52 T-cell activation, FIG. 7C depicts ELF-153 MPL CALRins5 T-cell activation, FIG. 7D depicts OCI-M2 MPL CALRwt T-cell activation, FIG. 7E depicts OCI-M2 MPL CALRdel52 T-cell activation, and FIG. 7F depicts OCI-M2 MPL CALRins5 T-cell activation. Abbreviations: CALR, calreticulin; CALRmut, mutant calreticulin; CD, cluster of differentiation; del, deletion; E:T ratio, effector-to-target ratio; ins, insertion; MPL, thrombopoietin receptor; SEM, standard error of the mean; spFv, stapled single chain fragment variable; wt, wild type.

FIGS. 8A-8C depict the effect of C3CRB73 on cytotoxicity in autologous T cell redirection assays using purified CD34⁺ and T cells isolated either from healthy donor or CALRmut MPN patients. CD34⁺ and T cells isolated from n=4 healthy donors or n=7 CALRmut MPN patients (n=3 CALRins5, n=1 CALRdel52, n=2 CALRdel34, n=1 CALRdel4) were tested in T-cell redirection assays with the indicated antibodies and target cell lines. Cytotoxicity to CD34⁺ cells was measured after 120 hours. The assay was conducted at an E:T ratio of 5:1; data were averaged (n=1-2 data points generated per donor, with cells isolated from 1 or 2 patient visits) and means±SEM are graphed. FIG. 8A depicts cells from healthy donors, FIG. 8B depicts cells from CALRmut patients, and FIG. 8C breaks out the data from FIG. 8B by calreticulin mutation type. Abbreviations: CALRmut, mutant calreticulin; CALR, calreticulin; CD, cluster of differentiation; del, deletion; E:T ratio, effector-to-target ratio; ins, insertion; MPN, myeloproliferative neoplasm; SEM, standard error of the mean; spFv, stapled single chain fragment variable.

FIG. 9 depicts the quantification of CALRmut in plasma of CALRmut MPN patients. Quantification of free CALRmut proteins in the plasma from CALRmut MPN patients (n=25), assayed by ELISA. Data represent as mean±standard deviation.

FIGS. 10A and 10B depict the effect of presence of recombinant CALRmut protein on C3CRB73 binding to CALRmut-positive or CALRmut-negative cancer cells. Flow cytometry was used to evaluate C3CRB73 binding to CALRmut-positive and CALRmut-negative cancer cells in vitro in presence of recombinant CALRdel52 or CALRins5 proteins. C3CRB73 was tested on ELF-153 (FIG. 10A) or OCI-M2 (FIG. 10B) parental cell lines or expressing MPL and wild-type CALR or CALRmut (ie, CALRdel52 or CALRins5) after 24-hour 37° C. incubation, with subsequent incubation with secondary AF647-labeled antibody. Geometric mean fluorescence intensity of AF647 was measured to determine antibody binding.

FIGS. 11A and 11B depict the effect of presence of recombinant CALRmut protein on C3CRB73 binding to CALRmut-negative MPL-positive CD34⁺ HSCs derived from healthy donors. Flow cytometry was used to evaluate C3CRB73 binding after 1 hour incubation at 37° C. to CALRmut-negative MPL-positive CD34⁺ HSCs isolated from healthy donors (n=2) in vitro in presence of recombinant CALRdel52 (FIG. 11A) or CALRins5 (FIG. 11B) proteins. C3CRB73 and NullxNull control antibody was labeled with AF647. After 1 hour incubation at 37° C. with C3CRB73-AF647 or NullxNull-AF647, cells were washed and analyzed by flow cytometry. Geometric mean fluorescence intensity of AF647 was measured to determine antibody binding.

FIGS. 12A and 12B depict the effect of C3CRB73 on cytotoxicity and CD8+ T-cell activation in T-cell redirection assays in presence of recombinant CALRdel52 protein using purified T cells and cancer cells expressing MPL (FIG. 12A) or MPL and CALRdel52 (FIG. 12B), conducted at 5:1 E:T ratio. T cells from n=3 healthy donors were tested in T cell redirection assays with the indicated antibodies and target cell lines. Cytotoxicity of cancer cells was measured after 72 hours. The percent T-cell activation was determined after 72 hours. The assay was conducted at an E:T ratio of 5:1; data were averaged (single point per condition per donor) and means±SEM are graphed

FIG. 13 depicts the effect of C3CRB73 on cytotoxicity in whole-blood T cell redirection assays with spiked CALRmut-positive or negative cancer cell lines, conducted at 1:1 E:T ratio. Whole blood from n=6 healthy donors or n=9 to 13 CALRmut patients was tested in T-cell redirection assays with the indicated antibodies and ELF-153 MPL or ELF-153 MPL CALRmut+ target cell lines. In addition, whole blood from n=4 CALRmut patients was tested in T-cell redirection assays with the indicated antibodies and ELF-153 parental cell line. The percent cytotoxicity was determined after 72 hours. The assay was conducted at an E:T ratio of 1:1; data were averaged (single point per condition per donor) and means±SEM are graphed.

FIG. 14 depicts the effect of C3CRB73 on CD8+ T cell activation in whole-blood T cell redirection assays conducted at 1:1 E:T ratio, spiked with ELF 153 MPL-positive or ELF 153 MPL-positive CALRmut-positive cancer cells. Whole blood from n=6 healthy donors or n=9 to 13 CALRmut patients was tested in T cell redirection assays with the indicated antibodies and ELF 153 MPL or ELF 153 MPL CALRmut+ target cell lines. In addition, whole blood from n=4 CALRmut patients was tested in T cell redirection assays with the indicated antibodies and ELF 153 parental cell line or in absence of spiked in cell lines (‘blood only’). The percent of activated T cells (defined as CD25+ or CD69+) of total CD8+ T cells was determined after 72 hours. The assay was conducted at an E:T ratio of 1:1; data were averaged (single point per condition per donor) and means±SEM are graphed.

FIGS. 15A and 15B depict SC tumor growth kinetics (FIG. 15A) and circulating CALRmut levels in plasma (FIG. 15B) of NSG mice inoculated bilaterally with ELF-153 MPL CALRdel52 and ELF-153 MPL xenografts. NSG mice were injected SC with bilateral ELF-153 MPL CALRdel52 and ELF-153 MPL tumors. Tumor volume was measured twice weekly, and results are presented as the mean tumor volume ±SEM for each group. Tumor volume curves are shown only when two thirds of animals in each group were still in study. Soluble CALRmut levels were determined by ELISA in plasma samples obtained on Day 24 and results are presented as individual measurements, mean concentrations ±SEM. Abbreviations: CALR, calreticulin; CALRmut, mutant calreticulin; del, deletion; ELISA, enzyme-linked immunosorbent assay; MPL, thrombopoietin receptor; NSG, non-obese diabetic (NOD) severe combined immunodeficiency (scid) gamma or NOD.Cg Prkdcscid Il2rgtm1Wjl/SzJ; SC, subcutaneous; SEM, standard error of the mean.

FIG. 16 depicts CALRmut expression in paired ELF-153 MPL CALRdel52 and ELF-153 MPL tumor samples. Photomicrographs of CALRmut IHC-stained ELF-153 MPL CALRdel52 (left panel) and ELF-153 MPL (right panel) tumors. In Mouse #1, 4 ng/mL (20 ng/mL with correction factor) of soluble CALRmut were detected. In Mouse #2, 8 ng/mL (40 ng/mL with correction factor) of soluble CALRmut were detected.

FIG. 17 depicts the antitumor efficacy of C3CRB73 in SC ELF-153 MPL CALRdel52 xenografts in T cell-humanized NSG mice. T cell-humanized (T cell injection indicated by red arrow) NSG mice injected SC with ELF-153 MPL CALRdel52 tumors were dosed IP with 1, 5, or 20 mg/kg C3CRB73 (dosing indicated by black arrows). Tumor volume was measured twice weekly, and results are presented as the mean tumor volume ±SEM for each group. Tumor volume curves are shown only when two thirds of animals in each group were still in study.

FIGS. 18A and 18B depict the antitumor efficacy of C3CRB73 in disseminated OCI M2 MPL CALRins5 xenografts in T cell-humanized NSG mice. FIG. 18A depicts animal body weight loss data and FIG. 18B depicts survival data. T cell-humanized (T cell injection indicated by red arrow) NSG mice injected IV with OCI-M2 MPL CALRins5 tumors were dosed IP with indicated doses and antibodies (dosing indicated by black arrows). Negative clinical signs and/or >20% body weight loss were used as a surrogate endpoint for death. Body weight curves are shown only when two thirds of animals in each group were still in study.

FIGS. 19A and 19B depict the mutational pattern of CALR mutations in MPN patients. The wide black bar represents exon 9 of CALR, the narrow bar the 3′ UTR of the gene, the thin line intronic and intergenic regions. Indicated in FIG. 19A are the cDNA sequences in the beginning and end of exon 9. Below the cDNA sequences are the amino acid sequences derived from the three alternative reading frames. FIG. 19B shows that the three reading frames result in different peptide compositions, especially with respect to the charge of amino acids.

FIG. 20 is a table that depicts alternative CALRmutxCD3 antibodies and their structures.

FIGS. 21A-21F depict binding of different CALRmutxCD3 bispecific antibodies to CALRmut-negative or CALRmut-positive cells. Flow cytometry was used to evaluate CALRmutxCD3 binding to CALRmut-negative and CALRmut-positive cancer cells in vitro. CALRmutxCD3 antibodies were tested on a panel of cell lines, engineered to express MPL and CALRmut, after 1 hour 37° C. incubation, with subsequent incubation with secondary AF647-labeled antibody. Geometric mean fluorescence intensity of AF647 was measured to determine antibody binding. FIG. 21A depicts ELF-153 MPL CALRwt binding, FIG. 21B depicts ELF-153 MPL CALRdel52 binding, FIG. 21C depicts ELF-153 MPL CALRins5 binding, FIG. 21D depicts OCI-M2 MPL CALRwt binding, FIG. 21E depicts OCI-M2 MPL CALRdel52 binding, and FIG. 21F depicts OCI-M2 MPL CALRins5 binding.

FIG. 22 depicts binding of different CALRmutxCD3 bispecific antibodies to pan CD3 T cells. Flow cytometry was used to evaluate CALRmutxCD3 binding to pan CD3 T cells in vitro, by 1 hour 37° C. incubation, with subsequent incubation with secondary AF647-labeled antibody. Geometric mean fluorescence intensity of AF647 was measured to determine antibody binding.

FIGS. 23A-23C depict the effect of CALRmutxCD3 on cytotoxicity in T-cell cytotoxicity assays using purified T cells and cancer cells expressing CALRmut, conducted at 5:1 E:T ratio. T cells from healthy donors were tested in T cell cytotoxicity assays with the indicated antibodies and target cell lines. Cytotoxicity of cancer cells was measured after 72 hours. Pairs of isogenic cell lines were used either expressing CALRmut or negative for tumor antigen. The assay was conducted at an E:T ratio of 5:1; data were averaged (single point per condition per donor) and means±SEM are graphed. FIG. 23A depicts cytotoxicity to ELF-153 MPL CALRwt cells, FIG. 23B depicts cytotoxicity to ELF-153 MPL CALRdel52 cells, FIG. 23C depicts cytotoxicity to ELF-153 MPL CALRins5 cells. FIGS. 23D-23F depict the effect of CALRmutxCD3 on T-cell activation in T-cell cytotoxicity assays. The percent T-cell activation was determined after 72 hours. The assay was conducted at an E:T ratio of 5:1; data were averaged (single point per condition per donor) and means±SEM are graphed. FIG. 23D depicts T-cell activation in presence of ELF-153 MPL CALRwt cells, FIG. 23E depicts T-cell activation in presence of ELF-153 MPL CALRdel52 cells. FIG. 23F depicts T-cell activation in the presence of ELF-153 MPL CALRins5 cells. Abbreviations: CALR, calreticulin; CALRmut, mutant calreticulin; CD, cluster of differentiation; del, deletion; E:T ratio, effector-to-target ratio; ins, insertion; MPL, thrombopoietin receptor; SEM, standard error of the mean; spFv, stapled single chain fragment variable; wt, wild type.

FIGS. 24A and 24E depict the effect of CALRmutxCD3 on cytotoxicity in T-cell CALRmut autologous cytotoxicity assays using purified T cells and CD34+ cells isolated from CALRmut patients (n=5-11), conducted at 5:1 E:T ratio. Cytotoxicity to CD34+ cells from CALRmut patients and T-cell activation was measured after 120 hours. Data were averaged (single point per condition per donor) and means±SEM are graphed. FIG. 24A depicts cytotoxicity to CD34+ cells isolated from CALRmut patients in presence of C3CRB73, C3CRB75 or NullxCD3 negative control antibody, FIGS. 24B-24D depict corresponding T-cell activation (FIG. 24B, early T-cell activation; FIG. 24C, mid T-cell activation, FIG. 24D, late T-cell activation) within time frame of the assay in presence of C3CRB73, C3CRB75 or NullxCD3 negative control antibody. FIG. 24E depicts cytotoxicity to CD34+ cells isolated from CALRmut patients in presence of C3CRB73, C3CRB126, C3CRB127, CALRB1508 or NullxCD3 negative control antibody, FIGS. 24F-24H depicts corresponding T-cell activation (FIG. 24F, early T-cell activation; FIG. 24G, mid T-cell activation, FIG. 24H, late T-cell activation) within time frame of the assay.

FIGS. 25A-25D depicts the effect of CALRmutxCD3 on cytotoxicity in whole-blood T-cell cytotoxicity assays with added CALRmut-positive or negative cancer cell line, conducted at 1:1 E:T ratio. Whole blood from n=4 healthy donors or n=5 CALRmut patients was tested in T-cell cytotoxicity assays with the indicated antibodies and ELF-153 MPL or ELF-153 MPL CALRins5 target cell lines. The percent cytotoxicity was determined after 72 hours. The data were averaged (single point per condition per donor) and means±SEM are graphed.

FIG. 26 is a table that depicts additional alternative CALRmutxCD3 antibodies and their structures.

FIGS. 27A-27F depict binding of different CALRmutxCD3 antibodies to CALRmut-negative or CALRmut-positive cells. Flow cytometry was used to evaluate CALRmutxCD3 binding to CALRmut-negative and CALRmut-positive cancer cells in vitro. CALRmutxCD3 antibodies were tested on a panel of cell lines, engineered to express MPL and CALRmut, after 1 hour 37° C. incubation, with subsequent incubation with secondary AF647-labeled antibody. Geometric mean fluorescence intensity of AF647 was measured to determine antibody binding. FIG. 27A depicts OCI-M2 MPL CALRwt binding, FIG. 27B depicts OCI-M2 MPL CALRdel52 binding, FIG. 27C depicts OCI-M2 MPL CALRins5 binding, FIG. 27D depicts ELF-153 MPL CALRwt binding, FIG. 27E depicts ELF-153 MPL CALRdel52 binding, and FIG. 27F depicts ELF-153 MPL CALRins5 binding.

FIGS. 28A-28C depicts the effect of CALRmutxCD3 on cytotoxicity in T cell cytotoxicity assays using purified T cells and cancer cells expressing CALRmut or negative for CALRmut, conducted at 5:1 E:T ratio. T cells from healthy donors were tested in T cell cytotoxicity assays with the indicated antibodies and target cell lines. Cytotoxicity of cancer cells was measured after 72 hours. Pairs of isogenic cell lines were used either expressing CALRmut or negative for tumor antigen. The data were averaged (single point per condition per donor) and means±SEM are graphed. FIG. 28A depicts cytotoxicity to ELF-153 MPL CALRwt cells, FIG. 28B depicts cytotoxicity to ELF-153 MPL CALRdel52 cells, FIG. 28C depicts cytotoxicity to ELF-153 MPL CALRins5 cells. FIGS. 28D-28F depicts the effect of CALRmutxCD3 on T-cell activation in T-cell cytotoxicity assays. The percent T-cell activation was determined after 72 hours. The assay was conducted at an E:T ratio of 5:1; data were averaged (single point per condition per donor) and means±SEM are graphed. FIG. 28D depicts T-cell activation in presence of ELF-153 MPL CALRwt cells, FIG. 28E depicts T-cell activation in presence of ELF-153 MPL CALRdel52 cells FIG. 28F T-cell activation in the presence of ELF-153 MPL CALRins5 cells. Abbreviations: CALR, calreticulin; CALRmut, mutant calreticulin; CD, cluster of differentiation; del, deletion; E:T ratio, effector-to-target ratio; ins, insertion; MPL, thrombopoietin receptor; SEM, standard error of the mean; spFv, stapled single chain fragment variable; wt, wild type.

FIGS. 29A-29C depict the effect of CALRmutxCD3 on cytotoxicity in T cell cytotoxicity assays using purified T cells and cancer cells expressing CALRmut, conducted at 5:1 E:T ratio. T cells from healthy donors were tested in T cell cytotoxicity assays with the indicated antibodies and target cell lines. Cytotoxicity of cancer cells was measured after 72 hours. Pairs of isogenic cell lines were used either expressing CALRmut or negative for tumor antigen. The data were averaged (single point per condition per donor) and means±SEM are graphed. FIG. 29A depicts cytotoxicity to OCI-M2 MPL CALRwt cells, FIG. 29B depicts cytotoxicity to OCI-M2 MPL CALRdel52 cells, FIG. 29C depicts cytotoxicity to OCI-M2 MPL CALRins5 cells. FIGS. 29D-29F depict the effect of CALRmutxCD3 on T-cell activation in T-cell cytotoxicity assays. The percent T-cell activation was determined after 72 hours. The assay was conducted at an E:T ratio of 5:1; data were averaged (single point per condition per donor) and means±SEM are graphed. FIG. 29D depicts T-cell activation in presence of OCI-M2 MPL CALRwt cells, FIG. 29E depicts T-cell activation in presence of OCI-M2 MPL CALRdel52 cells FIG. 29F T-cell activation in the presence of OCI-M2 MPL CALRins5 cells. Abbreviations: CALR, calreticulin; CALRmut, mutant calreticulin; CD, cluster of differentiation; del, deletion; E:T ratio, effector-to-target ratio; ins, insertion; MPL, thrombopoietin receptor; SEM, standard error of the mean; spFv, stapled single chain fragment variable; wt, wild type.

FIG. 30A depicts the effect of CALRmutxCD3 on cytotoxicity in T-cell CALRmut autologous cytotoxicity assays using purified T cells and CD34+ cells isolated from CALRmut patients (n=6), conducted at 5:1 E:T ratio. Cytotoxicity to CD34+ cells from CALRmut patients and T-cell activation was measured after 120 hours. Data were averaged (single point per condition per donor) and means±SEM are graphed. FIG. 28A depicts cytotoxicity to CD34+ cells isolated from CALRmut patients in presence of CALRmutxCD3 antibodies. FIG. 30B depicts corresponding T-cell activation determined as % of CD25-positive T-cells within time frame of the assay.

### DETAILED DESCRIPTION

The present disclosure provides embodiments for antibodies and antigen-binding fragments thereof that immunospecifically bind to a mutant calreticulin (CALRmut). In some embodiments, CALRmut comprises the amino acid sequence of SEQ ID NO: 1 or SEQ ID NO: 2.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +/−10% and remain within the scope of the disclosed embodiments. Additionally, although a value may be preceded by the term “about” the exact value is also provided for herein, i.e., without the term “about”.

“Antigen” refers to any molecule (e.g., protein, peptide, polysaccharide, glycoprotein, glycolipid, nucleic acid, portions thereof, or combinations thereof) capable of being bound by an antigen binding domain or a T-cell receptor that is capable of mediating an immune response. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, and killed or inactivated whole cells or lysates.

“Antigen binding fragment” or “antigen binding domain” refers to a portion of the protein that binds an antigen. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv), stapled single chain Fv (spFv), or diabody. In some embodiments, an antibody fragment includes a stapled single chain Fv (or spFv). Antigen binding fragments may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each HC is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes: IgA, IgD, IgE, IgG and IgM, depending on the heavy chain constant domain amino acid sequence. IgA and IgG are further sub-classified as the isotypes IgAQ1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

The term “antibody” also encompasses whole or antigen binding fragments of domain, or single domain, antibodies, which can also be referred to as “sdAb” or “VHH”. Domain antibodies comprise either V_{H} or V_{L} that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference in its entirety). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO 2004/041862 and WO 2016/065323, each of which is hereby incorporated by reference in its entirety.

As used herein, the term “bispecific antibody” refers to an antibody that binds no more than two epitopes or two antigens. In some embodiments, a bispecific antibody is characterized by a first variable heavy chain and variable light chain pair which has binding specificity for a first epitope (e.g., an epitope on a CALRmut antigen) and a second variable heavy chain and variable light chain pair that has binding specificity for a second epitope (e.g., an epitope on a T cell (e.g., CD3). In some embodiments, bispecific antibodies herein comprise two or more binding regions for either the first epitope, the second epitope, or both. For example, bispecific antibodies disclosed herein comprise two variable heavy chain/variable light chain pairs which bind specifically to a CALRmut antigen.

“Immunospecifically” when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸ M, as measured by a surface plasmon resonance assay or a cell binding assay. Phrases such as “anti-[antigen] antibody” (e.g., anti-CALRmut antibody) are meant to convey that the recited antibody specifically binds the recited antigen.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another, or another domain or peptide sequence can be between the two domains or sequences and such sequences would still be considered fused or linked to one another. In some embodiments, the various domains or proteins provided for herein are linked or fused directly to one another or a linker sequence(s), such as a glycine/serine, glycine/alanine linker or other types of peptide linkers generally known to link the two domains together. Two peptide sequences are linked directly if they are directly connected to one another or indirectly if there is a linker or other structure that links the two regions. A linker can be directly linked to two different peptide sequences or domains.

As used herein, the terms “variable region” and “variable domain” refer to the portions of the light and heavy chains of an antibody that include amino acid sequences of complementary determining regions (CDRs, e.g., CDR L1, CDR L2, CDR L3, CDR H1, CDR H2, and CDR H3) and framework regions (FRs). According to the methods used in this disclosure, the amino acid positions assigned to CDRs and FRs are defined according to Kabat (Sequences of Proteins of Immunological Interest, 5^{th} Ed. Public Health Service, National Institutes of Health, Bethesda, MD. (1991)). Using this numbering system, the actual linear amino acid sequence may contain fewer or additional amino acids corresponding to a shortening of, or insertion into, a CDR (defined further herein) or FR (defined further herein) of the variable region. For example, a heavy chain variable region may include a single inserted residue (i.e., residue 52a according to Kabat) after residue 52 of CDR H2 and inserted residues (i.e., residues 82a, 82b, 82c, etc. according to Kabat) after residue 82 of heavy chain FR. The Kabat numbering of residues may be determined for a given antibody by alignment at regions of homology of the sequence of the antibody with a “standard” Kabat numbered sequence.

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5^{th} Ed. Public Health Service, National Institutes of Health, Bethesda, Md., 1991, each of which is hereby incorporated by reference in its entirety), Chothia (Chothia et al. (1987) J Mol Biol 196: 901-17, which is hereby incorporated by reference in its entirety), IMGT (Lefranc et al. (2003) Dev Comp Immunol 27: 55-77, which is hereby incorporated by reference in its entirety) and AbM (Martin and Thornton J Bmol Biol 263: 800-15, 1996, which is hereby incorporated by reference in its entirety). The correspondence between the various delineations and variable region numbering is described (see e.g., Lefranc et al. (2003) Dev Comp Immunol 27: 55-77; Honegger and Pluckthun, J Mol Biol (2001) 309:657-70; International ImMunoGeneTics (IMGT) database; Web resources, http://www_imgt_org, each of which is hereby incorporated by reference in its entirety). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Encode” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Fab” or “Fab fragment” refers to an antibody fragment composed of VH, CH1, VL and CL domains.

“Fv” or “Fv fragment” refers to an antibody fragment composed of the VH and the VL domains from a single arm of the antibody.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“Stapled single chain Fv” or “spFv”, described in WO2021030657A1, which is hereby incorporated by reference in its entirety, refer to a scFv that comprises one or more disulfide bonds between the VH and the linker or the VL and the linker. Typically, the spFv may comprise one disulfide bond between the VH and the linker, one disulfide bond between the VL and the linker, or two disulfide bonds between the VH and the linker and the VL and the linker. scFv molecules which comprise disulfide bonds between the VH and the VL are excluded from the term “spFv”.

In some embodiments, antibodies described herein may comprise mutations (e.g., amino acid substitutions, additions, and/or deletions) outside of the CDRs (i.e., in framework regions (FRs)). An amino acid substitution, addition, and/or deletion can be a substitution, addition, and/or deletion of one or more amino acids (e.g., 1, 2, 3, 4, 5, 6, 7, 8, or more). An amino acid substitution, addition, and/or deletion can be a substitution, addition, and/or deletion of eight or fewer, seven or fewer, six or fewer, five or fewer, four or fewer, three or fewer, or two or fewer single amino acids. In some embodiments, antibodies described herein may include amino acid substitutions, additions, and/or deletions in the constant regions (e.g., Fc region) of the antibody that, e.g., lead to decreased effector function, e.g., decreased complement-dependent cytolysis (CDC) antibody-dependent cell-mediated cytolysis (ADCC), antibody-dependent cell-mediated phagocytosis (ADCP), and/or decreased B-cell killing. For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcgRs or the complement factors. The binding of IgG to the activating (FcgRI, FcgRIIa, FcgRIIIa and FcgRIIIb) and inhibitory (FcgRIIb) FcgRs or the first component of complement (Clq) depends on residues located in the hinge region and the CH2 domain. Mutations can be introduced in IgG1, IgG2 and IgG4 to reduce or silence Fc functionalities. Silencing mutations can include, but are not limited to IgG1 AA (F234A, L235A), IgG4 PAA (S228P, F234A, L235A), IgG2 AA (V234A, G237A), IgG1 FEA (L234F, L235E, D265A), or IgG1 FES (L234F/L235E/P331S). In some embodiments, the disclosed antibody or antigen-binding fragment thereof can contain the IgG1 AA (F234A, L235A) mutation. In some embodiments, the disclosed antibody or antigen-binding fragment thereof can contain the IgG4 PAA (S228P, F234A, L235A) mutation. In some embodiments, the disclosed antibody or antigen-binding fragment thereof can contain the IgG2 AA (V234A, G237A) mutation. In some embodiments, the disclosed antibody or antigen-binding fragment thereof can contain the IgG1 FEA (L234F, L235E, D265A) mutation. In some embodiments, the disclosed antibody or antigen-binding fragment thereof can contain the IgG1 FES (L234F/L235E/P331S) mutation. In some embodiments, the disclosed antibody or antigen-binding fragment thereof can contain the IgG1 L234A, L235A, and/or F405L mutations. In some embodiments, the disclosed antibody or antigen-binding fragment thereof can contain the S228P, L234A, L235A, F405L, and/or R409K mutations. In some embodiments, the disclosed antibody or antigen-binding fragment thereof can contain the IgG-AA Fc-L234A, L235A, and F405L.

The disclosed antibodies or antigen-binding fragments thereof can comprise an Fc region with one or more of the following properties: (a) reduced effector function when compared to the parent Fc; (b) reduced affinity to Fcg RI, Fcg RIIa, Fcg RIIb, Fcg RIIIb and/or Fcg RIIIa; (c) reduced affinity to FcgRI; (d) reduced affinity to FcgRIIa; I reduced affinity to FcgRIIb; (f) reduced affinity to Fcg RIIIb; or (g) reduced affinity to FcgRIIIa.

In some embodiments, the heavy chain of the antibody comprises a sequence having no more than 5, 4, 3, 2, or 1 single amino acid substitutions relative to any heavy chain sequence disclosed herein. In some embodiments, the light chain of the isolated antibody comprises a sequence having no more than 5, 4, 3, 2 or 1 single amino acid substitutions relative to any light chain sequence disclosed herein.

The term “CD3” refers to the human CD3 protein multi-subunit complex. The CD3 protein multi-subunit complex is composed to 6 distinctive polypeptide chains. These include a CD3γ chain (SwissProt P09693, which is hereby incorporated by reference in its entirety), a CD3δ chain (SwissProt P04234, which is hereby incorporated by reference in its entirety), two CD3ε chains (SwissProt P07766, which is hereby incorporated by reference in its entirety), and one CD3 ζ chain homodimer (SwissProt 20963, which is hereby incorporated by reference in its entirety), and which is associated with the T cell receptor α and β chain. The term “CD3” includes any CD3 variant, isoform and species homolog which is naturally expressed by cells (including T cells) or can be expressed on cells transfected with genes or cDNA encoding those polypeptides, unless noted.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation and were found to be an important source of IL-17 and to induce robust CD8⁺ cytotoxic T cell response. Also included are “regulatory T cells” or “Tregs” which refer to T cells that suppress an abnormal or excessive immune response and play a role in immune tolerance. Tregs are typically transcription factor Foxp3-positive CD4⁺T cells and can also include transcription factor Foxp3-negative regulatory T cells that are IL-10-producing CD4⁺T cells.

The terms “mutant calreticulin” or “CALRmut” refer to mutant calreticulin identified and found to be associated with myeloid malignancies; see, Klampfl et al., N Engl J Med 2013; 369:2379-2390 Dec. 19, 2013 and Nangalia et al., N Engl J Med 2013; 369:2391-2405; EP 14 18 4835.8; PCT/EP2014/069638; and U.S. application Ser. No. 14/486,973, which are hereby incorporated by reference in their entirety. The terms “CALR” or “CALRwt” may be used interchangeably and refers to wild type calreticulin.

As used herein, the term “percent (%) identity” refers to the percentage of amino acid (or nucleic acid) residues of a candidate sequence, e.g., an antibody, or antigen-binding fragment thereof, of the present disclosure, that are identical to the amino acid (or nucleic acid) residues of a reference sequence, e.g., a wild-type antibody, or antigen-binding fragment thereof, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

$$100 \times ( fraction of A / B )$$

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In some embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position. A position may be altered by a substitution, deletion, or insertion. A substitution, deletion, or insertion may comprise a certain number of amino acids (e.g., 1, 2, 3, 4, 5, 6, 7, 8, 9, 10, 11, 12, 13, 14, 15, or more). When describing a substitution, deletion, or insertion of no more than n amino acids, this is meant that the substitution, deletion, or insertion comprises, e.g., 1, 2, 3, 4, 5, 6, 7, 8, 9, 10, 11, 12, 13, 14, 15, or n amino acids. The number or substitutions, deletions, or insertions can comprise a percent of the total sequence (e.g., 1%, 5%, 10%, 15%, 20%, or more) where the number of substitutions, deletions, or insertions alters 5%, 10%, 15%, 20% or more, of the amino acids in the total sequence.

“Polynucleotide” or “nucleic acid” refers to a synthetic molecule comprising a chain of nucleotides covalently linked by a sugar-phosphate backbone or other equivalent covalent chemistry. cDNA is a typical example of a polynucleotide. Polynucleotide may be a DNA or a RNA molecule.

“Vector” refers to an expression vector that can be utilized in a biological system or in a reconstituted biological system to direct the translation of a polypeptide encoded by a polynucleotide sequence present in the expression vector. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “individual,” “subject,” or “patient,” can be used interchangeably, means any animal, including mammals, such as mice, rats, other rodents, rabbits, dogs, cats, swine, cattle, sheep, horses, or primates, such as humans. As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the phrase “in need thereof” means that the subject has been identified as having a need for the method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from X to Y” means 1, 2, 3, 4, or 5.

### Anti-CALRmut Antibodies

In some embodiments, the antibody, or an antigen-binding fragment thereof, comprises a heavy chain comprising a heavy chain complementarity determining region (CDR) 1 (CDR1) comprising the amino acid sequence of GIDLSNNAIS (SEQ ID NO: 200), a heavy chain CDR2 comprising the amino acid sequence of VIGNTGDTY (SEQ ID NO: 201), and a heavy chain CDR3 comprising the amino acid sequence of GPPSYSSSVKNI (SEQ ID NO: 202); and a light chain comprising a light chain CDR1 comprising the amino acid sequence of QSSQSVYNNNWLS (SEQ ID NO: 33), a light chain CDR2 comprising the amino acid sequence of DASKLES (SEQ ID NO: 23), and a light chain CDR3 comprising the amino acid sequence of AGGFTGNVYT (SEQ ID NO: 34).

In some embodiments, the antibody, or an antigen-binding fragment thereof, comprises a heavy chain comprising a heavy chain CDR1 comprising the amino acid sequence of GIDLSNN (SEQ ID NO: 260), a heavy chain CDR2 comprising the amino acid sequence of GNTGD (SEQ ID NO: 261), and a heavy chain CDR3 comprising the amino acid sequence of GPPSYSSSVKNI (SEQ ID NO: 202); and a light chain comprising a light chain CDR1 comprising the amino acid sequence of QSSQSVYNNNWLS (SEQ ID NO: 33), a light chain CDR2 comprising the amino acid sequence of DASKLES (SEQ ID NO: 23), and a light chain CDR3 comprising the amino acid sequence of AGGFTGNVYT (SEQ ID NO: 34).

In some embodiments, the antibody, or an antigen-binding fragment thereof, comprises a heavy chain comprising a heavy chain CDR comprising the amino acid sequence of NNAIS (SEQ ID NO: 81), a heavy chain CDR2 comprising the amino acid sequence of VIGNTGDTYYADSAKG (SEQ ID NO: 259), and a heavy chain CDR3 comprising the amino acid sequence of GPPSYSSSVKNI (SEQ ID NO: 202); and a light chain comprising a light chain CDR1 comprising the amino acid sequence of QSSQSVYNNNWLS (SEQ ID NO: 33), a light chain CDR2 comprising the amino acid sequence of DASKLES (SEQ ID NO: 23), and a light chain CDR3 comprising the amino acid sequence of AGGFTGNVYT (SEQ ID NO: 34).

In some embodiments, the antibody, or an antigen-binding fragment thereof, comprises a heavy chain comprising a heavy chain CDR1 comprising the amino acid sequence of GIDLSNNA (SEQ ID NO: 262), a heavy chain CDR2 comprising the amino acid sequence of IGNTGDT (SEQ ID NO: 263), and a heavy chain CDR3 comprising the amino acid sequence of VRGPPSYSSSVKNI (SEQ ID NO: 264); and a light chain comprising a light chain CDR1 comprising the amino acid sequence of QSSQSVYNNNW (SEQ ID NO: 256), a light chain CDR2 comprising the amino acid sequence of DAS, and a light chain CDR3 comprising the amino acid sequence of AGGFTGNVYT (SEQ ID NO: 34).

In some embodiments, the antibody, or an antigen-binding fragment thereof, comprises a heavy chain comprising a heavy chain CDR1 consisting of the amino acid sequence of SEQ ID NO: 200, a heavy chain CDR2 consisting of the amino acid sequence of SEQ ID NO: 201, and a heavy chain CDR3 consisting of the amino acid sequence of SEQ ID NO: 202. In some embodiments, the antibody, or an antigen-binding fragment thereof, comprises a light chain comprising a light chain CDR1 consisting of the amino acid sequence of SEQ ID NO: 33, a light chain CDR2 consisting of the amino acid sequence of SEQ ID NO: 23, and a light chain CDR3 consisting of the amino acid sequence of SEQ ID NO: 34.

In some embodiments, the antibody, or an antigen-binding fragment thereof, comprises a heavy chain comprising a heavy chain CDR1 consisting of the amino acid sequence of SEQ ID NO: 260, a heavy chain CDR2 consisting of the amino acid sequence of SEQ ID NO: 261, and a heavy chain CDR3 consisting of the amino acid sequence of SEQ ID NO: 202. In some embodiments, the antibody, or an antigen-binding fragment thereof, comprises a light chain comprising a light chain CDR1 consisting of the amino acid sequence of SEQ ID NO: 33, a light chain CDR2 consisting of the amino acid sequence of SEQ ID NO: 23, and a light chain CDR3 consisting of the amino acid sequence of SEQ ID NO: 34.

In some embodiments, the antibody, or an antigen-binding fragment thereof, comprises a heavy chain comprising a heavy chain CDR1 consisting of the amino acid sequence of SEQ ID NO: 81, a heavy chain CDR2 consisting of amino acid sequence of SEQ ID NO: 259, and a heavy chain CDR3 consisting of the amino acid sequence of SEQ ID NO: 202. In some embodiments, the antibody, or an antigen-binding fragment thereof, comprises a light chain comprising a light chain CDR1 consisting of the amino acid sequence of SEQ ID NO: 33, a light chain CDR2 consisting of the amino acid sequence of SEQ ID NO: 23, and a light chain CDR3 consisting of the amino acid sequence of SEQ ID NO: 34; or

In some embodiments, the antibody, or an antigen-binding fragment thereof, comprises a heavy chain comprising a heavy chain CDR1 consisting of the amino acid sequence of SEQ ID NO: 262, a heavy chain CDR2 consisting of the amino acid sequence of SEQ ID NO: 263, and a heavy chain CDR3 consisting of the amino acid sequence of SEQ ID NO: 264. In some embodiments, the antibody, or an antigen-binding fragment thereof, comprises a light chain comprising a light chain CDR1 consisting of the amino acid sequence of SEQ ID NO: 256, a light chain CDR2 consisting of the amino acid sequence of DAS, and a light chain CDR3 consisting of the amino acid sequence of SEQ ID NO: 34.

In some embodiments, the antibody, or antigen-binding fragment thereof, comprises a heavy chain variable region (VH) comprising an amino acid sequence that is at least 90%, 91%, 92%, 93%, 94%, 95%, 96%, 97%, 98%, or 99% identical to the amino acid sequence of EVQLLESGGGLVQPGGSLRLSCAVSGIDLSNNAISWVRQAPGKGLEYVGVIGNTGDTYY ADSAKGRFTISRDSSKTTLYLQMNSLRAEDTAVYFCVRGPPSYSSSVKNIWGQGTLVTV SS (SEQ ID NO: 203). In some embodiments, the antibody, or antigen-binding fragment thereof, comprises a VH comprising the amino acid sequence of SEQ ID NO: 203. In some embodiments, the antibody, or antigen-binding fragment thereof, comprises a VH consisting of the amino acid sequence of SEQ ID NO: 203.

In some embodiments, the antibody, or antigen-binding fragment thereof, comprises a light chain variable region (VL) comprising an amino acid sequence that is at least 90%, 91%, 92%, 93%, 94%, 95%, 96%, 97%, 98%, or 99% identical to the amino acid sequence of DIQMTQSPSSVSASVGDRVTITCQSSQSVYNNNWLSWLQQKPGKAPKRLIYDASKLESG VPSRFSGSGSGTDFTLTISSVQPEDAATYYCAGGFTGNVYTFGGGTKVEIK (SEQ ID NO: 205). In some embodiments, the antibody, or antigen-binding fragment thereof, comprises a VL comprising the amino acid sequence of SEQ ID NO: 205. In some embodiments, the antibody, or antigen-binding fragment thereof, comprises a VL consisting of the amino acid sequence of SEQ ID NO: 205.

In some embodiments, the antibody, or antigen-binding fragment thereof, comprises a heavy chain variable region (VH) comprising the amino acid sequence of SEQ ID NO: 203, and a light chain variable region (VL) comprising the amino acid sequence of SEQ ID NO: 205. In some embodiments, the antibody, or antigen-binding fragment thereof, comprises a heavy chain variable region (VH) consisting of the amino acid sequence of SEQ ID NO: 203, and a light chain variable region (VL) consisting of the amino acid sequence of SEQ ID NO: 205.

In some embodiments, the antibody, or antigen-binding fragment thereof, is an IgG. In some embodiments, the antibody, or antigen-binding fragment thereof, is an IgG1, IgG2, IgG3, or IgG4 isotype. In some embodiments, the antibody, or antigen-binding fragment thereof, is an IgG1 isotype, or comprises an IgG1 isotype Fc region.

In some embodiments, the antibody, or antigen-binding fragment thereof, further comprises L234A, L235A, and D265S substitutions in the Fc region. In some embodiments, these substitutions, known as AAS substitutions, in the Fc region are known to limit or abolish antibody interactions with Fc receptors.

In some embodiments, the antibody, or antigen-binding fragment thereof, further comprises knob-into-hole (KiH) substitution in the Fc region. Without wishing to be bound by theory, these substitutions are known to enhance heterodimerization of the antibody, and are disclosed, for example, in WO 1996/027011, which is incorporated by reference in its entirety. One Fc region comprises an T366W substitution (the “knob” Fc region), while the other Fc region comprises T366S, L368A, and Y407V substitutions (the “hole” Fc region).

In some embodiments, the antibody, or antigen-binding fragment thereof, further comprises H435R and Y436F substitutions in the Fc region. In some embodiments, these substitutions in the Fc region are known to disrupt protein A binding of monomeric and homodimerized of “hole” Fc regions.

In some embodiments, antibody, or antigen-binding fragment thereof, comprises a heavy chain comprising an amino acid sequence that is at least 90%, 91%, 92%, 93%, 94%, 95%, 96%, 97%, 98%, or 99% identical to the amino acid sequence of EVQLLESGGGLVQPGGSLRLSCAVSGIDLSNNAISWVRQAPGKGLEYVGVIGNTGDTYY ADSAKGRFTISRDSSKTTLYLQMNSLRAEDTAVYFCVRGPPSYSSSVKNIWGQGTLVTV SSASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPEAA GGPSVFLFPPKPKDTLMISRTPEVTCVVVSVSHEDPEVKFNWYVDGVEVHNAKTKPREE QYNSTYRVVSVLTVLHQDWLNGKEYKCKVSNKALPAPIEKTISKAKGQPREPQVYTLPP SREEMTKNQVSLSCAVKGFYPSDIAVEWESNGQPENNYKTTPPVLDSDGSFFLVSKLTV DKSRWQQGNVFSCSVMHEALHNRFTQKSLSLSPGK (SEQ ID NO: 204). In some embodiments, the heavy chain comprises the amino acid sequence of SEQ ID NO: 204. In some embodiments, the heavy chain consists of the amino acid sequence of SEQ ID NO: 204.

In some embodiments, the antibody, or antigen-binding fragment thereof, comprises a light chain comprising an amino acid sequence that is at least 90%, 91%, 92%, 93%, 94%, 95%, 96%, 97%, 98%, or 99% identical to the amino acid sequence of DIQMTQSPSSVSASVGDRVTITCQSSQSVYNNNWLSWLQQKPGKAPKRLIYDASKLESG VPSRFSGSGSGTDFTLTISSVQPEDAATYYCAGGFTGNVYTFGGGTKVEIKRTVAAPSVF IFPPSDEQLKSGTASVVCLLNNFYPREAKVQWKVDNALQSGNSQESVTEQDSKDSTYSL SSTLTLSKADYEKHKVYACEVTHQGLSSPVTKSFNRGEC (SEQ ID NO: 206). In some embodiments, the light chain comprises the amino acid sequence of SEQ ID NO: 206. In some embodiments, the light chain consists of the amino acid sequence of SEQ ID NO: 206.

In some embodiments, the antibody comprises a heavy chain comprising the amino acid sequence of SEQ ID NO: 204 and a light chain comprising the amino acid sequence of SEQ ID NO: 206. In some embodiments, the antibody comprises a heavy chain consisting of the amino acid sequence of SEQ ID NO: 204 and a light chain consisting of the amino acid sequence of SEQ ID NO: 206.

### Anti-CALRmut x CD3 Bispecific Antibodies

Bispecific antibodies disclosed herein comprise different antigen binding moieties, which may be fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of bispecific antibodies in recombinant production, it will thus be advantageous to introduce in the Fc domain of the bispecific antibody a modification promoting the association of the desired polypeptides.

Accordingly, in some embodiments, the Fc domain the bispecific antibody disclosed herein comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain to enforce heterodimerization, which are well described e.g., in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291, which are hereby incorporated by reference in their entirety. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed). These different approaches for improved heavy chain heterodimerization are contemplated as different alternatives in combination with the heavy-light chain modifications (e.g., VH and VL exchange/replacement in one binding arm and the introduction of substitutions of charged amino acids with opposite charges in the CH1/CL interface) in the bispecific antibody which reduce heavy/light chain mispairing and Bence Jones-type side products.

In some embodiments, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g., in U.S. Pat. Nos. 5,731,168; 7,695,936; Ridgway et al., Prot Eng 9, 617-621 (1996) and Carter, J Immunol Meth 248, 7-15 (2001), each of which is hereby incorporated by reference in its entirety. Generally, the method involves introducing a protuberance (“knob”) at the interface of a first polypeptide and a corresponding cavity (“hole”) in the interface of a second polypeptide, such that the protuberance can be positioned in the cavity to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, in the CH3 domain of the first subunit of the Fc domain of the bispecific antibody an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

In some embodiments, said amino acid residue having a larger side chain volume is selected from the group consisting of arginine (R), phenylalanine (F), tyrosine (Y), and tryptophan (W).

In some embodiments, said amino acid residue having a smaller side chain volume is selected from the group consisting of alanine (A), serine (S), threonine (T), and valine (V).

The protuberance and cavity can be made by altering the nucleic acid encoding the polypeptides, e.g., by site-specific mutagenesis, or by peptide synthesis.

In some embodiments, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In some embodiments, in the second subunit of the Fc domain additionally the threonine residue at position 366 is replaced with a serine residue (T366S) and the leucine residue at position 368 is replaced with an alanine residue (L368A) (numberings according to Kabat EU index).

In some embodiments, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001), which is hereby incorporated by reference in its entirety).

In some embodiments, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some embodiments, the antigen binding moiety that binds to the second antigen (e.g., an activating T cell antigen) is fused (optionally via the first antigen binding moiety, which binds to CALRmut, and/or a peptide linker) to the first subunit of the Fc domain (comprising the “knob” modification). Without wishing to be bound by theory, fusion of the antigen binding moiety that binds a second antigen, such as an activating T cell antigen, to the knob-containing subunit of the Fc domain will (further) minimize the generation of antibodies comprising two antigen binding moieties that bind to an activating T cell antigen (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, each of which is hereby incorporated by reference in its entirety.

In some embodiments, the heterodimerization approach described in EP 1870459, which is hereby incorporated by reference in its entirety, is used alternatively. This approach is based on the introduction of charged amino acids with opposite charges at specific amino acid positions in the CH3/CH3 domain interface between the two subunits of the Fc domain. In some embodiments, the bispecific antibody comprises the amino acid mutations R409D and K370E in one of the two CH3 domains (of the Fc domain) and the amino acid mutations D399K and E357K in the other one of the CH3 domains of the Fc domain (numbering according to Kabat EU index).

In some embodiments, the bispecific antibody comprises the amino acid mutation T366W in the CH3 domain of the first subunit of the Fc domain and the amino acid mutations T366S, L368A, and Y407V in the CH3 domain of the second subunit of the Fc domain, and additionally, the amino acid mutations R409D and K370E in the CH3 domain of the first subunit of the Fc domain and the amino acid mutations D399K and E357K in the CH3 domain of the second subunit of the Fc domain (numberings according to Kabat EU index).

In some embodiments, the bispecific antibody of the present disclosure comprises the amino acid mutations S354C and T366W in the CH3 domain of the first subunit of the Fc domain and the amino acid mutations Y349C, T366S, L368A, and Y407V in the CH3 domain of the second subunit of the Fc domain, or the bispecific antibody comprises the amino acid mutations Y349C and T366W in the CH3 domain of the first subunit of the Fc domain and the amino acid mutations S354C, T366S, L368A, and Y407V in the CH3 domains of the second subunit of the Fc domain, and additionally, the amino acid mutations R409D and K370E in the CH3 domain of the first subunit of the Fc domain and the amino acid mutations D399K and E357K in the CH3 domain of the second subunit of the Fc domain (all numberings according to Kabat EU index).

In some embodiments, the heterodimerization approach described in WO 2013/157953, which is hereby incorporated by reference in its entirety, is used alternatively. In some embodiments, a first CH3 domain comprises amino acid mutation T366K and a second CH3 domain comprises amino acid mutation L351D (numberings according to Kabat EU index). In further embodiments, the first CH3 domain comprises further amino acid mutation L351K. In further embodiments, the second CH3 domain comprises further an amino acid mutation selected from Y349E, Y349D and L368E (preferably L368E) (numberings according to Kabat EU index).

In some embodiments, the heterodimerization approach described in WO 2012/058768, which is hereby incorporated by reference in its entirety, is used alternatively. In some embodiments a first CH3 domain comprises amino acid mutations L351Y, Y407A and a second CH3 domain comprises amino acid mutations T366A, K409F. In a further embodiment the second CH3 domain comprises a further amino acid mutation at position T411, D399, 5400, F405, N390, or K392, e.g. selected from a) T411N, T411R, T411Q, T411K, T411D, T411E or T411W, b) D399R, D399W, D399Y or D399K, c) S400E, 5400D, 5400R, or 5400K, d) F4051, F405M, F405T, F4055, F405V or F405W, e) N390R, N390K or N390D, f) K392V, K392M, K392R, K392L, K392F or K392E (numberings according to Kabat EU index). In further embodiments, a first CH3 domain comprises amino acid mutations L351Y, Y407A and a second CH3 domain comprises amino acid mutations T366V, K409F. In further embodiments, a first CH3 domain comprises amino acid mutation Y407A and a second CH3 domain comprises amino acid mutations T366A, K409F. In further embodiments, the second CH3 domain further comprises amino acid mutations K392E, T411E, D399R and 5400R (numberings according to Kabat EU index).

In some embodiments, the heterodimerization approach described in WO 2011/143545, which is hereby incorporated by reference in its entirety, is used alternatively, e.g., with the amino acid modification at a position selected from the group consisting of 368 and 409 (numbering according to Kabat EU index).

In some embodiments, the heterodimerization approach described in WO 2011/090762, which is hereby incorporated by reference in its entirety, which also uses the knobs-into-holes technology described above, is used alternatively. In some embodiments, a first CH3 domain comprises amino acid mutation T366W and a second CH3 domain comprises amino acid mutation Y407A. In some embodiments, a first CH3 domain comprises amino acid mutation T366V and a second CH3 domain comprises amino acid mutation Y407T (numberings according to Kabat EU index).

In some embodiments, the bispecific antibody or its Fc domain is of IgG2 subclass and the heterodimerization approach described in WO 2010/129304, which is hereby incorporated by reference in its entirety, is used alternatively.

In some embodiments, a modification promoting association of the first and the second subunit of the Fc domain comprises a modification mediating electrostatic steering effects, e.g. as described in PCT publication WO 2009/089004, which is hereby incorporated by reference in its entirety. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g. glutamic acid I, or aspartic acid (D), preferably K392D or N392D) and a second CH3 domain comprises amino acid substitution of D399, E356, D356, or E357 with a positively charged amino acid (e.g. lysine (K) or arginine I, preferably D399K, E356K, D356K, or E357K, and more preferably D399K and E356K). In some embodiments, the first CH3 domain further comprises amino acid substitution of K409 or R409 with a negatively charged amino acid (e.g., glutamic acid I, or aspartic acid (D), preferably K409D or R409D). In some embodiments, the first CH3 domain further or alternatively comprises amino acid substitution of K439 and/or K370 with a negatively charged amino acid (e.g., glutamic acid I, or aspartic acid (D)) (all numberings according to Kabat EU index).

In some embodiments, the heterodimerization approach described in WO 2007/147901, which is hereby incorporated by reference in its entirety, is used alternatively. In some embodiments, a first CH3 domain comprises amino acid mutations K253E, D282K, and K322D and a second CH3 domain comprises amino acid mutations D239K, E240K, and K292D (numberings according to Kabat EU index).

In some embodiments, the heterodimerization approach described in WO 2007/110205, which is hereby incorporated by reference in its entirety, is used alternatively.

In some embodiments, the first subunit of the Fc domain comprises amino acid substitutions K392D and K409D, and the second subunit of the Fc domain comprises amino acid substitutions D356K and D399K (numbering according to Kabat EU index).

In some embodiments, the antibody is a bispecific antibody that binds to CALRmut and CD3.

In some embodiments, the bispecific antibody, or antigen-binding fragment thereof, comprises one binding arm that binds to CD3ε and another binding arm that binds to CALRmut. In some embodiments, the CD3ε binding arm comprises a stapled single chain fragment variable (spFv) chain. In some embodiments, the CD3ε binding arm is described in WO 2021/230388, which is hereby incorporated by reference in its entirety. In some embodiments, the CALRmut binding arm is any binding section disclosed herein.

In some embodiments, the bispecific antibody, or a bispecific antigen-binding fragment thereof, comprises a heavy chain, a light chain, and a spFv chain,
wherein the heavy chain comprises a heavy chain CDR1 comprising the amino acid sequence of SEQ ID NO: 200, a heavy chain CDR2 comprising the amino acid sequence of SEQ ID NO: 201, and a heavy chain CDR3 comprising the amino acid sequence of SEQ ID NO: 202;
wherein the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34;
wherein the spFv chain comprises a light chain CDR1 comprising the amino acid sequence of RARQSIGTAIH (SEQ ID NO: 214), a light chain CDR2 comprising the amino acid sequence of YASESIS (SEQ ID NO: 215), a light chain CDR3 comprising the amino acid sequence of QQSGSWPYT (SEQ ID NO: 216), a heavy chain CDR1 comprising the amino acid sequence of GFTFSRYNMN (SEQ ID NO: 210), a heavy chain CDR2 comprising the amino acid sequence of SISTSSNYIY (SEQ ID NO: 211), and a heavy chain CDR3 comprising the amino acid sequence of GWGPFDY (SEQ ID NO: 212);
wherein the heavy chain associates with the light chain to form an antigen binding site that immunospecifically binds to a mutant calreticulin (CALRmut); and
wherein the spFv chain immunospecifically binds to CD3.

In some embodiments, the bispecific antibody, or a bispecific antigen-binding fragment thereof, comprises a heavy chain, a light chain, and a spFv chain,
wherein the heavy chain comprises a heavy chain CDR1 comprising the amino acid sequence of SEQ ID NO: 260, a heavy chain CDR2 comprising the amino acid sequence of SEQ ID NO: 261, and a heavy chain CDR3 comprising the amino acid sequence of SEQ ID NO: 202;
wherein the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34;
wherein the spFv chain comprises a light chain CDR1 comprising the amino acid sequence of RARQSIGTAIH (SEQ ID NO: 214), a light chain CDR2 comprising the amino acid sequence of YASESIS (SEQ ID NO: 215), a light chain CDR3 comprising the amino acid sequence of QQSGSWPYT (SEQ ID NO: 216), a heavy chain CDR1 comprising the amino acid sequence of GFTFSRY (SEQ ID NO: 267), a heavy chain CDR2 comprising the amino acid sequence of STSSNY (SEQ ID NO: 268), and a heavy chain CDR3 comprising the amino acid sequence of GWGPFDY (SEQ ID NO: 212);
wherein the heavy chain associates with the light chain to form an antigen binding site that immunospecifically binds to a mutant calreticulin (CALRmut); and
wherein the spFv chain immunospecifically binds to CD3.

In some embodiments, the bispecific antibody, or a bispecific antigen-binding fragment thereof, comprises a heavy chain, a light chain, and a spFv chain,
wherein the heavy chain comprises a heavy chain CDR1 comprising the amino acid sequence of SEQ ID NO: 81, a heavy chain CDR2 comprising the amino acid sequence of SEQ ID NO: 259, and a heavy chain CDR3 comprising the amino acid sequence of SEQ ID NO: 202; wherein the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34;
wherein the spFv chain comprises a light chain CDR1 comprising the amino acid sequence of RARQSIGTAIH (SEQ ID NO: 214), a light chain CDR2 comprising the amino acid sequence of YASESIS (SEQ ID NO: 215), a light chain CDR3 comprising the amino acid sequence of QQSGSWPYT (SEQ ID NO: 216), a heavy chain CDR1 comprising the amino acid sequence of RYNMN (SEQ ID NO: 265), a heavy chain CDR2 comprising the amino acid sequence of SISTSSNYIYYADSVKG (SEQ ID NO: 266), and a heavy chain CDR3 comprising the amino acid sequence of GWGPFDY (SEQ ID NO: 212);
wherein the heavy chain associates with the light chain to form an antigen binding site that immunospecifically binds to a mutant calreticulin (CALRmut); and
wherein the spFv chain immunospecifically binds to CD3.

In some embodiments, the bispecific antibody, or a bispecific antigen-binding fragment thereof, comprises a heavy chain, a light chain, and a spFv chain,
wherein the heavy chain comprises a heavy chain CDR1 comprising the amino acid sequence of SEQ ID NO: 262, a heavy chain CDR2 comprising the amino acid sequence of SEQ ID NO: 263, and a heavy chain CDR3 comprising the amino acid sequence of SEQ ID NO: 264;
wherein the light chain comprises a light chain CDR1 comprising the amino acid sequence of SEQ ID NO: 256, a light chain CDR2 comprising the amino acid sequence of DAS, and a light chain CDR3 comprising the amino acid sequence of SEQ ID NO: 34;
wherein the spFv chain comprises a light chain CDR1 comprising the amino acid sequence of QSIGTA (SEQ ID NO: 272), a light chain CDR2 comprising the amino acid sequence of YAS, a light chain CDR3 comprising the amino acid sequence of QQSGSWPYT (SEQ ID NO: 216), a heavy chain CDR1 comprising the amino acid sequence of GFTFSRYN (SEQ ID NO: 269), a heavy chain CDR2 comprising the amino acid sequence of ISTSSNYI (SEQ ID NO: 270), and a heavy chain CDR3 comprising the amino acid sequence of TRGWGPFDY (SEQ ID NO: 271);
wherein the heavy chain associates with the light chain to form an antigen binding site that immunospecifically binds to a mutant calreticulin (CALRmut); and
wherein the spFv chain immunospecifically binds to CD3.

In some embodiments, the heavy chain CDR1 consists of the amino acid sequence of SEQ ID NO: 200, the heavy chain CDR2 consists of the amino acid sequence of SEQ ID NO: 201, and the heavy chain CDR3 consists of the amino acid sequence of SEQ ID NO: 202. In some embodiments, the light chain CDR1 consists of the amino acid sequence of SEQ ID NO: 33, the light chain CDR2 consists of the amino acid sequence of SEQ ID NO: 23, and the light chain CDR3 consists of the amino acid sequence of SEQ ID NO: 34. In some embodiments, the spFv light chain CDR1 consists of the amino acid sequence of SEQ ID NO: 214, the spFv light chain CDR2 consists of the amino acid sequence of SEQ ID NO: 215, the spFv light chain CDR3 consists of the amino acid sequence of SEQ ID NO: 216, the spFv heavy chain CDR1 consists of the amino acid sequence of SEQ ID NO: 210, the spFv heavy chain CDR2 consists of the amino acid sequence of SEQ ID NO: 211, and the spFv heavy chain CDR3 consists of the amino acid sequence of SEQ ID NO: 212.

In some embodiments, the heavy chain CDR1 consists of the amino acid sequence of SEQ ID NO: 260, the heavy chain CDR2 consists of the amino acid sequence of SEQ ID NO: 261, and the heavy chain CDR3 consists of the amino acid sequence of SEQ ID NO: 202. In some embodiments the light chain CDR1 consists of the amino acid sequence of SEQ ID NO: 33, the light chain CDR2 consists of the amino acid sequence of SEQ ID NO: 23, and the light chain CDR3 consists of the amino acid sequence of SEQ ID NO: 34. In some embodiments, the spFv light chain CDR1 consists of SEQ ID NO: 214, the spFv light chain CDR2 consist of the amino acid sequence of SEQ ID NO: 215, the spFv light chain CDR3 consists of the amino acid sequence of SEQ ID NO: 216, the spFv heavy chain CDR1 consists of the amino acid sequence of SEQ ID NO: 267, the spFv heavy chain CDR2 consists of the amino acid sequence of SEQ ID NO: 268, and the spFv heavy chain CDR3 consists of the amino acid sequence of SEQ ID NO: 212.

In some embodiments, the heavy chain CDR1 consists of the amino acid sequence of SEQ ID NO: 81, the heavy chain CDR2 consists of the amino acid sequence of SEQ ID NO: 259, and the heavy chain CDR3 consists of the amino acid sequence of SEQ ID NO: 202. In some embodiments the light chain CDR1 consists of the amino acid sequence of SEQ ID NO: 33, the light chain CDR2 consists of the amino acid sequence of SEQ ID NO: 23, and the light chain CDR3 consists of the amino acid sequence of SEQ ID NO: 34. In some embodiments, the spFv light chain CDR1 consists of SEQ ID NO: 214, the spFv light chain CDR2 consist of the amino acid sequence of SEQ ID NO: 215, the spFv light chain CDR3 consists of the amino acid sequence of SEQ ID NO: 216, the spFv heavy chain CDR1 consists of the amino acid sequence of SEQ ID NO: 265, the spFv heavy chain CDR2 consists of the amino acid sequence of SEQ ID NO: 266, and the spFv heavy chain CDR3 consists of the amino acid sequence of SEQ ID NO: 212.

In some embodiments, the heavy chain CDR1 consists of the amino acid sequence of SEQ ID NO: 262, the heavy chain CDR2 consists of the amino acid sequence of SEQ ID NO: 263, and the heavy chain CDR3 consists of the amino acid sequence of SEQ ID NO: 264. In some embodiments the light chain CDR1 consists of the amino acid sequence of SEQ ID NO: 256, the light chain CDR2 consists of the amino acid sequence of DAS, and the light chain CDR3 consists of the amino acid sequence of SEQ ID NO: 34. In some embodiments, the spFv light chain CDR1 consists of SEQ ID NO: 272, the spFv light chain CDR2 consist of the amino acid sequence of YAS, the spFv light chain CDR3 consists of the amino acid sequence of SEQ ID NO: 216, the spFv heavy chain CDR1 consists of the amino acid sequence of SEQ ID NO: 269, the spFv heavy chain CDR2 consists of the amino acid sequence of SEQ ID NO: 270, and the spFv heavy chain CDR3 consists of the amino acid sequence of SEQ ID NO: 271.

In some embodiments, the heavy chain that associates with the light chain to form an antigen binding site that immunospecifically binds to a mutant calreticulin (CALRmut) of the bispecific antibody, or bispecific antigen-binding fragment thereof, comprises a V_{H} comprising an amino acid sequence that is at least 90%, 91%, 92%, 93%, 94%, 95%, 96%, 97%, 98%, or 99% identical to the amino acid sequence of SEQ ID NO: 203. In some embodiments, the heavy chain VH comprises the amino acid sequence of SEQ ID NO: 203. In some embodiments, the heavy chain VH consists of the amino acid sequence of SEQ ID NO: 203. In some embodiments, the light chain of the bispecific antibody, or bispecific antigen-binding fragment thereof, comprises a light chain VL comprising an amino acid sequence that is at least 90%, 91%, 92%, 93%, 94%, 95%, 96%, 97%, 98%, or 99% identical to the amino acid sequence of SEQ ID NO: 205. In some embodiments, the light chain VL comprises the amino acid sequence of SEQ ID NO: 205. In some embodiments, the light chain VL consists of the amino acid sequence of SEQ ID NO: 205.

In some embodiments, the spFv chain of the bispecific antibody, or bispecific antigen-binding fragment thereof, comprises a spFv VH comprising an amino acid sequence that is at least 90%, 91%, 92%, 93%, 94%, 95%, 96%, 97%, 98%, or 99% identical to the amino acid sequence of EVQLVESGGGLVKPGGSLRLSCAASGFTFSRYNMNWVRQAPGKGLEWVSSISTSSNYIY YADSVKGRFTFSRDNAKNSLDLQMSGLRAEDTAIYYCTRGWGPFDYWGCGTLVTVSS (SEQ ID NO: 213). In some embodiments, the spFv VH comprises the amino acid sequence of SEQ ID NO: 213. In some embodiments, the spFv VH consists of the amino acid sequence of SEQ ID NO: 213. In some embodiments, the spFv chain of the bispecific antibody, or bispecific antigen-binding fragment thereof, comprises a spFv VL comprising an amino acid sequence that is at least 90%, 91%, 92%, 93%, 94%, 95%, 96%, 97%, 98%, or 99% identical to the amino acid sequence of DIQMTQSPSSLSASVGDRVTITCRARQSIGTAIHWYQQKPGCAPKLLIKYASESISGVPSR FSGSGSGTDFTLTISSLQPEDFATYYCQQSGSWPYTFGQGTKLEIK (SEQ ID NO: 217). In some embodiments, the spFv VL comprises the amino acid sequence of SEQ ID NO: 217. In some embodiments, the spFv VL consists of the amino acid sequence of SEQ ID NO: 217.

In some embodiments, the antibody, or antigen-binding fragment thereof, comprises a heavy chain VH comprising the amino acid sequence of SEQ ID NO: 203, and a light chain VL comprising the amino acid sequence of SEQ ID NO: 205. In some embodiments, the antibody, or antigen-binding fragment thereof, comprises a heavy chain VH consisting of the amino acid sequence of SEQ ID NO: 203, and a light chain VL consisting of the amino acid sequence of SEQ ID NO: 205.

In some embodiments, the antibody, or antigen-binding fragment thereof, comprises a heavy chain VH comprising the amino acid sequence of SEQ ID NO: 203, a light chain VL comprising the amino acid sequence of SEQ ID NO: 205, a spFv VH comprising the amino acid sequence of SEQ ID NO: 213, and a spFv VL comprising the amino acid sequence of SEQ ID NO: 217. In some embodiments, the antibody, or antigen-binding fragment thereof, comprises a heavy chain VH consisting of the amino acid sequence of SEQ ID NO: 203, a light chain VL consisting of the amino acid sequence of SEQ ID NO: 205, a spFv VH consisting of the amino acid sequence of SEQ ID NO: 213, and a spFv VL consisting of the amino acid sequence of SEQ ID NO: 217.

In some embodiments, the antibody, or antigen-binding fragment thereof, is an IgG. In some embodiments, the antibody, or antigen-binding fragment thereof, is an IgG1, IgG2, IgG3, or IgG4 isotype. In some embodiments, the antibody, or antigen-binding fragment thereof, is an IgG1 isotype, or comprises an IgG1 isotype Fc region.

In some embodiments, the antibody, or antigen-binding fragment thereof, further comprises L234A, L235A, and D265S substitutions in the Fc region. In some embodiments, these substitutions, known as AAS substitutions, in the Fc region are known to limit or abolish antibody interactions with Fc receptors.

In some embodiments, the antibody, or antigen-binding fragment thereof, further comprises knob-into-hole (KiH) substitution in the Fc region. One Fc region comprises an T366W substitution (the “knob” Fc region), while the other Fc region comprises T366S, L368A, and Y407V substitutions (the “hole” Fc region).

In some embodiments, the antibody, or antigen-binding fragment thereof, further comprises H435R and Y436F substitutions in the Fc region. In some embodiments, these substitutions in the Fc region are known to disrupt protein A binding of monomeric and homodimerized of “hole” Fc regions.

In some embodiments, the bispecific antibody, or bispecific antigen-binding fragment thereof, comprises a heavy chain comprising an amino acid sequence that is at least 90%, 91%, 92%, 93%, 94%, 95%, 96%, 97%, 98%, or 99% identical to the amino acid sequence of SEQ ID NO: 204. In some embodiments, the heavy chain comprises the amino acid sequence of SEQ ID NO: 204. In some embodiments, the heavy chain consists of the amino acid sequence of SEQ ID NO: 204.

In some embodiments, the bispecific antibody, or bispecific antigen-binding fragment thereof, comprises a light chain comprising an amino acid sequence that is at least 90%, 91%, 92%, 93%, 94%, 95%, 96%, 97%, 98%, or 99% identical to the amino acid sequence of SEQ ID NO: 206. In some embodiments, the heavy chain comprises the amino acid sequence of SEQ ID NO: 206. In some embodiments, the heavy chain consists of the amino acid sequence of SEQ ID NO: 206.

In some embodiments, the bispecific antibody, or bispecific antigen-binding fragment thereof, comprises a spFv chain comprising an amino acid sequence that is at least 90%, 91%, 92%, 93%, 94%, 95%, 96%, 97%, 98%, or 99% identical to the amino acid sequence of SEQ ID NO: 218. In some embodiments, the heavy chain comprises the amino acid sequence of SEQ ID NO: 218. In some embodiments, the heavy chain consists of the amino acid sequence of SEQ ID NO: 218.

In some embodiments, the bispecific antibody comprises a heavy chain comprising the amino acid sequence of SEQ ID NO: 204, a light chain comprising the amino acid sequence of SEQ ID NO: 206, and a spFv chain comprising the amino acid sequence of SEQ ID NO: 218, wherein the heavy chain associates with the light chain to form an antigen binding site that immunospecifically binds to a mutant calreticulin (CALRmut); and wherein the spFv chain immunospecifically binds to CD3.

In some embodiments, the bispecific antibody comprises a heavy chain consisting of the amino acid sequence of SEQ ID NO: 204, a light chain consisting of the amino acid sequence of SEQ ID NO: 206, and a spFv chain consisting of the amino acid sequence of SEQ ID NO: 218, wherein the heavy chain associates with the light chain to form an antigen binding site that immunospecifically binds to a mutant calreticulin (CALRmut); and wherein the spFv chain immunospecifically binds to CD3.

Also provided herein are several additional bispecific antibodies and bispecific antibody fragments that bind mutant calreticulin and CD3. In some embodiments, these bispecific antibodies and bispecific antibody fragments thereof comprise multiple binding regions of one or more antigens. For example, in some embodiments, bispecific antibodies and bispecific antibody fragments thereof comprise two separate CALRmut binding regions. In some embodiments, the two separate CALRmut binding regions are located on the same Fc subunit (i.e., both on “Chain A” or “Chain B”). In some embodiments, the two separate CALRmut binding regions are located on different Fc subunits (i.e., one region on “Chain A” and the other on “Chain B”). In some embodiments, the various binding regions are associated with their respective Fc subunit via a linker.

In some embodiments, a bispecific antibody, or a bispecific antigen-binding fragment thereof, is provided, comprising a Chain A and a Chain B,
wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein Chain B comprises a heavy chain and a light chain, wherein the heavy chain associates with the light chain to form a CALRmut antigen binding site,
wherein the CALRmut antigen binding site of Chain A and the CALRmut antigen binding site of Chain B immunospecifically binds to CALRmut,
wherein the spFv region immunospecifically binds to CD3,
wherein the Chain A CALRmut heavy binding fragment comprises a heavy complementarity determining region (CDR) 1 (CDR1) comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202;
wherein the Chain A spFv region comprises a light CDR1 comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain A CALRmut light binding fragment comprises a light CDR1 comprising the amino acid sequence of SEQ ID NO: 33, a CDR2 comprising the amino acid sequence of SEQ ID NO: 23, and a CDR3 comprising the amino acid sequence of SEQ ID NO: 34;
wherein the Chain B heavy chain comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202; and
wherein the Chain B light chain comprises a light CDR1 comprising the amino acid sequence of SEQ ID NO: 33, a CDR2 comprising the amino acid sequence of SEQ ID NO: 23, and a CDR3 comprising the amino acid sequence of SEQ ID NO: 34.

In some embodiments, the Chain A CALRmut heavy binding fragment comprises a VH comprising an amino acid sequence of SEQ ID NO: 203, the Chain A spFv region comprises a VL comprising an amino acid sequence of SEQ ID NO: 217, and a VH comprising an amino acid sequence of SEQ ID NO: 213, the Chain A CALRmut light binding fragment comprises a VL comprising an amino acid sequence of SEQ ID NO: 205, the Chain B heavy chain comprises a VH comprising an amino acid sequence of SEQ ID NO: 203, and the Chain B light chain comprises a VL comprising an amino acid sequence of SEQ ID NO: 205.

In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 289, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 206, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 204, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, the Chain A heavy chain comprises an amino acid sequence of SEQ ID NO: 289, the Chain A light chain comprises an amino acid sequence of SEQ ID NO: 206, the Chain B heavy chain comprises an amino acid sequence of SEQ ID NO: 204, and the Chain B light chain comprises an amino acid sequence of SEQ ID NO: 206.

In some embodiments, a bispecific antibody, or a bispecific antigen-binding fragment thereof, is provided comprising a Chain A and a Chain B,
wherein Chain A comprises a stapled single chain fragment variable (spFv) region that immunospecifically binds to CD3,
wherein Chain B comprises a heavy chain and a light chain, wherein the heavy chain associates with the light chain to form a mutant calreticulin (CALRmut) antigen binding site that immunospecifically binds to CALRmut,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B heavy chain comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275; and
wherein the Chain B light chain comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.

In some embodiments, the Chain A spFv region comprises a VL comprising an amino acid sequence of SEQ ID NO: 217, and a VH comprising an amino acid sequence of SEQ ID NO: 213, the Chain B heavy chain comprises a VH comprising an amino acid sequence of SEQ ID NO: 282, and the Chain B light chain comprises a VL comprising an amino acid sequence of SEQ ID NO: 284. In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, a bispecific antibody, or a bispecific antigen-binding fragment thereof, comprising a Chain A and a Chain B,
wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein Chain B comprises a heavy chain and a light chain, wherein the heavy chain associates with the light chain to form a CALRmut antigen binding site,
wherein the CALRmut antigen binding site of Chain A and the CALRmut antigen binding site of Chain B immunospecifically binds to CALRmut,
wherein the Chain A spFv region immunospecifically binds to CD3,
wherein the Chain A CALRmut heavy binding fragment comprises a heavy complementarity determining region (CDR) 1 (CDR1) comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275;
wherein the Chain A spFv region comprises a light CDR1 comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain A CALRmut light binding fragment comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72;
wherein the Chain B heavy chain comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275; and
wherein the Chain B light chain comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.

In some embodiments, the Chain A CALRmut heavy binding fragment comprises a VH comprising an amino acid sequence of SEQ ID NO: 282, the Chain A spFv region comprises a VL comprising an amino acid sequence of SEQ ID NO: 217, and a VH comprising an amino acid sequence of SEQ ID NO: 213, the Chain A CALRmut light binding fragment comprises a VL comprising an amino acid sequence of SEQ ID NO: 284, the Chain B heavy chain comprises a VH comprising an amino acid sequence of SEQ ID NO: 282, and the Chain B light chain comprises a VL comprising an amino acid sequence of SEQ ID NO: 284.

In some embodiments, the Chain A CALRmut heavy binding fragment comprises a VH comprising an amino acid sequence of SEQ ID NO: 282, the Chain A spFv region comprises a VL comprising an amino acid sequence of SEQ ID NO: 217, and a VH comprising an amino acid sequence of SEQ ID NO: 285, the Chain A CALRmut light binding fragment comprises a VL comprising an amino acid sequence of SEQ ID NO: 284, the Chain B heavy chain comprises a VH comprising an amino acid sequence of SEQ ID NO: 282, and the Chain B light chain comprises a VL comprising an amino acid sequence of SEQ ID NO: 284.

In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 292, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 291, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, the Chain A heavy chain comprises an amino acid sequence of SEQ ID NO: 292, the Chain A light chain comprises an amino acid sequence of SEQ ID NO: 291, the Chain B heavy chain comprises an amino acid sequence of SEQ ID NO: 290, and the Chain B light chain comprises an amino acid sequence of SEQ ID NO: 291.

In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 297, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 291, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, the Chain A heavy chain comprises an amino acid sequence of SEQ ID NO: 297, the Chain A light chain comprises an amino acid sequence of SEQ ID NO: 291, the Chain B heavy chain comprises an amino acid sequence of SEQ ID NO: 290, and the Chain B light chain comprises an amino acid sequence of SEQ ID NO: 291.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a stapled single chain fragment variable (spFv) region and a mutant calreticulin (CALRmut) heavy binding fragment, and a light chain comprising a CALRmut light binding fragment, and wherein the CALRmut heavy binding fragment is associated with a CALRmut light binding fragment to form a CALRmut antigen binding site,
wherein the Chain B spFv region and the Chain B CALRmut antigen binding site immunospecifically bind to CALRmut,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202, and
wherein the Chain B CALRmut light binding fragment comprises a light CDR1 comprising the amino acid sequence of SEQ ID NO: 33, a light CDR2 comprising the amino acid sequence of SEQ ID NO: 23, and a heavy CDR3 comprising the amino acid sequence of SEQ ID NO: 34.

In some embodiments, the Chain A spFv region comprises a VL comprising an amino acid sequence of SEQ ID NO: 217, and a VH comprising an amino acid sequence of SEQ ID NO: 213, wherein the Chain B spFv region comprises a VL comprising an amino acid sequence of SEQ ID NO: 286, and a VH comprising an amino acid sequence of SEQ ID NO: 287, wherein the Chain B CALRmut heavy binding fragment comprises a VH comprising an amino acid sequence of SEQ ID NO: 203, wherein the Chain B CALRmut light binding fragment comprises a VL comprising an amino acid sequence of SEQ ID NO: 205.

In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 293, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, Chain A comprises an amino acid sequence of SEQ ID NO: 218, the Chain B heavy chain comprises an amino acid sequence of SEQ ID NO: 293, and the Chain B light chain comprises an amino acid sequence of SEQ ID NO: 206.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein the Chain B CALRmut antigen binding site and the Chain B spFv region immunospecifically bind to CALRmut,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain B spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275, and
wherein the Chain B CALRmut light binding fragment comprises a light CDR1 comprising the amino acid sequence of SEQ ID NO: 33, a light CDR2 comprising the amino acid sequence of SEQ ID NO: 23, and a light CDR3 comprising the amino acid sequence of SEQ ID NO: 34.

In some embodiments, the Chain A spFv region comprises a VL comprising an amino acid sequence of SEQ ID NO: 217, and a VH comprising an amino acid sequence of SEQ ID NO: 213, the Chain B CALRmut heavy binding fragment comprises a VH comprising an amino acid sequence of SEQ ID NO: 203, the Chain B spFv region comprises a VL comprising an amino acid sequence of SEQ ID NO: 286, and a VH comprising an amino acid sequence of SEQ ID NO: 287, the Chain B CALRmut light binding fragment comprises a VL comprising an amino acid sequence of SEQ ID NO: 205.

In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 294, and the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, Chain A comprises an amino acid sequence of SEQ ID NO: 218, the Chain B heavy chain comprises an amino acid sequence of SEQ ID NO: 294, and the Chain B light chain comprises an amino acid sequence of SEQ ID NO: 206.

In some embodiments, a bispecific antibody, or a bispecific antigen-binding fragment thereof, comprising a Chain A and a Chain B,
wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
wherein the Chain A CALRmut antigen binding site and the Chain A spFv region immunospecifically bind to CALRmut,
wherein Chain B comprises a stapled single chain fragment variable (spFv) region that immunospecifically binds to CD3,
wherein the Chain A CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain A spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B CALRmut light binding fragment comprises a light CDR1 comprising the amino acid sequence of SEQ ID NO: 33, a light CDR2 comprising the amino acid sequence of SEQ ID NO: 23, and a light CDR3 comprising the amino acid sequence of SEQ ID NO: 34, and
wherein the Chain B spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212.

In some embodiments, the Chain A CALRmut heavy binding fragment comprises a VH comprising an amino acid sequence of SEQ ID NO: 203, the Chain A spFv region comprises a VL comprising an amino acid sequence of SEQ ID NO: 286, and a VH comprising an amino acid sequence of SEQ ID NO: 288, the Chain A CALRmut light binding fragment comprises a VL comprising an amino acid sequence of SEQ ID NO: 205, and the Chain B spFv region comprises a VL comprising an amino acid sequence of SEQ ID NO: 217, and a VH comprising an amino acid sequence of SEQ ID NO: 213.

In some embodiments, the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 295, the Chain A light chain comprises an amino acid sequence that is at least 90%, 91%, 92%, 93%, 94%, 95%, 96%, 97%, 98%, 99%, or 100% identical to the amino acid sequence of SEQ ID NO: 206, and Chain B comprises an amino acid sequence that is at least 90%, 91%, 92%, 93%, 94%, 95%, 96%, 97%, 98%, 99%, or 100% identical to the amino acid sequence of SEQ ID NO: 218.

In some embodiments, the Chain A heavy chain comprises an amino acid sequence of SEQ ID NO: 295, the Chain A light chain comprises an amino acid sequence of SEQ ID NO: 206, and Chain B comprises an amino acid sequence of SEQ ID NO: 218.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a first mutant calreticulin (CALRmut) heavy binding fragment and a second CALRmut heavy binding fragment, a first light chain comprising a first CALRmut light binding fragment, and a second light chain comprising a second CALRmut light binding fragment,
wherein the first CALRmut heavy binding fragment is associated with the first CALRmut light binding fragment to form a first CALRmut antigen binding site,
wherein the second CALRmut heavy binding fragment is associated with the second CALRmut light binding fragment to form a second CALRmut antigen binding site,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B first CALRmut heavy binding fragment comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B second CALRmut heavy binding fragment comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
wherein the Chain B first CALRmut light binding fragment comprises a light CDR1 comprising the amino acid sequence of SEQ ID NO: 70, a heavy CDR2 comprising the amino acid sequence of SEQ ID NO: 71, and a heavy CDR3 comprising the amino acid sequence of SEQ ID NO: 72, and
wherein the Chain B second CALRmut light binding fragment comprises a light CDR1 comprising the amino acid sequence of SEQ ID NO: 70, a heavy CDR2 comprising the amino acid sequence of SEQ ID NO: 71, and a heavy CDR3 comprising the amino acid sequence of SEQ ID NO: 72.

In some embodiments, the Chain A spFv region comprises a VL comprising an amino acid sequence of SEQ ID NO: 217, and a VH comprising an amino acid sequence of SEQ ID NO: 213, the Chain B first CALRmut heavy binding fragment comprises a VH comprising an amino acid sequence of SEQ ID NO: 282, the Chain B second CALRmut heavy binding fragment comprises a VH comprising an amino acid sequence of SEQ ID NO: 282, the Chain B first CALRmut light binding fragment comprises a VL comprising an amino acid sequence of SEQ ID NO: 284, and the Chain B second CALRmut light binding fragment comprises a VL comprising an amino acid sequence of SEQ ID NO: 284.

In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 298, the Chain B first light chain comprises an amino acid sequence that is at least 90%, 91%, 92%, 93%, 94%, 95%, 96%, 97%, 98%, 99%, or 100% identical to the amino acid sequence of SEQ ID NO: 291, and the Chain B second light chain comprises an amino acid sequence that is at least 90%, 91%, 92%, 93%, 94%, 95%, 96%, 97%, 98%, 99%, or 100% identical to the amino acid sequence of SEQ ID NO: 291.

In some embodiments, Chain A comprises an amino acid sequence of SEQ ID NO: 218, the Chain B heavy chain comprises an amino acid sequence of SEQ ID NO: 298, the Chain B first light chain comprises an amino acid sequence of SEQ ID NO: 291, and the Chain B second light chain comprises an amino acid sequence of SEQ ID NO: 291.

In some embodiments, a bispecific antibody, or a bispecific antigen-binding fragment thereof, comprising a Chain A and a Chain B,
wherein Chain A comprises a stapled single chain fragment variable (spFv) region that immunospecifically binds to CD3,
wherein Chain B comprises a heavy chain comprising a first mutant calreticulin (CALRmut) heavy binding fragment and a second CALRmut heavy binding fragment, a first light chain comprising a first CALRmut light binding fragment, and a second light chain comprising a second CALRmut light binding fragment,
wherein the first CALRmut heavy binding fragment is associated with the first CALRmut light binding fragment to form a first CALRmut antigen binding site,
wherein the second CALRmut heavy binding fragment is associated with the second CALRmut light binding fragment to form a second CALRmut antigen binding site,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
wherein the Chain B first CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain B second CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
wherein the Chain B first CALRmut light binding fragment comprises a light CDR1 comprising the amino acid sequence of SEQ ID NO: 33, a heavy CDR2 comprising the amino acid sequence of SEQ ID NO: 23, and a heavy CDR3 comprising the amino acid sequence of SEQ ID NO: 34, and
wherein the Chain B second CALRmut light binding fragment comprises a light CDR1 comprising the amino acid sequence of SEQ ID NO: 33, a heavy CDR2 comprising the amino acid sequence of SEQ ID NO: 23, and a heavy CDR3 comprising the amino acid sequence of SEQ ID NO: 34.

In some embodiments, the Chain A spFv region comprises a VL comprising an amino acid sequence of SEQ ID NO: 217, and a VH comprising an amino acid sequence of SEQ ID NO: 213, the Chain B first CALRmut heavy binding fragment comprises a VH comprising an amino acid sequence of SEQ ID NO: 203, the Chain B second CALRmut heavy binding fragment comprises a VH comprising an amino acid sequence of SEQ ID NO: 203, the Chain B first CALRmut light binding fragment comprises a VL comprising an amino acid sequence of SEQ ID NO: 205, and the Chain B second CALRmut light binding fragment comprises a VL comprising an amino acid sequence of SEQ ID NO: 205.

In some embodiments, Chain A comprises an amino acid sequence that is at least 90%, 91%, 92%, 93%, 94%, 95%, 96%, 97%, 98%, 99%, or 100% identical to the amino acid sequence of SEQ ID NO: 218, the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 296, the Chain B first light chain comprises an amino acid sequence that is at least 90%, 91%, 92%, 93%, 94%, 95%, 96%, 97%, 98%, 99%, or 100% identical to the amino acid sequence of SEQ ID NO: 206, and the Chain B second light chain comprises an amino acid sequence that is at least 90%, 91%, 92%, 93%, 94%, 95%, 96%, 97%, 98%, 99%, or 100% identical to the amino acid sequence of SEQ ID NO: 206.

In some embodiments, Chain A comprises an amino acid sequence of SEQ ID NO: 218, the Chain B heavy chain comprises an amino acid sequence of SEQ ID NO: 296, the Chain B first light chain comprises an amino acid sequence of SEQ ID NO: 206, and the Chain B second light chain comprises an amino acid of SEQ ID NO: 206.

### Vectors, Host Cells, and Antibody Production

The antibodies disclosed herein can be produced from a host cell. A host cell refers to a vehicle that includes the necessary cellular components, e.g., organelles, needed to express the polypeptides and constructs described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, etc.). The choice of nucleic acid vectors depends in part on the host cells to be used. Generally, host cells are of either prokaryotic (e.g., bacterial) or eukaryotic (e.g., mammalian) origin.

### Nucleic Acid Vector Construction and Host Cells

A nucleic acid sequence encoding the amino acid sequence of the antibodies disclosed herein may be prepared by a variety of methods known in the art. These methods include, but are not limited to, oligonucleotide-mediated (or site-directed) mutagenesis and PCR mutagenesis. A nucleic acid molecule encoding an antibody disclosed herein may be obtained using standard techniques, e.g., gene synthesis. Alternatively, a nucleic acid molecule encoding an antibody disclosed herein may be mutated to contain specific amino acid substitutions using standard techniques in the art, e.g., QuikChange™ mutagenesis. Nucleic acid molecules can be synthesized using a nucleotide synthesizer or PCR techniques.

In some embodiments, the nucleotide sequence for the anti-CALRmut heavy chain comprises:

(SEQ ID NO: 207)
ATGGCCAGGAAGTCCGCTCTGCTCGCTCTGGCACTTCTGCTTCTGGGATTTGGACCT
GCTTGGGCTGAGGTTCAGCTGCTGGAATCTGGCGGAGGATTGGTTCAGCCTGGCGGC
TCTCTGAGACTGTCTTGTGCTGTGTCTGGCATCGACCTGTCCAACAACGCCATCTCCT
GGGTCCGACAGGCTCCTGGCAAAGGCCTGGAATATGTGGGCGTGATCGGCAACACC
GGCGACACCTACTACGCCGATTCTGCCAAGGGCAGATTCACCATCTCTCGGGACTCC
TCCAAGACCACACTGTACCTGCAGATGAACTCCCTGAGAGCCGAGGATACCGCCGT
GTACTTTTGTGTGCGGGGACCTCCTAGCTACTCCTCCAGCGTGAAGAACATCTGGGG
CCAGGGCACACTGGTCACCGTTTCTTCTGCCTCCACCAAGGGCCCATCGGTCTTCCC
CCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCA
GCGTGGTGACCGTGCCCTCCAGCAGCTTGGGCACCCAGACCTACATCTGCAACGTGA
ATCACAAGCCCAGCAACACCAAGGTGGACAAGAAAGTTGAGCCCAAATCTTGTGAC
AAAACTCACACATGCCCACCGTGCCCAGCACCTGAAGCCGCCGGGGGACCGTCAGT
CTTCCTCTTCCCCCCAAAACCCAAGGACACCCTCATGATCTCCCGGACCCCTGAGGT
CACATGCGTGGTGGTGAGCGTGAGCCACGAAGACCCTGAGGTCAAGTTCAACTGGT
ACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAA
TGGCAAGGAGTACAAGTGCAAGGTGTCGAACAAAGCCCTCCCAGCCCCCATCGAGA
AAACCATCTCCAAAGCCAAAGGGCAGCCCCGAGAACCACAGGTGTACACCCTGCCC
CCATCCCGGGAGGAGATGACCAAGAACCAGGTCAGCCTGTCCTGCGCCGTCAAAGG
CTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCGTGAGCA
AGCTCACCGTGGACAAGAGCAGATGGCAGCAGGGGAACGTCTTCTCATGCTCCGTG
ATGCATGAGGCTCTGCACAACCGGTTCACGCAGAAGTCTCTCTCCCTGTCTCCGGGA
AAA.

In some embodiments, the nucleotide sequence for the anti-CALRmut light chain comprises:

(SEQ ID NO: 208)
ATGGCCAGGAAGTCCGCTCTGCTCGCTCTGGCACTTCTGCTTCTGGGATT
TGGACCTGCTTGGGCTGACATCCAGATGACCCAGTCTCCATCCTCCGTGT
CTGCCTCTGTGGGCGACAGAGTGACCATCACCTGTCAGTCCAGCCAATCC
GTGTACAACAACAACTGGCTGTCCTGGCTGCAGCAGAAGCCTGGCAAGGC
TCCCAAGAGACTGATCTACGACGCCTCCAAGCTGGAATCCGGCGTGCCCT
CCAGATTTTCCGGCTCTGGCTCTGGCACCGACTTTACCCTGACCATCTCC
TCCGTGCAGCCTGAGGATGCCGCCACCTATTATTGTGCTGGAGGCTTCAC
CGGCAACGTGTACACATTTGGCGGAGGCACCAAGGTGGAAATCAAGCGTA
CTGTGGCTGCACCATCTGTCTTCATCTTCCCGCCATCTGATGAGCAGTTG
AAATCTGGAACTGCCTCTGTTGTGTGCCTGCTGAATAACTTCTATCCCAG
AGAGGCCAAAGTACAGTGGAAGGTGGATAACGCCCTCCAATCGGGTAACT
CCCAGGAGAGTGTCACAGAGCAGGACAGCAAGGACAGCACCTACAGCCTC
AGCAGCACCCTGACGCTGAGCAAAGCAGACTACGAGAAACACAAAGTCTA
CGCCTGCGAAGTCACCCATCAGGGCCTGAGCTCGCCCGTCACAAAGAGCT
TCAACAGGGGAGAGTGT.

In some embodiments, the nucleotide sequence for the anti-CD3 spFv chain comprises:

(SEQ ID NO: 219)
ATGGCCAGGAAGTCCGCTCTGCTCGCTCTGGCACTTCTGCTTCTGGGATTTGGACCT
GCTTGGGCTGACATCCAGATGACCCAGTCTCCATCCTCTCTGTCCGCCTCTGTGGGC
GACAGAGTGACCATTACCTGCCGGGCCAGACAGTCTATCGGCACCGCTATCCACTG
GTATCAGCAGAAGCCTGGCTGTGCCCCTAAGCTGCTGATTAAGTACGCCTCCGAGTC
CATCTCCGGCGTGCCCTCCAGATTTTCTGGCTCTGGATCTGGCACCGACTTTACCCTG
ACAATCTCCAGCCTGCAGCCTGAGGACTTCGCCACCTACTACTGTCAGCAGTCCGGC
TCTTGGCCTTACACCTTTGGTCAGGGCACCAAGCTGGAAATCAAGGGCGGAGGTTCT
GGTGGATCTGGCGGATGTCCTCCTTGTGGTGGAAGCGGCGGAGAGGTGCAGCTGGT
TGAATCTGGCGGAGGACTGGTTAAGCCTGGCGGCTCTCTGAGACTGTCTTGTGCTGC
TTCTGGCTTCACCTTCAGCCGGTACAACATGAACTGGGTCCGACAGGCTCCTGGCAA
AGGCCTGGAATGGGTGTCCTCCATCTCCACCTCCAGCAACTACATCTACTACGCCGA
CTCCGTGAAGGGCAGATTCACCTTCTCCAGAGACAACGCCAAGAACTCCCTGGACCT
GCAGATGTCTGGCCTGAGAGCTGAGGACACCGCTATCTACTACTGCACCAGAGGCT
GGGGACCCTTCGATTATTGGGGCTGTGGAACCCTGGTCACCGTGTCATCTGAGCCCA
AATCTAGCGACAAAACTCACACATGCCCACCGTGCCCAGCACCTGAAGCCGCCGGG
GGACCGTCAGTCTTCCTCTTCCCCCCAAAACCCAAGGACACCCTCATGATCTCCCGG
ACCCCTGAGGTCACATGCGTGGTGGTGAGCGTGAGCCACGAAGACCCTGAGGTCAA
GTTCAACTGGTACGTGGACGGCGTGGAGGTGCATAATGCCAAGACAAAGCCGCGGG
AGGAGCAGTACAACAGCACGTACCGTGTGGTCAGCGTCCTCACCGTCCTGCACCAG
GACTGGCTGAATGGCAAGGAGTACAAGTGCAAGGTGTCGAACAAAGCCCTCCCAGC
CCCCATCGAGAAAACCATCTCCAAAGCCAAAGGGCAGCCCCGAGAACCACAGGTGT
ACACCCTGCCCCCATCCCGGGAGGAGATGACCAAGAACCAGGTCAGCCTGTGGTGC
CTGGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTCTT
CCTCTACAGCAAGCTCACCGTGGACAAGAGCAGATGGCAGCAGGGGAACGTCTTCT
CATGCTCCGTGATGCATGAGGCTCTGCACAACCACTACACGCAGAAGTCTCTCTCCC
TGTCTCCGGGAAAA.

Nucleic acid sequences encoding the disclosed antibodies may be inserted into a vector capable of replicating and expressing the nucleic acid molecules in prokaryotic or eukaryotic host cells. Many vectors are available in the art and can be used for the purpose of the disclosure. Each vector may contain various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells are used as host cells for the disclosure. Examples of mammalian cell types include, but are not limited to, human embryonic kidney (HEK) (e.g., HEK293, HEK 293F), Chinese hamster ovary (CHO), HeLa, COS, PC3, Vero, MC3T3, NSO, Sp2/0, VERY, BHK, MDCK, W138, BT483, Hs578T, HTB2, BT20, T47D, NSO (a murine myeloma cell line that does not endogenously produce any immunoglobulin chains), CRL7030, and HsS78Bst cells. In other embodiments, E. coli cells are used as host cells for the disclosure. Examples of E. coli strains include, but are not limited to, E. coli 294 (ATCC® 31,446), E. coli λ 1776 (ATCC® 31,537, E. coli BL21 (DE3) (ATCC® BAA-1025), and E. coli RV308 (ATCC® 31,608). Different host cells have characteristic and specific mechanisms for the posttranslational processing and modification of protein products. Appropriate cell lines or host systems may be chosen to ensure the correct modification and processing of the antibody expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2^{nd} ed. 2004 (Jul. 20, 2004) and Vladimir Voynov and Justin A. Caravella (eds.) Therapeutic Proteins: Methods and Protocols (Methods in Molecular Biology) Humana Press; 2^{nd} ed. 2012 (Jun. 28, 2012).

### Protein Production, Recovery, and Purification

Host cells used to produce the disclosed antibodies may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., about 37° C., and CO2 levels, such as 5 to 10% (about 8%). The pH of the medium is generally from about 6.8 to 7.4, e.g., 7.0, depending mainly on the host organism. If an inducible promoter is used in the expression vector of the disclosure, protein expression is induced under conditions suitable for the activation of the promoter.

Protein recovery typically involves disrupting the host cell, generally by such means as osmotic shock, sonication, or lysis. Once the cells are disrupted, cell debris may be removed by centrifugation or filtration. The proteins may be further purified. An antibody disclosed herein may be purified by any method known in the art of protein purification, for example, by protein A affinity, other chromatography (e.g., ion exchange, affinity, and size-exclusion column chromatography), centrifugation, differential solubility, or by any other standard technique for the purification of proteins. In some instances, an antibody disclosed herein can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from the influenza hemagglutinin protein. In some embodiments, the purified antibody comprises a heavy chain comprising a C-terminal lysine. In some embodiments, the purified antibody comprises a heavy chain that does not comprise a C-terminal lysine. In some embodiments, the purified antibody comprises a population of purified antibodies, wherein the population of antibodies comprises a population of heavy chains that comprise a C-terminal lysine, and a population of heavy chains that do not comprise a C-terminal lysine.

Alternatively, the disclosed antibodies can be produced by the cells of a subject (e.g., a human), e.g., in the context of therapy, by administrating a vector (e.g., a retroviral vector, adenoviral vector, poxviral vector (e.g., vaccinia viral vector, such as Modified Vaccinia Ankara (MVA)), adeno-associated viral vector, and alphaviral vector) containing a nucleic acid molecule encoding the antibodies of the disclosure. The vector, once inside a cell of the subject (e.g., by transformation, transfection, electroporation, calcium phosphate precipitation, direct microinjection, infection, etc.) will promote expression of the disclosed antibody, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions, Preparations, and Kits

The present disclosure features pharmaceutical compositions that include one or more anti-antibodies described herein. In addition to a therapeutically effective amount of the antibody, the pharmaceutical compositions may contain one or more pharmaceutically acceptable carriers or excipients, which can be formulated by methods known to those skilled in the art.

Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2^{nd} ed.) Taylor & Francis Group, CRC Press (2006).

In some embodiments, any antibody disclosed herein, or antigen-binding fragment thereof, may be packaged in a kit. In some embodiments, the kit comprises a polynucleotide encoding any antibody, or antigen-binding fragment, disclosed herein. In some embodiments, the kit comprises any pharmaceutical composition comprising any antibody, or antigen-binding fragment, disclosed herein. In some embodiments, the kit comprises one or more of any antibody, or antigen-binding fragment, disclosed herein, any pharmaceutical composition disclosed herein, a polynucleotide disclosed herein, or any combination thereof.

### Methods of Treatment and Indications

In some embodiments, the antibody, or antigen-binding fragment thereof, is administered as part of a pharmaceutical composition. In some embodiments, the pharmaceutical composition comprises a therapeutically effective amount of the antibody, or antigen-binding fragment thereof.

As used herein, the term “therapeutically effective amount” refers to an amount, e.g., pharmaceutical dose, effective in inducing a desired biological effect in a subject or patient or in treating a patient having a condition or disorder described herein. It is also to be understood that a “therapeutically effective amount” may be interpreted as an amount giving a desired therapeutic effect, either taken in one dose or in any dosage or route, taken alone or in combination with other therapeutic agents.

As used herein, the term “no more than” refers to an amount that is less than or equal to. This may be an amount in integers. For example, no more than two substitutions can refer to 0, 1, or 2 substitutions.

As used herein, the terms “treatment” or “treating” refer to reducing, decreasing, decreasing the risk of, or decreasing the side effects of a particular disease or condition. Reducing, decreasing, decreasing the risk of, or decreasing the side effects of are relative to a subject who did not receive treatment, e.g., a control, a baseline, or a known control level or measurement.

In some embodiments, a method of redirecting a T cell to a cell expressing a mutant calreticulin is provided, the method comprising administering to a subject in need thereof a therapeutically effective amount of any antibody, or antigen-binding fragment thereof, disclosed herein to redirect a T cell to the cell expressing a mutant calreticulin.

Myeloproliferative neoplasms (MPNs) are a group of rare blood cancers in which abnormal and uncontrolled excess red blood cells, white blood cells or platelets are produced in the bone marrow. In some embodiments, the MPN is selected from the group consisting of chronic myelogenous leukemia, polycythemia vera, primary myelofibrosis, secondary myelofibrosis, secondary acute myeloid leukemia, essential thrombocythemia, chronic neutrophilic leukemia, and chronic eosinophilic leukemia.

MPNs are characterized by cytokine-independent activation of the Janus kinase (JAK)-signal transducer and activator of transcription (STAT) signaling pathway. Somatic mutations in hematopoietic stem cells in JAK2, thrombopoietin receptor (also known as MPL), and CALR (calreticulin) are phenotypic drivers in the pathogenesis of MPN. These mutations lead to pathological and constitutive activation of the JAK2-STAT5 pathway, resulting in clonal expansion of myeloid progenitors that no longer need cytokine activation for cell proliferation and differentiation. CALR is the second most frequently mutated gene, after JAK2, in MPN. Thus, in some embodiments, MPN is characterized by the presence of a mutant calreticulin (CALRmut).

CALR wild-type protein is a major chaperone and Ca2+-binding protein of the endoplasmic reticulum (ER). It plays major roles in protein folding, regulation of cell survival, and immune response. Its protein structure has 3 domains: (i) an amino domain, which is essential for chaperone function via its lectin binding sites and contains an ER signal peptide sequence, (ii) a proline-rich P domain, which binds to calcium and has a chaperone lectin-binding site, (iii) and a carboxyl domain, which also functions in calcium binding and includes an ER-retention signal (KDEL motif).

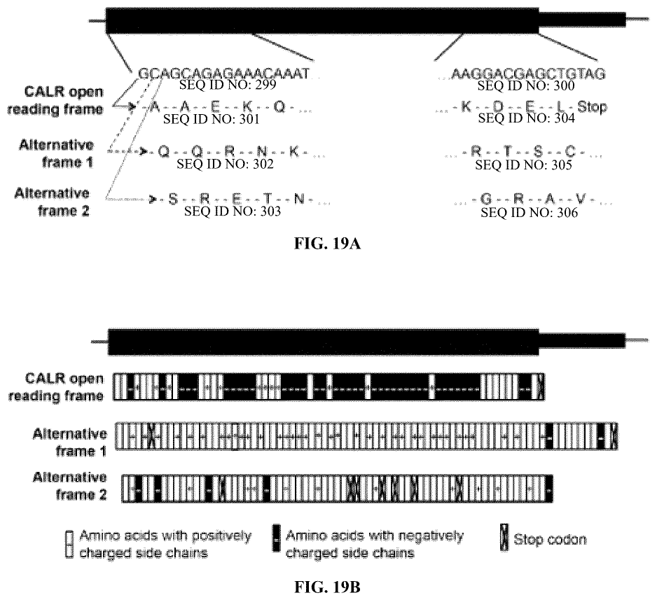

All CALR mutations are insertions or deletions resulting in a frameshift in the last exon (i.e., Exon 9) of the gene, resulting in loss of the KDEL motif and generation of a mutant-specific 36 amino acid positively charged C terminal tail (shared between the majority of known CALR mutations) (see FIG. 19A and FIG. 19B). Due to loss of the KDEL motif, CALRmut is not confined to the ER and through interaction of MPL is trafficked to the cell surface. Thus far, more than 50 different types of mutations in CALR have been detected, but a 52 base pair deletion (CALRdel52; Type 1 mutation) and a 5 base pair insertion (CALRins5; Type 2 mutation) are the most frequent types, overall being found in more than 80% of all patients with CALRmut.

The following are exemplary C-terminal amino acid residues/sequences of mutant calreticulin proteins:

```
(SEQ ID NO: 220)
TRRMMRTKMRMRRMRRTRRKMRRKMSPARPRTSCREACLQGWTEA;
(SEQ ID NO: 221)
NCRRMMRTKMRMRRMRRTRRKMRRKMSPARPRTSCREACLQGWTEA;
(SEQ ID NO: 222)
QRTRRMMRTKMRMRRMRRTRRKMRRKMSPARPRTSCREACLQGWTEA;
(SEQ ID NO: 223)
RRRQRTRRMMRTKMRMRRMRRTRRKMRRKMSPARPRTSCREACLQGWTEA;
(SEQ ID NO: 224)
GQRTRRMMRTKMRMRRMRRTRRKMRRKMSPARPRTSCREACLQGWTEA;
(SEQ ID NO: 225)
RRQRTRRMMRTKMRMRRMRRTRRKMRRKMSPARPRTSCREACLQGWTEA;
  (SEQ ID NO: 226)
RRMMRTKMRMRRMRRTRRKMRRKMSPARPRTSCREACLQGWTEA;
(SEQ ID NO: 228)
RQRTRRMMRTKMRMRRMRRTRRKMRRKMSPARPRTSCREACLQGWTEA;
(SEQ ID NO: 229)
MCRRMMRTKMRMRRMRRTRRKMRRKMSPARPRTSCREACLQGWTEA;
(SEQ ID NO: 230)
DQRQRTRRMMRTKMRMRRMRRTRRKMRRKMSPARPRTSCREACLQGWTEA;
(SEQ ID NO: 231)
RRRRQRTRRMMRTKMRMRRMRRTRRKMRRKMSPARPRTSCREACLQGWTEA;
(SEQ ID NO: 232)
QRRRQRTRRMMRTKMRMRRMRRTRRKMRRKMSPARPRTSCREACLQGWTEA;
(SEQ ID NO: 234)
RRRERTRRMMRTKMRMRRMRRTRRKMRRKMSPARPRTSCREACLQGWTEA;
(SEQ ID NO: 235)
QRRQRTRRMMRTKMRMRRMRRTRRKMRRKMSPARPRTSCREACLQGWTEA;
(SEQ ID NO: 236)
RRQWTRRMMRTKMRMRRMRRTRRKMRRKMSPARPRTSCREACLQGWTEA;
  (SEQ ID NO: 237)
RMMRTKMRMRRMRRTRRKMRRKMSPARPRTSCREACLQGWTEA;
(SEQ ID NO: 239)
GRRQRTRRMMRTKMRMRRMRRTRRKMRRKMSPARPRTSCREACLQGWTEA;
(SEQ ID NO: 240)
AFKRTRRMMRTKMRMRRMRRTRRKMRRKMSPARPRTSCREACLQGWTEA;
(SEQ ID NO: 241)
NAKRRRRQRTRRMMRTKMRMRRMRRTRRKMRRKMSPARPRTSCREACLQGWTE;
(SEQ ID NO: 242)
CVRRRRQRTRRMMRTKMRMRRMRRTRRKMRRKMSPARPRTSCREACLQGWTEA;
(SEQ ID NO: 243)
RRQRTRRMMRTKMRMRRMRRTRRKMRRKMSPARPRTSCREACLQGWTEA;
(SEQ ID NO: 244)
RQRTRRMMRTKMRMRRMRRTRRKMRRKMSPARPRTSCREACLQGWTEA;
(SEQ ID NO: 246)
CFAKRRRRQRTRRMMRTKMRMRRMRRTRRKMRRKMSPARPRTSCREACLQGWT;
(SEQ ID NO: 248)
PPLCLRRMMRTKMRMRRMRRTRRKMRRKMSPARPRTSCREACLQGWTEA;
(SEQ ID NO: 249)
DHPCRRMMRTKMRMRRMRRTRRKMRRKMSPARPRTSCREACLQGWTEA;
(SEQ ID NO: 250)
GNCRRMMRTKMRMRRMRRTRRKMRRKMSPARPRTSCREACLQGWTEA;
(SEQ ID NO: 251)
CRRMMRTKMRMRRMRRTRRKMRRKMSPARPRTSCREACLQGWTEA;
(SEQ ID NO: 253)
TCRRMMRTKMRMRRMRRTRRKMRRKMSPARPRTSCREACLQGWTEA;
and
(SEQ ID NO: 254)
ICRRMMRTKMRMRRMRRTRRKMRRKMSPARPRTSCREACLQGWTEA.
```

It is envisaged that the herein provided antibodies can specifically bind to a fragment or part of the C-terminus of mutant calreticulin protein. In some embodiments, the herein provided antibodies specifically bind to RRKMSPARPRTSCREACLQGWTEA (SEQ ID NO: 2).

CALRmut lacks a transmembrane domain and requires binding to the extracellular domain of MPL for activation of downstream signaling and for its oncogenic transformation. CALRmut binding to MPL depends on binding to both immature asparagine-linked glycan at residue N117 and to several negatively charged patches of MPL in the ER. This leads to the formation of a tight complex and charge-driven interactions providing the specificity of CALRmut for MPL. CALRmut protects N117-linked glycans of MPL from further processing in the Golgi apparatus. The tetrameric complex formed between 2 CALRmut and 2 MPL proteins is shuttled through the Golgi apparatus to the cell membrane, leading to constitutive activation of downstream kinase JAK2. MPL in complex with CALRmut remains in an immature glycosylation status on the cell surface. Recent genetic and focused pharmacological screens identified the N-glycosylation pathway, more particularly immature glycosylation of MPL, as essential for CALRmut-driven oncogenesis. In contrast in CALR wild-type cells, MPL is expressed in a mature glycosylation state on the cell membrane that cannot be bound or stimulated by soluble CALRmut.

In some embodiments, a method of inhibiting the growth or proliferation of a myeloproliferative neoplasm (MPN) is provided, the method comprising administering to a subject in need thereof a therapeutically effective amount of any antibody, or antigen-binding fragment thereof, disclosed herein to inhibit the growth or proliferation of the MPN.

In some embodiments, a method of treating a MPN is provided, the method comprising administering to a subject in need thereof any antibody, or antigen-binding fragment thereof, disclosed herein to the subject for a time sufficient to treat the cancer.

### Enumerated Embodiments

The following examples are illustrative, but not limiting, of the compounds, compositions and methods described herein. Other suitable modifications and adaptations known to those skilled in the art are within the scope of the following embodiments.
1. An antibody, or an antigen-binding fragment thereof, comprising:
   a) a heavy chain comprising a heavy chain complementarity determining region (CDR) 1 (CDR1) comprising the amino acid sequence of SEQ ID NO: 200, a heavy chain CDR2 comprising the amino acid sequence of SEQ ID NO: 201, and a heavy chain CDR3 comprising the amino acid sequence of SEQ ID NO: 202; and
   a light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34;
   b) a heavy chain comprising a heavy chain complementarity determining region (CDR) 1 (CDR1) comprising the amino acid sequence of SEQ ID NO: 260, a heavy chain CDR2 comprising the amino acid sequence of SEQ ID NO: 261, and a heavy chain CDR3 comprising the amino acid sequence of SEQ ID NO: 202; and
   a light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34;
   c) a heavy chain comprising a heavy chain complementarity determining region (CDR) 1 (CDR1) comprising the amino acid sequence of SEQ ID NO: 81, a heavy chain CDR2 comprising the amino acid sequence of SEQ ID NO: 259, and a heavy chain CDR3 comprising the amino acid sequence of SEQ ID NO: 202; and
   a light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; or
   d) a heavy chain comprising a heavy chain complementarity determining region (CDR) 1 (CDR1) comprising the amino acid sequence of SEQ ID NO: 262, a heavy chain CDR2 comprising the amino acid sequence of SEQ ID NO: 263, and a heavy chain CDR3 comprising the amino acid sequence of SEQ ID NO: 264; and
   a light chain comprising a light chain CDR1 comprising the amino acid sequence of SEQ ID NO: 256, a light chain CDR2 comprising the amino acid sequence of DAS, and a light chain CDR3 comprising the amino acid sequence of SEQ ID NO: 34.
2. The antibody, or antigen-binding fragment thereof, of embodiment 1, comprising a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 203.
3. The antibody, or antigen-binding fragment thereof, of embodiment 1 or 2, comprising a VH comprising the amino acid sequence of SEQ ID NO: 203.
4. The antibody, or antigen-binding fragment thereof, of any one of embodiments 1 to 3, comprising a light chain variable region (VL) comprising an amino acid sequence that is at least 90%, 91%, 92%, 93%, 94%, 95%, 96%, 97%, 98%, or 99% identical to the amino acid sequence of SEQ ID NO: 205.
5. The antibody, or antigen-binding fragment thereof, of any one of embodiments 1 to 4, comprising a VL comprising the amino acid sequence of SEQ ID NO: 205.
6. The antibody, or antigen-binding fragment thereof, of any one of embodiments 1 to 5, comprising a heavy chain variable region (VH) comprising the amino acid sequence of SEQ ID NO: 203, and a light chain variable region (VL) comprising the amino acid sequence of SEQ ID NO: 205.
7. The antibody, or antigen-binding fragment thereof, of any one of embodiments 1 to 6, wherein the antibody, or antigen-binding fragment thereof, is an IgG.
8. The antibody, or antigen-binding fragment thereof, of any one of embodiments 1 to 7, wherein the antibody, or antigen-binding fragment thereof, comprises an IgG1 isotype Fc region.
9. The antibody, or antigen-binding fragment thereof, of embodiment 8, further comprising L234A, L235A, and D265S substitutions in the Fc region.
10. The antibody, or antigen-binding fragment thereof, of embodiment 8 or 9, further comprising knob-into-hole (KiH) substitutions.
11. The antibody, or antigen-binding fragment thereof, of any one of embodiments 8 to 10, further comprising H435R and Y436F substitutions in the Fc region.
12. The antibody, or antigen-binding fragment thereof, of any one of embodiments 1 to 11, wherein the heavy chain comprises an amino acid sequence that is at least 90%, 91%, 92%, 93%, 94%, 95%, 96%, 97%, 98%, or 99% identical to the amino acid sequence of SEQ ID NO: 204.
13. The antibody, or antigen-binding fragment thereof, of any one of embodiments 1 to 12, wherein the heavy chain comprises the amino acid sequence of SEQ ID NO: 204.
14. The antibody, or antigen-binding fragment thereof, of any one of embodiments 1 to 13, wherein the light chain comprises an amino acid sequence that is at least 90%, 91%, 92%, 93%, 94%, 95%, 96%, 97%, 98%, or 99% identical to the amino acid sequence of SEQ ID NO: 206.
15. The antibody, or antigen-binding fragment thereof, of any one of embodiments 1 to 14, wherein the light chain comprises the amino acid sequence of SEQ ID NO: 206.
16. An antibody comprising a heavy chain comprising the amino acid sequence of SEQ ID NO: 204 and a light chain comprising the amino acid sequence of SEQ ID NO: 206.
17. The antibody, or antigen-binding fragment thereof, of any one of embodiments 1 to 16 wherein the antibody, or antigen-binding fragment thereof, immunospecifically binds to a mutant calreticulin (CALRmut).
18. The antibody, or antigen-binding fragment thereof, of embodiment 17, wherein the CALRmut comprises an amino acid sequence of SEQ ID NO: 1 or SEQ ID NO: 2.
19. A cell expressing the antibody, or antigen-binding fragment thereof, of any one of embodiments 1 to 18.
20. The cell of embodiment 19, wherein the cell is a hybridoma.
21. The cell of embodiment 19 or 20, wherein the antibody, or antigen-binding fragment thereof, is recombinantly produced.
22. A synthetic polynucleotide encoding the heavy chain and light chain of the antibody, or antigen-binding fragment thereof, of any one of embodiments 1 to 18.
23. A vector comprising the synthetic polynucleotide of embodiment 22.
24. A pharmaceutical composition comprising the antibody, or antigen-binding fragment thereof, of any one of embodiments 1 to 18 and a pharmaceutically acceptable carrier.
25. A method for generating the antibody, or antigen-binding fragment thereof, of any one of embodiments 1 to 18 by culturing the cell of any one of embodiments 19 to 21.
26. A method of redirecting a T cell to a cell expressing a mutant calreticulin, the method comprising administering to a subject in need thereof a therapeutically effective amount of the antibody, or antigen-binding fragment thereof, of any one of embodiments 1 to 18 to redirect a T cell to the cell expressing a mutant calreticulin.
27. A method of inhibiting the growth or proliferation of a myeloproliferative neoplasm (MPN), the method comprising administering to a subject in need thereof a therapeutically effective amount of the antibody, or antigen-binding fragment thereof, of any one of embodiments 1 to 18 to inhibit the growth or proliferation of the MPN.
28. A method of treating a MPN, the method comprising administering to a subject in need thereof the antibody, or antigen-binding fragment thereof, of any one of embodiments 1 to 19, to the subject for a time sufficient to treat the cancer.
29. The method of embodiment 27 or 28, wherein the MPN is characterized by the presence of a mutant calreticulin.
30. The method of any one of embodiments 27 to 29, wherein the MPN is selected from the group consisting of chronic myelogenous leukemia, polycythemia vera, primary myelofibrosis, secondary myelofibrosis, secondary acute myeloid leukemia, essential thrombocythemia, chronic neutrophilic leukemia, and chronic eosinophilic leukemia.
31. A kit comprising the antibody, or antigen-binding fragment thereof, of any one of embodiments 1 to 18 and packaging for the same.
32. A kit comprising the polynucleotide of embodiment 22 and packaging for the same.
33. A kit comprising the antibody, or antigen-binding fragment thereof, of any one of embodiments 1 to 18, the polynucleotide of embodiment 22, and packaging for the same.
34. A bispecific antibody, or a bispecific antigen-binding fragment thereof, comprising a heavy chain, a light chain, and a stapled single chain fragment variable (spFv) chain,
   wherein the heavy chain associates with the light chain to form an antigen binding site that immunospecifically binds to a mutant calreticulin (CALRmut);
   wherein the spFv chain immunospecifically binds to CD3; and
   a) wherein the heavy chain comprises a heavy chain complementarity determining region (CDR) 1 (CDR1) comprising the amino acid sequence of SEQ ID NO: 200, a heavy chain CDR2 comprising the amino acid sequence of SEQ ID NO: 201, and a heavy chain CDR3 comprising the amino acid sequence of SEQ ID NO: 202;
   wherein the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; and
   wherein the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 216, a heavy chain CDR1 comprising the amino acid sequence of SEQ ID NO: 210, a heavy chain CDR2 comprising the amino acid sequence of SEQ ID NO: 211, and a heavy chain CDR3 comprising the amino acid sequence of SEQ ID NO: 212;
   b) wherein the heavy chain comprises a heavy chain CDR1 comprising the amino acid sequence of SEQ ID NO: 260, a heavy chain CDR2 comprising the amino acid sequence of SEQ ID NO: 261, and a heavy chain CDR3 comprising the amino acid sequence of SEQ ID NO: 202;
   wherein the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; and
   wherein the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 216, a heavy chain CDR1 comprising the amino acid sequence of SEQ ID NO: 267, a heavy chain CDR2 comprising the amino acid sequence of SEQ ID NO: 268, and a heavy chain CDR3 comprising the amino acid sequence of SEQ ID NO: 212;
   c) wherein the heavy chain comprises a heavy chain CDR1 comprising the amino acid sequence of SEQ ID NO: 81, a heavy chain CDR2 comprising the amino acid sequence of SEQ ID NO: 259, and a heavy chain CDR3 comprising the amino acid sequence of SEQ ID NO: 202;
   wherein the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; and
   wherein the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 216, a heavy chain CDR1 comprising the amino acid sequence of SEQ ID NO: 265, a heavy chain CDR2 comprising the amino acid sequence of SEQ ID NO: 266, and a heavy chain CDR3 comprising the amino acid sequence of SEQ ID NO: 212; or
   d) wherein the heavy chain comprises a heavy chain CDR1 comprising the amino acid sequence of SEQ ID NO: 262, a heavy chain CDR2 comprising the amino acid sequence of SEQ ID NO: 263, and a heavy chain CDR3 comprising the amino acid sequence of SEQ ID NO: 264;
   wherein the light chain comprises a light chain CDR1 comprising the amino acid sequence of SEQ ID NO: 256, a light chain CDR2 comprising the amino acid sequence of DAS, and a light chain CDR3 comprising the amino acid sequence of SEQ ID NO: 34; and
   wherein the spFv chain comprises a light chain CDR1 comprising the amino acid sequence of SEQ ID NO: 272, a light chain CDR2 comprising the amino acid sequence of YAS, a light chain CDR3 comprising the amino acid sequence of SEQ ID NO: 216, a heavy chain CDR1 comprising the amino acid sequence of SEQ ID NO: 269, a heavy chain CDR2 comprising the amino acid sequence of SEQ ID NO: 270, and a heavy chain CDR3 comprising the amino acid sequence of SEQ ID NO: 271.
35. The bispecific antibody, or bispecific antigen-binding fragment thereof, of embodiment 34, wherein the heavy chain that associates with the light chain to form an antigen binding site comprises a VH comprising an amino acid sequence that is at least 90%, 91%, 92%, 93%, 94%, 95%, 96%, 97%, 98%, or 99% identical to the amino acid sequence of SEQ ID NO: 203.
36. The bispecific antibody, or bispecific antigen-binding fragment thereof, of embodiment 34 or 35, wherein the VH comprises the amino acid sequence of SEQ ID NO: 203.
37. The bispecific antibody, or bispecific antigen-binding fragment thereof, of any one of embodiments 34 to 36, wherein the light chain that associates with the heavy chain to form an antigen binding site comprises a VL comprising an amino acid sequence that is at least 90%, 91%, 92%, 93%, 94%, 95%, 96%, 97%, 98%, or 99% identical to the amino acid sequence of SEQ ID NO: 205.
38. The bispecific antibody, or bispecific antigen-binding fragment thereof, of any one of embodiments 34 to 37, wherein the VL comprises the amino acid sequence of SEQ ID NO: 205.
39. The antibody, or antigen-binding fragment thereof, of any one of embodiments 34 to 38, comprising a heavy chain variable region (VH) comprising the amino acid sequence of SEQ ID NO: 203, and a light chain variable region (VL) comprising the amino acid sequence of SEQ ID NO: 205.
40. The bispecific antibody, or bispecific antigen-binding fragment thereof, of any one of embodiments 34 to 39, wherein the bispecific antibody, or bispecific antigen-binding fragment thereof, is an IgG.
41. The bispecific antibody, or bispecific antigen-binding fragment thereof, of any one of embodiments 34 to 40, wherein the bispecific antibody, or bispecific antigen-binding fragment thereof, comprises an IgG1 isotype Fc region.
42. The antibody, or antigen-binding fragment thereof, of embodiment 41, further comprising L234A, L235A, and D265S substitutions in the Fc region.
43. The antibody, or antigen-binding fragment thereof, of embodiment 41 or 42, further comprising knob-into-hole (KiH) substitutions.
44. The antibody, or antigen-binding fragment thereof, of any one of embodiments 41 to 43, further comprising H435R and Y436F substitutions in the Fc region.
45. The bispecific antibody, or bispecific antigen-binding fragment thereof, of any one of embodiments 34 to 44, wherein the heavy chain comprises an amino acid sequence that is at least 90%, 91%, 92%, 93%, 94%, 95%, 96%, 97%, 98%, or 99% identical to the amino acid sequence of SEQ ID NO: 204.
46. The bispecific antibody, or bispecific antigen-binding fragment thereof, of any one of embodiments 34 to 45, wherein the heavy chain comprises the amino acid sequence of SEQ ID NO: 204.
47. The bispecific antibody, or bispecific antigen-binding fragment thereof, of any one of embodiments 34 to 46, wherein the light chain comprises an amino acid sequence that is at least 90%, 91%, 92%, 93%, 94%, 95%, 96%, 97%, 98%, or 99% identical to the amino acid sequence of SEQ ID NO: 206.
48. The bispecific antibody, or bispecific antigen-binding fragment thereof, of any one of embodiments 34 to 47, wherein the light chain comprises the amino acid sequence of SEQ ID NO: 206.
49. The bispecific antibody, or bispecific antigen-binding fragment thereof, of any one of embodiments 34 to 48, wherein the spFv chain comprises an amino acid sequence that is at least 90%, 91%, 92%, 93%, 94%, 95%, 96%, 97%, 98%, or 99% identical to the amino acid sequence of SEQ ID NO: 218.
50. The bispecific antibody, or bispecific antigen-binding fragment thereof, of any one of embodiments 34 to 49, wherein the spFv chain comprises the amino acid sequence of SEQ ID NO: 218.
51. A bispecific antibody comprising a heavy chain comprising the amino acid sequence of SEQ ID NO: 204, a light chain comprising the amino acid sequence of SEQ ID NO: 206, and a spFv chain comprising the amino acid sequence of SEQ ID NO: 218, wherein the heavy chain associates with the light chain to form an antigen binding site that immunospecifically binds to a mutant calreticulin (CALRmut); and wherein the spFv chain immunospecifically binds to CD3.
52. The bispecific antibody, or bispecific antigen-binding fragment thereof, of any one of embodiments 34 to 51, wherein the CALRmut comprises an amino acid sequence of SEQ ID NO: 1 or SEQ ID NO: 2.
53. A cell expressing the bispecific antibody, or bispecific antigen-binding fragment thereof, of any one of embodiments 34 to 52.
54. The cell of embodiment 55, wherein the cell is a hybridoma.
55. The cell of embodiment 53 or 54, wherein the antibody, or bispecific antigen-binding fragment thereof, is recombinantly produced.
56. At least one synthetic polynucleotide encoding the heavy chain, the light chain, and/or the spFv chain of the bispecific antibody, or bispecific antigen-binding fragment thereof, of any one of embodiments 34 to 52.
57. A vector comprising the synthetic polynucleotide of embodiment 56.
58. A pharmaceutical composition comprising bispecific antibody, or bispecific antigen-binding fragment thereof, of any one of embodiments 34 to 52 and a pharmaceutically acceptable carrier.
59. A method for generating the bispecific antibody, or bispecific antigen-binding fragment thereof, of any one of embodiments 34 to 52 by culturing the cell of any one of embodiments 53 to 55.
60. A method of redirecting a T cell to a cell expressing a mutant calreticulin, the method comprising administering to a subject in need thereof a therapeutically effective amount of the bispecific antibody, or bispecific antigen-binding fragment thereof, of any one of embodiments 34 to 52 to redirect a T cell to the cell expressing a mutant calreticulin.
61. A method of inhibiting the growth or proliferation of a myeloproliferative neoplasm (MPN), the method comprising administering to a subject in need thereof a therapeutically effective amount of the bispecific antibody, or bispecific antigen-binding fragment thereof, of any one of embodiments 34 to 52 to inhibit the growth or proliferation of the MPN.
62. A method of treating a MPN, the method comprising administering to a subject in need thereof the bispecific antibody, or bispecific antigen-binding fragment thereof, of any one of embodiments 34 to 52, to the subject for a time sufficient to treat the cancer.
63. The method of embodiment 61 or 62 wherein the MPN is characterized by the presence of a mutant calreticulin.
64. The method of any one of embodiments 61 to 63, wherein the MPN is selected from the group consisting of chronic myelogenous leukemia, polycythemia vera, primary myelofibrosis, essential thrombocythemia, chronic neutrophilic leukemia, and chronic eosinophilic leukemia.
65. A kit comprising the bispecific antibody, or bispecific antigen-binding fragment thereof, of any one of embodiments 34 to 52 and packaging for the same.
66. A kit comprising the polynucleotide of embodiment 56 and packaging for the same.
67. A kit comprising the bispecific antibody, or bispecific antigen-binding fragment thereof, of any one of embodiments 34 to 52, the polynucleotide of embodiment 56, and packaging for the same.
68. The method of any one of claims 26 to 30, wherein soluble mutant calreticulin is present in the blood of the subject.
69. The method of any one of claims 60 to 64, wherein soluble mutant calreticulin is present in the blood of the subject.
70. A bispecific antibody, or a bispecific antigen-binding fragment thereof, comprising a Chain A and a Chain B,
   wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
   wherein Chain B comprises a heavy chain and a light chain, wherein the heavy chain associates with the light chain to form a CALRmut antigen binding site,
   wherein the CALRmut antigen binding site of Chain A and the CALRmut antigen binding site of Chain B immunospecifically binds to CALRmut,
   wherein the spFv region immunospecifically binds to CD3,
   wherein the Chain A CALRmut heavy binding fragment comprises a heavy complementarity determining region (CDR) 1 (CDR1) comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202;
   wherein the Chain A spFv region comprises a light CDR1 comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
   wherein the Chain A CALRmut light binding fragment comprises a light CDR1 comprising the amino acid sequence of SEQ ID NO: 33, a CDR2 comprising the amino acid sequence of SEQ ID NO: 23, and a CDR3 comprising the amino acid sequence of SEQ ID NO: 34;
   wherein the Chain B heavy chain comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202; and
   wherein the Chain B light chain comprises a light CDR1 comprising the amino acid sequence of SEQ ID NO: 33, a CDR2 comprising the amino acid sequence of SEQ ID NO: 23, and a CDR3 comprising the amino acid sequence of SEQ ID NO: 34.
71. The bispecific antibody, or bispecific antigen-binding fragment thereof, of embodiment 70, wherein:
   the Chain A CALRmut heavy binding fragment comprises a VH comprising an amino acid sequence of SEQ ID NO: 203,
   the Chain A spFv region comprises a VL comprising an amino acid sequence of SEQ ID NO: 217, and a VH comprising an amino acid sequence of SEQ ID NO: 213,
   the Chain A CALRmut light binding fragment comprises a VL comprising an amino acid sequence of SEQ ID NO: 205,
   the Chain B heavy chain comprises a VH comprising an amino acid sequence of SEQ ID NO: 203, and
   the Chain B light chain comprises a VL comprising an amino acid sequence of SEQ ID NO: 205.
72. The bispecific antibody, or bispecific antigen-binding fragment thereof, of embodiment 70 or 71, wherein:
   the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 289,
   the Chain A light chain comprises an amino acid sequence that is at least 90%, 91%, 92%, 93%, 94%, 95%, 96%, 97%, 98%, 99%, or 100% identical to the amino acid sequence of SEQ ID NO: 206,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 204, and
   the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.
73. A bispecific antibody, or a bispecific antigen-binding fragment thereof, comprising a Chain A and a Chain B,
   wherein Chain A comprises a stapled single chain fragment variable (spFv) region that immunospecifically binds to CD3,
   wherein Chain B comprises a heavy chain and a light chain, wherein the heavy chain associates with the light chain to form a mutant calreticulin (CALRmut) antigen binding site that immunospecifically binds to CALRmut,
   wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
   wherein the Chain B heavy chain comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275; and
   wherein the Chain B light chain comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.
74. The bispecific antibody, or bispecific antigen-binding fragment thereof, of embodiment 73, wherein:
   the Chain A spFv region comprises a VL comprising an amino acid sequence of SEQ ID NO: 217, and a VH comprising an amino acid sequence of SEQ ID NO: 213,
   the Chain B heavy chain comprises a VH comprising an amino acid sequence of SEQ ID NO: 282, and
   the Chain B light chain comprises a VL comprising an amino acid sequence of SEQ ID NO: 284.
75. The bispecific antibody, or bispecific antigen-binding fragment thereof, of embodiment 73 or 74, wherein:
   Chain A comprises an amino acid sequence that is at least 90%, 91%, 92%, 93%, 94%, 95%, 96%, 97%, 98%, 99%, or 100% identical to the amino acid sequence of SEQ ID NO: 218,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and
   the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.
76. A bispecific antibody, or a bispecific antigen-binding fragment thereof, comprising a Chain A and a Chain B,
   wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
   wherein Chain B comprises a heavy chain and a light chain, wherein the heavy chain associates with the light chain to form a CALRmut antigen binding site,
   wherein the CALRmut antigen binding site of Chain A and the CALRmut antigen binding site of Chain B immunospecifically binds to CALRmut,
   wherein the Chain A spFv region immunospecifically binds to CD3,
   wherein the Chain A CALRmut heavy binding fragment comprises a heavy complementarity determining region (CDR) 1 (CDR1) comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275;
   wherein the Chain A spFv region comprises a light CDR1 comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
   wherein the Chain A CALRmut light binding fragment comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72;
   wherein the Chain B heavy chain comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275; and
   wherein the Chain B light chain comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.
77. The bispecific antibody, or bispecific antigen-binding fragment thereof, of embodiment 76, wherein:
   the Chain A CALRmut heavy binding fragment comprises a VH comprising an amino acid sequence of SEQ ID NO: 282,
   the Chain A spFv region comprises a VL comprising an amino acid sequence of SEQ ID NO: 217, and a VH comprising an amino acid sequence of SEQ ID NO: 213,
   the Chain A CALRmut light binding fragment comprises a VL comprising an amino acid sequence of SEQ ID NO: 284,
   the Chain B heavy chain comprises a VH comprising an amino acid sequence of SEQ ID NO: 282, and
   the Chain B light chain comprises a VL comprising an amino acid sequence of SEQ ID NO: 284.
78. The bispecific antibody, or bispecific antigen-binding fragment thereof, of embodiment 76, wherein:
   the Chain A CALRmut heavy binding fragment comprises a VH comprising an amino acid sequence of SEQ ID NO: 282,
   the Chain A spFv region comprises a VL comprising an amino acid sequence of SEQ ID NO: 217, and a VH comprising an amino acid sequence of SEQ ID NO: 285,
   the Chain A CALRmut light binding fragment comprises a VL comprising an amino acid sequence of SEQ ID NO: 284,
   the Chain B heavy chain comprises a VH comprising an amino acid sequence of SEQ ID NO: 282, and
   the Chain B light chain comprises a VL comprising an amino acid sequence of SEQ ID NO: 284.
79. The bispecific antibody, or bispecific antigen-binding fragment thereof, of embodiment 76 or 77, wherein:
   the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 292,
   the Chain A light chain comprises an amino acid sequence that is at least 90%, 91%, 92%, 93%, 94%, 95%, 96%, 97%, 98%, 99%, or 100% identical to the amino acid sequence of SEQ ID NO: 291,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and
   the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.
80. The bispecific antibody, or bispecific antigen-binding fragment thereof, of embodiment 76 or 77, wherein:
   the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 297,
   the Chain A light chain comprises an amino acid sequence that is at least 90%, 91%, 92%, 93%, 94%, 95%, 96%, 97%, 98%, 99%, or 100% identical to the amino acid sequence of SEQ ID NO: 291,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 290, and
   the Chain B light chain comprises an amino acid sequence that is at least 90%, 91%, 92%, 93%, 94%, 95%, 96%, 97%, 98%, 99%, or 100% identical to the amino acid sequence of SEQ ID NO: 291.
81. A bispecific antibody, or a bispecific antigen-binding fragment thereof, comprising a Chain A and a Chain B,
   wherein Chain A comprises a stapled single chain fragment variable (spFv) region that immunospecifically binds to CD3,
   wherein Chain B comprises a heavy chain comprising a stapled single chain fragment variable (spFv) region and a mutant calreticulin (CALRmut) heavy binding fragment, and a light chain comprising a CALRmut light binding fragment, and wherein the CALRmut heavy binding fragment is associated with a CALRmut light binding fragment to form a CALRmut antigen binding site,
   wherein the Chain B spFv region and the Chain B CALRmut antigen binding site immunospecifically bind to CALRmut,
   wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
   wherein the Chain B spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
   wherein the Chain B CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202, and
   wherein the Chain B CALRmut light binding fragment comprises a light CDR1 comprising the amino acid sequence of SEQ ID NO: 33, a light CDR2 comprising the amino acid sequence of SEQ ID NO: 23, and a heavy CDR3 comprising the amino acid sequence of SEQ ID NO: 34.
82. The bispecific antibody, or bispecific antigen-binding fragment thereof, of embodiment 81, wherein:
   the Chain A spFv region comprises a VL comprising an amino acid sequence of SEQ ID NO: 217, and a VH comprising an amino acid sequence of SEQ ID NO: 213,
   wherein the Chain B spFv region comprises a VL comprising an amino acid sequence of SEQ ID NO: 286, and a VH comprising an amino acid sequence of SEQ ID NO: 287,
   wherein the Chain B CALRmut heavy binding fragment comprises a VH comprising an amino acid sequence of SEQ ID NO: 203,
   wherein the Chain B CALRmut light binding fragment comprises a VL comprising an amino acid sequence of SEQ ID NO: 205.
83. The bispecific antibody, or bispecific antigen-binding fragment thereof, of embodiment 81 or 82, wherein:
   Chain A comprises an amino acid sequence that is at least 90%, 91%, 92%, 93%, 94%, 95%, 96%, 97%, 98%, 99%, or 100% identical to the amino acid sequence of SEQ ID NO: 218,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 293, and
   the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.
84. A bispecific antibody, or a bispecific antigen-binding fragment thereof, comprising a Chain A and a Chain B,
   wherein Chain A comprises a stapled single chain fragment variable (spFv) region that immunospecifically binds to CD3,
   wherein Chain B comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
   wherein the Chain B CALRmut antigen binding site and the Chain B spFv region immunospecifically bind to CALRmut,
   wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
   wherein the Chain B CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
   wherein the Chain B spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275, and
   wherein the Chain B CALRmut light binding fragment comprises a light CDR1 comprising the amino acid sequence of SEQ ID NO: 33, a light CDR2 comprising the amino acid sequence of SEQ ID NO: 23, and a light CDR3 comprising the amino acid sequence of SEQ ID NO: 34.
85. The bispecific antibody, or bispecific antigen-binding fragment thereof, of embodiment 84, wherein:
   the Chain A spFv region comprises a VL comprising an amino acid sequence of SEQ ID NO: 217, and a VH comprising an amino acid sequence of SEQ ID NO: 213,
   the Chain B CALRmut heavy binding fragment comprises a VH comprising an amino acid sequence of SEQ ID NO: 203,
   the Chain B spFv region comprises a VL comprising an amino acid sequence of SEQ ID NO: 286, and a VH comprising an amino acid sequence of SEQ ID NO: 287,
   the Chain B CALRmut light binding fragment comprises a VL comprising an amino acid sequence of SEQ ID NO: 205.
86. The bispecific antibody, or bispecific antigen-binding fragment thereof, of embodiment 84 or 85, wherein:
   Chain A comprises an amino acid sequence that is at least 90%, 91%, 92%, 93%, 94%, 95%, 96%, 97%, 98%, 99%, or 100% identical to the amino acid sequence of SEQ ID NO: 218,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 294, and
   the Chain B light chain comprises an amino acid sequence that is at least 90%, 91%, 92%, 93%, 94%, 95%, 96%, 97%, 98%, 99%, or 100% identical to the amino acid sequence of SEQ ID NO: 206.
87. A bispecific antibody, or a bispecific antigen-binding fragment thereof, comprising a Chain A and a Chain B,
   wherein Chain A comprises a heavy chain comprising a mutant calreticulin (CALRmut) heavy binding fragment and a stapled single chain fragment variable (spFv) region and a light chain comprising a CALRmut light binding fragment, and wherein the CALRmut heavy binding fragment is associated with a CALRmut light binding fragment to form a CALRmut antigen binding site,
   wherein the Chain A CALRmut antigen binding site and the Chain A spFv region immunospecifically bind to CALRmut,
   wherein Chain B comprises a stapled single chain fragment variable (spFv) region that immunospecifically binds to CD3,
   wherein the Chain A CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
   wherein the Chain A spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
   wherein the Chain B CALRmut light binding fragment comprises a light CDR1 comprising the amino acid sequence of SEQ ID NO: 33, a light CDR2 comprising the amino acid sequence of SEQ ID NO: 23, and a light CDR3 comprising the amino acid sequence of SEQ ID NO: 34, and
   wherein the Chain B spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212.
88. The bispecific antibody, or bispecific antigen-binding fragment thereof, of embodiment 87, wherein:
   the Chain A CALRmut heavy binding fragment comprises a V_{H} comprising an amino acid sequence of SEQ ID NO: 203,
   the Chain A spFv region comprises a VL comprising an amino acid sequence of SEQ ID NO: 286, and a V_{H} comprising an amino acid sequence of SEQ ID NO: 288,
   the Chain A CALRmut light binding fragment comprises a VL comprising an amino acid sequence of SEQ ID NO: 205, and
   the Chain B spFv region comprises a VL comprising an amino acid sequence of SEQ ID NO: 217, and a V_{H} comprising an amino acid sequence of SEQ ID NO: 213.
89. The bispecific antibody, or bispecific antigen-binding fragment thereof, of embodiment 87 or 88, wherein:
   the Chain A heavy chain comprises an amino acid sequence that is at least 90%, 91%, 92%, 93%, 94%, 95%, 96%, 97%, 98%, 99%, or 100% identical to the amino acid sequence of SEQ ID NO: 295,
   the Chain A light chain comprises an amino acid sequence that is at least 90%, 91%, 92%, 93%, 94%, 95%, 96%, 97%, 98%, 99%, or 100% identical to the amino acid sequence of SEQ ID NO: 206, and
   Chain B comprises an amino acid sequence that is at least 90%, 91%, 92%, 93%, 94%, 95%, 96%, 97%, 98%, 99%, or 100% identical to the amino acid sequence of SEQ ID NO: 218.
90. A bispecific antibody, or a bispecific antigen-binding fragment thereof, comprising a Chain A and a Chain B,
   wherein Chain A comprises a stapled single chain fragment variable (spFv) region that immunospecifically binds to CD3,
   wherein Chain B comprises a heavy chain comprising a first mutant calreticulin (CALRmut) heavy binding fragment and a second CALRmut heavy binding fragment, a first light chain comprising a first CALRmut light binding fragment, and a second light chain comprising a second CALRmut light binding fragment,
   wherein the first CALRmut heavy binding fragment is associated with the first CALRmut light binding fragment to form a first CALRmut antigen binding site,
   wherein the second CALRmut heavy binding fragment is associated with the second CALRmut light binding fragment to form a second CALRmut antigen binding site,
   wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
   wherein the Chain B first CALRmut heavy binding fragment comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
   wherein the Chain B second CALRmut heavy binding fragment comprises a heavy CDR1 comprising the amino acid sequence of SEQ ID NO: 273, a heavy CDR2 comprising the amino acid sequence of SEQ ID NO: 274, and a heavy CDR3 comprising the amino acid sequence of SEQ ID NO: 275,
   wherein the Chain B first CALRmut light binding fragment comprises a light CDR1 comprising the amino acid sequence of SEQ ID NO: 70, a heavy CDR2 comprising the amino acid sequence of SEQ ID NO: 71, and a heavy CDR3 comprising the amino acid sequence of SEQ ID NO: 72, and
   wherein the Chain B second CALRmut light binding fragment comprises a light CDR1 comprising the amino acid sequence of SEQ ID NO: 70, a heavy CDR2 comprising the amino acid sequence of SEQ ID NO: 71, and a heavy CDR3 comprising the amino acid sequence of SEQ ID NO: 72.
91. The bispecific antibody, or bispecific antigen-binding fragment thereof, of embodiment 90, wherein:
   the Chain A spFv region comprises a VL comprising an amino acid sequence of SEQ ID NO: 217, and a VH comprising an amino acid sequence of SEQ ID NO: 213,
   the Chain B first CALRmut heavy binding fragment comprises a VH comprising an amino acid sequence of SEQ ID NO: 282,
   the Chain B second CALRmut heavy binding fragment comprises a VH comprising an amino acid sequence of SEQ ID NO: 282,
   the Chain B first CALRmut light binding fragment comprises a VL comprising an amino acid sequence of SEQ ID NO: 284, and
   the Chain B second CALRmut light binding fragment comprises a VL comprising an amino acid sequence of SEQ ID NO: 284.
92. The bispecific antibody, or bispecific antigen-binding fragment thereof, of embodiment 90 or 91, wherein:
   Chain A comprises an amino acid sequence that is at least 90%, 91%, 92%, 93%, 94%, 95%, 96%, 97%, 98%, 99%, or 100% identical to the amino acid sequence of SEQ ID NO: 218,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 298,
   the Chain B first light chain comprises an amino acid sequence that is at least 90%, 91%, 92%, 93%, 94%, 95%, 96%, 97%, 98%, 99%, or 100% identical to the amino acid sequence of SEQ ID NO: 291, and
   the Chain B second light chain comprises an amino acid sequence that is at least 90%, 91%, 92%, 93%, 94%, 95%, 96%, 97%, 98%, 99%, or 100% identical to the amino acid sequence of SEQ ID NO: 291.
93. A bispecific antibody, or a bispecific antigen-binding fragment thereof, comprising a Chain A and a Chain B,
   wherein Chain A comprises a stapled single chain fragment variable (spFv) region that immunospecifically binds to CD3,
   wherein Chain B comprises a heavy chain comprising a first mutant calreticulin (CALRmut) heavy binding fragment and a second CALRmut heavy binding fragment, a first light chain comprising a first CALRmut light binding fragment, and a second light chain comprising a second CALRmut light binding fragment,
   wherein the first CALRmut heavy binding fragment is associated with the first CALRmut light binding fragment to form a first CALRmut antigen binding site,
   wherein the second CALRmut heavy binding fragment is associated with the second CALRmut light binding fragment to form a second CALRmut antigen binding site,
   wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 210, a heavy CDR2 comprising the amino acid sequence of SEQ ID NO: 211, and a heavy CDR3 comprising the amino acid sequence of SEQ ID NO: 212,
   wherein the Chain B first CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
   wherein the Chain B second CALRmut heavy binding fragment comprises a heavy CDR1 comprising the amino acid sequence of SEQ ID NO: 200, a heavy CDR2 comprising the amino acid sequence of SEQ ID NO: 201, and a heavy CDR3 comprising the amino acid sequence of SEQ ID NO: 202,
   wherein the Chain B first CALRmut light binding fragment comprises a light CDR1 comprising the amino acid sequence of SEQ ID NO: 33, a heavy CDR2 comprising the amino acid sequence of SEQ ID NO: 23, and a heavy CDR3 comprising the amino acid sequence of SEQ ID NO: 34, and
   wherein the Chain B second CALRmut light binding fragment comprises a light CDR1 comprising the amino acid sequence of SEQ ID NO: 33, a heavy CDR2 comprising the amino acid sequence of SEQ ID NO: 23, and a heavy CDR3 comprising the amino acid sequence of SEQ ID NO: 34.
94. The bispecific antibody, or bispecific antigen-binding fragment thereof, of embodiment 93, wherein:
   the Chain A spFv region comprises a VL comprising an amino acid sequence of SEQ ID NO: 217, and a V_{H} comprising an amino acid sequence of SEQ ID NO: 213,
   the Chain B first CALRmut heavy binding fragment comprises a V_{H} comprising an amino acid sequence of SEQ ID NO: 203,
   the Chain B second CALRmut heavy binding fragment comprises a V_{H} comprising an amino acid sequence of SEQ ID NO: 203,
   the Chain B first CALRmut light binding fragment comprises a VL comprising an amino acid sequence of SEQ ID NO: 205, and
   the Chain B second CALRmut light binding fragment comprises a VL comprising an amino acid sequence of SEQ ID NO: 205.
95. The bispecific antibody, or bispecific antigen-binding fragment thereof, of embodiment 93 or 94, wherein:
   Chain A comprises an amino acid sequence that is at least 90%, 91%, 92%, 93%, 94%, 95%, 96%, 97%, 98%, 99%, or 100% identical to the amino acid sequence of SEQ ID NO: 218,
   the Chain B heavy chain comprises an amino acid sequence that is at least 90%, 91%, 92%, 93%, 94%, 95%, 96%, 97%, 98%, 99%, or 100% identical to the amino acid sequence of SEQ ID NO: 296,
   the Chain B first light chain comprises an amino acid sequence that is at least 90%, 91%, 92%, 93%, 94%, 95%, 96%, 97%, 98%, 99%, or 100% identical to the amino acid sequence of SEQ ID NO: 206, and
   the Chain B second light chain comprises an amino acid sequence that is at least 90%, 91%, 92%, 93%, 94%, 95%, 96%, 97%, 98%, 99%, or 100% identical to the amino acid sequence of SEQ ID NO: 206.
96. The bispecific antibody, or bispecific antigen-binding fragment thereof, of any one of embodiments 70 to 95, wherein the CALRmut comprises an amino acid sequence of SEQ ID NO: 1 or SEQ ID NO: 2.
97. A cell expressing the bispecific antibody, or bispecific antigen-binding fragment thereof, of any one of embodiments 70 to 95.
98. The cell of embodiment 97, wherein the cell is a hybridoma.
99. The cell of embodiment 97 or 98, wherein the bispecific antibody, or bispecific antigen-binding fragment thereof, is recombinantly produced.
100. At least one synthetic polynucleotide encoding the Chain A and/or the Chain B of the bispecific antibody, or bispecific antigen-binding fragment thereof, of any one of embodiments 34 to 52.
101. A vector comprising the synthetic polynucleotide of embodiment 100
102. A pharmaceutical composition comprising bispecific antibody, or bispecific antigen-binding fragment thereof, of any one of embodiments 70 to 95 and a pharmaceutically acceptable carrier.
103. A method for generating the bispecific antibody, or bispecific antigen-binding fragment thereof, of any one of embodiments 70 to 95 by culturing the cell of any one of embodiments 97 to 99.
104. A method of redirecting a T cell to a cell expressing a mutant calreticulin, the method comprising administering to a subject in need thereof a therapeutically effective amount of the bispecific antibody, or bispecific antigen-binding fragment thereof, of any one of embodiments 70 to 95 to redirect a T cell to the cell expressing a mutant calreticulin.
105. A method of inhibiting the growth or proliferation of a myeloproliferative neoplasm (MPN), the method comprising administering to a subject in need thereof a therapeutically effective amount of the bispecific antibody, or bispecific antigen-binding fragment thereof, of any one of embodiments 70 to 95 to inhibit the growth or proliferation of the MPN.
106. A method of treating a MPN, the method comprising administering to a subject in need thereof the bispecific antibody, or bispecific antigen-binding fragment thereof, of any one of embodiments 70 to 95, to the subject for a time sufficient to treat the cancer.
107. The method of embodiment 105 or 106 wherein the MPN is characterized by the presence of a mutant calreticulin.
108. The method of any one of embodiments 105 to 107, wherein the MPN is selected from the group consisting of chronic myelogenous leukemia, polycythemia vera, primary myelofibrosis, essential thrombocythemia, chronic neutrophilic leukemia, and chronic eosinophilic leukemia.
109. The method of any one of embodiments 104 to 108, wherein soluble mutant calreticulin is present in the blood of the subject.
110. A kit comprising the bispecific antibody, or bispecific antigen-binding fragment thereof, of any one of embodiments 70 to 95 and packaging for the same.
111. A kit comprising the polynucleotide of embodiment 100 and packaging for the same.
112. A kit comprising the bispecific antibody, or bispecific antigen-binding fragment thereof, of any one of embodiments 70 to 95, the polynucleotide of embodiment 100, and packaging for the same.

### EXAMPLES

### Example 1: Attempted Generation of CALRmut x CD3 Bispecific Antibodies

Wildtype (AJ) mice (Jackson Laboratory) were immunized subcutaneously (SC)/intradermally (each ear, hock, axillary area, and both sides at the base of the tail) with (1) a tandem repeat peptide, (2) soluble MPL/CALRmut complex, or (3) DNA-CALRmut/DNA-membrane bound MPL/CALRmut complex. Briefly, on Days 0, 14, 28, 42, 77, mice received SC/intradermal injections of recombinant peptides loaded onto nanoparticles. Each injection contained 40 μg of peptide. Immune sera samples were collected on days 0, 28, 77 and tested for binding to peptides via enzyme-linked immunosorbent assay (ELISA), enabling identification of responding mice. On day 89, selected mice were given a final boost of peptides+nanoparticles+Cd40 4 days prior to hybridoma fusion. Spleen, lymph nodes collected, mandibular, accessory mandibular, superficial parotid, proper axillary, accessory axillary, subiliac, sciatic, popliteal, gastric, pancreaticoduodenal, jejunal, and medial iliac (see LN collection diagram) cells were harvested and placed in PBS. Primary screening was done on OCI-M2 parental cells and OCI-M2 cells expressing MPL and CALR mutant. Confirmatory screening was done on OCI-M2 parental cells and OCI-M2 cells expressing MPL and CALR mutant or CALR wild-type proteins.

Only campaigns using tandem repeat peptides yielded a confirmed set of CALR mutant specific binders as determined by cell-binding assays. These binders were then reformatted into CD3-redirecting bispecific antibodies. None of the reformatted bispecific antibodies showed significant cytotoxic activity with cell lines expressing CALRmut. Therefore, none of the anti-CALR mutant binders from this immunization campaign moved forward in the screening process.

### Example 2: Generation of Anti-CALRmut Monoclonal Antibodies

### A. Immunization of Animals

A new anti-CALRmut antibody immunization campaign was undertaken to identify binders that would exhibit suitable cytotoxic activity when reformatted into CD3-redirecting bispecific antibodies. For this immunization, 2 New Zealand White Rabbits were selected based on low background signal of the pre-immune serum (tested in SDS page/Western blot analysis with cellular lysates from a CALRmut-expressing cell line). The rabbits were immunized with a synthesized 22 amino acid CALRmut-specific peptide (coupled to keyhole limpet hemocyanin (KLH) at the N-terminus). Four subcutaneous injections were applied, and the immune serum was tested for reactivity with the 22aa CALRmut peptide by ELISA. The rabbit with the best reactivity to the peptide was chosen for splenectomy and total splenocyte isolation after receiving an additional hyper boost injection. The splenocytes of this animal were used for RNA isolation.

The 22 amino acid CALRmut-specific peptide is KMSPARPRTSCREACLQGWTEA (SEQ ID NO: 1) with an acetylation of the terminal amino-group and a C-terminal —OH group. The rabbits were injected subcutaneously with 200 g peptide/injection/animal (concentration: 0.57 mg/mL; total volume: 4.80 mL+/−0.35 mL) according to the immunization schedule in Table 1.

TABLE 1
Rabbit Immunization schedule

| | |
|---|---|
| Day 0 | Pre-immune bleed |
| | First immunization |
| Day 21 | Boost 1 |
| Day 31 | Small bleed |
| Day 49 | Boost 2 |
| Day 59 | Large bleed |
| | Serum test for rabbit selection |
| Day 77 | Boost 3 |
| Day 109 | Hyper boost |
| Day 113 | Final bleed |
| | Splenectomy |
| | Total splenocyte isolation |

### B. Phage Display Screening

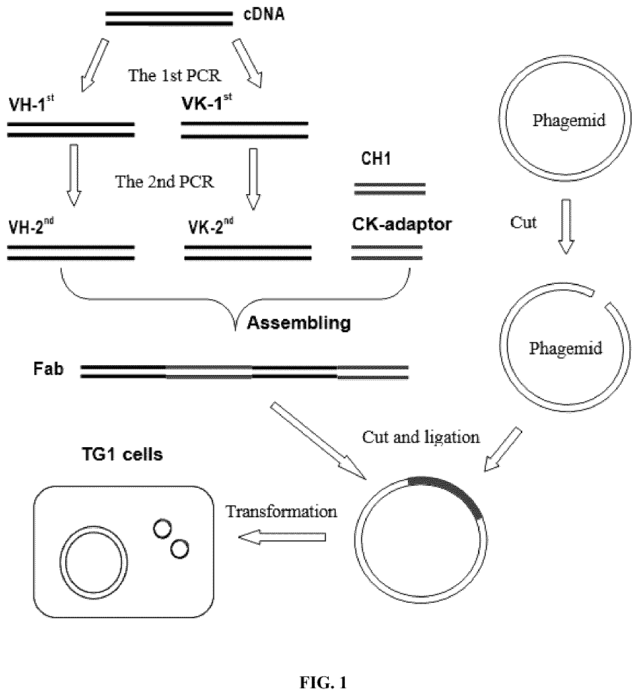

First, RNA was isolated from the splenocytes derived from the rabbit immunization and then was used as template for the construction of a rabbit/human chimeric Fab library. The rabbit VK and VH genes were PCR amplified as single bands, while human CK-adapter and human CH1 were PCR amplified with a pre-made vector template. To facilitate the assembling of Fab fragments, the 2nd round of PCR was conducted to add overlapping regions flanking VK and VH. Then with a three-fragments-overlap PCR, the whole Fab fragments were prepared. The pre-made phagemid and Fab fragments were gel purified before being subjected to enzyme cutting reaction separately. After that, they were gel-purified and quantified by Nanodrop. Next, high-performance ligation with T4 DNA ligase was performed and the ligation mix was desalted before being electro-transformed into TG1 cells, to construct the end library. The end library of 1.5×10⁹ diversity was constructed with 8/10 positive insertion. An overview of this process is shown in FIG. 1.

Next, recombinant CALRdel52 protein (SEQ ID NO: 5) and the 22 amino acid CALRmut peptide (SEQ ID NO: 1) were used for selecting specific binders from the immune rabbit/human Fab library prepared above. 40 clones were picked, and binding to the CALRdel53 target protein was confirmed in a QC phage ELISA and shown in Table 2. By DNA sequencing, 15 unique Fabs were identified and specific binding to the CALRmut target peptide was confirmed by phage ELISA and by QC ELISA after soluble expression of Fab-AP (Fab-conjugated to Alkaline Phosphatase). The various CDR, VH and VL amino acid sequences of all 15 clones are listed in Table 3.

TABLE 2
QC monoclonal phage ELISA (3-E-1) binding data

| Clone No. | Coating: CALR | No coating |
|---|---|---|
| 1 | 2.920 | 0.122 |
| 2 | 2.874 | 0.113 |
| 3 | 2.823 | 0.119 |
| 4 | 2.544 | 0.126 |
| 5 | 2.565 | 0.120 |
| 6 | 2.736 | 0.133 |
| 7 | 2.822 | 0.155 |
| 8 | 0.696 | 0.145 |
| 9 | 0.243 | 0.150 |
| 10 | 2.179 | 0.107 |
| 11 | 2.847 | 0.124 |
| 12 | 2.935 | 0.135 |
| 13 | 2.521 | 0.123 |
| 14 | 2.778 | 0.106 |
| 15 | 1.982 | 0.146 |
| 16 | 2.942 | 0.169 |
| 17 | 2.232 | 0.120 |
| 18 | 1.910 | 0.140 |
| 19 | 2.739 | 0.120 |
| 20 | 2.687 | 0.123 |
| 21 | 2.693 | 0.152 |
| 22 | 1.831 | 0.175 |
| 23 | 2.908 | 0.144 |
| 24 | 1.957 | 0.170 |
| 25 | 1.115 | 0.136 |
| 26 | 1.435 | 0.159 |
| 27 | 2.995 | 0.130 |
| 28 | 2.202 | 0.250 |
| 29 | 2.700 | 0.103 |
| 30 | 1.572 | 0.117 |
| 31 | 1.966 | 0.110 |
| 32 | 2.596 | 0.099 |
| 33 | 2.383 | 0.105 |
| 34 | 2.810 | 0.169 |
| 35 | 2.272 | 0.145 |
| 36 | 2.068 | 0.099 |
| 37 | 2.300 | 0.132 |
| 38 | 2.906 | 0.127 |
| 39 | 2.959 | 0.142 |
| 40 | 2.175 | 0.128 |
| M13KO7 | 0.225 | 0.188 |
| 1% M-PBS | 0.174 | 0.074 |

Direct coating of M13KO7: 2.860

TABLE 3
CDR, VH, and VL amino acid sequences for the 15 selected clones

| SEQ | | |
|---|---|---|
| ID NO. | Description | Sequence |
| 10 | Clone 1 Heavy CDR1 | IYGMS |
| 11 | Clone 1 Heavy CDR2 | IIYIEGITYYANWAKGRFT |
| 12 | Clone 1 Heavy CDR3 | GVGRI |
| 13 | Clone 1 VH | QEQLKESGGRLVTPGTPLTLTCAVSGFSLDIYGMS |
| | | WVRQAPGKGLEWIGIIYIEGITYYANWAKGRFTISK |
| | | TSSTTLELKMTSLTTEDTAMYFCVAGVGRIWGPGT |
| | | LVTVSS |
| 14 | Clone 1 Light CDR1 | QASQSLYNNNNLA |
| 15 | Clone 1 Light CDR2 | PASTLAS |
| 16 | Clone 1 Light CDR3 | LGEFSCSSADCYA |
| 17 | Clone 1 VL | YVMMTQTPSSVSAAVGGTVTINCQASQSLYNNNN |
| | | LAWFQQKPGQPPKRLIYPASTLASGVSSRFKGSGSG |
| | | TQFTLTISDVQRDDAATYFCLGEFSCSSADCYAFGG |
| | | GTEVVVK |
| 81 | Clone 2 Heavy CDR1 | NNAIS |
| 19 | Clone 2 Heavy CDR2 | VIGNTGTTYYASWAKGRFT |
| 20 | Clone 2 Heavy CDR3 | GPPSYSNSVKNI |
| 21 | Clone 2 VH | QSVEESGGRLVTPGTPLTLTCTVSGIDLSNNAISWV |
| | | RQAPGKGLEYIGVIGNTGTTYYASWAKGRFTISKTS |
| | | STTVDLKMTSLTASDTATYFCVRGPPSYSNSVKNI |
| | | WGPGTLVTVSS |
| 22 | Clone 2 Light CDR1 | QSSQSVYDKNWLS |
| 23 | Clone 2 Light CDR2 | DASKLES |
| 24 | Clone 2 Light CDR3 | AGGFTGNVYA |
| 25 | Clone 2 VL | DPMLTQTPSSVSAAVGGTVTIGCQSSQSVYDKNWL |
| | | SWYQQKPGQPPSRLIYDASKLESGVPSRFSGSGSGT |
| | | QFTLTIGGVQCDDAATYYCAGGFTGNVYAFGGGT |
| | | KLEIK |
| 26 | Clone 3 Heavy CDR1 | NNAVS |
| 27 | Clone 3 Heavy CDR2 | VIGNTGSTYYANWAKGRFT |
| 28 | Clone 3 Heavy CDR3 | VRGPPSYSNSVKNI |
| 29 | Clone 3 VH | QEQLKESGGRLVAPGTPLRLTCRVSGIDLSNNAVS |
| | | WVRQAPGKGLEYIGVIGNTGSTYYANWAKGRFTIS |
| | | KTSSTTVDLKMTSLTADDTATYFCVRGPPSYSNSV |
| | | KNIWGPGTLVTVSS |
| 22 | Clone 3 Light CDR1 | QSSQSVYDKNWLS |
| 23 | Clone 3 Light CDR2 | DASKLES |
| 24 | Clone 3 Light CDR3 | AGGFTGNVYA |
| 30 | Clone 3 VL | DPMLTQTPSSVSAAVGGTVTIGCQSSQSVYDKNWL |
| | | SWYQQKPGQPPSRLIYDASKLESGVPSRFSGSGSGT |
| | | QFTLTISGVQCDDAATYYCAGGFTGNVYAFGGGTE |
| | | VVVK |
| 26 | Clone 4 Heavy CDR1 | NNAIS |
| 31 | Clone 4 Heavy CDR2 | VIGNTGDTYYASWAKGRFT |
| 20 | Clone 4 Heavy CDR3 | GPPSYSNSVKNI |
| 32 | Clone 4 VH | QEQLEESGGRLVTPGTPLTLTCKVSGIDLSNNAISW |
| | | VRQAPGKGLEYIGVIGNTGDTYYASWAKGRFTISK |
| | | TSSTTVDLKMTSLTTEDTATYFCVRGPPSYSNSVKN |
| | | IWGPGTLVTISS |
| 33 | Clone 4 Light CDR1 | QSSQSVYNNNWLS |
| 23 | Clone 4 Light CDR2 | DASKLES |
| 34 | Clone 4 Light CDR3 | AGGFTGNVYT |
| 35 | Clone 4 VL | DPMLTQTPSSVSAAVGGTVTINCQSSQSVYNNNWL |
| | | SWLQQKPGQPPNRLIYDASKLESGVPSRFSGSGSGT |
| | | QFTLTISGVQRDDAATYYCAGGFTGNVYTFGGGTE |
| | | VVVK |
| 26 | Clone 5 Heavy CDR1 | NNAVS |
| 19 | Clone 5 Heavy CDR2 | VIGNTGTTYYASWAKGRFT |
| 20 | Clone 5 Heavy CDR3 | GPPSYSNSVKNI |
| 36 | Clone 5 VH | QEQLKESGGRLVAPGTPLRLTCTVSGIDLSNNAVS |
| | | WVRQAPGKGLEYIGVIGNTGTTYYASWAKGRFTIS |
| | | KTTSTTVDLKMTSLTTDDTATYFCVRGPPSYSNSV |
| | | KNIWGPGTLVTVSS |
| 22 | Clone 5 Light CDR1 | QSSQSVYDKNWLS |
| 23 | Clone 5 Light CDR2 | DASKLES |
| 37 | Clone 5 Light CDR3 | AGGFTGNVYG |
| 38 | Clone 5 VL | DPVLTQTPSSVSAAVGGTVTIGCQSSQSVYDKNWL |
| | | SWYQQKPGQPPSRLIYDASKLESGVPSRFSGSGSGT |
| | | QFTLTISGVQCDDAATYYCAGGFTGNVYGFGGGTE |
| | | VVVK |
| 26 | Clone 7 Heavy CDR1 | NNAVS |
| 39 | Clone 7 Heavy CDR2 | VIGNTGTTYYANWAKGRFT |
| 40 | Clone 7 Heavy CDR3 | GPPSYSNSVRNI |
| 41 | Clone 7 VH | QQLEESGGRLVAPGTPLRLTCTVSGIDLSNNAVSW |
| | | VRQAPGKGLEYIGVIGNTGTTYYANWAKGRFTISK |
| | | TSSTTVDLKMTSLTTDDTATYFCVRGPPSYSNSVRN |
| | | IWGPGTLVTVSS |
| 42 | Clone 7 Light CDR1 | QSSQSVYDNNWLS |
| 43 | Clone 7 Light CDR2 | DASKLAS |
| 44 | Clone 7 Light CDR3 | AGGYTGNVYS |
| 45 | Clone 7 VL | DPMLTQTPSSVSAAVGGTVTIGCQSSQSVYDNNWL |
| | | SWYQQKPGQPPNRLIYDASKLASGVPSRFSGSGSGT |
| | | QFTLTISGVKCDDAATYYCAGGYTGNVYSFGGGT |
| | | MVEIK |
| 46 | Clone 10 Heavy CDR1 | TVSGIDLSNNAIS |
| 31 | Clone 10 Heavy CDR2 | VIGNTGDTYYASWAKGRFT |
| 20 | Clone 10 Heavy CDR3 | GPPSYSNSVKNI |
| 47 | Clone 10 VH | QSVEESGGRLVTPGTPLTLTCTVSGIDLSNNAISWV |
| | | RQAPGKGLEYIGVIGNTGDTYYASWAKGRFTISKTS |
| | | STTVDLKMTSLTTEDTATYFCVRGPPSYSNSVKNIW |
| | | GPGTLVTISS |
| 22 | Clone 10 Light CDR1 | QSSQSVYDKNWLS |
| 23 | Clone 10 Light CDR2 | DASKLES |
| 24 | Clone 10 Light CDR3 | AGGFTGNVYA |
| 48 | Clone 10 VL | DPMLTQTPSSVSAAVGGTVTIGCQSSQSVYDKNWL |
| | | SWYQQKPGQPPSRLIYDASKLESGVPSRFSGSGSGT |
| | | QFTLTISGVQCDDVATYYCAGGFTGNVYAFGGGTE |
| | | VVVK |
| 49 | Clone 11 Heavy CDR1 | TVSGIDLSNNAVS |
| 39 | Clone 11 Heavy CDR2 | VIGNTGTTYYANWAKGRFT |
| 20 | Clone 11 Heavy CDR3 | GPPSYSNSVKNI |
| 50 | Clone 11 VH | QEQLKESGGRLVAPGTPLRLTCTVSGIDLSNNAVS |
| | | WVRQAPGKGLEYIGVIGNTGTTYYANWAKGRFTIS |
| | | KTSSTTVDLKMTSLTTDDTATYFCVRGPPSYSNSVK |
| | | NIWGPGTLVTVSS |
| 51 | Clone 11 Light CDR1 | QASQSVYNNNWLT |
| 43 | Clone 11 Light CDR2 | DASKLAS |
| 52 | Clone 11 Light CDR3 | AAGWTGNVYS |
| 53 | Clone 11 VL | DPMLTQTPSPVSAAVGGTVTINCQASQSVYNNNWL |
| | | TWYQQKPGQPPNRLIYDASKLASGVPSRFSGSGFGT |
| | | QFTLTISGVQCDDAATYYCAAGWTGNVYSFGGGT |
| | | EVVVK |
| 81 | Clone 15 Heavy CDR1 | NNAIS |
| 19 | Clone 15 Heavy CDR2 | VIGNTGTTYYASWAKGRFT |
| 20 | Clone 15 Heavy CDR3 | GPPSYSNSVKNI |
| 54 | Clone 15 VH | QQLEESGGRLVTPGTPLTLTCTVSGIDLSNNAISWV |
| | | RQAPGKGLEYIGVIGNTGTTYYASWAKGRFTISKTS |
| | | STTVDLKMTSLTASDTATYFCVRGPPSYSNSVKNI |
| | | WGPGTLVTISS |
| 33 | Clone 15 Light CDR1 | QSSQSVYNNNWLS |
| 23 | Clone 15 Light CDR2 | DASKLES |
| 34 | Clone 15 Light CDR3 | AGGFTGNVYT |
| 55 | Clone 15 VL | DVVMTQTPSSVSAAVGGTVTINCQSSQSVYNNNW |
| | | LSWLQQKPGQPPNRLIYDASKLESGVPSRFSGSGSG |
| | | TQFTLTISGVQCDDAATYYCAGGFTGNVYTFGGGT |
| | | EVVVK |
| 56 | Clone 16 Heavy CDR1 | NYYMS |
| 57 | Clone 16 Heavy CDR2 | GISSRAGTWYASWAKGRFT |
| 58 | Clone 16 Heavy CDR3 | GLKI |
| 59 | Clone 16 VH | QEQLKESGGRLVTPGTPLTLTCTASGFSLSNYYMS |
| | | WVRQAPGKGLEWIGGISSRAGTWYASWAKGRFTIS |
| | | GTSTTMDLKITSPTTEDTATYFCARGLKIWGPGTLV |
| | | TVSS |
| 60 | Clone 16 Light CDR1 | ASQSVYDDNNLA |
| 15 | Clone 16 Light CDR2 | PASTLAS |
| 61 | Clone 16 Light CDR3 | QGEFSCTSADCVA |
| 62 | Clone 16 VL | DPVLTQTESPVSAAVGGTVTINCQASQSVYDDNNL |
| | | AWYQQKPGQPPKRLIYPASTLASGVSSRFSGSGSGS |
| | | QFTLTISDVQCDDAATYYCQGEFSCTSADCVAFGG |
| | | GTEVVVK |
| 63 | Clone 17 Heavy CDR1 | NNAMS |
| 39 | Clone 17 Heavy CDR2 | VIGNTGTTYYANWAKGRFT |
| 20 | Clone 17 Heavy CDR3 | GPPSYSNSVKNI |
| 64 | Clone 17 VH | QEQLEESGGRLVTPGTPLTLTCTVSGIDLSNNAMSW |
| | | VRQAPGKGLEYIGVIGNTGTTYYANWAKGRFTISK |
| | | TSSATVDLKMTSLTTDDTATYFCVRGPPSYSNSVK |
| | | NIWGPGTLVTVSS |
| 22 | Clone 17 Light CDR1 | QSSQSVYDKNWLS |
| 23 | Clone 17 Light CDR2 | DASKLES |
| 34 | Clone 17 Light CDR3 | AGGFTGNVYT |
| 65 | Clone 17 VL | DVMLTQTPSSVSAAVGGTVTINCQSSQSVYDKNWL |
| | | SWLQQKPGQPPSRLIYDASKLESGVPSRFSGSGSGT |
| | | QFTLTISGVQCDDAATYYCAGGFTGNVYTFGGGTK |
| | | VVVK |
| 66 | Clone 18 Heavy CDR1 | SYAMS |
| 67 | Clone 18 Heavy CDR2 | VIDSGGTTYYATWAKS |
| 68 | Clone 18 Heavy CDR3 | ESYAGYDGYGHPIPYGMDP |
| 69 | Clone 18 VH | QSVEESGGRLVTPGTPLTITCTVSGFSLSSYAMSWV |
| | | RQAPGKGLEWIGVIDSGGTTYYATWAKSRFTISKT |
| | | QTKVALKITSPTTEDTATYFCARESYAGYDGYGHPI |
| | | PYGMDPWGPGTLVTVSS |
| 70 | Clone 18 Light CDR1 | QASQSLYNNKNLA |
| 71 | Clone 18 Light CDR2 | YASALAS |
| 72 | Clone 18 Light CDR3 | QGEFNCNSADCNS |
| 73 | Clone 18 VL | DVVMTQTPSSVSAAVGGTVTINCQASQSLYNNKNL |
| | | AWYQQKPGQPPKLLIYYASALASGVSSRFKGSGSG |
| | | TQFTLTISGVQCDDAATYYCQGEFNCNSADCNSFG |
| | | GGTEVVVK |
| 46 | Clone 20 Heavy CDR1 | TVSGIDLSNNAIS |
| 31 | Clone 20 Heavy CDR2 | VIGNTGDTYYASWAKGRFT |
| 28 | Clone 20 Heavy CDR3 | VRGPPSYSNSVKNI |
| 75 | Clone 20 VH | QSLEESGGRLVTPGTPLTLTCTVSGIDLSNNAISWV |
| | | RQAPGKGLEYIGVIGNTGDTYYASWAKGRFTISKTS |
| | | STTVDLKMTSLTTEDTATYFCVRGPPSYSNSVKNIW |
| | | GPGTLVTISS |
| 22 | Clone 20 Light CDR1 | QSSQSVYDKNWLS |
| 23 | Clone 20 Light CDR2 | DASKLES |
| 24 | Clone 20 Light CDR3 | AGGFTGNVYA |
| 76 | Clone 20 VL | DPMLTQTPSSVSAAVGGTVTIGCQSSQSVYDKNWL |
| | | SWYQQKPGQPPSRLIYDASKLESGVPSRFSGSGSGT |
| | | QFTLTISGVQCDDAATYYCAGGFTGNVYAFGGGTK |
| | | LEIK |
| 26 | Clone 21 Heavy CDR1 | NNAVS |
| 39 | Clone 21 Heavy CDR2 | VIGNTGTTYYANWAKGRFT |
| 20 | Clone 21 Heavy CDR3 | GPPSYSNSVKNI |
| 77 | Clone 21 VH | QEQLKESGGRLVAPGTPLRLTCTVSGIDLSNNAVS |
| | | WVRQAPGKGLEYIGVIGNTGTTYYANWAKGRFTIS |
| | | KTSSTTVDLKMTSLTTDDTATYFCVRGPPSYSNSVK |
| | | NIWGPGTLVTISS |
| 22 | Clone 21 Light CDR1 | QSSQSVYDKNWLS |
| 23 | Clone 21 Light CDR2 | DASKLES |
| 24 | Clone 21 Light CDR3 | AGGFTGNVYA |
| 78 | Clone 21 VL | DVVMTQTPSSVSAAVGGTVTIGCQSSQSVYDKNW |
| | | LSWYQQKPGQPPSRLIYDASKLESGVPSRFSGSGSG |
| | | TQFTLTISGVQCDDAATYYCAGGFTGNVYAFGGGT |
| | | MVEIK |
| 63 | Clone 22 Heavy CDR1 | NNAMS |
| 19 | Clone 22 Heavy CDR2 | VIGNTGTTYYASWAKGRFT |
| 28 | Clone 22 Heavy CDR3 | VRGPPSYSNSVKNI |
| 79 | Clone 22 VH | QQLEESGGRLVTPGTPLTLTCTVSGIDLSNNAMSW |
| | | VRQAPGKGLEYIGVIGNTGTTYYASWAKGRFTISK |
| | | TSSTTVDLKMTSLTTEDTATYFCVRGPPSYSNSVKN |
| | | IWGPGTLVTIS |
| 33 | Clone 22 Light CDR1 | QSSQSVYNNNWLS |
| 23 | Clone 22 Light CDR2 | DASKLES |
| 34 | Clone 22 Light CDR3 | AGGFTGNVYT |
| 80 | Clone 22 VL | DPVLTQTPSSVSAAVGGTVTINCQSSQSVYNNNWL |
| | | SWLQQKPGQPPNRLIYDASKLESGVPSRFSGSGSGT |
| | | QFTLTISGVQCDDAATYYCAGGFTGNVYTFGGGTE |
| | | VVVK |

Finally, the 15 identified clones were constructed as rabbit/mouse chimeric IgGs (mouse IgG2a) and the plasmids were cloned using general cloning methods such as PCR, enzyme cutting, ligation and transformation. The DNA sequences of all the clones were validated.

### C. Screening of Antibodies by ELISA

For specificity-testing of the 15 above identified clones from the phage display, different antigens (including CALRins5, CALRdel52, CALRwt, 24aa mutant peptide, see Table 4 for full list) were used for coating the ELISA plates. A peptide alanine walking library covering the last 22aa of the mutant calreticulin protein sequence (SEQ ID NO: 1) was also used for coating the ELISA plates. Then, the binding of the identified antibodies to the different peptides was determined. The following method was used:
Dilute protein/peptide in PBS and use 50 μl of dilution mix to obtain 0.05 μg protein or peptide per well.
Overnight incubation at 4° C.
Wash 3× with 200 μL wash buffer (PBS+0.05% Tween 20).
For blocking, use 200 μL blocking solution (PBS+1-2% BSA)/well.
Incubation at 37° C. for 1-2 h.
Wash 3× with 200 μL wash buffer.
Prepare Ab-dilutions in blocking buffer and use 100 μL/well. For specificity-testing use 0.125 μg/mL as starting concentration and additional 1:3 serial dilutions, if needed. For peptide alanine walking library use 0.05-0.1 μg/mL. A mouse IgG2a isotype (Southern Biotech, 0103-01) is included as negative control. Add blocking buffer to the blank control well. Prepare all the samples and controls at least in duplicates.
Incubation at RT for 1-2 h.
Wash 3× with 200 μL wash buffer.
As secondary detection Ab, use an anti-mouse-IgG-HRP (Southern Biotech, 1030-05). Dilute Ab in blocking buffer (1:6.000-1:8000) and add 100 μL/well.
Incubation at RT for h.
Wash 3× with 200 μL wash buffer.
For development, add 100 μL/well of TMB solution and incubate 5-10 min at RT.
Add 50-100 μL stop solution/well to stop the enzymatic reaction.
Read plate at 450/620 nm on a Microplate reader.
For analysis calculate 450-620 nm values—blank value (average) and plot average values.

TABLE 4
Antigens used for Phage Display

| | | SEQ |
|---|---|---|
| Name | Sequence | ID NO: |
| 24 amino acid | RRKMSPARPRTSCREACLQGWTEA | 2 |
| CALRmut peptide | | |
| CALRins5-flag | EPAVYFKEQFLDGDGWTSRWIESKHKSDFGKFVLSSGK | 3 |
| (produced in | FYGDEEKDKGLQTSQDARFYALSASFEPFSNKGQTLVV | |
| Expi cells) | QFTVKHEQNIDCGGGYVKLFPNSLDQTDMHGDSEYNI | |
| | MFGPDICGPGTKKVHVIFNYKGKNVLINKDIRCKDDEFT | |
| | HLYTLIVRPDNTYEVKIDNSQVESGSLEDDWDFLPPKKI | |
| | KDPDASKPEDWDERAKIDDPTDSKPEDWDKPEHIPDPD | |
| | AKKPEDWDEEMDGEWEPPVIQNPEYKGEWKPRQIDNP | |
| | DYKGTWIHPEIDNPEYSPDPSIYAYDNFGVLGLDLWQV | |
| | KSGTIFDNFLITNDEAYAEEFGNETWGVTKAAEKQMKD | |
| | KQDEEQRLKEEEEDKKRKEEEEAEDNCRRMMRTKMR | |
| | MRRMRRTRRKMRRKMSPARPRTSCREACLQGWTEAD | |
| | YKDDDDK | |
| CALRwt-flag | EPAVYFKEQFLDGDGWTSRWIESKHKSDFGKFVLSSGK | 4 |
| (produced in | FYGDEEKDKGLQTSQDARFYALSASFEPFSNKGQTLVV | |
| Expi cells) | QFTVKHEQNIDCGGGYVKLFPNSLDQTDMHGDSEYNI | |
| | MFGPDICGPGTKKVHVIFNYKGKNVLINKDIRCKDDEFT | |
| | HLYTLIVRPDNTYEVKIDNSQVESGSLEDDWDFLPPKKI | |
| | KDPDASKPEDWDERAKIDDPTDSKPEDWDKPEHIPDPD | |
| | AKKPEDWDEEMDGEWEPPVIQNPEYKGEWKPRQIDNP | |
| | DYKGTWIHPEIDNPEYSPDPSIYAYDNFGVLGLDLWQV | |
| | KSGTIFDNFLITNDEAYAEEFGNETWGVTKAAEKQMKD | |
| | KQDEEQRLKEEEEDKKRKEEEEAEDKEDDEDKDEDEE | |
| | DEEDKEEDEEEDVPGQAKDELDYKDDDDK | |
| HIS-CALRdel52 | MRGSHHHHHHGMASMTGGQQMGRDLYDDDDKDRW | 5 |
| (produced in | GSELEAEPAVYFKEQFLDGDGWTSRWIESKHKSDFGKF | |
| E. coli) | VLSSGKFYGDEEKDKGLQTSQDARFYALSASFEPFSNK | |
| | GQTLVVQFTVKHEQNIDCGGGYVKLFPNSLDQTDMHG | |
| | DSEYNIMFGPDICGPGTKKVHVIFNYKGKNVLINKDIRC | |
| | KDDEFTHLYTLIVRPDNTYEVKIDNSQVESGSLEDDWD | |
| | FLPPKKIKDPDASKPEDWDERAKIDDPTDSKPEDWDKP | |
| | EHIPDPDAKKPEDWDEEMDGEWEPPVIQNPEYKGEWK | |
| | PRQIDNPDYKGTWIHPEIDNPEYSPDPSIYAYDNFGVLG | |
| | LDLWQVKSGTIFDNFLITNDEAYAEEFGNETWGVTKAA | |
| | EKQMKDKQDEEQRTRRMMRTKMRMRRMRRTRRKMR | |
| | RKMSPARPRTSCREACLQGWTEA | |
| HIS-CALRdel52 | MGSHHHHHHGSSGEPAVYFKEQFLDGDGWTSRWIESK | 6 |
| (C163S) | HKSDFGKFVLSSGKFYGDEEKDKGLQTSQDARFYALSA | |
| | SFEPFSNKGQTLVVQFTVKHEQNIDCGGGYVKLFPNSL | |
| | DQTDMHGDSEYNIMFGPDICGPGTKKVHVIFNYKGKN | |
| | VLINKDIRSKDDEFTHLYTLIVRPDNTYEVKIDNSQVES | |
| | GSLEDDWDFLPPKKIKDPDASKPEDWDERAKIDDPTDS | |
| | KPEDWDKPEHIPDPDAKKPEDWDEEMDGEWEPPVIQN | |
| | PEYKGEWKPRQIDNPDYKGTWIHPEIDNPEYSPDPSIYA | |
| | YDNFGVLGLDLWQVKSGTIFDNFLITNDEAYAEEFGNE | |
| | TWGVTKAAEKQMKDKQDEEQRTRRMMRTKMRMRRM | |
| | RRTRRKMRRKMSPARPRTSCREACLQGWTEA | |

### D. Binding of Antibodies on UT-7/TPO Cells

A megakaryocytic cell line that was CRISPR-Cas9-modified for the expression of the mutated calreticulin-MPL receptor at the endogenous locus (clone: UT-7/B5) was used as target cells to test the specific-binding of the 15 identified antibodies from the phage display. UT-7/TPO parental cells were included as a wtCALR-expres sing negative control. The method is as follows:
Transfer 300-500,000 cells into FACS tubes and wash 1× with 2 mL 1×PBS.
Optional: add 20 μL of FcgR-blocking reagent (eBioscience, 14-9161-71), mix by vortexing and incubate for 10-15 min (on ice).
Dilute mutCALR-specific unlabeled rabbit/mouse IgG2a chimeric Abs at a concentration of 0.125-0.25 μg in 100 μL of staining buffer (RPMI+10% FBS) and add to the samples, mix by vortexing and incubate for 15-20 min (on ice). A mouse IgG2a isotype (Southern Biotech, 0103-01) is included as negative control.
Wash 2× with 2 mL 1×PBS.
Use anti-mouse IgG-PE (eBioscience, 12-4010-82) as secondary detection antibody. For this, prepare the Ab-dilution in staining buffer (according to manufacturer's instruction), add 100 μL of the staining mix per sample, mix by vortexing and incubate for 15-20 min in the dark (on ice).
Wash 2× with 2 mL 1×PBS.
Fix cells by adding 100 μL of fixation buffer (4% PFA or eBioscience, 14-9161-71) and incubate for 15-20 min in the dark (on ice).
Wash 2× with 2 mL 1×PBS.
Measure cells on FACS machine (CytoFLEX Flow Cytometer, Beckman-Coulter or BD LSRFortessa™ Cell Analyzer) or store at 4° C. in the dark if it cannot be measured immediately.
Perform FACS analysis using FlowJo Software: live gating of cells (based on FSC/SSC)->singlets (FSC-W/FSC-A)->histogram overlay of mutCALR Ab staining (PE channel).

### Example 3: Production of CALRmut x CD3 Bispecific Antibodies

The 15 Fv sequences provided in Example 2 above were analyzed. Sequence alignment showed that both the VH and VL domains can be grouped into four families. Family 1 contains clone 1; Family 2 contains clone 16, and Family 3 contains clone 18. Family 4 contained a larger number of sequences and could be subdivided into 3 additional groups: 4a, 4b, and 4c. Family 4a contains clones 2, 10, and 20; Family 4b contains clones 3, 5, 7, 11, and 21; and Family 4c contains clones 5, 15, 17, and 22. One select sequence was chosen from each of Family 1 (clone 1), Family 2 (clone 16), Family 3 (clone 18), Family 4a (clone 10), Family 4b (clone 7), and Family 4c (clone 4). Additionally, clones 16 and 18 contained framework deletions that could cause misfolding of those VH domains. Therefore, modified sequences were prepared that corrected for the deletions.

These select Fab clones were paired with high affinity CD3 binding arms, medium affinity CD3 binding arms, or Null controls to generate a bispecific antibody to both mutant calreticulin (CALRmut) and CD3 with a IgG1 isotype, as shown in Table 5 below. The two sequences with a corrected framework from clones 16 and 18 are labeled as MP1016C and MP1018C respectively. The antibodies were then tested with cancer cell binding and cytotoxicity assays to determine lead candidates.

The protocol used for the generation of the bispecific antibodies was previously described in Atwell et al. J. Mol. Biol. 270: 26-35. 1997

TABLE 5
Rabbit chimeric CALRmut-Fab × CD3 molecules
for evaluation with in vitro cytotoxicity studies

| | | SEQ | |
|---|---|---|---|
| | | ID | Antibody |
| CD3-arm | Rabbit CALRmut-Fab arm | NO: | Ref. |
| CD3W245-HL- | MP1001 (adapted from Clone 1) | 100 | CALRB114 |
| scFv-high | MP1004 (adapted from Clone 4) | 101 | CALRB115 |
| affinity | MP1007 (adapted from Clone 7) | 102 | CALRB116 |
| | MP1010 (adapted from Clone 10) | 103 | CALRB117 |
| | MP1016C (adapted from Clone 16) | 104 | CALRB118 |
| | MP1016 (adapted from Clone 16) | 105 | CALRB119 |
| | MP1018C (adapted from Clone 18) | 106 | CALRB120 |
| | MP1018 (adapted from Clone 18) | 107 | CALRB121 |
| CD3W245-LH- | MP1001 (adapted from Clone 1) | 100 | CALRB74 |
| scFv-medium | MP1004 (adapted from Clone 4) | 101 | CALRB75 |
| affinity | MP1007 (adapted from Clone 7) | 102 | CALRB76 |
| | MP1010 (adapted from Clone 10) | 103 | CALRB77 |
| | MP1016C (adapted from Clone 16) | 104 | CALRB78 |
| | MP1016 (adapted from Clone 16) | 105 | CALRB79 |
| | MP1018C (adapted from Clone 18) | 106 | CALRB80 |
| | MP1018 (adapted from Clone 18) | 107 | CALRB81 |
| Null -LH scFv | MP1001 (adapted from Clone 1) | 100 | CALRB98 |
| control | MP1004 (adapted from Clone 4) | 101 | CALRB99 |
| | MP1007 (adapted from Clone 7) | 102 | CALRB100 |
| | MP1010 (adapted from Clone 10) | 103 | CALRB101 |
| | MP1016C (adapted from Clone 16) | 104 | CALRB102 |
| | MP1016 (adapted from Clone 16) | 105 | CALB103 |
| | MP1018C (adapted from Clone 18) | 106 | CALRB104 |
| | MP1018 (adapted from Clone 18) | 107 | CALRB105 |

### A. Cancer Cell-Binding Assay

Binding capacity of CALRmutxCD3 and NullxCD3 antibodies were evaluated using engineered cancer cell lines: OCI-M2 MPL CALRwt, OCI-M2 MPL CALRdel52, OCI-M2 MPL CALRins5. The cells were harvested by centrifugation for 5 minutes at 1,000 revolutions per minute (rpm) and resuspended in complete culture medium. The OCI-M2 cells were mixed with Trypan Blue and counted using the Countess Automated Cell Counter (Invitrogen). Cell suspensions were centrifuged for 5 minutes at 900 rpm and resuspended to 2×10⁶ viable cells/mL in complete culture medium. Cells were aliquoted to 96 well round bottom plates at 100 μL/well of each cell suspension and centrifugated for 5 minutes at 1,500 rpm. Fc block was added to each well at a final concentration of 2.5 μg/mL, followed by incubation for 10 minutes at room temperature (RT).

CALRmutxCD3 and NullxCD3 antibodies were made at a 10× concentration of 1000 ng/ml in complete culture medium. The final antibody concentrations tested in the assay were: 1000, 100, 10 and 0 ng/ml.

After centrifugation for 5 minutes at 1,500 rpm, 180 μL/well of complete culture medium and 20 μL/well of each antibody dilution was added to the cells followed by an incubation for 1 or 24 hours at 37° C. and 5% CO₂. After cells were washed once with Dulbecco's phosphate-buffered saline (DPBS), viability was assessed by incubating the cells in Fixable Viability Dye eFluor 450 (diluted 1:1000 in DPBS) for 30 minutes at 4° C. in the dark. Cells were washed twice with 100 μL/well of stain buffer and resuspended in 100 μL/well of secondary antibody (Alexa Fluor 647 goat anti-human IgG, 1/375 dilution in stain buffer), followed by incubation for 30 minutes at 4° C. in the dark. Cells were washed twice with 100 μL/well of stain buffer, centrifuged for 5 minutes at 1,500 rpm and resuspended in 150 μL/well of stain buffer. Samples were acquired using a FACS Lyric flow cytometer (BD Biosciences), with 35,000 events/sample collected.

Data was analyzed using FlowJo (Version 10.8.1). The population of viable single cells was gated, and geometric mean of fluorescence intensity (GeoMean) was measured. The data was graphed using GraphPadPrism (version 9.3.1).

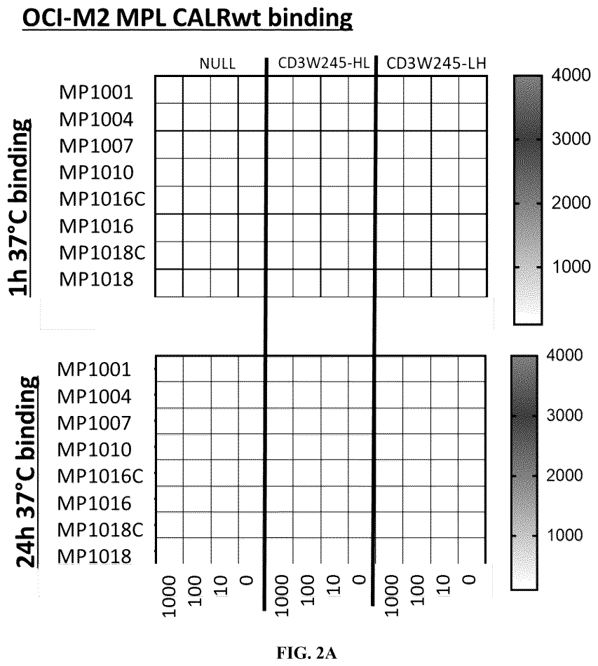

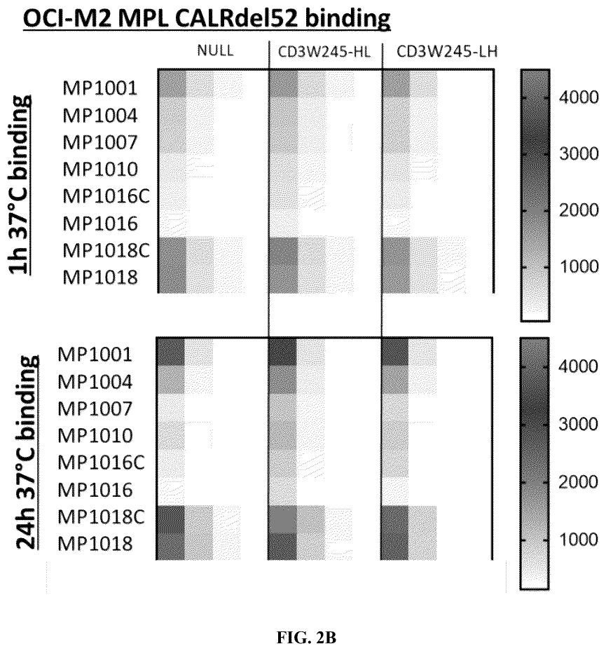

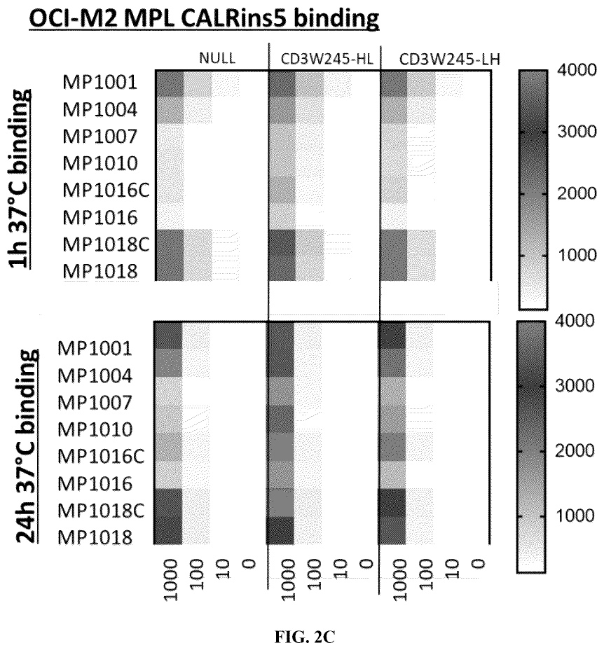

All tested CALRmutxCD3 antibodies do not bind to target negative cell line (FIG. 2A, binding to OCI-M2 MPL CALRwt). CALRmutxCD3 bind to CALRmut-expressing cells. MP1001, MP1004 and MP10018, MP1018C showed the strongest binding to CALRmut-positive cells (FIG. 2B, binding to OCI-M2 MPL CALRdel52; and FIG. 2C, binding to OCI-M2 MPL CALRins5), especially after 24-hours of incubation.

### B. Cytotoxicity Assay

OCI-M2 MPL CALRwt, OCI-MPL CALRdel52 and OCI-M2 MPL CALRins5, K562 MPL CALRwt, K562 MPL CALRdel52, K562 MPL CALRins5 cell lines were harvested by centrifugation at 1,000 rpm for 5 minutes and resuspended in complete culture medium. The OCI-M2 and K562 cells were counted as described previously. The cells were diluted in complete culture medium to a final concentration of 222,000 cells/mL.

Purified frozen human pan T cells from healthy donors were thawed, counted and resuspended in complete culture medium to a final concentration of 1.1×10⁶ viable cells/mL.

CALRmutxCD3 antibodies were made at a 10× concentration of 1000 ng/ml in complete culture medium. A total of eleven, 3-fold serial dilutions were made. The final antibody concentrations tested in the assay ranged from 1000 ng/ml to 10 ng/ml.

Pan T cells were diluted to achieve a 5:1 effector to target (E:T) ratio and dispensed to the wells of a 96 well U-bottom plate, followed by 90 μL/well of the target cells and 20 μL/well of diluted antibody or complete culture medium. Single wells per condition were tested for each donor. Plates were incubated at 37° C., 5% CO₂ for 48 or 72 hours.

Cells were harvested to evaluate viability of tumor cells and T-cell activation. After cells were washed once in DPBS, viability was assessed by incubating the cells in Fixable Viability Dye eFluor 780 (diluted 1:1,000 in DPBS) for 30 minutes at 4° C., followed by centrifugation at 400× gravity (g) at RT for 5 minutes. Cells were resuspended in stain cocktail (see Table 6 for K562 and Table 7 for OCI-M2) prepared in stain buffer, incubated for 30 minutes at 4° C., and washed twice with 200 μL/well of stain buffer. Cell pellets were resuspended in 150 μL/well of stain buffer and sample acquisition was performed on a FACSLyric or FACSVerse flow cytometer (BD Biosciences). Data analysis was performed using FlowJo.

TABLE 6
Flow Cytometry Panel for T-cell Redirection
Assay Using Pan T Cells and K562 cells

| | | | | Dilution |
|---|---|---|---|---|
| | | | | or volume |
| Marker/channel | Clone | Source | Catalog # | per well |
| Fixable Viability | NA | Thermo- | 65-0865-14 | 1:1,000 |
| Dye eFluor 780 | | Fisher | | |
| CD8/PerCP-Cy5.5 | RPA-T8 | BD | 560662 | 2 μL |
| | | Biosciences | | |
| CD25/BV421 | M-A251 | BD | 562442 | 1 μL |
| | | Biosciences | | |

BV, brilliant violet; CD, cluster of differentiation; NA, not applicable; PerCP-Cy5.5, peridinin-chlorophyll-protein complex - cyanine5.5.

TABLE 7
Flow Cytometry Panel for T-cell Redirection
Assay Using Pan T Cells and OCI-M2 cells

| | | | | Dilution |
|---|---|---|---|---|
| | | | | or volume |
| Marker/channel | Clone | Source | Catalog # | per well |
| Fixable Viability | NA | Thermo- | 65-0865-14 | 1:1,000 |
| Dye eFluor 780 | | Fisher | | |
| CD8/PerCP-Cy5.5 | RPA-T8 | BD | 560662 | 2 μL |
| | | Biosciences | | |
| CD69/PE-Cy7 | FN50 | BD | 561928 | 1 μL |
| | | Biosciences | | |
| CD25/BV421 | M-A251 | BD | 562442 | 1 μL |
| | | Biosciences | | |

BV, brilliant violet; CD, cluster of differentiation; NA, not applicable; PE-Cy7, phycoerythrin-cyanine 7; PerCP-Cy5.5, peridinin-chlorophyll-protein complex - cyanine5.5.

To assess cytotoxicity, the absolute count of the eGFP cancer cells was used to determine the number of viable cancer cells in each well. To measure the level of T-cell activation, the absolute percentage of CD25 (and CD69, for co-culture with K562 cells) expression for CD8⁺ T cells was used.

The percentage of cytotoxicity was calculated using the formula:

$$100 - ( ( \frac{x}{y} ) \times 100 )$$

where P1 population which includes all the events except debris, X absolute #of viable cancer cells divided by absolute #in P1 and Y C average of absolute #viable cancer cells in the untreated wells.

The data were graphed using Prism. Data from independent experiments were pooled and fitted using nonlinear regression variable slope (4 parameters) to the observed data to estimate the EC₅₀. Average of EC50 (ng/ml) values from 2 independent donors are listed in Table 8 and Table 9.

TABLE 8
Cytotoxicity EC₅₀ Values Observed for CALRmutxCD3 bispecific antibodies
in T-cell Redirection Assays Conducted with OCI-M2 MPL CALRmut
Cancer Cell Lines at 5:1 E:T Ratios After 72 hours.

| | Cytotoxicity EC50 (ng/ml) | | | | | |
|---|---|---|---|---|---|---|
| | OCI-M2 MPL | | OCI-M2 MPL | | OCI-M2 MPL | |
| | CALRdel52 | | CALRins5 | | CALRwt | |
| CALRmut | CD3W254- | CD3W254- | CD3W254- | CD3W254- | CD3W254- | CD3W254- |
| Fab | HL | LH | HL | LH | HL | LH |
| MP1001 | 16.8 | 25.3 | 23.5 | 32.2 | − | − |
| MP1004 | 50.8 | 114.6 | 43.1 | 129.4 | − | − |
| MP1007 | 80.2 | 89.7 | 39.2 | 189.1 | − | − |
| MP1010 | 59.8 | 183.2 | 48.1 | 189.4 | − | − |
| MP1016C | 505.3 | 58.1 | 13.6 | 79.2 | + | + |
| MP1016 | 504.3 | 112.3 | 22.5 | 91.1 | + | − |
| MP1018C | 9.1 | 20.5 | 14.5 | 21.2 | − | − |
| MP1018 | 7.8 | 19.6 | 12.2 | 17.1 | − | − |

TABLE 9
Cytotoxicity EC₅₀ Values Observed for CALRmutxCD3 bispecific
antibodies in T-cell Redirection Assays Conducted with K562
MPL CALRmut Cancer Cell Lines at 5:1 E:T Ratios After 72 hours.

| | Cytotoxicity EC50 (ng/ml) | | | | | |
|---|---|---|---|---|---|---|
| | K562 MPL | | K562 MPL | | K562 MPL | |
| | CALRdel52 | | CALRins5 | | CALRwt | |
| CALRmut | CD3W254- | CD3W254- | CD3W254- | CD3W254- | CD3W254- | CD3W254- |
| Fab | HL | LH | HL | LH | HL | LH |
| MP1001 | 11.2 | 52.1 | 16.3 | 30.4 | − | − |
| MP1004 | 34.8 | 192.5 | 54.1 | 115.5 | − | − |
| MP1007 | 24.3 | 63.3 | 95.3 | 106.9 | − | − |
| MP1010 | 40.3 | 194.9 | 74.1 | 414.3 | − | − |
| MP1016C | 9.9 | 39.3 | 15.7 | 64.8 | − | − |
| MP1016 | 6.4 | 44.4 | 10.4 | 41.6 | − | − |
| MP1018C | 7.7 | 40.1 | 11.9 | 43.4 | − | − |
| MP1018 | 3.8 | 45.7 | 10.9 | 50.9 | − | − |

Functional in vitro characterization confirmed that majority of CALRmutxCD3 antibodies showed selectivity to CALRmut-expressing cancer cell lines. CALRmutxCD3 with MP1016C and MP1016 as CALRmut engaging arm also showed some cytotoxicity at the top concentration tested to CALRmut-negative cells. CALRmutxCD3 with MP1001, MP1004, MP1018, and MP1018C as CALRmut-engaging arm showed the most potent activity across all tested antibodies.

### Example 4: CALRmut x CD3 Bispecific IgG Antibody Profile

CALRmut x CD3 is a bispecific human IgGA-AAS molecule against mutant calreticulin (CALRmut) and CD3. The bispecific antibody is an IgG1-based bispecific antibody that can simultaneously bind to CD3e (CD3ε; Uniprot ID: P07766) on T cells and to CALRmut on tumor cells. The antibody is referred to as C3CRB73 and its general molecular architecture is shown in FIG. 3. The antibody features mutations of L234A, L235A, and D265S (i.e., AAS) in the fragment crystallizable (Fc) constant region to abolish interaction with Fc receptors, and heterodimerization is enhanced using the knob-into-hole (KiH) mutations. The molecule comprises an anti-CD3ε spFv fused to the N terminus of the ‘knob’ Fc region (i.e., T366W). The ‘hole’ chain (i.e., T366S, L368A, Y407V) features an anti-CALRmut Fab and contains ‘RF’ mutations (i.e., H435R, Y436F) to disrupt Protein A binding of monomeric and homodimerized hole chains. This antibody was designed and selected as the lead therapeutic to target CALRmut for the treatment of myeloproliferative neoplasms (MPNs) based on in vitro and in vivo characterization.

### A. Structure

C3CRB73 was generated by co-expression of the anti-CD3ε scFv “knob” heavy chain (HC) with the anti-CALRmut Fab heavy chain containing the “hole” and RF mutations paired with its cognate light chain (LC). The anti-CD3ε variable region (v-region) was discovered by immunizing human antibody transgenic mice [Ablexis] with recombinant CD3ε protein. The original v-region from which the CD3W245 v-region was generated and called clone 065_D03 (VR000006495), identified by binding to Jurkat cells, and its v-region was featured in the monoclonal antibody (mAb) CD3B815. This v-region featured a human heavy chain variable domain paired with a mouse light chain variable domain. The LC v-region was humanized in scFv format since the v-region maintained binding to recombinant CD3ε in a single chain variable region (scFv) format. Briefly, the LC from CD3B815 was grafted onto the human IGKV1-39*01 germline and two positions (Y49K and L78V) were identified for human to mouse back mutation out of seven positions tested in a binary library. From these, only the Y49K mutation was selected based on E. coli expression and lowest in silico immunogenicity potential compared with other variants. The LC variable domain from CD3B815 also contained an NS motif at positions 92-93, which represents a potential post-translational modification (PTM)-risk motif for Asn deamidation. Through screening mutant libraries at both the N and S residues the Asn residue was mutated as N92G along with mutation of Y49K. This humanized, PTM de-risked variable region was named VR000030277 for use in scFv format. Upon addition of mutations necessary for the ‘stapling’ design, this new variable region was named VR000070964 and is featured as the anti-CD3ε binding spFv component of C3CRB73.

The rabbit monoclonal antibody anti-CALRmut v-region, MP1004, and the rabbit v-region was designated as clone MP1004 (VR000047016). The v-regions were humanized. This v-region featured a rabbit heavy chain variable domain paired with a rabbit light chain variable domain. Both v-regions were humanized into scFv format. Briefly, the VH from MP1004 was grafted onto the human IGHV3-23*01 germline and the VL from MP1004 was grafted onto the human IGKV1-39*01 germline. In the VH, seven positions were identified for human to rabbit backmutation. In the VL, five positions were identified for human to rabbit backmutation. The final pairs of VH and VL humanized frameworks were selected based on E. coli expression, binding and lowest in silico immunogenicity potential compared with other variants. The humanized variable region was designated with a molecular name CALRB249 (VR000048378). An ‘NS’ PTM motif in CDR-H3 was mitigated with an N104S mutation. The new v-region was designated as VR000061221 with a molecular name of CALRB421 and featured as the anti-CALRmut Fab component in C3CRB73.

The amino acid sequences for C3CRB73 heavy chains and light chain, as deduced from the cDNA sequence and confirmed by peptide mapping and mass spectrometry, are shown below with complementarity-determining regions (CDRs) listed according to ABM definition. The CD3 heavy chain sequence includes the stapled CD3 ‘spFv’ arm. The heterodimerization mutations (KiH, T366W in the CD3 spFv aim and T366S, L368A and Y407V in the CALRmut heavy chain) and H435R and Y436F mutations in the CALRmut heavy chain are present, as is the N104S mutation in CDR-H3 in the CALRmut heavy chain.

TABLE 10
Amino Acid Sequences for C3CRB73 CALRmut x CD3 Bispecific Antibody

| SEQ ID NO | Description | Sequence |
|---|---|---|
| 200 | CALRmut Heavy CDR1 | GIDLSNNAIS |
| | AbM | |
| 201 | CALRmut Heavy CDR2 | VIGNTGDTY |
| | AbM | |
| 202 | CALRmut Heavy CDR3 | GPPSYSSSVKNI |
| | AbM | |
| 260 | CALRmut Heavy CDR1 | GIDLSNN |
| | Chothia | |
| 261 | CALRmut Heavy CDR2 | GNTGD |
| | Chothia | |
| 202 | CALRmut Heavy CDR3 | GPPSYSSSVKNI |
| | Chothia | |
| 81 | CALRmut Heavy CDR1 | NNAIS |
| | Kabat | |
| 259 | CALRmut Heavy CDR2 | VIGNTGDTYYADSAKG |
| | Kabat | |
| 202 | CALRmut Heavy CDR3 | GPPSYSSSVKNI |
| | Kabat | |
| 262 | CALRmut Heavy CDR1 | GIDLSNNA |
| | IMGT | |
| 263 | CALRmut Heavy CDR2 | IGNTGDT |
| | IMGT | |
| 264 | CALRmut Heavy CDR3 | VRGPPSYSSSVKNI |
| | IMGT | |
| 203 | CALRmut VH | EVQLLESGGGLVQPGGSLRLSCAVSGIDLS |
| | | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSS |
| 204 | CALRmut Heavy Chain | EVQLLESGGGLVQPGGSLRLSCAVSGIDLS |
| | | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSSASTKGPSVFPLAPSSKSTSGGTAALG |
| | | CLVKDYFPEPVTVSWNSGALTSGVHTFPA |
| | | VLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTC |
| | | VVVSVSHEDPEVKFNWYVDGVEVHNAKT |
| | | KPREEQYNSTYRVVSVLTVLHQDWLNGK |
| | | EYKCKVSNKALPAPIEKTISKAKGQPREPQ |
| | | VYTLPPSREEMTKNQVSLSCAVKGFYPSDI |
| | | AVEWESNGQPENNYKTTPPVLDSDGSFFL |
| | | VSKLTVDKSRWQQGNVFSCSVMHEALHN |
| | | RFTQKSLSLSPGK |
| 33 | CALRmut Light CDR1 | QSSQSVYNNNWLS |
| | AbM | |
| 23 | CALRmut Light CDR2 | DASKLES |
| | AbM | |
| 34 | CALRmut Light CDR3 | AGGFTGNVYT |
| | AbM | |
| 33 | CALRmut Light CDR1 | QSSQSVYNNNWLS |
| | Chothia | |
| 23 | CALRmut Light CDR2 | DASKLES |
| | Chothia | |
| 34 | CALRmut Light CDR3 | AGGFTGNVYT |
| | Chothia | |
| 33 | CALRmut Light CDR1 | QSSQSVYNNNWLS |
| | Kabat | |
| 23 | CALRmut Light CDR2 | DASKLES |
| | Kabat | |
| 34 | CALRmut Light CDR3 | AGGFTGNVYT |
| | Kabat | |
| 256 | CALRmut Light CDR1 | QSSQSVYNNNW |
| | IMGT | |
| — | CALRmut Light CDR2 | DAS |
| | IMGT | |
| 34 | CALRmut Light CDR3 | AGGFTGNVYT |
| | IMGT | |
| 205 | CALRmut VL | DIQMTQSPSSVSASVGDRVTITCQSSQSVY |
| | | NNNWLSWLQQKPGKAPKRLIYDASKLES |
| | | GVPSRFSGSGSGTDFTLTISSVQPEDAATY |
| | | YCAGGFTGNVYTFGGGTKVEIK |
| 206 | CALRmut Light Chain | DIQMTQSPSSVSASVGDRVTITCQSSQSVY |
| | | NNNWLSWLQQKPGKAPKRLIYDASKLES |
| | | GVPSRFSGSGSGTDFTLTISSVQPEDAATY |
| | | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |
| 210 | CD3 spFv Heavy CDR1 | GFTFSRYNMN |
| | AbM | |
| 211 | CD3 spFv Heavy CDR2 | SISTSSNYIY |
| | AbM | |
| 212 | CD3 spFv Heavy CDR3 | GWGPFDY |
| | AbM | |
| 267 | CD3 spFv Heavy CDR1 | GFTFSRY |
| | Chothia | |
| 268 | CD3 spFv Heavy CDR2 | STSSNY |
| | Chothia | |
| 212 | CD3 spFv Heavy CDR3 | GWGPFDY |
| | Chothia | |
| 265 | CD3 spFv Heavy CDR1 | RYNMN |
| | Kabat | |
| 266 | CD3 spFv Heavy CDR2 | SISTSSNYIYYADSVKG |
| | Kabat | |
| 212 | CD3 spFv Heavy CDR3 | GWGPFDY |
| | Kabat | |
| 269 | CD3 spFv Heavy CDR1 | GFTFSRYN |
| | IMGT | |
| 270 | CD3 spFv Heavy CDR2 | ISTSSNYI |
| | IMGT | |
| 271 | CD3 spFv Heavy CDR3 | TRGWGPFDY |
| | IMGT | |
| 213 | CD3 spFv VH | EVOLVESGGGLVKPGGSLRLSCAASGFTF |
| | | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 214 | CD3 spFv Light CDR1 | RARQSIGTAIH |
| | AbM | |
| 215 | CD3 spFv Light CDR2 | YASESIS |
| | AbM | |
| 216 | CD3 spFv Light CDR3 | QQSGSWPYT |
| | AbM | |
| 214 | CD3 spFv Light CDR1 | RARQSIGTAIH |
| | Chothia | |
| 215 | CD3 spFv Light CDR2 | YASESIS |
| | Chothia | |
| 216 | CD3 spFv Light CDR3 | QQSGSWPYT |
| | Chothia | |
| 214 | CD3 spFv Light CDR1 | RARQSIGTAIH |
| | Kabat | |
| 215 | CD3 spFv Light CDR2 | YASESIS |
| | Kabat | |
| 216 | CD3 spFv Light CDR3 | QQSGSWPYT |
| | Kabat | |
| 272 | CD3 spFv Light CDR1 | QSIGTA |
| | IMGT | |
| — | CD3 spFv Light CDR2 | YAS |
| | IMGT | |
| 216 | CD3 spFv Light CDR3 | QQSGSWPYT |
| | IMGT | |
| 217 | CD3 spFv VL | DIQMTQSPSSLSASVGDRVTITCRARQSIG |
| | | TAIHWYQQKPGCAPKLLIKYASESISGVPS |
| | | RFSGSGSGTDFTLTISSLQPEDFATYYCQQ |
| | | SGSWPYTFGQGTKLEIK |
| 218 | CD3 spFv | DIQMTQSPSSLSASVGDRVTITCRARQSIG |
| | | TAIHWYQQKPGCAPKLLIKYASESISGVPS |
| | | RFSGSGSGTDFTLTISSLQPEDFATYYCQQ |
| | | SGSWPYTFGQGTKLEIKGGGSGGSGGCPP |
| | | CGGSGGEVQLVESGGGLVKPGGSLRLSCA |
| | | ASGFTFSRYNMNWVRQAPGKGLEWVSSI |
| | | STSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCL VKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |

B. In silico Immunogenicity Risk Assessment

The antibody sequences were analyzed for potential immunogenicity using the T-regulatory (Treg) adjusted scores from the EpiVax EpiMatrix in silico immunogenicity prediction program (Table 11). The EpiVax program computationally calculates the binding potential to the most common HLA molecules within each of the “supertypes”. The report provides results that are representative of >90% of human populations worldwide without the necessity of testing each haplotype individually. The EpiVax score is calculated by aggregating the EpiMatrix scores of all predicted T-cell epitopes contained within a given protein sequence and adjusting for expected T-cell epitope content and protein length. Although based on extensive in silico analysis, the EpiVax EpiMatrix score has limited experimental ability to predict immunogenicity in patients (Table 12). Results indicate that both the anti-CD3 arm v-region and the anti-CALRmut v-region have acceptable scores. Thus, C3CRB73 is not considered to present a significant risk for immunogenicity.

TABLE 11
Immunogenicity assessment

| Description | EpiVax score |
|---|---|
| CD3 spFv VL | −2.39 |
| CD3 spFv VH | −42.70 |
| CALRmut VL | −34.58 |
| CALRmut VH | −10.91 |

TABLE 12
EpiVax score criteria and interpretation
for potential immunogenicity

| EpiVax score | Immunogenicity criteria |
|---|---|
| <−20 | Ideal |
| −20 to +20 | Acceptable |
| >+20 | At risk |

### C. Protein Expression, Cell Culture, and Purification

C3CRB73 was expressed in ExpiCHO-S cells (ThermoFisher; Cat #A29127) by transient transfection with purified plasmid DNAs for the spFv chain (“HC1”, SEQ ID NO: 219) heavy chain (“HC2”, SEQ ID NO: 207), and light chain (“LC2”, SEQ ID NO: 208), using the ExpiFectamine CHO transfection kit (ThermoFisher; Cat #A29131). ExpiCHO-S cells were maintained in suspension in ExpiCHO Expression Medium (ThermoFisher; Cat #A2910001) in an orbital shaking incubator set to 37° C., 8% CO2 and 125 rpm. The cells were passaged into 4×2 L sterile, vented, non-baffled Erlenmeyer flasks (Corning 431255) with 400 mL starting culture volume per flask.

On the day of transfection (Culture Day 0), the flask cultures were between 3.87-8.99×106 viable cells/mL with a minimum viability of 98.2%. For the transient transfection, 200 μg of PBD000160518.001, 200 μg PBD000151680.002, and 400 μg PBD000127166.002 (HC1:HC2:LC2 chain ratio of 1:1:2) were diluted in 64 mL of OptiPRO medium (ThermoFisher Cat #12309019). 5.12 mL of ExpiFectamine CHO transfection reagent was diluted in another 64 mL of OptiPRO medium. The plasmid DNA plus OptiPRO medium mixture and the ExpiFectamine CHO reagent plus OptiPRO medium mixture were combined and incubated at room temperature for one minute. This mixture was then equivalently distributed across the 4×2 L flasks of cells, ˜33 mL/flask. The transfected cells were returned to the orbital shaking incubator.

After overnight incubation (Culture Day 1), 240 mL of ExpiCHO Feed and 4.8 mL ExpiFectamine CHO Enhancer were combined and then distributed equivalently across the 4 flasks. Cells were further incubated until harvest (Culture Day 7). The culture supernatant from the transiently transfected cells (5.59×10\hat{ }6 viable cells/mL, 63.4% viability) was clarified by centrifugation (15 min, 5000 rpm (5316 RCF)) followed by 0.2 μm filtration. C3CRB73 was then purified from the clarified supernatant.

The filtered cell culture supernatant was loaded onto a pre-equilibrated, (1×DPBS, pH 7.2) pre-packed 5 mL MabSelect PrismA column (Cytiva GE17549854) at 5 mL/min using an AKTA Pure25. After loading, the column was washed with 1×DPBS, pH 7.2 until UV stabilized near baseline. The protein was eluted with 0.1M sodium acetate, pH 3.45, and neutralized inline by the addition of 2.5 M Tris HCl, pH 7.5 to 15% (v/v) final volume, filtered (0.2 μm), and dialyzed into 20 mM MES, pH 5.5. The dialyzed eluate was further purified by cation exchange chromatography (CEX) using a custom Capto S ImpAct column (GE Healthcare, 2.6 cm×36 cm, CV=193 mL) pre-equilibrated into buffer A (20 mM MES, pH 6.5). The protein was loaded at 10 mL/min and then eluted from the column with a gradient of buffer B (20 mM MES, pH 6.5, 1 M NaCl: 0-5% buffer B over 5 min, 5-35% buffer B over 200 min). The peak fractions containing only monomeric protein were pooled, loaded onto a pre-equilibrated (1×DPBS, pH 7.2) prepacked 5 mL CH1-XL column (Thermo Fisher, 494346205) at 5 mL/min using an AKTA Xpress. The protein was eluted with 0.1M sodium acetate, pH 3.45, and fractions containing protein were neutralized by the addition of 2.5 M Tris HCl, pH 7.5 to 15% (v/v) final volume, combined, filtered (0.2 μm), and dialyzed into 20 mM MES, pH 5.5. The dialyzed eluate was further purified by CEX using four 4.6 mL HiScreen Capto S ImpAct columns daisy-chained together (Cytiva 17-3717-47) pre-equilibrated into buffer A (20 mM MES, pH 6.5). The protein was loaded at 1 mL/min and then eluted from the column with a gradient of buffer B (20 mM MES, pH 6.5, 1 M NaCl: 7.5-37.5% buffer B over 500 min) The peak fractions containing only monomeric protein were pooled, dialyzed into 10 mM L-Histidine, pH 6.5 and filtered (0.2 μm).

The concentration of purified protein was determined by absorbance at 280 nm on a Dropsense spectrophotometer using the calculated extinction coefficient (Table 1). The quality of the purified protein was assessed by SDS-PAGE and analytical size exclusion HPLC (Agilent HPLC system). The endotoxin level was measured using a turbidimetric LAL assay (Pyrotell®-T, Associates of Cape Cod; Falmouth, MA).

### D. Intrinsic Design Properties of Bispecific Antibody

Table 13 below provides a summary of biophysical assessments and results of C3CRB73.

TABLE 13
Intrinsic design properties of C3CRB73 CALRmut × CD3 bispecific antibody

| | | |
|---|---|---|
| mAb TMP characteristic | Result | Comments |

Binding characteristics

| SPR protein binding | K_{D} = 60.2 ± 6.01 pM | Measured for CALRW12.007 |
|---|---|---|
| human CALRdel52 | | |
| SPR protein binding | K_{D} = 33.1 ± 4.23 pM | Measured for CALRW28.004 |
| human CALRins5 | | |
| SPR protein binding | K_{D} = 214 ± 14 pM | Measured for CD3W220.002 |
| human CD3 | | |
| SPR/Kinexa protein | N/A | N/A |
| binding (Orthologs) | | |

| FcRn binding | K_{D} human FcRn = 1.9 μM | N/A |
|---|---|---|
| | K_{D} cyno FcRn = 0.9 μM | |

| | | |
|---|---|---|
| Fc receptor binding | Not determined | N/A |
| Specificity | No binding to wild-type | Measured for CALRW14.001 for |
| | CALR up to 300 nM | C3CRB7.013 |

Protein characterization

| Intact mAb (MS), release | 126260.7 | Da | N/A |
|---|---|---|---|
| Glycoform profile (MS) | G0F/G0F = 100 | | N/A |
| | G0F/G1F = 37.3 | | |
| | G1F/G1F = 9.4 | | |
| | G2F/G1F = 2.3 | | |
| | G2F/G2F = 0.7 | | |
| | ——/—— = 0 | | |
| | G0F/—— = 0 | | |
| | M5/M5 = 0 | | |
| | G0F/G0F − 2N = 0 | | |
| | G0/G0 = 0 | | |
| | G0F/M5 = 0 | | |
| | G0F/G0 = 0 | | |
| | G0F/G0F − N = 9.0 | | |
| | G0F/G0F + N = 0 | | |
| In silico N-link gly (non- | None observed | | Sequence-based prediction |
| Fc) | | | |
| O-link gly sites | None detected | | Sequence-based prediction |
| Glycation sites | 1.4% LC1 | | N/A |
| Free Cys (predicted | None observed | | Sequence-based prediction |
| unpaired Cys) | | | |
| N-terminal | None observed | | Sequence-based prediction |
| elongation/truncation | | | |
| (MS) | | | |
| Conformational stability | Tₒₙₛₑₜ = 62.9° C. | | N/A |
| | Tₘ1 = 72.7° C. | | |
| | Tₘ2 = 77.4° C. | | |
| | Tₘ3 = N/A | | |
| % Purity (2-step | 96.6% monomer | | N/A |
| purification) (AUC) | | | |
| Serum stability (SEC-FDS) | 93.1% monomer | | After 7-day incubation at 37° C. |
| Non-specific binding | No non-specific binding | | N/A |
| (SPR) | observed | | |
| Isoelectric point (cIEF) | pI = 9.16 | | N/A |
| | % acidic = 35.3 | | |
| | % basic = 6.0 | | |
| Relative hydrophobicity | Hydrophobic index = 0.53 | | N/A |
| (aHIC) | | | |

Stability at high concentration

| Concentration achieved | 166 | mg/mL | 10 mM histidine pH 6.5 |
|---|---|---|---|
| Percent recovery | 71% | | N/A |
| Viscosity | 2.6 | cP | At 100 mg/mL |
| Initial purity (SEC) | 98.5% monomer | | N/A |
| Stability over 2 weeks, 4° C. | 97.3% monomer | | At 150 mg/mL |
| (SEC) | | | |
| Stability over 2 weeks, | 96.1% monomer | | At 150 mg/mL |
| 25° C. (SEC) | | | |
| Stability over 2 weeks, | 94.7% monomer | | At 150 mg/mL |
| 40° C. (SEC) | | | |

Chemical (PTM) and physical stability

| Intact MS after forced | No changes in mass of the | N/A | |
|---|---|---|---|
| degradation | main species under stress | | |
| | conditions except Chem | | |
| | Ox stress due to | | |
| | oxidation. | | |
| Size after forced | Release = 96.5% | N/A | |
| degradation (aSEC) | Physiological = 96.9% | | |
| | pH 8.5 = 96.6% | | |
| | pH 5.0 = 96.6% | | |
| | Chem Ox = 96.0% | | |
| | Metal Ox = 96.4% | | |
| | Thermal = 96.1% | | |
| Size after forced | Rh (nm)/PD (%): | N/A | |
| degradation (DLS) | Release = 4.48/11.59 | | |
| | Physiological = | | |
| | 4.39/10.75 | | |
| | pH 8.5 = 4.86/6.41 | | |
| | pH 5.0 = 4.67/9.92 | | |
| | Chem Ox = 4.81/7.37 | | |
| | Metal Ox = 4.58/10.79 | | |
| | Thermal = 4.86/7.19 | | |
| Purity after forced | % Purity NR/R | N/A | |
| degradation (R and NR | Release = 97.45/100.0 | | |
| GXII) | Physiological = | | |
| | 97.32/100.0 | | |
| | pH 8.5 = 97.32/99.60 | | |
| | pH 5.0 = 97.86/100.0 | | |
| | Chem Ox = 97.39/100.0 | | |
| | Metal Ox = 97.30/99.92 | | |
| | Thermal = 97.47/100.0 | | |
| Binding after forced | K_{D}, M/% Active | for Flag/6xhis-CALRdel52 | |
| degradation for | Release = 6.02 × 10⁻¹¹/100 | | |
| CALRW12.007 (SPR) | Physiological = | | |
| | 1.71 × 10⁻¹¹/98 | | |
| | pH 8.5 = 2.51 × 10⁻¹⁰/104 | | |
| | pH 5.0 = 1.14 × 10⁻¹⁰/103 | | |
| | Chem Ox = 7.00 × 10⁻¹¹/94 | | |
| | Metal Ox = 6.30 × 10⁻¹¹/97 | | |
| | Thermal = 6.64 × 10⁻¹¹/98 | | |
| Binding after forced | K_{D}, M/% Active | For Avi/his-CalR-Ins5 | |
| degradation for | Release = 3.31 × 10⁻¹¹/100 | | |
| CALRW28.004 (SPR) | Physiological = | | |
| | 6.22 × 10⁻¹¹/99 | | |
| | pH 8.5 = 3.13 × 10⁻¹¹/97 | | |
| | pH 5.0 = 3.42 × 10⁻¹¹/98 | | |
| | Chem Ox = 4.08 × 10⁻¹¹/94 | | |
| | Metal Ox = 3.56 × 10⁻¹¹/94 | | |
| | Thermal = 3.57 × 10⁻¹¹/94 | | |
| Binding after forced | K_{D}, M/% Active | Measured for | CALR-00323 |
| degradation for | Release = | CD3EG-HSA-6xHis | |
| CD3W220.002 (SPR) | 2.14 × 10⁻¹⁰/100 | | |
| | Physiological = | | |
| | 3.01 × 10⁻¹⁰/96 | | |
| | pH 8.5 = | | |
| | 2.80 × 10⁻¹⁰/93 | | |
| | pH 5.0 = | | |
| | 2.31 × 10⁻¹⁰/100 | | |
| | Chem Ox = | | |
| | 3.06 × 10⁻¹⁰/95 | | |
| | Metal Ox = | | |
| | 2.72 × 10⁻¹⁰/91 | | |
| | Thermal = | | |
| | 2.25 × 10⁻¹⁰/94 | | |
| Basal oxidation level | 1.2% W225 in | N/A | E054751 |
| | HCDR3 | | |
| Met/Trp (change on | 6% W94 in LCDR3 | N/A | E054751 |
| metal or chemical | and 10.1% W225 in | | |
| oxidative stress) | HCDR3 upon Metal | | |
| | Ox stress | | |
| Basal deamidation level | 0.5% N181 in | N/A | E054751 |
| | HCDR2 | | |
| Deamidation sites | 2.1% N181 in | N/A | E054751 |
| (change on stress) | HCDR2 upon high | | |
| | pH stress | | |
| Basal isomerization | None | N/A | E054751 |
| level | | | |
| Isomerization sites | None | N/A | E054751 |
| (change on stress) | | | |
| Clipping (DP, NP in | None observed | N/A | CALR-00324 |
| 1° seq) | | | |

aHIC, analytical hydrophobic interaction chromatography; aSEC, analytical size-exclusion chromatography; AUC, analytical ultracentrifugation; CALR, calreticulin; CALRmut, mutant calreticulin; CD, cluster of differentiation; Chem, chemical; cIEF, capillary isoelectric focusing; cyno, cynomolgus monkey; Cys, cysteine; D, aspartic acid; del, deletion; DLS, dynamic light scattering; Fc, fragment crystallizable; FcRn, neonatal Fc receptor; FDS, fluorescence detection system; gly, glycosylation; HCDR, heavy-chain complementarity determining region; ins, insertion; K_{D}, equilibrium dissociation constant; LC, light chain; LCDR, light-chain complementarity determining region; mAb, monoclonal antibody; Met, methionine; MS, mass spectrometry; N, asparagine; N/A, not assessed; NP, asparagine/proline; NR GXII, non-reduced capillary electrophoresis; Ox, oxidation; P, proline; PD, polydispersity; pI, isoelectric point; PTM, post-translational modification; R GXII, reduced capillary electrophoresis; Rh, hydrodynamic radius; SEC, size-exclusion chromatography; SPR, surface plasmon resonance; Tₘ, melting temperature; TMP, target medicinal product; Tₒₙₛₑₜ, temperature of onset of melting; Trp, tryptophan.

### Example 5: Target Arm and CD3 Arm Binding Characterization of C3CRB73 CALRmut x CD3 Bispecific Antibody to CALRmut Cancer Cells

### A. Binding to CALRmut Cancer Cells

To evaluate the selective binding of C3CRB73 to CALRmut cells, pairs of the isogenic cell lines expressing MPL and CALRmut (CALRdel52 or CALRins5) were generated in the background of ELF-153 (cell line derived from MF patient that progressed to sAML, i.e., AML M7) or OCI-M2 (cell line derived from erythroleukemia patient, i.e., AML M6) cells lines. Expression of MPL and CALRmut were confirmed on the cell surface of these cells as shown in Table 14.

TABLE 14
Cell Surface Expression of MPL and CALRmut in cellular models

| Cell | | ELF-153 | ELF-153 | OCI-M2 | OCI-M2 | OCI-M2 |
|---|---|---|---|---|---|---|
| surface | ELF-153 | MPL | MPL | MPL | MPL | MPL |
| level | MPL | CALRdel52 | CALRins5 | CALRwt | CALRdel52 | CALRins5 |
| CALRmut | 3^{a} | 807 | 2,920 | 109^{a} | 3,254 | 17,304 |
| MPL | 2,791 | 4,913 | 2,728 | 8,587 | 6,959 | 28,975 |

CALR, calreticulin; CALRmut, mutant calreticulin; del, deletion; ins, insertion; MPL, thrombopoietin receptor; wt, wild type.

Cell surface levels of CALRmut and MPL expressed as ΔGeoMean is shown for 6 hematologic tumor cell lines engineered to express CALRmut and MPL. The experiment was performed once, and single points were tested for each condition.^{a} Values considered as background, cells were considered negative for expression if the value was less than 2× the value of the isotype control.

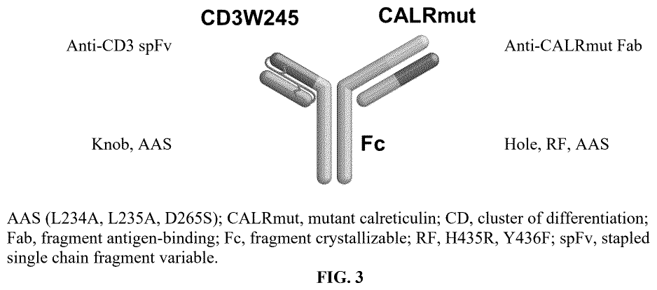

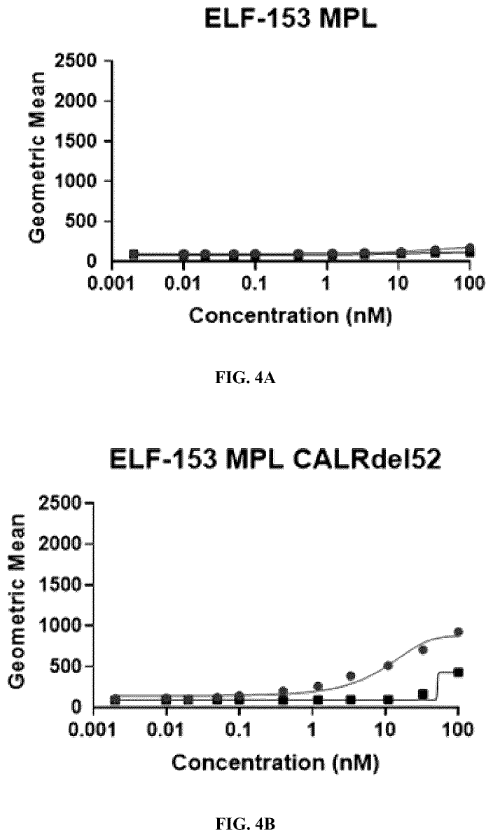

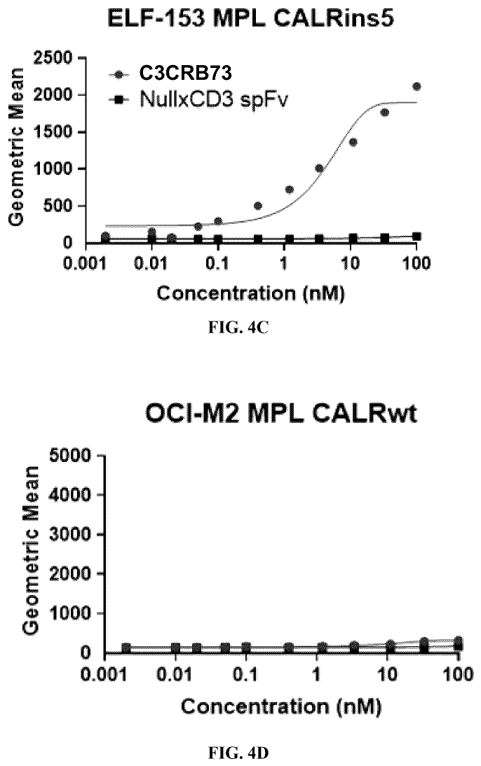

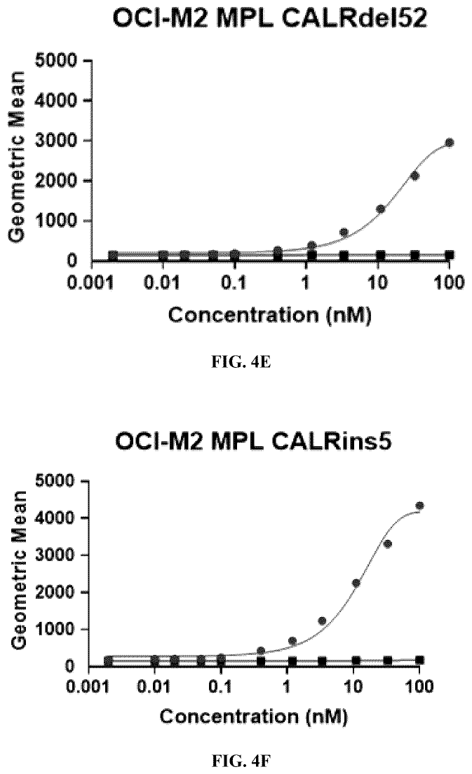

C3CRB73 showed selective binding to cells expressing CALRmut and no binding to target-negative cells (FIGS. 4A-4F). Further, C3CRB73 did not bind to OCI-M2 cells overexpressing MPL and wild-type CALR. The NullxCD3 spFv control molecule did not bind to any of the cancer cell lines tested. Overall, these results are in line with the Retrogenix off-target screen showing that the CALRmut-binding arm of C3CRB73 is selective to CALRmut.

### B. Binding to CD34+ Cells Derived From CALRmut MPN Patients

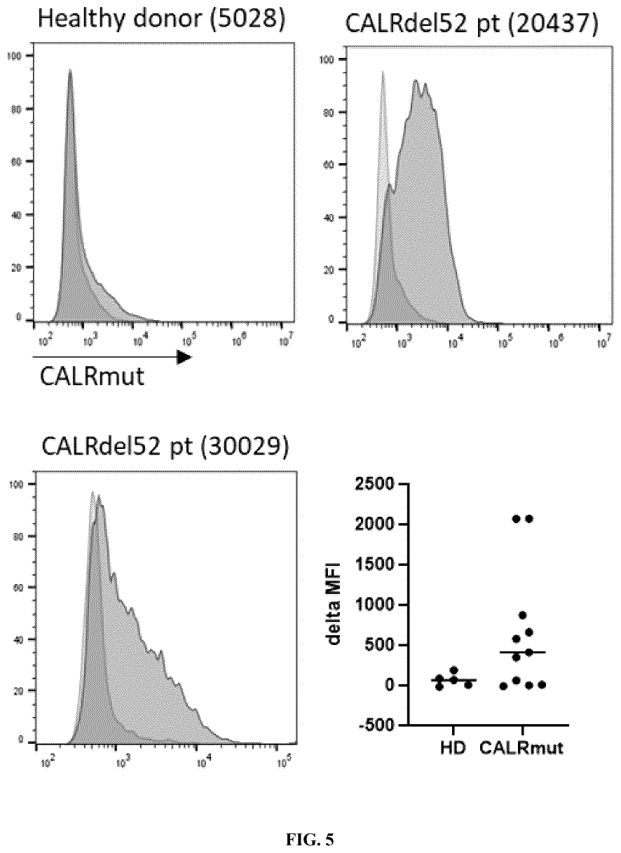

The acquisition of CALR mutations in CD34+ HSCs provides selective advantage over normal HSCs. C3CRB73 binding to CALRmut-derived and healthy-donor-derived CD34+ cells was assessed. No binding to healthy donor CD34+ cells was observed (FIG. 5). C3CRB73 binding to CALRmut-derived CD34+ cells was observed for 7 out of 11 CALRmut donors. Lack of binding to some of the CALRmut-patient derived CD34+ cells was likely related to assay limitations (i.e., increased background related to CD34+ cell paraformaldehyde fixation), contributing to lower signal detection by flow cytometry. CD34+ cells from 1 of the 4 donors that did not show bispecific antibody binding was tested in the autologous cytotoxicity assay. Although C3CRB73 did not bind to these cells, it led to T cell-mediated cytotoxicity with maximal cytotoxicity of 68%. CD34+ cells from the other 3 donors that did not show binding were not assessed in the cytotoxicity studies.

### C. Primary T Cell Binding

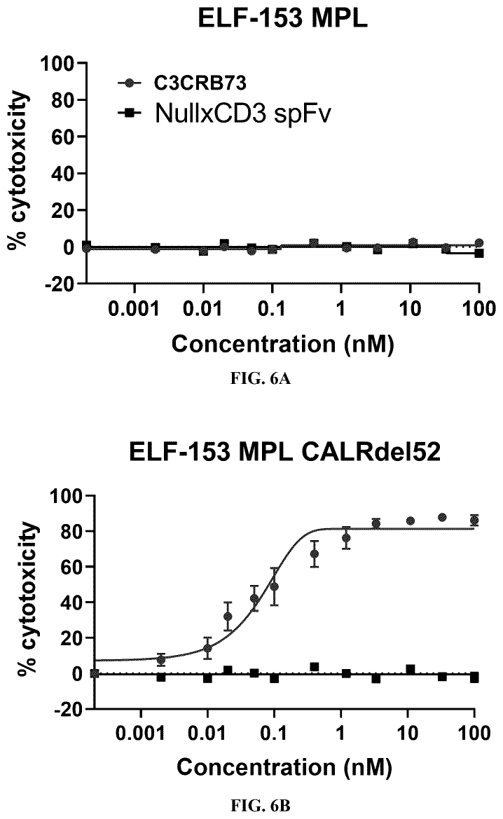

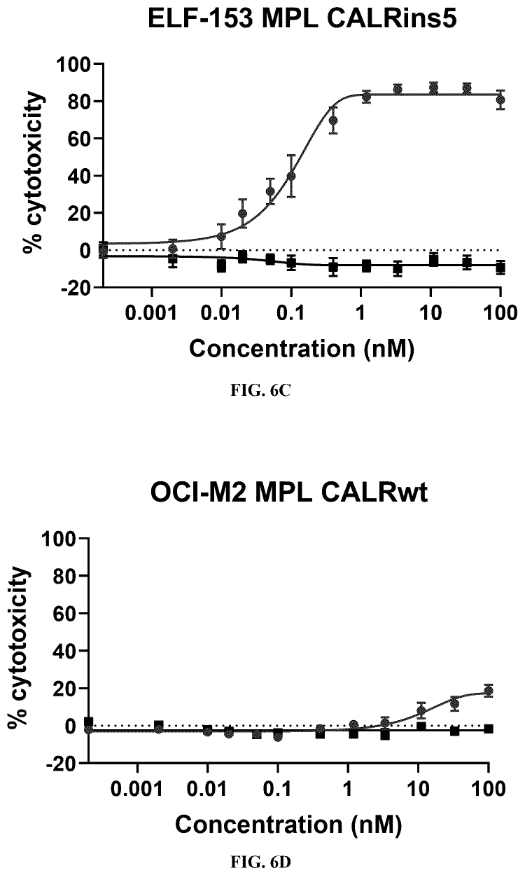

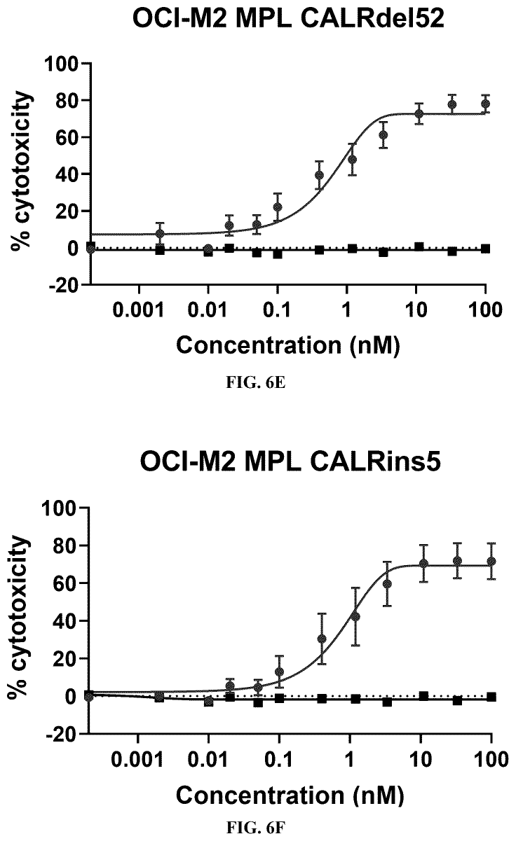

Flow cytometry was used to evaluate the CD3 arm binding of C3CRB73 to CD3+ pan T cells from 6 different healthy human donors. C3CRB73 and NullxCD3 spFv were tested after 1 hour 37° C. incubation with pan T cells. C3CRB73 showed consistent binding profiles across primary T cell populations expressing endogenous CD3 with an average EC50 of 23.75 nM (FIG. 6). NullxCD3 spFv showed comparable binding to C3CRB73.

### Example 6: In Vitro Evaluation of C3CRB73 CALRmut x CD3 Bispecific Antibody Activity

A. C3CRB73-induced T cell-mediated Cytotoxicity of CALRmut-engineered Cancer Cells

T cell redirection assays were performed to evaluate the ability of C3CRB73 to induce CALRmut-selective cytotoxicity of cell lines engineered to express the most common CALRmuts, ie, CALRdel52 and CALRins5. T cells from 6 to 10 healthy donors were used at 5:1 E:T ratios. After 72 hours, C3CRB73 elicited concentration-dependent cytotoxicity of the cell lines expressing MPL CALRdel52 or MPL CALRins5 (FIGS. 6A-6F and Table 15). No cytotoxicity was observed for the ELF-153 MPL cell line, while minimal cytotoxicity to OCI-M2 MPL CALRwt cells was observed at the highest concentrations. No cytotoxicity was observed in presence of the NullxCD3 spFv negative control. These results demonstrate that C3CRB73 can induce selective cytotoxicity of CALRmut cell lines expressing different levels of CALRmut on the cell surface.

TABLE 15
C3CRB73-induced T cell-mediated cytotoxicity: EC50
and maximum cytotoxicity values with CALRmut cancer
cell lines at 5:1 E:T ratios at 72 hours.

| | ELF-153 | ELF-153 | OCI-M2 | OCI-M2 |
|---|---|---|---|---|
| | MPL | MPL | MPL | MPL |
| | CALRdel52 | CALRins5 | CALRdel52 | CALRins5 |
| EC₅₀ (nM) | 0.034 | 0.094 | 0.61 | 1.69 |
| Maximal | 88.9 | 86.5 | 82.7 | 73.7 |
| cytotoxicity | | | | |
| (%) | | | | |

CALRmut, mutant calreticulin;
EC₅₀, 50% effective concentration;
E:T ratio, effector-to-target ratio;
NLME, non-linear mixed effects

Values are representative of n=6 to 10 individual healthy donors. Data from independent experiments were pooled and represented as mean. A NLME model was used for EC₅₀ value estimation across the donors. Separate NLME models were fit for each time treatment. The NLME model was used to estimate dose-response profile, using the 4 parameters of the standard 4-parameter logistic regression model as the fixed effects (minimum, maximum, slope, log EC₅₀), and using donor as a random effect. Results are reported on the original scale. All analyses were performed in R Version 4.0.3.
B. C3CRB73-induced T cell Activation in Vitro in Presence of CALRmut Cancer Cell Lines

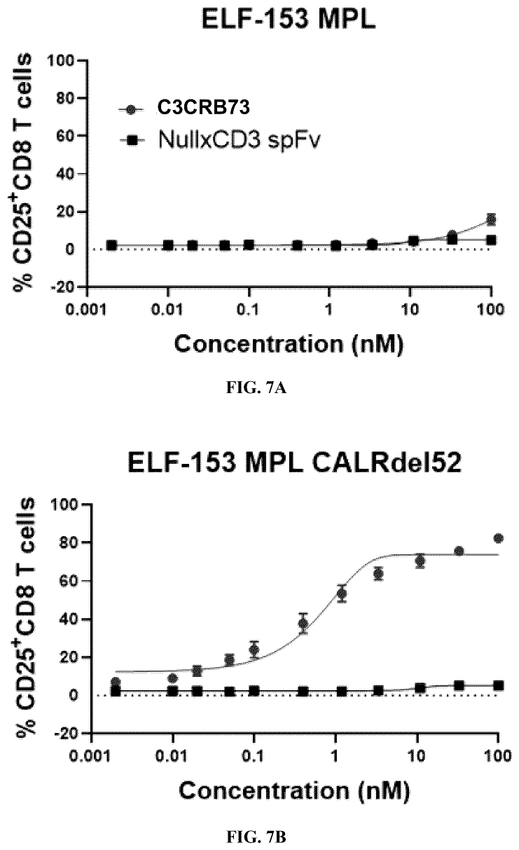

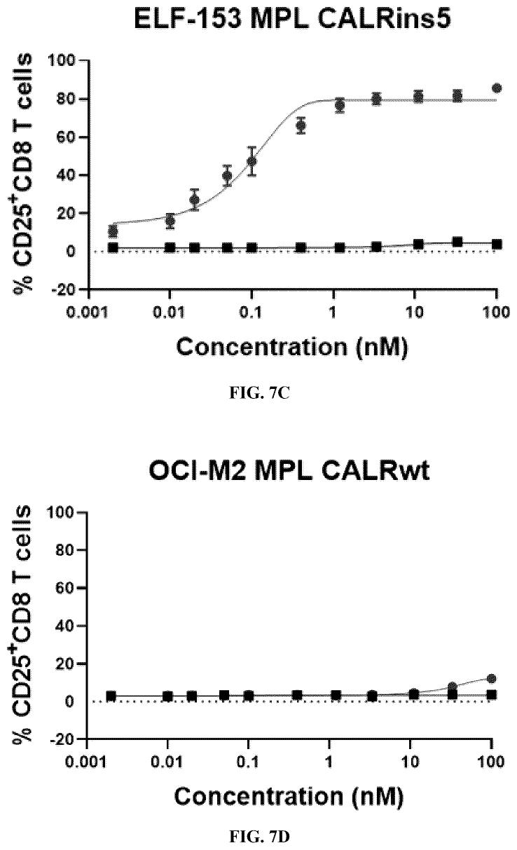

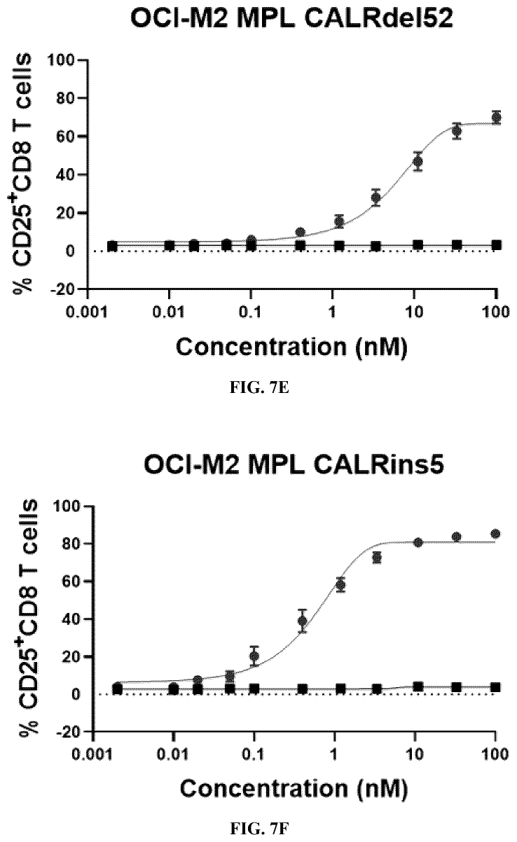

CD25 expression as a measure of T-cell activation was monitored in the cytotoxicity assays described herein. C3CRB73 elicited concentration-dependent increases in the activation of T cells in the presence of cell lines expressing CALRmut after 72 hours but did not elicit activation in coculture with target-negative cell lines despite some minimal T-cell activation observed at the top concentration tested (i.e., 100 nM) (FIGS. 7A-7F). The NullxCD3 spFv negative control antibody induced no T-cell activation.

C. C3CRB73-induced T cell-mediated Cytotoxicity of CALRmut CD34+ Cells Derived from CALRmut MPN Patient Cells in Autologous Setting

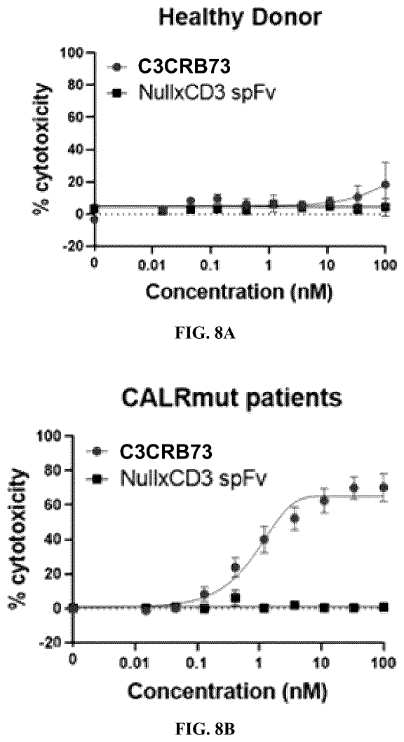

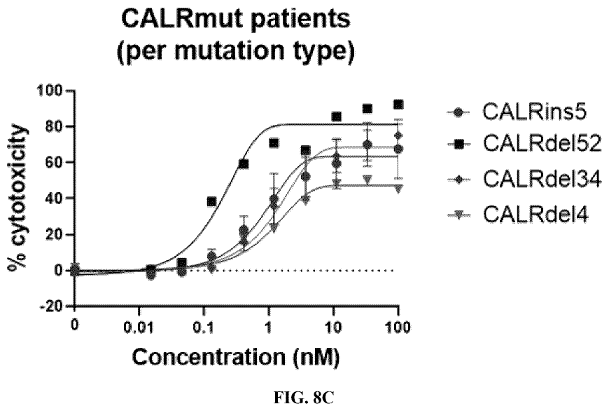

To evaluate the impact of C3CRB73 in a more physiologically relevant setting, T cell redirection assays were conducted in an autologous setting using CD34+ cells isolated from CALRmut MPN patients as target cells together with T cells isolated from the same patient. Cells were isolated from patients harboring the following mutations: CALRdel52, CALRins5, CALRdel34, or CALRdel4. For comparison, CD34+ and T cells isolated from healthy donors were also used in these experiments (Table 16). The studies were conducted using an E:T ratio of 5:1. C3CRB73 elicited concentration-dependent cytotoxicity of patient-derived CALRmut CD34+ cells (FIGS. 8A-8C), with an EC50 value of 0.94 nM, 90% effective concentration (EC90) value of 7.58 nM, and 69% of maximum cytotoxicity. C3CRB73-mediated cytotoxicity was observed against all CALRmut CD34+ cells and was independent of the type of CALR mutation. No cytotoxicity was observed to healthy donor CD34+ cells despite a minimal effect at the top concentration tested (i.e., 100 nM). The NullxCD3 spFv negative control antibody did not induce cytotoxicity.

TABLE 16
Information on CALRmut MPN patients from which CD34+ and
T cells were isolated for autologous T cell redirection studies.

| | | Mutational | | |
|---|---|---|---|---|
| Patient | | burden | | |
| ID | Mutation | (%) | Diagnosis | Treatment |
| 2027 | CALRins5 | 42.2% | PMF | Ruxolitinib |
| 2046 | CALRins5 | 38.5% | PMF | Ruxolitinib > |
| | | | | HSCT |
| 4003 | CALRins5 | 46.9% | SMF, post-ET | No treatment |
| 2034 | CALRdel52 | 41.1% | PMF | Ruxolitinib > |
| | | | | fedratinib |
| 3011 | CALRdel4 | 43.6% | PMF | ND |
| 2041 | CALRdel34 | 43.0% | PMF | Thromboreductin |
| 4009 | CALRdel34 | 43.5% | ET | Thromboreductin |
| 5011 HD | na | na | na | na |
| 5016 HD | na | na | na | na |
| 5017 HD | na | na | na | na |

CALR, calreticulin; CALRmut, mutant calreticulin; CD, cluster of differentiation; del, deletion; ET, essential thrombocythemia; HD, healthy donor; HSCT, hematopoetic stem cell transplantation; ins, insertion; MPN, myeloproliferative neoplasm; na, not applicable; ND, not defined; PMF, primary myelofibrosis; sMF, secondary myelofibrosis.

Although previous studies reported exhaustion of T cells isolated from CALRmut MPN patients, the current results confirm that T cells in presence of C3CRB73 are capable to engage CALRmut-patient-derived T cells to mediate cytotoxicity to CALRmut-expressing CD34+ cells in an autologous setting even in the patients with advanced MPN stage (i.e., PMF and secondary myelofibrosis [sMF]). In addition, while C3CRB73 was evaluated ex vivo in a limited number of patient-derived samples so far, these results suggest that prior JAK1/2 inhibitor treatment does not affect C3CRB73 T cell-mediated cytotoxicity.

### Example 7: Effect of CALRmut x CD3 Bispecific Antibody on CALRmut Downstream Signaling

The oncogenic potential of human CALR mutations is dependent on binding to MPL and its downstream JAK2-STAT5 signaling. To evaluate the effect of C3CRB73 on CALRmut signaling in cancer cells, phosphorylated [p]STAT5 levels were assessed in ELF-153 MPL and ELF-153 MPL CALRmut cells in the presence or absence of recombinant CALRins5 protein. Thrombopoietin (TPO) stimulation was used as positive control for downstream of MPL signaling. Treatment with C3CRB73 did not impact the pSTAT5 levels detected on any of the cell lines, independent of presence of recombinant CALRins5 protein. Similarly, C3CRB73 did not impact pSTAT5 levels detected on any of the cell lines in the presence of TPO stimulation.

To further investigate the effect of C3CRB73 on CALRmut-driven downstream signaling, the proliferation upon treatment with C3CRB73 in ELF-153 MPL and ELF-153 MPL CALRmut cells in the presence of recombinant CALRmut protein was assessed. ELF-153 MPL CALRdel52 and ELF-153 MPL CALRins5 cells had higher proliferative rates as compared to ELF-153 MPL cells, confirming the oncogenic function of CALRmut in these cell lines. In the tested range of concentrations, C3CRB73 did not have any effect on proliferation of either ELF-153 MPL or ELF-153 MPL CALRmut cell lines, independent of the presence of recombinant CALRins5 protein or TPO stimulation.

Altogether, these results suggested that C3CRB73 does not exhibit either antagonistic or agonistic activity on CALRmut and its downstream signaling.

### Example 8: Soluble CALRmut Derisking with CALRmut x CD3 Bispecific Antibody

Soluble CALRmut protein can be found in CALRmut MPN patient plasma at a mean concentration of 25.64 ng/mL. Of the plasma CALRmut, 60% is found in complex with soluble transferrin receptor 1 (sTFRC) that acts as a carrier protein and increases the CALRmut half-life. Plasma levels of soluble CALRmut were directly correlated to the allele burdens in the blood. Soluble CALRmut levels were significantly higher in patients with a diagnosis of PMF and pre-PMF when compared to patients with a diagnosis of ET.

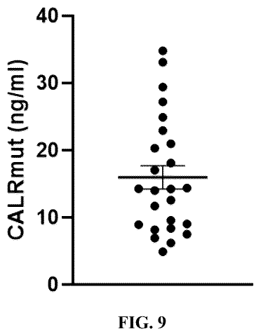

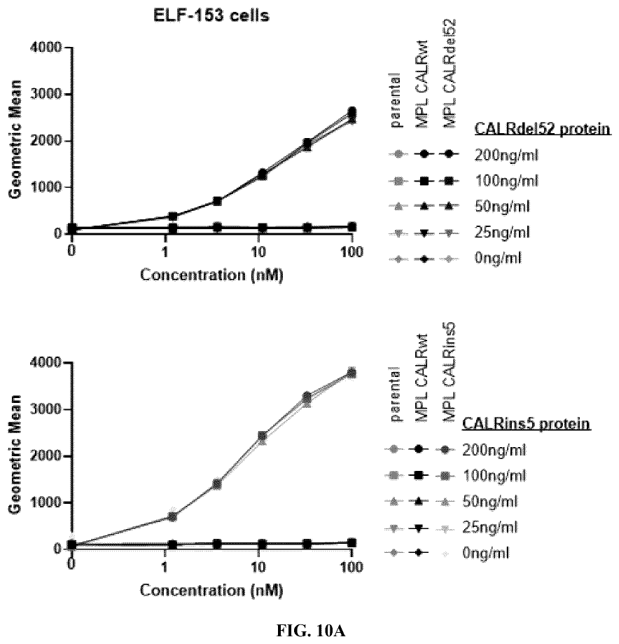

The mean plasma CALRmut concentration detected in 25 CALRmut MPN patient-derived samples used for functional studies with C3CRB73 was 16 ng/mL (FIG. 9). This value is slightly lower compared to the literature reference likely due to the use of a different enzyme-linked immunosorbent assay (ELISA) set-up, different capture and detection antibodies, and a different batch of CALRmut protein that was used as the standard curve.

There are two potential liabilities related to presence of soluble CALRmut in the circulation:
i. Soluble CALRmut could be a potential ‘sink’ for the antibody and prevent its efficacy; and
ii. Soluble CALRmut could bind in trans to MPL-expressing cells and lead to cytotoxicity of MPL-expressing cells. To derisk the presence of soluble CALRmut. Several experiments were executed either in the presence of recombinant CALRmut proteins or patient-derived CALRmut.

### A. Effect of Recombinant CALRmut Protein on Binding of C3CRB73 to Target-Positive and Target-Negative Cells

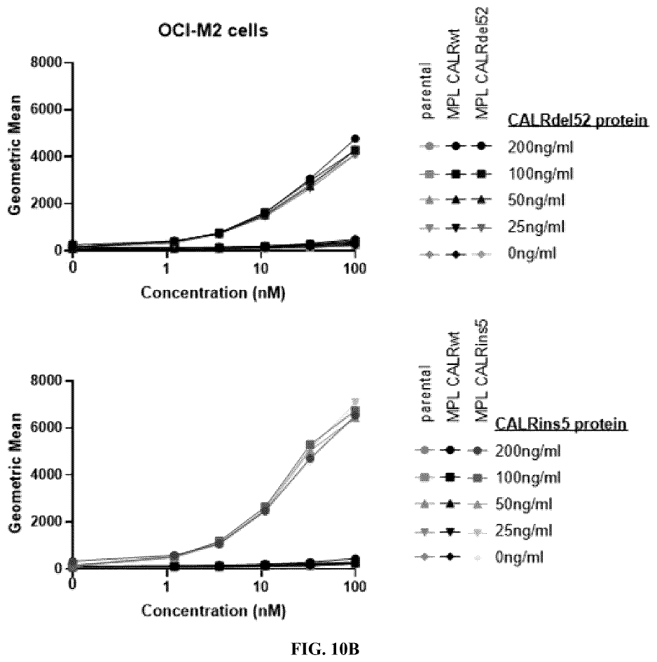

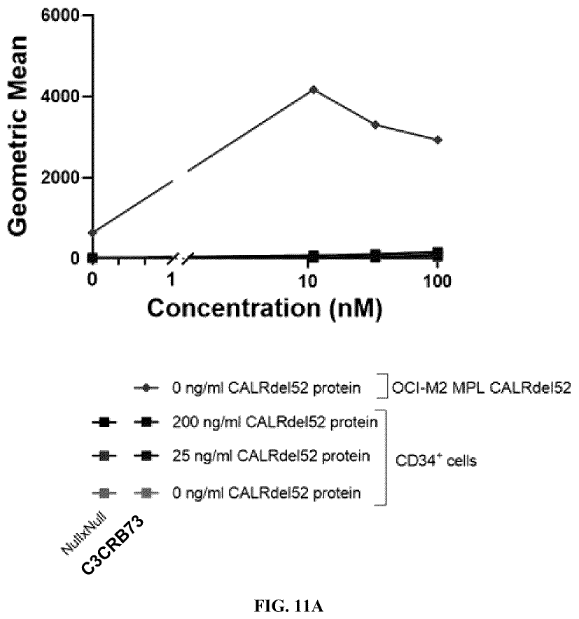

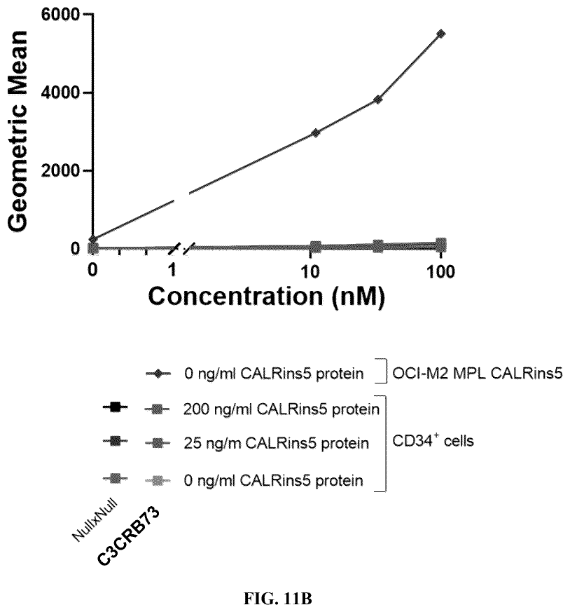

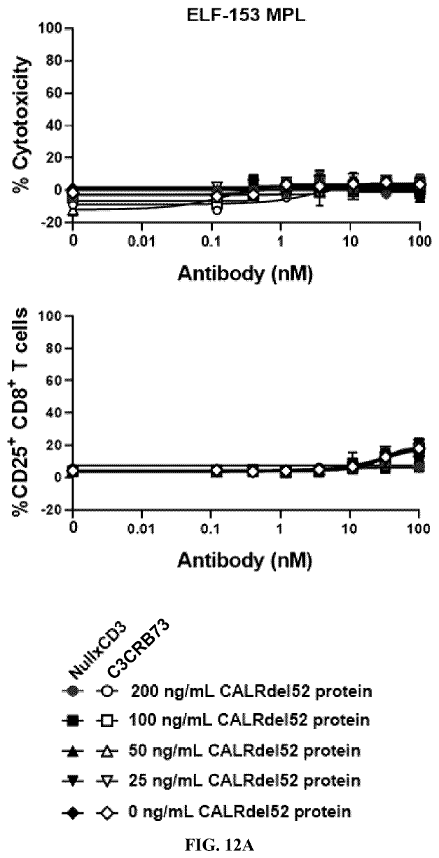

To evaluate whether soluble CALRmut could affect C3CRB73 binding to CALRmut-positive or CALRmut-negative MPL-expressing cell lines, recombinant CALRdel52 or CALRins5 proteins were spiked into the experiment at a concentration range (i.e., 0, 25, 50, 100, 200 ng/mL). Binding was assessed by flow cytometry after 1 or 24 hours of incubation with C3CRB73 and spiked CALRmut proteins. Presence of recombinant CALRmut protein did not affect the binding profile of C3CRB73, and the antibody maintained selective binding to cells expressing CALRmut and no binding to target-negative cells (FIGS. 10A and 10B). Similar binding profiles were observed at 1 and 24 hours.

### B. Effect of Recombinant CALRmut Protein on C3CRB73 Binding to Healthy Donor CD34+ Cells

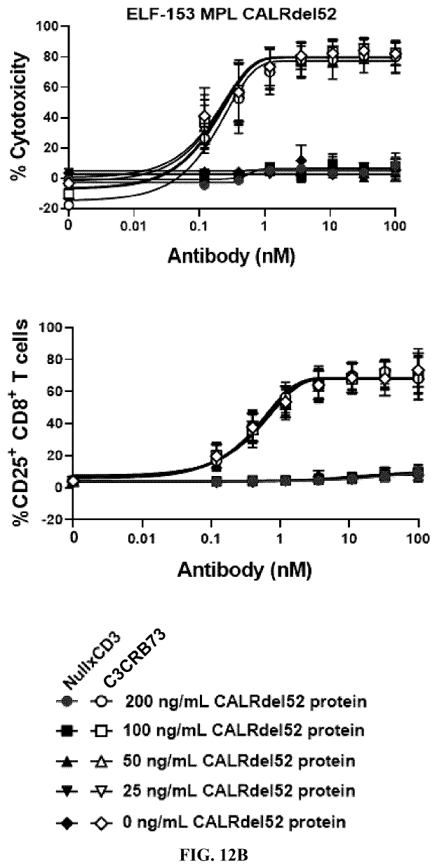

CALRmut binds to MPL in the ER, traffics together to the cell surface where it activates the MPL receptor in an autocrine manner. Paracrine MPL stimulation in CALR wild-type cells cocultured with CALR mutant cells or their conditioned media is not detected, because MPL exists in a mature glycosylated form on CALR wild-type cells, which has been described as unable to stably bind CALRmut. In humans, MPL expression is restricted to CD34+ cells, the megakaryocytic lineage, and platelets, with the highest expression reported in CD34+ HSCs. To evaluate whether soluble CALRmut induces binding of C3CRB73 to CALR wild-type MPL-expressing CD34+ cells, recombinant CALRdel52 or CALRins5 proteins were added to a culture of CD34+ at a concentration range (i.e., 0, 25, 200 ng/mL) in the presence of increasing concentrations of C3CRB73. After 1 hour of incubation, binding of C3CRB73 to CD34+ cells was assessed using flow cytometry. Engineered cancer cell lines expressing both MPL and CALRmut (i.e., OCI-M2 MPL CALRdel52 and OCI-M2 MPL CALRins5) were included as positive control for C3CRB73 binding. While C3CRB73 binding to OCI M2 MPL CALRmut cells was confirmed, C3CRB73 did not bind to healthy CD34+ s (FIG. 11). Also, presence of recombinant CALRmut protein up to 200 ng/mL did not affect C3CRB73 binding to CD34+ cells. These results suggested that presence of soluble CALRmut up to 200 ng/mL does not induce binding of C3CRB73 to MPL-expressing cells.

### C. Effect of Recombinant CALRmut Protein on C3CRB73 T Cell-Mediated Cytotoxicity to Target-Positive and Target-Negative Cells

T cell redirection assays were performed to evaluate the effect of recombinant CALRmut protein (i.e., CALRdel52 or CALRins5) at a range of concentrations on C3CRB73 T cell-mediated cytotoxicity to ELF-153 MPL or ELF-153 MPL CALRmut cells lines expressing either CALRdel52 or CALRins5. T cells from 3 healthy donors were used at 5:1 E:T ratios. Presence of recombinant CALRmut protein did not affect the potency of C3CRB73 T cell-mediated cytotoxicity to CALRmut-expressing cell lines (FIGS. 12A and 12B). Moreover, no cytotoxicity was observed to ELF-153 MPL cells in the presence of recombinant CALRmut protein. T cell activation was evaluated in parallel by CD25 expression on CD8+ T cells. Presence of recombinant CALRmut protein also did not affect C3CRB73-mediated T cell activation in coculture with any above-mentioned cancer cell lines.

D. Effect of C3CRB73 in CALRmut Whole-Blood T Cell Redirection Assay Co-Cultured with ELF 153 MPL CALRmut+ Cells

To evaluate the impact of soluble CALRmut on JNJ-88549968 in a more physiologically relevant setting, T-cell redirection assays were conducted using CALRmut patient-derived whole blood. Whole blood from healthy donors was used as a control condition lacking the soluble CALRmut component. The studies were conducted using an E:T ratio of 1:1, with T cells as effectors and target cells comprising labeled ELF-153 parental, ELF-153 MPL, ELF-153 MPL CALRdel52, or ELF-153 MPL CALRins5 cancer cells in the donor blood. Tumor cells were evaluated for cytotoxicity. T-cell activation was evaluated by CD69 and CD25 expression on CD8⁺ T cells. Supernatant cytokine analysis was also performed. Soluble CALRmut levels and the number of circulating CALRmut CD34⁺ cells in the blood of CALRmut MPN patients were quantified at the start of the assay (Table 17).

TABLE 17
Initial soluble CALRmut levels and number of circulating
CALRmut CD34+ cells in the blood of CALRmut MPN patients

| | | | | | CD34⁺ in | Soluble |
|---|---|---|---|---|---|---|
| | | | | | 100 μL | CALRmut |
| Patient | | Mutational | | | whole | level |
| ID | Mutation | burden | Diagnosis | Treatment | blood | (ng/mL) |
| 304811 | ins5 | 49.6% | ET | Ruxolitinib | 9,332 | 14.29 |
| 302714 | ins5 | 42.2% | PMF | Ropeginterferon- | 2,268 | 4.92 |
| | | | | α-2b | | |
| 301113 | del4 | 49.7% | PMF | Ropeginterferon- | 23,532 | 14.39 |
| | | | | α-2b | | |
| 30477 | ins5 | 36.2% | ET | Thromboreductin | 1,399 | 6.20 |
| 302912 | del52 | 50.2% | PMF | Thromoboreductin | 7,352 | 8.95 |
| 300511 | del19 | 49.6% | ET | Hydroxyurea | 22,976 | 7.53 |
| 30455 | del52 | 34.1% | ET | Thromboreductin | 4,772 | 6.94 |
| 302814 | ins5 | 44.4% | ET | Ropeginterferon- | 1,894 | 11.73 |
| | | | | α-2b | | |
| 301415 | ins5 | 44.8% | ND | Ropeginterferon- | 2,354 | 8.18 |
| | | | | α-2b and | | |
| | | | | thromboreductin | | |
| 400414 | del34 | 44.8 | ND | No treatment | 8,301 | 12.58 |
| 20028 | ins5 | 49.7 | ND | Thromboreductin | 1,681 | 9.59 |
| 20594 | ND | ND | ND | Ropeginterferon- | 1,635 | 9.06 |
| | | | | α-2b | | |
| 302913 | del52 | 50.2 | ND | Thromboreductin | 6,337 | 14.26 |

CALRmut, mutant calreticulin; CD, cluster of differentiation; del, deletion; ET, essential thrombocythemia; ins, insertion; MPN, myeloproliferative neoplasm; ND, not defined; PMF, primary myelofibrosis.

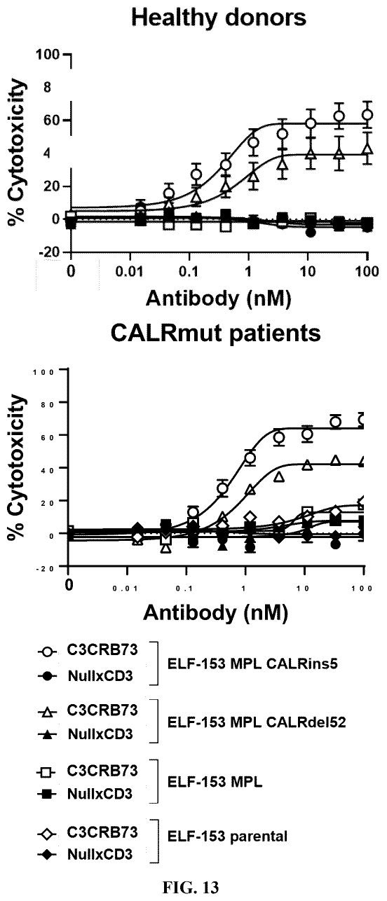

C3CRB73 elicited concentration-dependent T cell-mediated cytotoxicity of ELF 153 MPL CALRmut cells (both CALRdel52- and CALRins5-expressing cell lines) with similar potency in whole blood of healthy donors and CALRmut MPN patients (FIG. 13, Table 18). Minimal cytotoxicity at higher concentrations was observed to ELF 153 MPL cells in the presence of C3CRB73 in whole blood from CALRmut patients, but this effect was target independent as the NullxCD3 spFv control showed the same effect.

TABLE 18
C3CRB73-induced T cell-mediated cytotoxicity to CALRmut
cell lines in the whole blood from healthy donors and
CALRmut patients: EC50 and maximal cytotoxicity values

| | Healthy donors | | CALRmut MPN patients | |
|---|---|---|---|---|
| | ELF-153 | ELF-153 | ELF-153 | ELF-153 |
| | MPL | MPL | MPL | MPL |
| | CALRdel52 | CALRins5 | CALRdel52 | CALRins5 |
| EC₅₀ (nM) | 0.51 | 0.32 | 0.66 | 0.64 |
| Maximal | 42.9 | 65.9 | 43.0 | 64.7 |
| cytotoxicity | | | | |
| (%) | | | | |

CALR, calreticulin; CALRmut, mutant calreticulin; del, deletion; EC₅₀, 50% effective concentration; ins, insertion; MPL, thrombopoietin receptor; MPN, myeloproliferative neoplasm; NLME, non-linear mixed effects.

Values are representative of n=6 individual healthy donors or n=9 to 13 CALRmut patients. Data from independent experiments were pooled and represented as mean. A NLME model was used for EC₅₀ value estimation across the donors. Separate NLME models were fit for each time treatment. The NLME model was used to estimate dose-response profile, using the 4 parameters of the standard 4-parameter logistic regression model as the fixed effects (minimum, maximum, slope, log EC₅₀), and using donor as a random effect. Results are reported on the original scale. All analyses were performed in R Version 4.0.3.

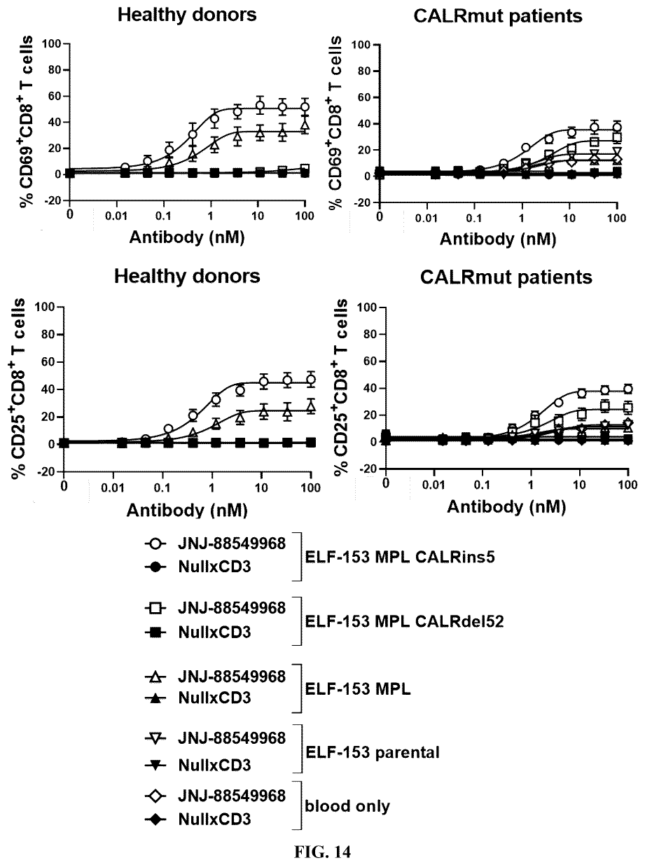

Also, CD8+ T cell activation was comparable in the whole blood from CALRmut MPN patients and healthy donors in the presence of ELF 153 MPL CALRdel52 or ELF 153 MPL CALRins5 cells (FIG. 14).

In the presence of C3CRB73, a minor increase in CD8⁺ T-cell activation in whole blood of CALRmut MPN patients in the absence of cancer cells or in the presence of ELF-153 parental or ELF-153 MPL cancer cells was observed. This increase in T-cell activation was likely due to the presence of CALRmut CD34⁺ cells in the blood (see Table 17) and differential T-cell activation status in patients. The EC₅₀ and maximum CD8⁺ T-cell activation values are reported in Table 19. Similar data were observed for CD4⁺ T cells (data not shown).

Altogether, these data suggested that the presence of soluble CALRmut derived from CALRmut MPN patients does not impair cytotoxic activity of C3CRB73 towards CALRmut-expressing cancer cells, nor mediate cytotoxicity of MPL-expressing cells, sufficiently derisking an impact of soluble CALRmut.

TABLE 19
CD8+ T cell activation EC50 values observed for
C3CRB73 in whole-blood T cell redirection assays conducted
at 1:1 E:T ratio, spiked with ELF 153 MPL-positive
or ELF 153 MPL-positive CALRmut-positive cancer cells

| Healthy donors | | CALRmut patients | |
|---|---|---|---|
| ELF-153 | ELF-153 | ELF-153 | ELF-153 |
| MPL | MPL | MPL | MPL |
| CALRdel52 | CALRins5 | CALRdel52 | CALRins5 |

% CD69⁺ CD8 T cells

| | | | | |
|---|---|---|---|---|
| EC₅₀ (nM) | 0.88 | 0.41 | 3.06 | 1.08 |
| Maximal T-cell | 35.5 | 52.9 | 30.4 | 39.9 |
| activation (%) | | | | |

% CD25⁺ CD8 T cells

| | | | | |
|---|---|---|---|---|
| EC₅₀ (nM) | 1.0 | 0.53 | 2.46 | 1.68 |
| Maximal T-cell | 26.6 | 47.7 | 24.4 | 39.6 |
| activation (%) | | | | |

CALRmut, mutant calreticulin; CD, cluster of differentiation; EC₅₀, 50% effective concentration; E:T ratio, effector-to-target ratio; MPL, thrombopoietin receptor; NLME, non-linear mixed effects.

Values are representative of n=6 individual healthy donors or n=9 CALRmut patients. Data from independent experiments were pooled and represented as mean. A NLME model was used for EC₅₀ value estimation across the donors. Separate NLME models were fit for each time treatment. The NLME model was used to estimate dose-response profile, using the 4 parameters of the standard 4-parameter logistic regression model as the fixed effects (minimum, maximum, slope, log EC₅₀), and using donor as a random effect. Results are reported on the original scale. All analyses were performed in R Version 4.0.3.

To further characterize T cell activation induced by C3CRB73, supernatants from the whole-blood T cell redirection assay were analyzed for cytokine levels and EC50 and max values are reported. Cytokine secretion varied across donors (Table 20). Overall, low cytokine levels were detected in the supernatants from T cell redirection assays performed with whole blood derived from CALRmut patients. Interleukin (IL) 2, IL 4, IL 10, and IL 12p70 levels were below the standard curve in 4 or more tested donors and thus were excluded from the analysis.

TABLE 20
Cytokine level EC50 and maximum cytotoxicity values

| | | Healthy donor | | CALRmut MPN patient | |
|---|---|---|---|---|---|
| | | ELF-153 | ELF-153 | ELF-153 | ELF-153 |
| Cyto- | | MPL | MPL | MPL | MPL |
| kine | Value | CALRdel52 | CALRins5 | CALRdel52 | CALRins5 |
| IFN-γ | EC₅₀ (nM) | 1.23 | 1.60 | 3.07 | 2.9 |
| | Max value | 1.67 × 10³ | 2.01 × 10⁴ | 697 | 4.64 × 10³ |
| | (pg/mL) | | | | |
| IL-1β | EC₅₀ (nM) | 1.83 | 1.08 | 2.30 | Inesti- |
| | | | | | mable |
| | Max value | 40.57 | 151 | 29.26 | Inesti- |
| | (pg/mL) | | | | mable |
| IL-2 | EC₅₀ (nM) | ND | ND | ND | ND |
| | Max value | ND | ND | ND | ND |
| | (pg/mL) | | | | |
| IL-4 | EC₅₀ (nM) | ND | ND | ND | ND |
| | Max value | ND | ND | ND | ND |
| | (pg/mL) | | | | |
| IL-8 | EC₅₀ (nM) | ND | ND | ND | ND |
| | Max value | ND | ND | ND | ND |
| | (pg/mL) | | | | |
| IL-6 | EC₅₀ (nM) | ND | 4.91 | Inesti- | 2.07 |
| | | | | mable | |
| | Max value | ND | 4.55 × 10³ | Inesti- | 848 |
| | (pg/mL) | | | mable | |
| IL-10 | EC₅₀ (nM) | ND | ND | ND | ND |
| | Max value | ND | ND | ND | ND |
| | (pg/mL) | | | | |
| IL-13 | EC₅₀ (nM) | 3.11 | 1.24 | 4.29 | 1.47 |
| | Max value | 95.63 | 187 | 109 | 148 |
| | (pg/mL) | | | | |
| TNF-α | EC₅₀ (nM) | 2.15 | 1.70 | 3.16 | 1.20 |
| | Max value | 52.07 | 239 | 36.54 | 72.29 |
| | (pg/mL) | | | | |

CALR, calreticulin; CALRmut, mutant calreticulin; del, deletion; ins, insertion; IFN, interferon; IL, interleukin; MPL, thrombopoietin receptor; MPN, myeloproliferative neoplasm; ND; not detectable (data points below or above standard curve) in 4 or more donors; TNF, tumor necrosis factor.

Whole blood from n=6 individual healthy donors or n=8 CALRmut patients was tested in T-cell redirection assays in the presence of C3CRB73 and ELF-153 MPL-positive or ELF-153 MPL-positive CALRmut-positive target cell lines. Secreted cytokines were measured after 72 hours. Means and medians were calculated based on donor-specific fitting; model-estimated fitting results are also shown. Some values were excluded due to non-sigmoidal behavior and deemed inestimable.

Overall, these results suggest that presence of CALRmut in patient circulation poses no risk for C3CRB73 activity.

### E. Assessment of Soluble CALRmut Binding in Trans to MPL In Vivo

To assess if soluble CALRmut, secreted from CALRmut-expressing tumors, can bind in trans to MPL on wild-type tumors, female NSG (i.e., non-obese diabetic [NOD] severe combined immunodeficiency [scid] gamma or NOD.Cg-Prkdcscid Il2rgtm1Wjl/SzJ) mice were inoculated bilaterally SC with 3×106 ELF-153 MPL CALRdel52 (left flank) and 10×106 ELF-153 MPL (right flank) tumor cells.

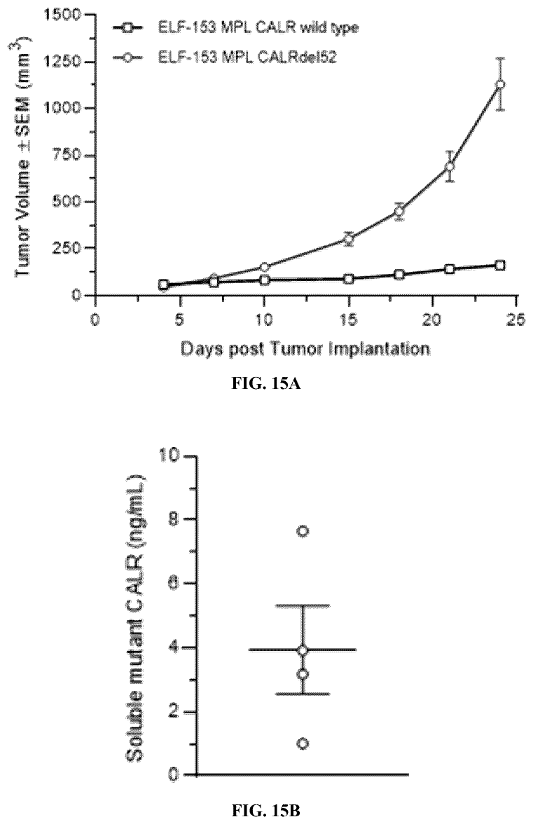

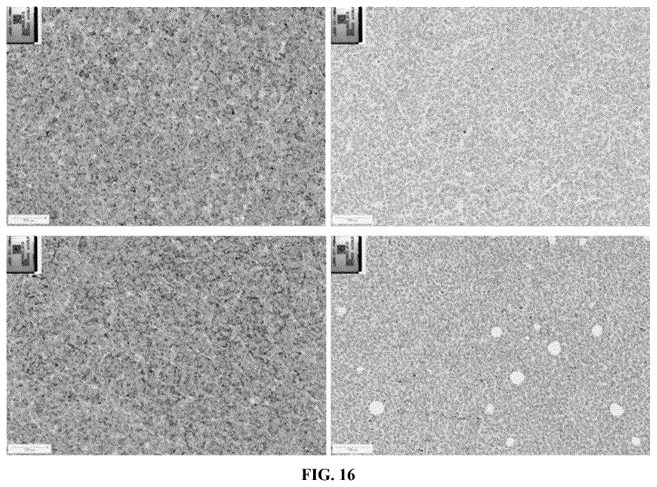

ELF-153 MPL CALRdel52 tumors grew faster than ELF-153 MPL tumors, confirming the oncogenic driver function of CALRdel52 in vivo (FIG. 15A). Additionally, soluble CALRmut was detected in the circulation of mice bearing ELF-153 MPL CALRdel52 (FIG. 15B). Detected levels of soluble CALRmut in the mice were 4 ng/mL.

Anti-CALRmut immunohistochemistry (IHC) was performed on paired tumors. CALRmut-positive staining by IHC was detected in ELF-153 MPL CALRdel52 tumors, while no staining was observed in paired ELF-153 MPL tumors from the same mouse, suggesting lack of binding of soluble CALRmut to MPL receptors in trans (FIG. 16).

### Example 9: In Vivo Pharmacology Studies of CALRmut x CD3 Bispecific Antibody

A. C3CRB73-induced T cell-mediated Efficacy of CALRmut Xenografts

The antitumor efficacy of C3CRB73 was evaluated in SC human megakaryoplastic leukemia ELF-153 MPL CALRdel52 and disseminated acute myeloid leukemia (AML) OCI-M2 MPL CALRins5 xenografts in T cell-humanized mice. For all studies, female NSG mice (Charles River Labs, Lyon, France) were used to provide a suitable host for reconstituting a human immune system using human donor CD3+ pan T cells. Mice were inoculated SC with 3×106 ELF 153 MPL CALRdel52 or intravenously (IV) with 0.5×106 OCI-M2 MPL CALRins5 tumor cells prior to T cell engraftment. The tumor implantation day was designated as Day 0 of the study.

Expanded human pan T cells were inoculated intraperitoneally (IP) into NSG mice to humanize the immune system. Humanization of mice with CD3-expressing T cells provided effector cells to facilitate C3CRB73-mediated cytotoxicity to human CALRmut-expressing tumor cells. T cell-humanized mice were given Fc block antibody and intravenous immunoglobulin (IVIg) IP at least 30 minutes prior to C3CRB73 dosing to correct for the low Ig environment in NSG mice.

In the prevention study, NSG mice were randomly assigned into groups of n=10 animals, implanted SC with ELF-153 MPL CALRdel52 cells, humanized with pan T cells on Day 3 post tumor cell implantation, and treated twice weekly for a total of 8 doses with 1, 5, or 20 mg/kg C3CRB73 IP starting on Day 4 post cell implantation.

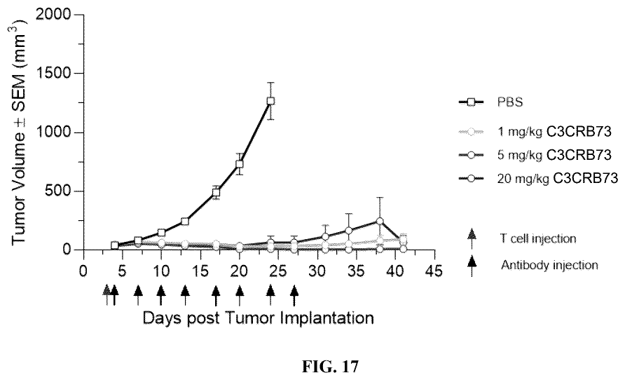

Percent tumor growth inhibition (TGI) of SC ELF-153 MPL CALRdel52 xenografts was calculated on Day 24 post tumor implantation, when two thirds of vehicle-treated (i.e., phosphate-buffered saline [PBS]) control animals remained on study. Statistically significant TGI was observed with C3CRB73 at 1, 5, and 20 mg/kg resulting in 97.4% (p<0.0001), 99.3% (p<0.0001), and 94.9% (p<0.0001) TGI, respectively, as compared with vehicle-treated controls (FIG. 17). No signs of graft-versus-host disease (GvHD)-related morbidity due to the humanization with T cells was observed in this study.

Using the same ELF-153 MPL CALRdel52 cellular model, C3CRB73 was also evaluated in a regression model. Similar to a positive control CALRmutxCD3 antibody (that in previous studies led to tumor regression), C3CRB73 led to temporary tumor stasis with regrowth at later time points. Within this model, interstudy variability and aggressive tumor growth was observed, which likely contributed to this partial response.

In the efficacy study, NSG mice were randomized into groups of n=10 animals and humanized with T cells 6 days post OCI M2 MPL CALRins5 tumor cell implantation. IP treatment with 1, 5, or 20 mg/kg C3CRB73 was initiated on Day 7 post tumor cell implantation twice weekly for a total of 10 doses. Mice were monitored daily for negative clinical signs (i.e., lethargy, ruffled and matted coat, hunched posture, hind limb paralysis, cyanotic extremities, or dyspnea) or excessive body weight loss (i.e., >20%) as compared to initial body weight. Animals showing negative clinical signs or excessive body weight loss, were removed from the study and humanely euthanized.

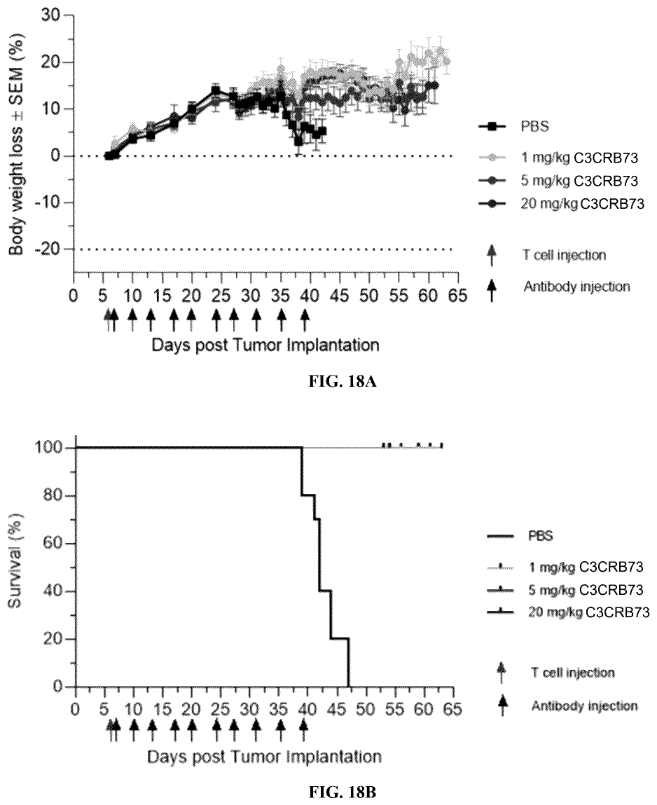

All animals in the vehicle-treated (i.e., PBS) control group were removed from study by Day 47 due to significant body weight loss and tumor burden morbidity (hind limb paralysis). Mice treated with 1, 5, or 20 mg/kg of C3CRB73 remained on study until termination of the experiment (Day 63) or were removed from study due to GvHD caused by human T cells reacting to mouse tissue antigens. Treatment with C3CRB73 significantly increased lifespan (ILS) with >50% ILS (p<0.05) for all dose levels as compared to vehicle-treated control mice (FIGS. 18A and 18B). As defined by National Cancer Institute criteria, >25% ILS is considered biologically significant.

Increasing levels of soluble CALRmut protein in plasma has been reported to correlate with the disease progression. In the OCI-M2 MPL CALRins5 disseminated model, limited soluble CALRmut levels (˜0.2 ng/mL) were detected in plasma of animals treated with 1, 5, and 20 mg/kg C3CRB73, while soluble CALRmut levels of 2.55 ng/mL were measured in PBS-treated control mice. These results further confirm antitumor activity of C3CRB73.

In both studies, treatment with 1, 5, or 20 mg/kg C3CRB73 did not show signs of body weight loss or other signs of gross toxicity. First signs of GvHD-related morbidity due to the humanization with T cells was observed on Day 53.

### B. Summary and Conclusions

Taken together, the in vitro and in vivo results shown in Examples 5, 6, 7, 8, and 9 document C3CRB73's ability to induce potent and CALRmut-specific cytotoxicity to cancer cells.

In vitro, C3CRB73 led to CALRmut-specific cytotoxicity to engineered cancer cell lines expressing the 2 predominant CALR mutations (i.e., CALRdel52 or CALRins5). No impact on cancer cell viability of target-negative cell lines was observed. Moreover, C3CRB73 also mediated CALRmut-specific cytotoxicity when using CALRmut-patient-derived CD34+ cells and T cells in an autologous system. C3CRB73 led to T cell-mediated cytotoxicity of CALRmut-positive cells in the whole blood setting with T cell activation with low cytokine secretion. C3CRB73 did not have agonistic nor antagonistic activity on CALRmut signaling.

In vivo, C3CRB73 prevented tumor growth in the ELF-153 MPL CALRdel52 SC xenograft model and significantly increased lifespan in the OCI-M2 MPL CALRins5 disseminated xenograft model.

Evaluation of the impact of soluble CALRmut on C3CRB73 activity suggested that presence of CALRmut in patient blood does not affect the activity of C3CRB73.

### Example 10: Alternative Anti-CALRmut Antibodies

### A. Binding to CALRmut Cancer Cells

To evaluate the selective binding of additional ant-CALRmut x CD3 antibodies to CALRmut cells, pairs of the isogenic cell lines expressing MPL and CALRmut (CALRdel52 or CALRins5) were generated in the background of ELF-153 (cell line derived from MF patient that progressed to sAML, i.e., AML M7) or OCI-M2 (cell line derived from erythroleukemia patient, i.e., AML M6) cells lines. Expression of MPL and CALRmut were confirmed on the cell surface of these cells as shown in Table 14. Cell surface binding to CALRmut expressed as ΔGeoMean is shown for 6 hematologic tumor cell lines engineered to express CALRmut and MPL. The experiment was performed once or twice, and single points were tested for each condition.

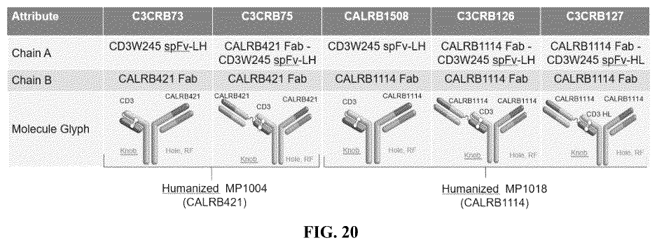

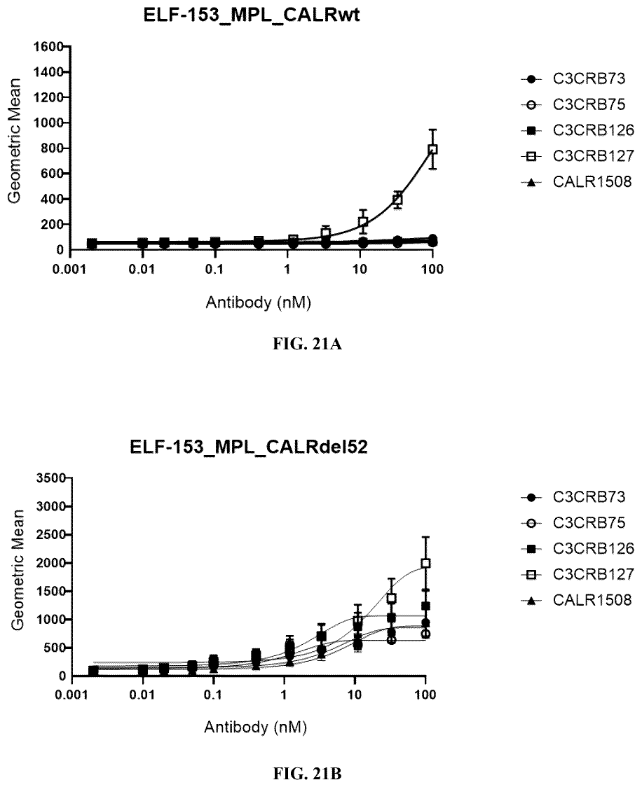

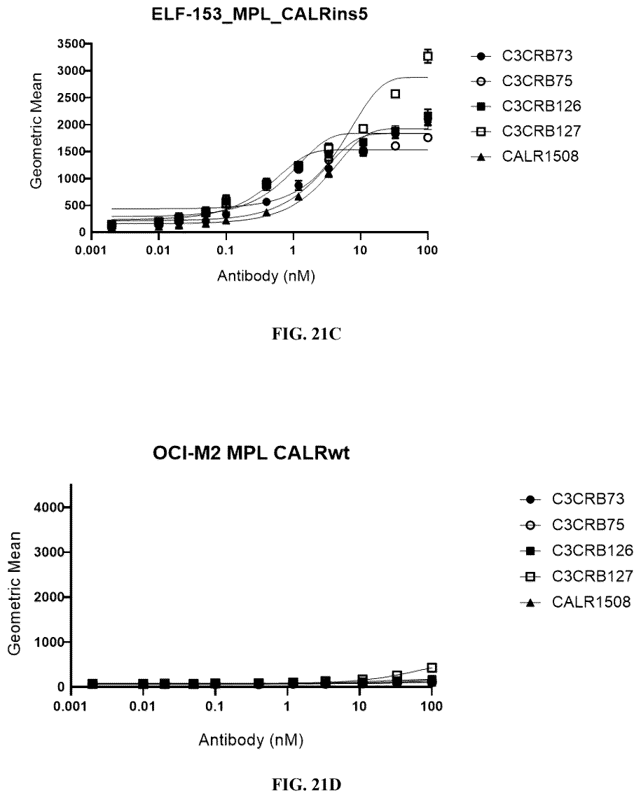

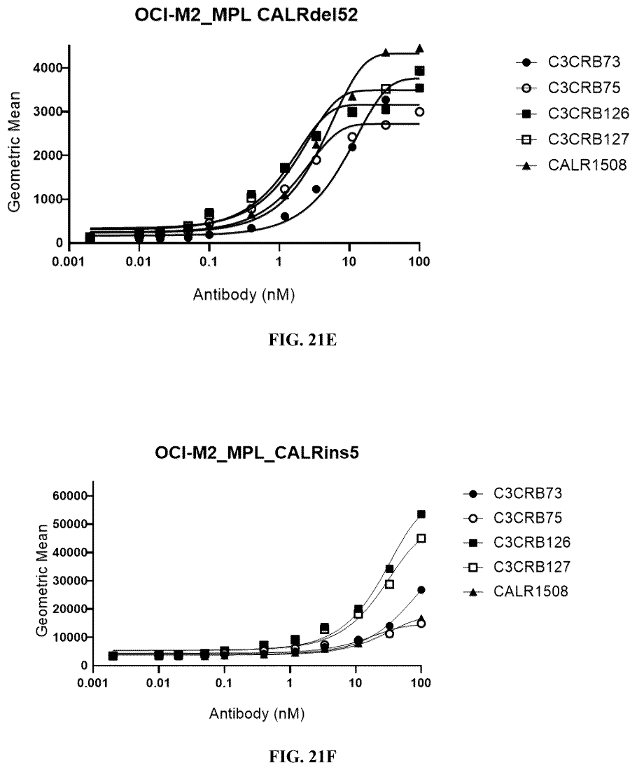

All CALRmutxCD3 antibodies listed in Tables 21-24 and FIG. 20 showed selective binding to cells expressing CALRmut and no binding to target-negative cells (FIGS. 21A-21F). The NullxCD3 spFv control molecule did not bind to any of the cancer cell lines tested.

TABLE 21
Sequences for C3CRB75 CALRmut x CD3 Bispecific Antibody

| SEQ ID NO | Description | Sequence |
|---|---|---|
| 200 | C3CRB75 Chain A, | GIDLSNNAIS |
| | CALRB421 Heavy | |
| | CDR1 AbM | |
| 201 | C3CRB75 Chain A, | VIGNTGDTY |
| | CALRB421 Heavy | |
| | CDR2 AbM | |
| 202 | C3CRB75 Chain A, | GPPSYSSSVKNI |
| | CALRB421 Heavy | |
| | CDR3 AbM | |
| 260 | C3CRB75 Chain A, | GIDLSNN |
| | CALRB421 Heavy | |
| | CDR1 Chothia | |
| 261 | C3CRB75 Chain A, | GNTGD |
| | CALRB421 Heavy | |
| | CDR2 Chothia | |
| 202 | C3CRB75 Chain A, | GPPSYSSSVKNI |
| | CALRB421 Heavy | |
| | CDR3 Chothia | |
| 81 | C3CRB75 Chain A, | NNAIS |
| | CALRB421 Heavy | |
| | CDR1 Kabat | |
| 259 | C3CRB75 Chain A, | VIGNTGDTYYADSAKG |
| | CALRB421 Heavy | |
| | CDR2 Kabat | |
| 202 | C3CRB75 Chain A, | GPPSYSSSVKNI |
| | CALRB421 Heavy | |
| | CDR3 Kabat | |
| 262 | C3CRB75 Chain A, | GIDLSNNA |
| | CALRB421 Heavy | |
| | CDR1 IMGT | |
| 263 | C3CRB75 Chain A, | IGNTGDT |
| | CALRB421 Heavy | |
| | CDR2 IMGT | |
| 264 | C3CRB75 Chain A, | VRGPPSYSSSVKNI |
| | CALRB421 Heavy | |
| | CDR3 IMGT | |
| 203 | C3CRB75 Chain A, | EVQLLESGGGLVQPGGSLRLSCAVSGIDLS |
| | CALRB421 VH | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSS |
| 214 | C3CRB75 Chain A, CD3 | RARQSIGTAIH |
| | Light CDR1 AbM | |
| 215 | C3CRB75 Chain A, CD3 | YASESIS |
| | Light CDR2 AbM | |
| 216 | C3CRB75 Chain A, CD3 | QQSGSWPYT |
| | Light CDR3 AbM | |
| 214 | C3CRB75 Chain A, CD3 | RARQSIGTAIH |
| | Light CDR1 Chothia | |
| 215 | C3CRB75 Chain A, CD3 | YASESIS |
| | Light CDR2 Chothia | |
| 216 | C3CRB75 Chain A, CD3 | QQSGSWPYT |
| | Light CDR3 Chothia | |
| 214 | C3CRB75 Chain A, CD3 | RARQSIGTAIH |
| | Light CDR1 Kabat | |
| 215 | C3CRB75 Chain A, CD3 | YASESIS |
| | Light CDR2 Kabat | |
| 216 | C3CRB75 Chain A, CD3 | QQSGSWPYT |
| | Light CDR3 Kabat | |
| 272 | C3CRB75 Chain A, CD3 | QSIGTA |
| | Light CDR1 IMGT | |
| n/a | C3CRB75 Chain A, CD3 | YAS |
| | Light CDR2 IMGT | |
| 216 | C3CRB75 Chain A, CD3 | QQSGSWPYT |
| | Light CDR3 IMGT | |
| 217 | C3CRB75 Chain A, CD3 | DIQMTQSPSSLSASVGDRVTITCRARQSIG |
| | VL | TAIHWYQQKPGCAPKLLIKYASESISGVPS |
| | | RFSGSGSGTDFTLTISSLOPEDFATYYCQQ |
| | | SGSWPYTFGQGTKLEIK |
| 210 | C3CRB75 Chain A, CD3 | GFTFSRYNMN |
| | Heavy CDR1 AbM | |
| 211 | C3CRB75 Chain A, CD3 | SISTSSNYIY |
| | Heavy CDR2 AbM | |
| 212 | C3CRB75 Chain A, CD3 | GWGPFDY |
| | Heavy CDR3 AbM | |
| 267 | C3CRB75 Chain A, CD3 | GFTFSRY |
| | Heavy CDR1 Chothia | |
| 268 | C3CRB75 Chain A, CD3 | STSSNY |
| | Heavy CDR2 Chothia | |
| 212 | C3CRB75 Chain A, CD3 | GWGPFDY |
| | Heavy CDR3 Chothia | |
| 265 | C3CRB75 Chain A, CD3 | RYNMN |
| | Heavy CDR1 Kabat | |
| 266 | C3CRB75 Chain A, CD3 | SISTSSNYIYYADSVKG |
| | Heavy CDR2 Kabat | |
| 212 | C3CRB75 Chain A, CD3 | GWGPFDY |
| | Heavy CDR3 Kabat | |
| 269 | C3CRB75 Chain A, CD3 | GFTFSRYN |
| | Heavy CDR1 IMGT | |
| 270 | C3CRB75 Chain A, CD3 | ISTSSNYI |
| | Heavy CDR2 IMGT | |
| 271 | C3CRB75 Chain A, CD3 | TRGWGPFDY |
| | Heavy CDR3 IMGT | |
| 213 | C3CRB75 Chain A, CD3 | EVOLVESGGGLVKPGGSLRLSCAASGFTF |
| | VH | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 289 | C3CRB75 Chain A | EVQLLESGGGLVQPGGSLRLSCAVSGIDLS |
| | Heavy Chain | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSSASTKGPSVFPLAPSSKSTSGGTAALG |
| | | CLVKDYFPEPVTVSWNSGALTSGVHTFPA |
| | | VLQSSGLYSLSSVVTVPSSSLGTQTYICNV |
| | | NHKPSNTKVDKKVEPKSCDGGGGSGGGG |
| | | SDIQMTQSPSSLSASVGDRVTITCRARQSI |
| | | GTAIHWYQQKPGCAPKLLIKYASESISGVP |
| | | SRFSGSGSGTDFTLTISSLQPEDFATYYCQ |
| | | QSGSWPYTFGQGTKLEIKGGGSGGSGGCP |
| | | PCGGSGGEVOLVESGGGLVKPGGSLRLSC |
| | | AASGFTFSRYNMNWVRQAPGKGLEWVSS |
| | | ISTSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |
| 33 | C3CRB75 Chain A, | QSSQSVYNNNWLS |
| | CALRB421 Light CDR1 | |
| | AbM | |
| 23 | C3CRB75 Chain A, | DASKLES |
| | CALRB421 Light CDR2 | |
| | AbM | |
| 34 | C3CRB75 Chain A, | AGGFTGNVYT |
| | CALRB421 Light CDR3 | |
| | AbM | |
| 33 | C3CRB75 Chain A, | QSSQSVYNNNWLS |
| | CALRB421 Light CDR1 | |
| | Chothia | |
| 23 | C3CRB75 Chain A, | DASKLES |
| | CALRB421 Light CDR2 | |
| | Chothia | |
| 34 | C3CRB75 Chain A, | AGGFTGNVYT |
| | CALRB421 Light CDR3 | |
| | Chothia | |
| 33 | C3CRB75 Chain A, | QSSQSVYNNNWLS |
| | CALRB421 Light CDR1 | |
| | Kabat | |
| 23 | C3CRB75 Chain A, | DASKLES |
| | CALRB421 Light CDR2 | |
| | Kabat | |
| 34 | C3CRB75 Chain A, | AGGFTGNVYT |
| | CALRB421 Light CDR3 | |
| | Kabat | |
| 256 | C3CRB75 Chain A, | QSVYNNNW |
| | CALRB421 Light CDR1 | |
| | IMGT | |
| n/a | C3CRB75 Chain A, | DAS |
| | CALRB421 Light CDR2 | |
| | IMGT | |
| 34 | C3CRB75 Chain A, | AGGFTGNVYT |
| | CALRB421 Light CDR3 | |
| | IMGT | |
| 205 | C3CRB75 Chain A, | DIQMTQSPSSVSASVGDRVTITCQSSQSVY |
| | CALRB421 VL | NNNWLSWLQQKPGKAPKRLIYDASKLES |
| | | GVPSRFSGSGSGTDFTLTISSVQPEDAATY |
| | | YCAGGFTGNVYTFGGGTKVEIK |
| 206 | C3CRB75 Chain A Light | DIQMTQSPSSVSASVGDRVTITCQSSQSVY |
| | Chain | NNNWLSWLQQKPGKAPKRLIYDASKLES |
| | | GVPSRFSGSGSGTDFTLTISSVQPEDAATY |
| | | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |
| 200 | C3CRB75 Chain B, | GIDLSNNAIS |
| | CALRB421 Heavy | |
| | CDR1 AbM | |
| 201 | C3CRB75 Chain B, | VIGNTGDTY |
| | CALRB421 Heavy | |
| | CDR2 AbM | |
| 202 | C3CRB75 Chain B, | GPPSYSSSVKNI |
| | CALRB421 Heavy | |
| | CDR3 AbM | |
| 260 | C3CRB75 Chain B, | GIDLSNN |
| | CALRB421 Heavy | |
| | CDR1 Chothia | |
| 261 | C3CRB75 Chain B, | GNTGD |
| | CALRB421 Heavy | |
| | CDR2 Chothia | |
| 202 | C3CRB75 Chain B, | GPPSYSSSVKNI |
| | CALRB421 Heavy | |
| | CDR3 Chothia | |
| 81 | C3CRB75 Chain B, | NNAIS |
| | CALRB421 Heavy | |
| | CDR1 Kabat | |
| 259 | C3CRB75 Chain B, | VIGNTGDTYYADSAKG |
| | CALRB421 Heavy | |
| | CDR2 Kabat | |
| 202 | C3CRB75 Chain B, | GPPSYSSSVKNI |
| | CALRB421 Heavy | |
| | CDR3 Kabat | |
| 262 | C3CRB75 Chain B, | GIDLSNNA |
| | CALRB421 Heavy | |
| | CDR1 IMGT | |
| 263 | C3CRB75 Chain B, | IGNTGDT |
| | CALRB421 Heavy | |
| | CDR2 IMGT | |
| 264 | C3CRB75 Chain B, | VRGPPSYSSSVKNI |
| | CALRB421 Heavy | |
| | CDR3 IMGT | |
| 203 | C3CRB75 Chain B, | EVQLLESGGGLVQPGGSLRLSCAVSGIDLS |
| | CALRB421 VH | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSS |
| 204 | C3CRB75 Chain B | EVQLLESGGGLVQPGGSLRLSCAVSGIDLS |
| | Heavy Chain | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSSASTKGPSVFPLAPSSKSTSGGTAALG |
| | | CLVKDYFPEPVTVSWNSGALTSGVHTFPA |
| | | VLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTC |
| | | VVVSVSHEDPEVKFNWYVDGVEVHNAKT |
| | | KPREEQYNSTYRVVSVLTVLHQDWLNGK |
| | | EYKCKVSNKALPAPIEKTISKAKGQPREPQ |
| | | VYTLPPSREEMTKNQVSLSCAVKGFYPSDI |
| | | AVEWESNGQPENNYKTTPPVLDSDGSFFL |
| | | VSKLTVDKSRWQQGNVFSCSVMHEALHN |
| | | RFTQKSLSLSPGK |
| 33 | C3CRB75 Chain B, | QSSQSVYNNNWLS |
| | CALRB421 Light CDR1 | |
| | AbM | |
| 23 | C3CRB75 Chain B, | DASKLES |
| | CALRB421 Light CDR2 | |
| | AbM | |
| 34 | C3CRB75 Chain B, | AGGFTGNVYT |
| | CALRB421 Light CDR3 | |
| | AbM | |
| 33 | C3CRB75 Chain B, | QSSQSVYNNNWLS |
| | CALRB421 Light CDR1 | |
| | Chothia | |
| 23 | C3CRB75 Chain B, | DASKLES |
| | CALRB421 Light CDR2 | |
| | Chothia | |
| 34 | C3CRB75 Chain B, | AGGFTGNVYT |
| | CALRB421 Light CDR3 | |
| | Chothia | |
| 33 | C3CRB75 Chain B, | QSSQSVYNNNWLS |
| | CALRB421 Light CDR1 | |
| | Kabat | |
| 23 | C3CRB75 Chain B, | DASKLES |
| | CALRB421 Light CDR2 | |
| | Kabat | |
| 34 | C3CRB75 Chain B, | AGGFTGNVYT |
| | CALRB421 Light CDR3 | |
| | Kabat | |
| 256 | C3CRB75 Chain B, | QSVYNNNW |
| | CALRB421 Light CDR1 | |
| | IMGT | |
| n/a | C3CRB75 Chain B, | DAS |
| | CALRB421 Light CDR2 | |
| | IMGT | |
| 34 | C3CRB75 Chain B, | AGGFTGNVYT |
| | CALRB421 Light CDR3 | |
| | IMGT | |
| 205 | C3CRB75 Chain B, | DIQMTQSPSSVSASVGDRVTITCQSSQSVY |
| | CALRB421 VL | NNNWLSWLQQKPGKAPKRLIYDASKLES |
| | | GVPSRFSGSGSGTDFTLTISSVQPEDAATY |
| | | YCAGGFTGNVYTFGGGTKVEIK |
| 206 | C3CRB75 Chain B Light | DIQMTQSPSSVSASVGDRVTITCQSSQSVY |
| | Chain | NNNWLSWLQQKPGKAPKRLIYDASKLES |
| | | GVPSRFSGSGSGTDFTLTISSVQPEDAATY |
| | | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |

TABLE 22
Sequences for CALRB1508 CALRmut x CD3 Bispecific Antibody

| SEQ ID NO | Description | Sequence |
|---|---|---|
| 214 | CALRB1508 Chain A, | RARQSIGTAIH |
| | CD3 Light CDR1 AbM | |
| 215 | CALRB1508 Chain A, | YASESIS |
| | CD3 Light CDR2 AbM | |
| 216 | CALRB1508 Chain A, | QQSGSWPYT |
| | CD3 Light CDR3 AbM | |
| 214 | CALRB1508 Chain A, | RARQSIGTAIH |
| | CD3 Light CDR1 | |
| | Chothia | |
| 215 | CALRB1508 Chain A, | YASESIS |
| | CD3 Light CDR2 | |
| | Chothia | |
| 216 | CALRB1508 Chain A, | QQSGSWPYT |
| | CD3 Light CDR3 | |
| | Chothia | |
| 214 | CALRB1508 Chain A, | RARQSIGTAIH |
| | CD3 Light CDR1 Kabat | |
| 215 | CALRB1508 Chain A, | YASESIS |
| | CD3 Light CDR2 Kabat | |
| 216 | CALRB1508 Chain A, | QQSGSWPYT |
| | CD3 Light CDR3 Kabat | |
| 272 | CALRB1508 Chain A, | QSIGTA |
| | CD3 Light CDR1 IMGT | |
| n/a | CALRB1508 Chain A, | YAS |
| | CD3 Light CDR2 IMGT | |
| 216 | CALRB1508 Chain A, | QQSGSWPYT |
| | CD3 Light CDR3 IMGT | |
| 217 | CALRB1508 Chain A, | DIQMTQSPSSLSASVGDRVTITCRARQSIG |
| | CD3 VL | TAIHWYQQKPGCAPKLLIKYASESISGVPS |
| | | RFSGSGSGTDFTLTISSLQPEDFATYYCQQ |
| | | SGSWPYTFGQGTKLEIK |
| 210 | CALRB1508 Chain A, | GFTFSRYNMN |
| | CD3 Heavy CDR1 AbM | |
| 211 | CALRB1508 Chain A, | SISTSSNYIY |
| | CD3 Heavy CDR2 AbM | |
| 212 | CALRB1508 Chain A, | GWGPFDY |
| | CD3 Heavy CDR3 AbM | |
| 267 | CALRB1508 Chain A, | GFTFSRY |
| | CD3 Heavy CDR1 | |
| | Chothia | |
| 268 | CALRB1508 Chain A, | STSSNY |
| | CD3 Heavy CDR2 | |
| | Chothia | |
| 212 | CALRB1508 Chain A, | GWGPFDY |
| | CD3 Heavy CDR3 | |
| | Chothia | |
| 265 | CALRB1508 Chain A, | RYNMN |
| | CD3 Heavy CDR1 Kabat | |
| 266 | CALRB1508 Chain A, | SISTSSNYIYYADSVKG |
| | CD3 Heavy CDR2 Kabat | |
| 212 | CALRB1508 Chain A, | GWGPFDY |
| | CD3 Heavy CDR3 Kabat | |
| 269 | CALRB1508 Chain A, | GFTFSRYN |
| | CD3 Heavy CDR1 | |
| | IMGT | |
| 270 | CALRB1508 Chain A, | ISTSSNYI |
| | CD3 Heavy CDR2 | |
| | IMGT | |
| 271 | CALRB1508 Chain A, | TRGWGPFDY |
| | CD3 Heavy CDR3 | |
| | IMGT | |
| 213 | CALRB1508 Chain A, | EVOLVESGGGLVKPGGSLRLSCAASGFTF |
| | CD3 VH | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 218 | CALRB1508 Chain A | DIQMTQSPSSLSASVGDRVTITCRARQSIG |
| | Heavy Chain | TAIHWYQQKPGCAPKLLIKYASESISGVPS |
| | | RFSGSGSGTDFTLTISSLQPEDFATYYCQQ |
| | | SGSWPYTFGQGTKLEIKGGGSGGSGGCPP |
| | | CGGSGGEVQLVESGGGLVKPGGSLRLSCA |
| | | ASGFTFSRYNMNWVRQAPGKGLEWVSSI |
| | | STSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |
| 273 | CALRB1508 Chain B, | GFSLSSYAMS |
| | CALRB1114 Heavy | |
| | CDR1 AbM | |
| 274 | CALRB1508 Chain B, | VIDSGGTTY |
| | CALRB1114 Heavy | |
| | CDR2 AbM | |
| 275 | CALRB1508 Chain B, | ESYAGYDYYGHPIPYGMEP |
| | CALRB1114 Heavy | |
| | CDR3 AbM | |
| 276 | CALRB1508 Chain B, | GFSLSSY |
| | CALRB1114 Heavy | |
| | CDR1 Chothia | |
| 277 | CALRB1508 Chain B, | DSGGT |
| | CALRB1114 Heavy | |
| | CDR2 Chothia | |
| 275 | CALRB1508 Chain B, | ESYAGYDYYGHPIPYGMEP |
| | CALRB1114 Heavy | |
| | CDR3 Chothia | |
| 66 | CALRB1508 Chain B, | SYAMS |
| | CALRB1114 Heavy | |
| | CDR1 Kabat | |
| 278 | CALRB1508 Chain B, | VIDSGGTTYYAQKFQG |
| | CALRB1114 Heavy | |
| | CDR2 Kabat | |
| 275 | CALRB1508 Chain B, | ESYAGYDYYGHPIPYGMEP |
| | CALRB1114 Heavy | |
| | CDR3 Kabat | |
| 279 | CALRB1508 Chain B, | GFSLSSYA |
| | CALRB1114 Heavy | |
| | CDR1 IMGT | |
| 280 | CALRB1508 Chain B, | IDSGGTT |
| | CALRB1114 Heavy | |
| | CDR2 IMGT | |
| 281 | CALRB1508 Chain B, | ARESYAGYDYYGHPIPYGMEP |
| | CALRB1114 Heavy | |
| | CDR3 IMGT | |
| 282 | CALRB1508 Chain B, | QVQLVQSGAEVKKPGASVKVSCKASGFS |
| | CALRB1114 VH | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSS |
| 290 | CALRB1508 Chain B | QVQLVQSGAEVKKPGASVKVSCKASGFS |
| | Heavy Chain | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSSASTKGPSVFPLAPSS |
| | | KSTSGGTAALGCLVKDYFPEPVTVSWNSG |
| | | ALTSGVHTFPAVLQSSGLYSLSSVVTVPSS |
| | | SLGTQTYICNVNHKPSNTKVDKKVEPKSC |
| | | DKTHTCPPCPAPEAAGGPSVFLFPPKPKDT |
| | | LMISRTPEVTCVVVSVSHEDPEVKFNWYV |
| | | DGVEVHNAKTKPREEQYNSTYRVVSVLT |
| | | VLHQDWLNGKEYKCKVSNKALPAPIEKTI |
| | | SKAKGQPREPQVYTLPPSREEMTKNQVSL |
| | | SCAVKGFYPSDIAVEWESNGQPENNYKTT |
| | | PPVLDSDGSFFLVSKLTVDKSRWQQGNVF |
| | | SCSVMHEALHNRFTQKSLSLSPGK |
| 70 | CALRB1508 Chain B, | QASQSLYNNKNLA |
| | CALRB1114 Light | |
| | CDR1 AbM | |
| 71 | CALRB1508 Chain B, | YASALAS |
| | CALRB1114 Light | |
| | CDR2 AbM | |
| 72 | CALRB1508 Chain B, | QGEFNCNSADCNS |
| | CALRB1114 Light | |
| | CDR3 AbM | |
| 70 | CALRB1508 Chain B, | QASQSLYNNKNLA |
| | CALRB1114 Light | |
| | CDR1 Chothia | |
| 71 | CALRB1508 Chain B, | YASALAS |
| | CALRB1114 Light | |
| | CDR2 Chothia | |
| 72 | CALRB1508 Chain B, | QGEFNCNSADCNS |
| | CALRB1114 Light | |
| | CDR3 Chothia | |
| 70 | CALRB1508 Chain B, | QASQSLYNNKNLA |
| | CALRB1114 Light | |
| | CDR1 Kabat | |
| 71 | CALRB1508 Chain B, | YASALAS |
| | CALRB1114 Light | |
| | CDR2 Kabat | |
| 72 | CALRB1508 Chain B, | QGEFNCNSADCNS |
| | CALRB1114 Light | |
| | CDR3 Kabat | |
| 283 | CALRB1508 Chain B, | QSLYNNKN |
| | CALRB1114 Light | |
| | CDR1 IMGT | |
| n/a | CALRB1508 Chain B, | YAS |
| | CALRB1114 Light | |
| | CDR2 IMGT | |
| 72 | CALRB1508 Chain B, | QGEFNCNSADCNS |
| | CALRB1114 Light | |
| | CDR3 IMGT | |
| 284 | CALRB1508 Chain B, | DIQMTQSPSTLSASVGDRVTITCQASQSLY |
| | CALRB1114 VL | NNKNLAWYQQKPGKAPKLLIYYASALAS |
| | | GVPSRFSGSGSGTEFTLTISSLQPDDFATYY |
| | | CQGEFNCNSADCNSFGGGTKVEIK |
| 291 | CALRB1508 Chain B | DIQMTQSPSTLSASVGDRVTITCQASQSLY |
| | Light Chain | NNKNLAWYQQKPGKAPKLLIYYASALAS |
| | | GVPSRFSGSGSGTEFTLTISSLQPDDFATYY |
| | | CQGEFNCNSADCNSFGGGTKVEIKRTVAA |
| | | PSVFIFPPSDEQLKSGTASVVCLLNNFYPR |
| | | EAKVQWKVDNALQSGNSQESVTEQDSKD |
| | | STYSLSSTLTLSKADYEKHKVYACEVTHQ |
| | | GLSSPVTKSFNRGEC |

TABLE 23
Sequences for C3CRB126 CALRmut x CD3 Bispecific Antibody

| SEQ ID NO | Description | Sequence |
|---|---|---|
| 273 | C3CRB126 Chain A, | GFSLSSYAMS |
| | CALRB1114 Heavy | |
| | CDR1 AbM | |
| 274 | C3CRB126 Chain A, | VIDSGGTTY |
| | CALRB1114 Heavy | |
| | CDR2 AbM | |
| 275 | C3CRB126 Chain A, | ESYAGYDYYGHPIPYGMEP |
| | CALRB1114 Heavy | |
| | CDR3 AbM | |
| 276 | C3CRB126 Chain A, | GFSLSSY |
| | CALRB1114 Heavy | |
| | CDR1 Chothia | |
| 277 | C3CRB126 Chain A, | DSGGT |
| | CALRB1114 Heavy | |
| | CDR2 Chothia | |
| 275 | C3CRB126 Chain A, | ESYAGYDYYGHPIPYGMEP |
| | CALRB1114 Heavy | |
| | CDR3 Chothia | |
| 66 | C3CRB126 Chain A, | SYAMS |
| | CALRB1114 Heavy | |
| | CDR1 Kabat | |
| 278 | C3CRB126 Chain A, | VIDSGGTTYYAQKFQG |
| | CALRB1114 Heavy | |
| | CDR2 Kabat | |
| 275 | C3CRB126 Chain A, | ESYAGYDYYGHPIPYGMEP |
| | CALRB1114 Heavy | |
| | CDR3 Kabat | |
| 279 | C3CRB126 Chain A, | GFSLSSYA |
| | CALRB1114 Heavy | |
| | CDR1 IMGT | |
| 280 | C3CRB126 Chain A, | IDSGGTT |
| | CALRB1114 Heavy | |
| | CDR2 IMGT | |
| 281 | C3CRB126 Chain A, | ARESYAGYDYYGHPIPYGMEP |
| | CALRB1114 Heavy | |
| | CDR3 IMGT | |
| 282 | C3CRB126 Chain A, | QVQLVQSGAEVKKPGASVKVSCKASGFS |
| | CALRB1114 VH | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSS |
| 214 | C3CRB126 Chain A, | RARQSIGTAIH |
| | CD3 Light CDR1 AbM | |
| 215 | C3CRB126 Chain A, | YASESIS |
| | CD3 Light CDR2 AbM | |
| 216 | C3CRB126 Chain A, | QQSGSWPYT |
| | CD3 Light CDR3 AbM | |
| 214 | C3CRB126 Chain A, | RARQSIGTAIH |
| | CD3 Light CDR1 | |
| | Chothia | |
| 215 | C3CRB126 Chain A, | YASESIS |
| | CD3 Light CDR2 | |
| | Chothia | |
| 216 | C3CRB126 Chain A, | QQSGSWPYT |
| | CD3 Light CDR3 | |
| | Chothia | |
| 214 | C3CRB126 Chain A, | RARQSIGTAIH |
| | CD3 Light CDR1 Kabat | |
| 215 | C3CRB126 Chain A, | YASESIS |
| | CD3 Light CDR2 Kabat | |
| 216 | C3CRB126 Chain A, | QQSGSWPYT |
| | CD3 Light CDR3 Kabat | |
| 272 | C3CRB126 Chain A, | QSIGTA |
| | CD3 Light CDR1 IMGT | |
| n/a | C3CRB126 Chain A, | YAS |
| | CD3 Light CDR2 IMGT | |
| 216 | C3CRB126 Chain A, | QQSGSWPYT |
| | CD3 Light CDR3 IMGT | |
| 217 | C3CRB126 Chain A, | DIQMTQSPSSLSASVGDRVTITCRARQSIG |
| | CD3 VL | TAIHWYQQKPGCAPKLLIKYASESISGVPS |
| | | RFSGSGSGTDFTLTISSLQPEDFATYYCQQ |
| | | SGSWPYTFGQGTKLEIK |
| 210 | C3CRB126 Chain A, | GFTFSRYNMN |
| | CD3 Heavy CDR1 AbM | |
| 211 | C3CRB126 Chain A, | SISTSSNYIY |
| | CD3 Heavy CDR2 AbM | |
| 212 | C3CRB126 Chain A, | GWGPFDY |
| | CD3 Heavy CDR3 AbM | |
| 267 | C3CRB 126 Chain A, | GFTFSRY |
| | CD3 Heavy CDR1 | |
| | Chothia | |
| 268 | C3CRB126 Chain A, | STSSNY |
| | CD3 Heavy CDR2 | |
| | Chothia | |
| 212 | C3CRB126 Chain A, | GWGPFDY |
| | CD3 Heavy CDR3 | |
| | Chothia | |
| 265 | C3CRB126 Chain A, | RYNMN |
| | CD3 Heavy CDR1 Kabat | |
| 266 | C3CRB126 Chain A, | SISTSSNYIYYADSVKG |
| | CD3 Heavy CDR2 Kabat | |
| 212 | C3CRB126 Chain A, | GWGPFDY |
| | CD3 Heavy CDR3 Kabat | |
| 269 | C3CRB126 Chain A, | GFTFSRYN |
| | CD3 Heavy CDR1 | |
| | IMGT | |
| 270 | C3CRB126 Chain A, | ISTSSNYI |
| | CD3 Heavy CDR2 | |
| | IMGT | |
| 271 | C3CRB126 Chain A, | TRGWGPFDY |
| | CD3 Heavy CDR3 | |
| | IMGT | |
| 213 | C3CRB126 Chain A, | EVQLVESGGGLVKPGGSLRLSCAASGFTF |
| | CD3 VH | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 292 | C3CRB126 Chain A | QVQLVQSGAEVKKPGASVKVSCKASGFS |
| | Heavy Chain | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSSASTKGPSVFPLAPSS |
| | | KSTSGGTAALGCLVKDYFPEPVTVSWNSG |
| | | ALTSGVHTFPAVLQSSGLYSLSSVVTVPSS |
| | | SLGTQTYICNVNHKPSNTKVDKKVEPKSC |
| | | GGGGSGGGGSDIQMTQSPSSLSASVGDRV |
| | | TITCRARQSIGTAIHWYQQKPGCAPKLLIK |
| | | YASESISGVPSRFSGSGSGTDFTLTISSLQPE |
| | | DFATYYCQQSGSWPYTFGQGTKLEIKGGG |
| | | SGGSGGCPPCGGSGGEVOLVESGGGLVKP |
| | | GGSLRLSCAASGFTFSRYNMNWVRQAPG |
| | | KGLEWVSSISTSSNYIYYADSVKGRFTFSR |
| | | DNAKNSLDLQMSGLRAEDTAIYYCTRGW |
| | | GPFDYWGCGTLVTVSSEPKSSDKTHTCPP |
| | | CPAPEAAGGPSVFLFPPKPKDTLMISRTPE |
| | | VTCVVVSVSHEDPEVKFNWYVDGVEVHN |
| | | AKTKPREEQYNSTYRVVSVLTVLHQDWL |
| | | NGKEYKCKVSNKALPAPIEKTISKAKGQP |
| | | REPQVYTLPPSREEMTKNQVSLWCLVKGF |
| | | YPSDIAVEWESNGQPENNYKTTPPVLDSD |
| | | GSFFLYSKLTVDKSRWQQGNVFSCSVMH |
| | | EALHNHYTQKSLSLSPGK |
| 70 | C3CRB126 Chain A, | QASQSLYNNKNLA |
| | CALRB1114 Light | |
| | CDR1 AbM | |
| 71 | C3CRB126 Chain A, | YASALAS |
| | CALRB1114 Light | |
| | CDR2 AbM | |
| 72 | C3CRB126 Chain A, | QGEFNCNSADCNS |
| | CALRB1114 Light | |
| | CDR3 AbM | |
| 70 | C3CRB 126 Chain A, | QASQSLYNNKNLA |
| | CALRB1114 Light | |
| | CDR1 Chothia | |
| 71 | C3CRB126 Chain A, | YASALAS |
| | CALRB1114 Light | |
| | CDR2 Chothia | |
| 72 | C3CRB126 Chain A, | QGEFNCNSADCNS |
| | CALRB1114 Light | |
| | CDR3 Chothia | |
| 70 | C3CRB126 Chain A, | QASQSLYNNKNLA |
| | CALRB1114 Light | |
| | CDR1 Kabat | |
| 71 | C3CRB 126 Chain A, | YASALAS |
| | CALRB1114 Light | |
| | CDR2 Kabat | |
| 72 | C3CRB126 Chain A, | QGEFNCNSADCNS |
| | CALRB1114 Light | |
| | CDR3 Kabat | |
| 283 | C3CRB126 Chain A, | QSLYNNKN |
| | CALRB1114 Light | |
| | CDR1 IMGT | |
| n/a | C3CRB126 Chain A, | YAS |
| | CALRB1114 Light | |
| | CDR2 IMGT | |
| 72 | C3CRB 126 Chain A, | QGEFNCNSADCNS |
| | CALRB1114 Light | |
| | CDR3 IMGT | |
| 284 | C3CRB126 Chain A, | DIQMTQSPSTLSASVGDRVTITCQASQSLY |
| | CALRB1114 VL | NNKNLAWYQQKPGKAPKLLIYYASALAS |
| | | GVPSRFSGSGSGTEFTLTISSLQPDDFATYY |
| | | CQGEFNCNSADCNSFGGGTKVEIK |
| 291 | C3CRB126 Chain A | DIQMTQSPSTLSASVGDRVTITCQASQSLY |
| | Light Chain | NNKNLAWYQQKPGKAPKLLIYYASALAS |
| | | GVPSRFSGSGSGTEFTLTISSLQPDDFATYY |
| | | CQGEFNCNSADCNSFGGGTKVEIKRTVAA |
| | | PSVFIFPPSDEQLKSGTASVVCLLNNFYPR |
| | | EAKVQWKVDNALQSGNSQESVTEQDSKD |
| | | STYSLSSTLTLSKADYEKHKVYACEVTHQ |
| | | GLSSPVTKSFNRGEC |
| 273 | C3CRB126 Chain B, | GFSLSSYAMS |
| | CALRB1114 Heavy | |
| | CDR1 AbM | |
| 274 | C3CRB126 Chain B, | VIDSGGTTY |
| | CALRB1114 Heavy | |
| | CDR2 AbM | |
| 275 | C3CRB126 Chain B, | ESYAGYDYYGHPIPYGMEP |
| | CALRB1114 Heavy | |
| | CDR3 AbM | |
| 276 | C3CRB126 Chain B, | GFSLSSY |
| | CALRB1114 Heavy | |
| | CDR1 Chothia | |
| 277 | C3CRB126 Chain B, | DSGGT |
| | CALRB1114 Heavy | |
| | CDR2 Chothia | |
| 275 | C3CRB126 Chain B, | ESYAGYDYYGHPIPYGMEP |
| | CALRB1114 Heavy | |
| | CDR3 Chothia | |
| 66 | C3CRB126 Chain B, | SYAMS |
| | CALRB1114 Heavy | |
| | CDR1 Kabat | |
| 278 | C3CRB126 Chain B, | VIDSGGTTYYAQKFQG |
| | CALRB1114 Heavy | |
| | CDR2 Kabat | |
| 275 | C3CRB126 Chain B, | ESYAGYDYYGHPIPYGMEP |
| | CALRB1114 Heavy | |
| | CDR3 Kabat | |
| 279 | C3CRB126 Chain B, | GFSLSSYA |
| | CALRB1114 Heavy | |
| | CDR1 IMGT | |
| 280 | C3CRB 126 Chain B, | IDSGGTT |
| | CALRB1114 Heavy | |
| | CDR2 IMGT | |
| 281 | C3CRB126 Chain B, | ARESYAGYDYYGHPIPYGMEP |
| | CALRB1114 Heavy | |
| | CDR3 IMGT | |
| 282 | C3CRB126 Chain B, | QVQLVQSGAEVKKPGASVKVSCKASGFS |
| | CALRB1114 VH | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSS |
| 290 | C3CRB126 Chain B | QVQLVQSGAEVKKPGASVKVSCKASGFS |
| | Heavy Chain | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSSASTKGPSVFPLAPSS |
| | | KSTSGGTAALGCLVKDYFPEPVTVSWNSG |
| | | ALTSGVHTFPAVLQSSGLYSLSSVVTVPSS |
| | | SLGTQTYICNVNHKPSNTKVDKKVEPKSC |
| | | DKTHTCPPCPAPEAAGGPSVFLFPPKPKDT |
| | | LMISRTPEVTCVVVSVSHEDPEVKFNWYV |
| | | DGVEVHNAKTKPREEQYNSTYRVVSVLT |
| | | VLHQDWLNGKEYKCKVSNKALPAPIEKTI |
| | | SKAKGQPREPQVYTLPPSREEMTKNQVSL |
| | | SCAVKGFYPSDIAVEWESNGQPENNYKTT |
| | | PPVLDSDGSFFLVSKLTVDKSRWQQGNVF |
| | | SCSVMHEALHNRFTQKSLSLSPGK |
| 70 | C3CRB126 Chain B, | QASQSLYNNKNLA |
| | CALRB1114 Light | |
| | CDR1 AbM | |
| 71 | C3CRB126 Chain B, | YASALAS |
| | CALRB1114 Light | |
| | CDR2 AbM | |
| 72 | C3CRB126 Chain B, | QGEFNCNSADCNS |
| | CALRB1114 Light | |
| | CDR3 AbM | |
| 70 | C3CRB126 Chain B, | QASQSLYNNKNLA |
| | CALRB1114 Light | |
| | CDR1 Chothia | |
| 71 | C3CRB126 Chain B, | YASALAS |
| | CALRB1114 Light | |
| | CDR2 Chothia | |
| 72 | C3CRB126 Chain B, | QGEFNCNSADCNS |
| | CALRB1114 Light | |
| | CDR3 Chothia | |
| 70 | C3CRB126 Chain B, | QASQSLYNNKNLA |
| | CALRB1114 Light | |
| | CDR1 Kabat | |
| 71 | C3CRB126 Chain B, | YASALAS |
| | CALRB1114 Light | |
| | CDR2 Kabat | |
| 72 | C3CRB126 Chain B, | QGEFNCNSADCNS |
| | CALRB1114 Light | |
| | CDR3 Kabat | |
| 283 | C3CRB126 Chain B, | QSLYNNKN |
| | CALRB1114 Light | |
| | CDR1 IMGT | |
| n/a | C3CRB126 Chain B, | YAS |
| | CALRB1114 Light | |
| | CDR2 IMGT | |
| 72 | C3CRB126 Chain B, | QGEFNCNSADCNS |
| | CALRB1114 Light | |
| | CDR3 IMGT | |
| 284 | C3CRB126 Chain B, | DIQMTQSPSTLSASVGDRVTITCQASQSLY |
| | CALRB1114 VL | NNKNLAWYQQKPGKAPKLLIYYASALAS |
| | | GVPSRFSGSGSGTEFTLTISSLQPDDFATYY |
| | | CQGEFNCNSADCNSFGGGTKVEIK |
| 291 | C3CRB126 Chain B | DIQMTQSPSTLSASVGDRVTITCQASQSLY |
| | Light Chain | NNKNLAWYQQKPGKAPKLLIYYASALAS |
| | | GVPSRFSGSGSGTEFTLTISSLQPDDFATYY |
| | | CQGEFNCNSADCNSFGGGTKVEIKRTVAA |
| | | PSVFIFPPSDEQLKSGTASVVCLLNNFYPR |
| | | EAKVQWKVDNALQSGNSQESVTEQDSKD |
| | | STYSLSSTLTLSKADYEKHKVYACEVTHQ |
| | | GLSSPVTKSFNRGEC |

TABLE 24
Sequences for C3CRB127 CALRmut x CD3 Bispecific Antibody

| SEQ ID NO | Description | Sequence |
|---|---|---|
| 273 | C3CRB127 Chain A, | GFSLSSYAMS |
| | CALRB1114 Heavy | |
| | CDR1 AbM | |
| 274 | C3CRB127 Chain A, | VIDSGGTTY |
| | CALRB1114 Heavy | |
| | CDR2 AbM | |
| 275 | C3CRB127 Chain A, | ESYAGYDYYGHPIPYGMEP |
| | CALRB1114 Heavy | |
| | CDR3 AbM | |
| 276 | C3CRB127 Chain A, | GFSLSSY |
| | CALRB1114 Heavy | |
| | CDR1 Chothia | |
| 277 | C3CRB127 Chain A, | DSGGT |
| | CALRB1114 Heavy | |
| | CDR2 Chothia | |
| 275 | C3CRB127 Chain A, | ESYAGYDYYGHPIPYGMEP |
| | CALRB1114 Heavy | |
| | CDR3 Chothia | |
| 66 | C3CRB127 Chain A, | SYAMS |
| | CALRB1114 Heavy | |
| | CDR1 Kabat | |
| 278 | C3CRB127 Chain A, | VIDSGGTTYYAQKFQG |
| | CALRB1114 Heavy | |
| | CDR2 Kabat | |
| 275 | C3CRB127 Chain A, | ESYAGYDYYGHPIPYGMEP |
| | CALRB1114 Heavy | |
| | CDR3 Kabat | |
| 279 | C3CRB127 Chain A, | GFSLSSYA |
| | CALRB1114 Heavy | |
| | CDR1 IMGT | |
| 280 | C3CRB127 Chain A, | IDSGGTT |
| | CALRB1114 Heavy | |
| | CDR2 IMGT | |
| 281 | C3CRB127 Chain A, | ARESYAGYDYYGHPIPYGMEP |
| | CALRB1114 Heavy | |
| | CDR3 IMGT | |
| 282 | C3CRB127 Chain A, | QVQLVQSGAEVKKPGASVKVSCKASGFS |
| | CALRB1114 VH | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSS |
| 210 | C3CRB127 Chain A, | GFTFSRYNMN |
| | CD3 Heavy CDR1 AbM | |
| 211 | C3CRB127 Chain A, | SISTSSNYIY |
| | CD3 Heavy CDR2 AbM | |
| 212 | C3CRB127 Chain A, | GWGPFDY |
| | CD3 Heavy CDR3 AbM | |
| 267 | C3CRB127 Chain A, | GFTFSRY |
| | CD3 Heavy CDR1 | |
| | Chothia | |
| 268 | C3CRB127 Chain A, | STSSNY |
| | CD3 Heavy CDR2 | |
| | Chothia | |
| 212 | C3CRB127 Chain A, | GWGPFDY |
| | CD3 Heavy CDR3 | |
| | Chothia | |
| 265 | C3CRB127 Chain A, | RYNMN |
| | CD3 Heavy CDR1 Kabat | |
| 266 | C3CRB127 Chain A, | SISTSSNYIYYADSVKG |
| | CD3 Heavy CDR2 Kabat | |
| 212 | C3CRB127 Chain A, | GWGPFDY |
| | CD3 Heavy CDR3 Kabat | |
| 269 | C3CRB127 Chain A, | GFTFSRYN |
| | CD3 Heavy CDR1 | |
| | IMGT | |
| 270 | C3CRB127 Chain A, | ISTSSNYI |
| | CD3 Heavy CDR2 | |
| | IMGT | |
| 271 | C3CRB127 Chain A, | TRGWGPFDY |
| | CD3 Heavy CDR3 | |
| | IMGT | |
| 285 | C3CRB127 Chain A, | EVOLVESGGGLVKPGGSLRLSCAASGFTF |
| | CD3 VH | SRYNMNWVRQAPGCGLEWVSSISTSSNYI |
| | | YYADSVKGRFTFSRDNAKNSLDLQMSGL |
| | | RAEDTAIYYCTRGWGPFDYWGQGTLVTV |
| | | SS |
| 214 | C3CRB127 Chain A, | RARQSIGTAIH |
| | CD3 Light CDR1 AbM | |
| 215 | C3CRB 127 Chain A, | YASESIS |
| | CD3 Light CDR2 AbM | |
| 216 | C3CRB127 Chain A, | QQSGSWPYT |
| | CD3 Light CDR3 AbM | |
| 214 | C3CRB127 Chain A, | RARQSIGTAIH |
| | CD3 Light CDR1 | |
| | Chothia | |
| 215 | C3CRB127 Chain A, | YASESIS |
| | CD3 Light CDR2 | |
| | Chothia | |
| 216 | C3CRB127 Chain A, | QQSGSWPYT |
| | CD3 Light CDR3 | |
| | Chothia | |
| 214 | C3CRB127 Chain A, | RARQSIGTAIH |
| | CD3 Light CDR1 Kabat | |
| 215 | C3CRB127 Chain A, | YASESIS |
| | CD3 Light CDR2 Kabat | |
| 216 | C3CRB127 Chain A, | QQSGSWPYT |
| | CD3 Light CDR3 Kabat | |
| 272 | C3CRB127 Chain A, | QSIGTA |
| | CD3 Light CDR1 IMGT | |
| n/a | C3CRB127 Chain A, | YAS |
| | CD3 Light CDR2 IMGT | |
| 216 | C3CRB127 Chain A, | QQSGSWPYT |
| | CD3 Light CDR3 IMGT | |
| 217 | C3CRB127 Chain A, | DIQMTQSPSSLSASVGDRVTITCRARQSIG |
| | CD3 VL | TAIHWYQQKPGCAPKLLIKYASESISGVPS |
| | | RFSGSGSGTDFTLTISSLQPEDFATYYCQQ |
| | | SGSWPYTFGQGTKLEIK |
| 297 | C3CRB127 Chain A | QVQLVQSGAEVKKPGASVKVSCKASGFS |
| | Heavy Chain | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSSASTKGPSVFPLAPSS |
| | | KSTSGGTAALGCLVKDYFPEPVTVSWNSG |
| | | ALTSGVHTFPAVLQSSGLYSLSSVVTVPSS |
| | | SLGTQTYICNVNHKPSNTKVDKKVEPKSC |
| | | GGGGSGGGGSEVQLVESGGGLVKPGGSL |
| | | RLSCAASGFTFSRYNMNWVRQAPGCGLE |
| | | WVSSISTSSNYIYYADSVKGRFTFSRDNAK |
| | | NSLDLQMSGLRAEDTAIYYCTRGWGPFD |
| | | YWGQGTLVTVSSGGGSGGSGGCPPCGGS |
| | | GGDIQMTQSPSSLSASVGDRVTITCRARQS |
| | | IGTAIHWYQQKPGKAPKLLIKYASESISGV |
| | | PSRFSGSGSGTDFTLTISSLQPEDFATYYCQ |
| | | QSGSWPYTFGCGTKLEIKEPKSSDKTHTCP |
| | | PCPAPEAAGGPSVFLFPPKPKDTLMISRTP |
| | | EVTCVVVSVSHEDPEVKFNWYVDGVEVH |
| | | NAKTKPREEQYNSTYRVVSVLTVLHQDW |
| | | LNGKEYKCKVSNKALPAPIEKTISKAKGQ |
| | | PREPQVYTLPPSREEMTKNQVSLWCLVKG |
| | | FYPSDIAVEWESNGQPENNYKTTPPVLDS |
| | | DGSFFLYSKLTVDKSRWQQGNVFSCSVM |
| | | HEALHNHYTQKSLSLSPGK |
| 70 | C3CRB127 Chain A, | QASQSLYNNKNLA |
| | CALRB1114 Light | |
| | CDR1 AbM | |
| 71 | C3CRB127 Chain A, | YASALAS |
| | CALRB1114 Light | |
| | CDR2 AbM | |
| 72 | C3CRB127 Chain A, | QGEFNCNSADCNS |
| | CALRB1114 Light | |
| | CDR3 AbM | |
| 70 | C3CRB127 Chain A, | QASQSLYNNKNLA |
| | CALRB1114 Light | |
| | CDR1 Chothia | |
| 71 | C3CRB127 Chain A, | YASALAS |
| | CALRB1114 Light | |
| | CDR2 Chothia | |
| 72 | C3CRB127 Chain A, | QGEFNCNSADCNS |
| | CALRB1114 Light | |
| | CDR3 Chothia | |
| 70 | C3CRB127 Chain A, | QASQSLYNNKNLA |
| | CALRB1114 Light | |
| | CDR1 Kabat | |
| 71 | C3CRB127 Chain A, | YASALAS |
| | CALRB1114 Light | |
| | CDR2 Kabat | |
| 72 | C3CRB127 Chain A, | QGEFNCNSADCNS |
| | CALRB1114 Light | |
| | CDR3 Kabat | |
| 283 | C3CRB127 Chain A, | QSLYNNKN |
| | CALRB1114 Light | |
| | CDR1 IMGT | |
| n/a | C3CRB127 Chain A, | YAS |
| | CALRB1114 Light | |
| | CDR2 IMGT | |
| 72 | C3CRB127 Chain A, | QGEFNCNSADCNS |
| | CALRB1114 Light | |
| | CDR3 IMGT | |
| 284 | C3CRB127 Chain A, | DIQMTQSPSTLSASVGDRVTITCQASQSLY |
| | CALRB1114 VL | NNKNLAWYQQKPGKAPKLLIYYASALAS |
| | | GVPSRFSGSGSGTEFTLTISSLQPDDFATYY |
| | | CQGEFNCNSADCNSFGGGTKVEIK |
| 291 | C3CRB 127 Chain A | DIQMTQSPSTLSASVGDRVTITCQASQSLY |
| | Light Chain | NNKNLAWYQQKPGKAPKLLIYYASALAS |
| | | GVPSRFSGSGSGTEFTLTISSLQPDDFATYY |
| | | CQGEFNCNSADCNSFGGGTKVEIKRTVAA |
| | | PSVFIFPPSDEQLKSGTASVVCLLNNFYPR |
| | | EAKVQWKVDNALQSGNSQESVTEQDSKD |
| | | STYSLSSTLTLSKADYEKHKVYACEVTHQ |
| | | GLSSPVTKSFNRGEC |
| 273 | C3CRB127 Chain B, | GFSLSSYAMS |
| | CALRB1114 Heavy | |
| | CDR1 AbM | |
| 274 | C3CRB127 Chain B, | VIDSGGTTY |
| | CALRB1114 Heavy | |
| | CDR2 AbM | |
| 275 | C3CRB127 Chain B, | ESYAGYDYYGHPIPYGMEP |
| | CALRB1114 Heavy | |
| | CDR3 AbM | |
| 276 | C3CRB127 Chain B, | GFSLSSY |
| | CALRB1114 Heavy | |
| | CDR1 Chothia | |
| 277 | C3CRB127 Chain B, | DSGGT |
| | CALRB1114 Heavy | |
| | CDR2 Chothia | |
| 275 | C3CRB127 Chain B, | ESYAGYDYYGHPIPYGMEP |
| | CALRB1114 Heavy | |
| | CDR3 Chothia | |
| 66 | C3CRB127 Chain B, | SYAMS |
| | CALRB1114 Heavy | |
| | CDR1 Kabat | |
| 278 | C3CRB127 Chain B, | VIDSGGTTYYAQKFQG |
| | CALRB1114 Heavy | |
| | CDR2 Kabat | |
| 275 | C3CRB127 Chain B, | ESYAGYDYYGHPIPYGMEP |
| | CALRB1114 Heavy | |
| | CDR3 Kabat | |
| 216 | C3CRB127 Chain B, | QQSGSWPYT |
| | CALRB1114 Heavy | |
| | CDR1 IMGT | |
| 280 | C3CRB127 Chain B, | IDSGGTT |
| | CALRB1114 Heavy | |
| | CDR2 IMGT | |
| 281 | C3CRB127 Chain B, | ARESYAGYDYYGHPIPYGMEP |
| | CALRB1114 Heavy | |
| | CDR3 IMGT | |
| 282 | C3CRB127 Chain B, | QVQLVQSGAEVKKPGASVKVSCKASGFS |
| | CALRB1114 VH | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSS |
| 290 | C3CRB127 Chain B | QVQLVQSGAEVKKPGASVKVSCKASGFS |
| | Heavy Chain | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSSASTKGPSVFPLAPSS |
| | | KSTSGGTAALGCLVKDYFPEPVTVSWNSG |
| | | ALTSGVHTFPAVLQSSGLYSLSSVVTVPSS |
| | | SLGTQTYICNVNHKPSNTKVDKKVEPKSC |
| | | DKTHTCPPCPAPEAAGGPSVFLFPPKPKDT |
| | | LMISRTPEVTCVVVSVSHEDPEVKFNWYV |
| | | DGVEVHNAKTKPREEQYNSTYRVVSVLT |
| | | VLHQDWLNGKEYKCKVSNKALPAPIEKTI |
| | | SKAKGQPREPQVYTLPPSREEMTKNQVSL |
| | | SCAVKGFYPSDIA VEWESNGQPENNYKTT |
| | | PPVLDSDGSFFLVSKLTVDKSRWQQGNVF |
| | | SCSVMHEALHNRFTQKSLSLSPGK |
| 70 | C3CRB127 Chain B, | QASQSLYNNKNLA |
| | CALRB1114 Light | |
| | CDR1 AbM | |
| 71 | C3CRB127 Chain B, | YASALAS |
| | CALRB1114 Light | |
| | CDR2 AbM | |
| 72 | C3CRB127 Chain B, | QGEFNCNSADCNS |
| | CALRB1114 Light | |
| | CDR3 AbM | |
| 70 | C3CRB127 Chain B, | QASQSLYNNKNLA |
| | CALRB1114 Light | |
| | CDR1 Chothia | |
| 71 | C3CRB127 Chain B, | YASALAS |
| | CALRB1114 Light | |
| | CDR2 Chothia | |
| 72 | C3CRB127 Chain B, | QGEFNCNSADCNS |
| | CALRB1114 Light | |
| | CDR3 Chothia | |
| 70 | C3CRB127 Chain B, | QASQSLYNNKNLA |
| | CALRB1114 Light | |
| | CDR1 Kabat | |
| 71 | C3CRB127 Chain B, | YASALAS |
| | CALRB1114 Light | |
| | CDR2 Kabat | |
| 72 | C3CRB 127 Chain B, | QGEFNCNSADCNS |
| | CALRB1114 Light | |
| | CDR3 Kabat | |
| 283 | C3CRB127 Chain B, | QSLYNNKN |
| | CALRB1114 Light | |
| | CDR1 IMGT | |
| n/a | C3CRB127 Chain B, | YAS |
| | CALRB1114 Light | |
| | CDR2 IMGT | |
| 72 | C3CRB127 Chain B, | QGEFNCNSADCNS |
| | CALRB1114 Light | |
| | CDR3 IMGT | |
| 284 | C3CRB127 Chain B, | DIQMTQSPSTLSASVGDRVTITCQASQSLY |
| | CALRB1114 VL | NNKNLAWYQQKPGKAPKLLIYYASALAS |
| | | GVPSRFSGSGSGTEFTLTISSLQPDDFATYY |
| | | CQGEFNCNSADCNSFGGGTKVEIK |
| 291 | C3CRB127 Chain B | DIQMTQSPSTLSASVGDRVTITCQASQSLY |
| | Light Chain | NNKNLAWYQQKPGKAPKLLIYYASALAS |
| | | GVPSRFSGSGSGTEFTLTISSLQPDDFATYY |
| | | CQGEFNCNSADCNSFGGGTKVEIKRTVAA |
| | | PSVFIFPPSDEQLKSGTASVVCLLNNFYPR |
| | | EAKVQWKVDNALQSGNSQESVTEQDSKD |
| | | STYSLSSTLTLSKADYEKHKVYACEVTHQ |
| | | GLSSPVTKSFNRGEC |

### B. Primary T Cell Binding

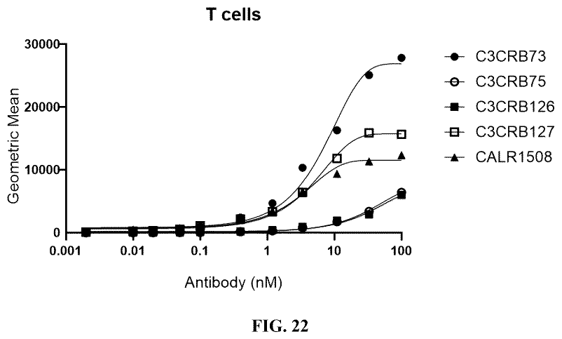

Flow cytometry was used to evaluate the CD3 arm binding of CALRmutxCD3 antibodies listed in Table 21 to CD3+ pan T cells from healthy human donor. CALRmutxCD3 were tested after 1 hour 37° C. incubation with pan T cells. All antibodies showed concentration-dependent binding profiles but with different max Geometric mean value (FIG. 22).

### C. CALRmutxCD3-Induced T Cell-Mediated Cytotoxicity of CALRmut-Engineered Cancer Cells

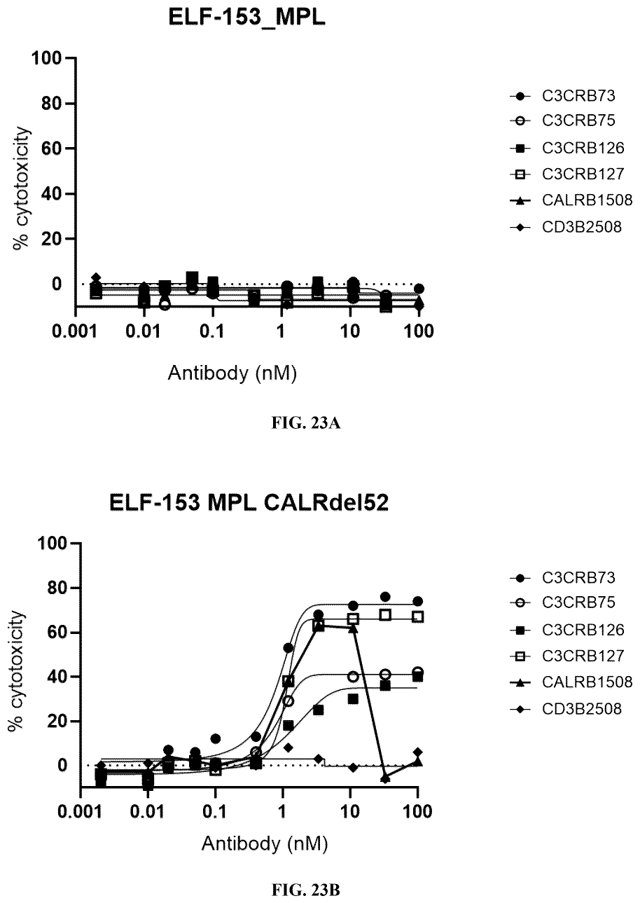

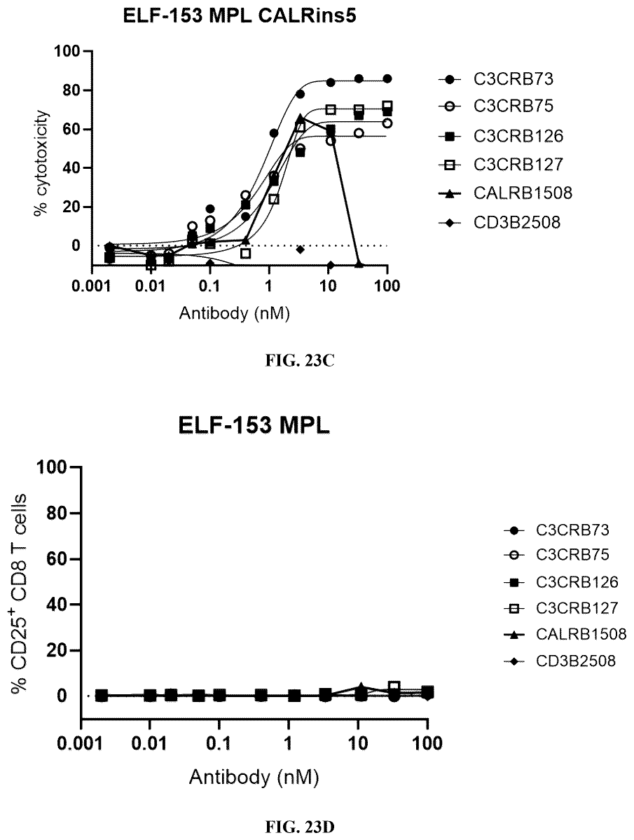

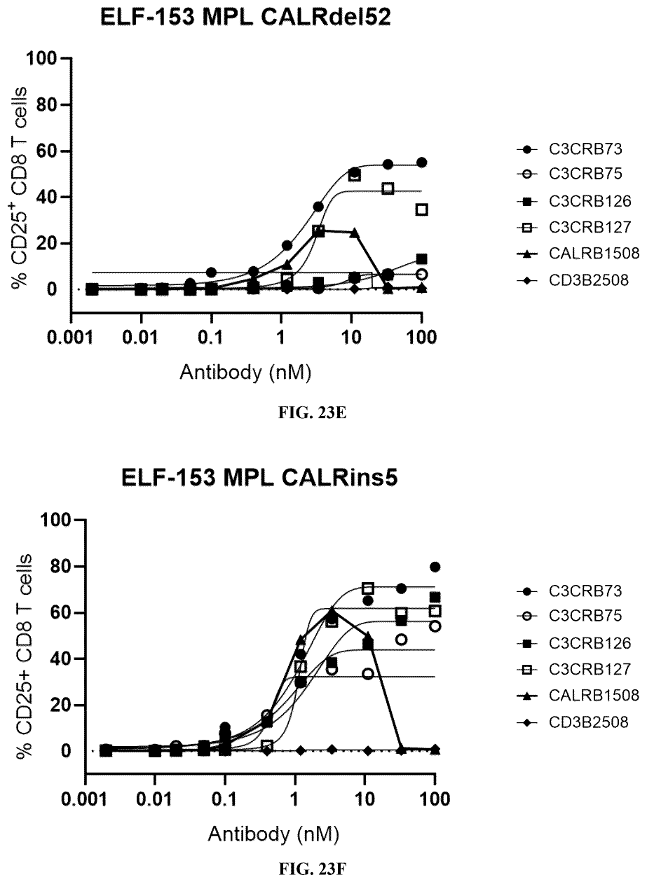

T-cell cytotoxicity assays were performed to evaluate the ability of CALRmutxCD3 to induce CALRmut-selective cytotoxicity of cell lines engineered to express the most common CALRmuts, i.e., CALRde152 and CALRins5. T cells from healthy donors were used at 5:1 E:T ratios. After 72 hours, CALRmutxCD3 antibodies elicited concentration-dependent cytotoxicity of the cell lines expressing MPL CALRde152 or MPL CALRins5 (FIGS. 23B and 23C). No cytotoxicity was observed for the ELF-153 MPL CALRwt cell line (FIG. 23A). No cytotoxicity was observed in presence of the NullxCD3 spFv (CD31B2508) negative control. These results demonstrate that CALRmutxCD3 can induce selective cytotoxicity of CALRmut cell lines expressing different levels of CALRmut on the cell surface.

CD25 expression as a measure of T-cell activation was monitored in the cytotoxicity assays described herein. CALRmutxCD3 elicited concentration-dependent increases in the activation of T cells in the presence of cell lines expressing CALRmut after 72 hours but did not elicit activation in coculture with target-negative cell lines (FIGS. 23D-23F). The NullxCD3 spFv (CD3B2508) negative control antibody induced no T-cell activation.

D. CALRmutxCD3-Induced T Cell-Mediated Cytotoxicity of CALRmut CD34+ Cells Derived from CALRmut MPN Patient Cells in Autologous Setting

To evaluate the impact of CALRmutxCD3 in a more physiologically relevant setting, T-cell cytotoxicity assays were conducted in an autologous setting using CD34+ cells isolated from CALRmut MPN patients as target cells together with T cells isolated from the same patient.

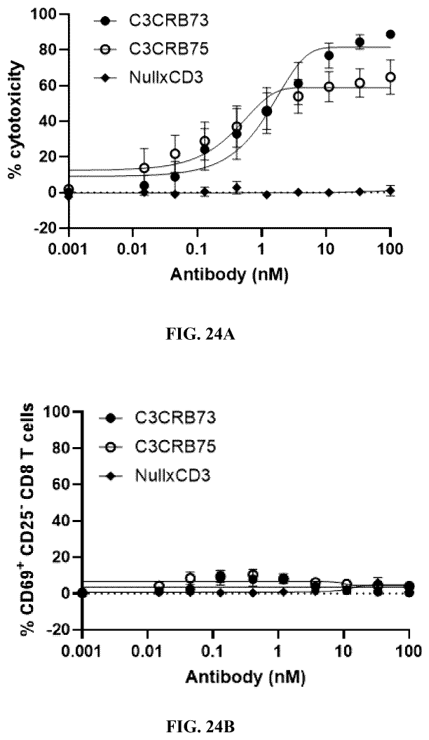

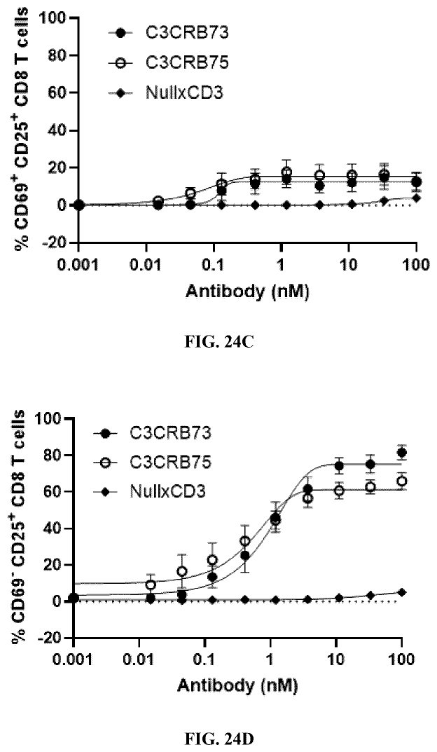

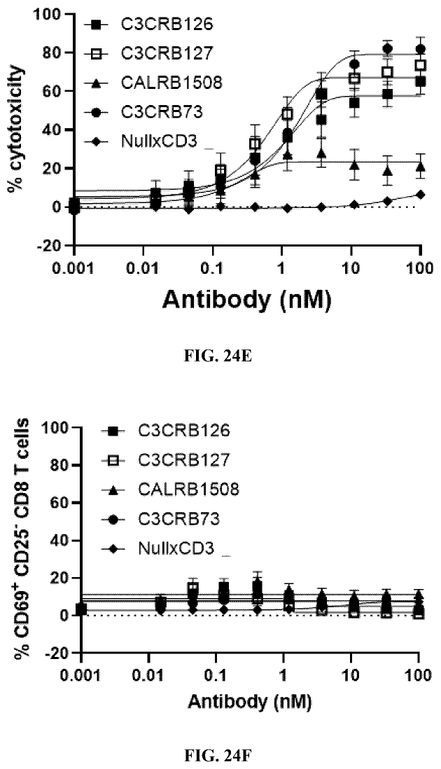

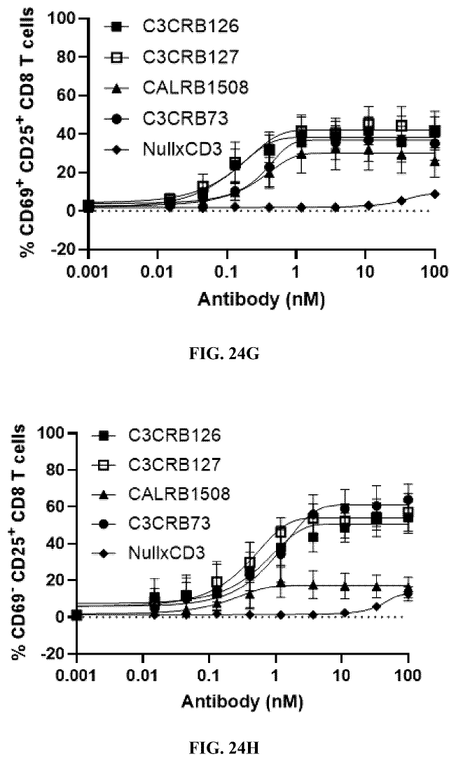

The studies were conducted using an E:T ratio of 5:1. CALRmutxCD3 antibodies, listed in Table 21, elicited concentration-dependent cytotoxicity of patient-derived CALRmut CD34+ cells (FIGS. 24A and 24E). T-cell activation was assessed in parallel by measuring % CD25− or CD69-positivity on the surface of CD8 T cells. CALRmutxCD3 antibodies elicited concentration-dependent T-cell activation in autologous cytotoxicity assays (FIGS. 24B-24D and FIGS. 24F-24H).

Although previous studies reported exhaustion of T cells isolated from CALRmut MPN patients, the current results confirm that T cells in the presence of CALRmutxCD3 antibodies are capable of engaging CALRmut-patient-derived T cells to mediate cytotoxicity to CALRmut-expressing CD34+ cells in an autologous setting.

E. Effect of CALRmutxCD3 in CALRmut Whole-Blood T Cell Redirection Assay Cocultured with ELF-153 MPL or ELF 153 MPL CALRins5 Cells

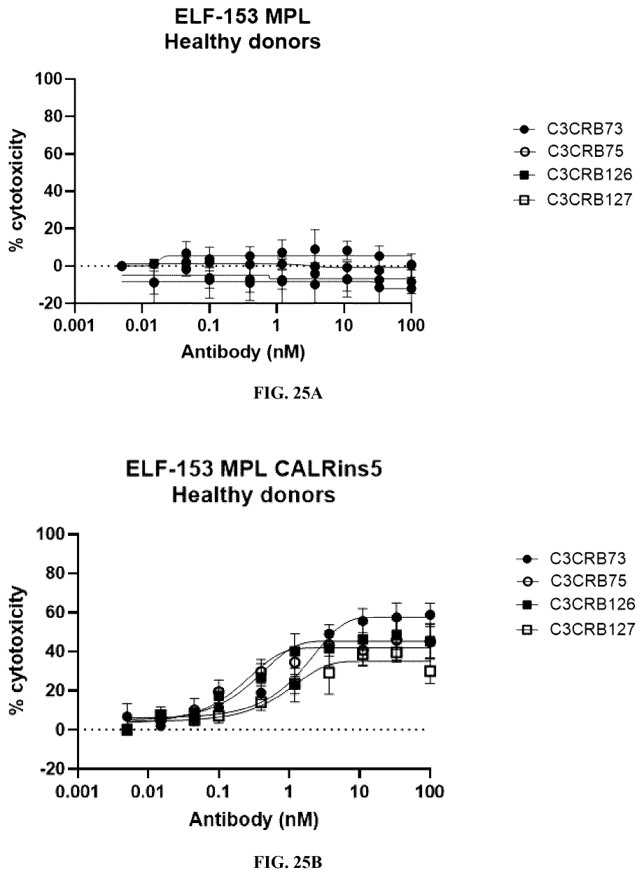

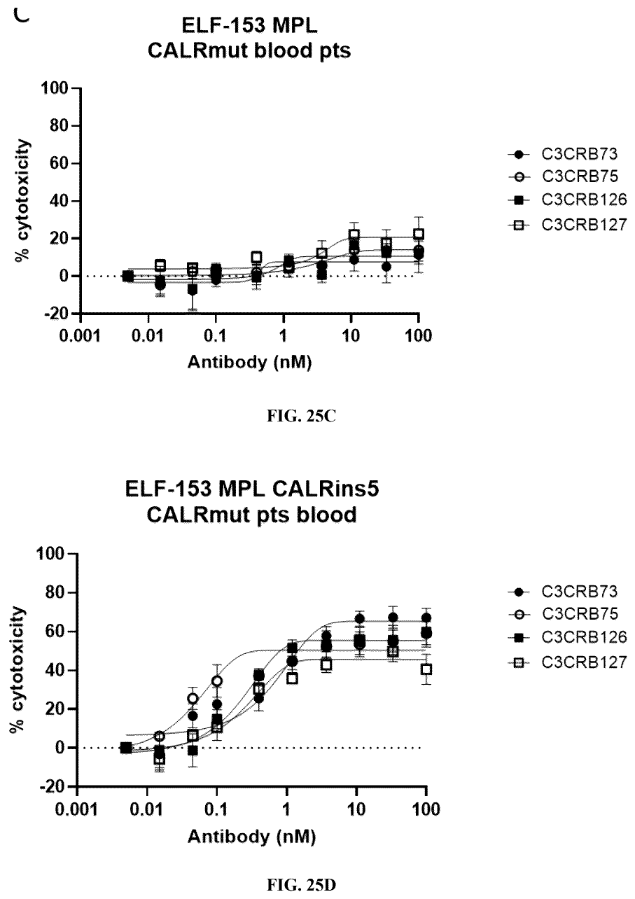

To evaluate the impact of soluble CALRmut on CALrmutxCD3 bispecfic antibodies in a physiologically relevant setting, T-cell cytotoxicity assays were conducted using CALRmut patient-derived whole blood. Whole blood from healthy donors was used as a control condition lacking the soluble CALRmut component (FIG. 25A). The studies were conducted using an E:T ratio of 1:1, with T cells as effectors and target cells comprising labeled ELF-153 MPL, or ELF-153 MPL CALRins5 cancer cells in the donor blood. Tumor cells were evaluated for cytotoxicity.

CALRmutxCD3 antibodies elicited concentration-dependent T cell-mediated cytotoxicity of ELF-153 MPL CALRins5 cells with similar potency in whole blood of healthy donors and CALRmut MPN patients (FIGS. 25B and 25D). Minimal cytotoxicity at higher concentrations was observed to ELF 153 MPL cells in the presence of CALRmutxCD3 antibodies in whole blood from CALRmut patients (FIG. 25 C).

Altogether, these data suggested that the presence of soluble CALRmut derived from CALRmut MPN patients does not impair cytotoxic activity of CALRmutxCD3 towards CALRmut-expressing cancer cells.

### Example 11: Additional Alternative Anti-CALRmut Antibodies

### A. Binding to CALRmut Cancer Cells

To evaluate the selective binding of CALRmutxCD3 antibodies to CALRmut cells, pairs of the isogenic cell lines expressing MPL and CALRmut (CALRdel52 or CALRins5) were generated in the background of ELF-153 (cell line derived from MF patient that progressed to sAML, i.e., AML M7) or OCI-M2 (cell line derived from erythroleukemia patient, i.e., AML M6) cells lines. Expression of MPL and CALRmut were confirmed on the cell surface of these cells as shown in Table 14.

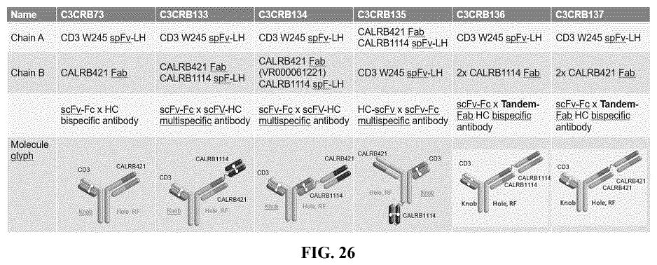

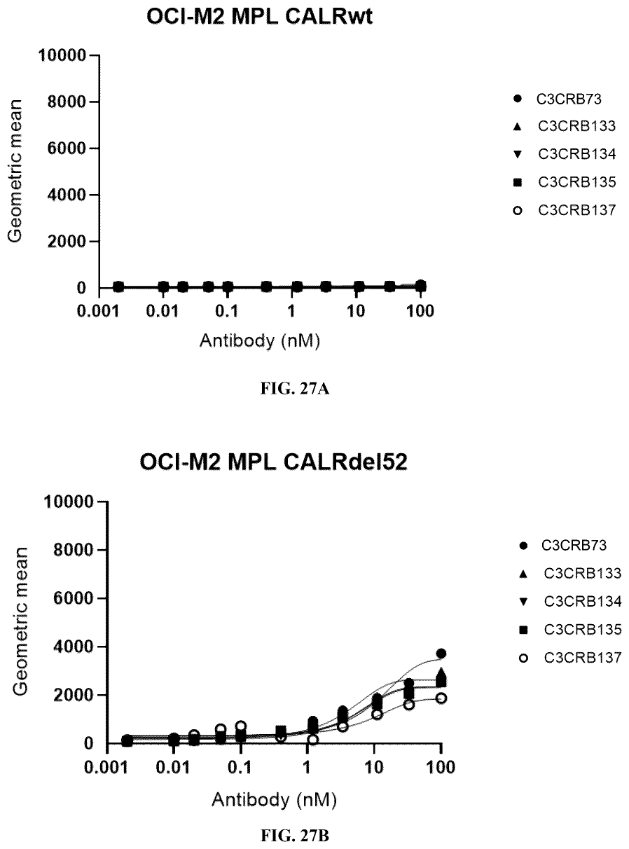

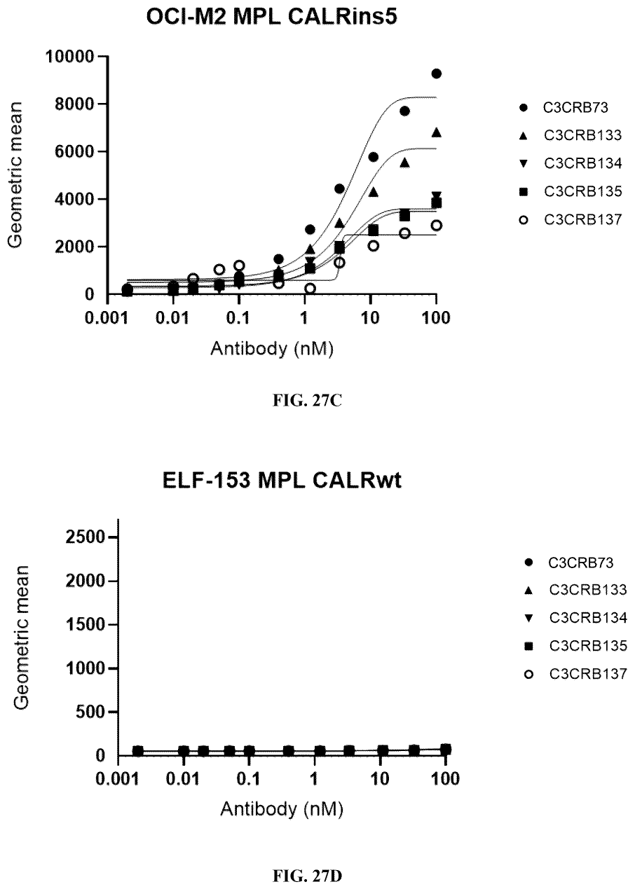

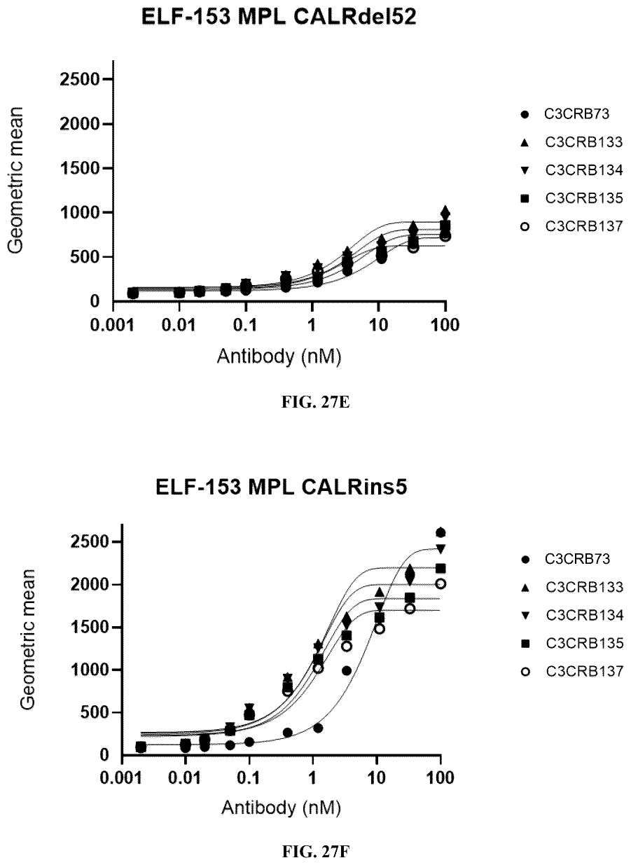

All CALRmutxCD3 antibodies listed in Tables 25-29 and FIG. 26 showed selective binding to cells expressing CALRmut and no binding to target-negative cells (FIGS. 27A-27F).

TABLE 25
Sequences for C3CRB133 CALRmut x CD3 Bispecific Antibody

| SEQ ID NO | Description | Sequence |
|---|---|---|
| 214 | C3CRB133 Chain A, | RARQSIGTAIH |
| | CD3 Light CDR1 AbM | |
| 215 | C3CRB133 Chain A, | YASESIS |
| | CD3 Light CDR2 AbM | |
| 216 | C3CRB133 Chain A, | QQSGSWPYT |
| | CD3 Light CDR3 AbM | |
| 214 | C3CRB133 Chain A, | RARQSIGTAIH |
| | CD3 Light CDR1 | |
| | Chothia | |
| 215 | C3CRB133 Chain A, | YASESIS |
| | CD3 Light CDR2 | |
| | Chothia | |
| 216 | C3CRB133 Chain A, | QQSGSWPYT |
| | CD3 Light CDR3 | |
| | Chothia | |
| 214 | C3CRB133 Chain A, | RARQSIGTAIH |
| | CD3 Light CDR1 Kabat | |
| 215 | C3CRB133 Chain A, | YASESIS |
| | CD3 Light CDR2 Kabat | |
| 216 | C3CRB133 Chain A, | QQSGSWPYT |
| | CD3 Light CDR3 Kabat | |
| 272 | C3CRB133 Chain A, | QSIGTA |
| | CD3 Light CDR1 IMGT | |
| n/a | C3CRB133 Chain A, | YAS |
| | CD3 Light CDR2 IMGT | |
| 216 | C3CRB133 Chain A, | QQSGSWPYT |
| | CD3 Light CDR3 IMGT | |
| 217 | C3CRB133 Chain A, | DIQMTQSPSSLSASVGDRVTITCRARQSIG |
| | CD3 VL | TAIHWYQQKPGCAPKLLIKYASESISGVPS |
| | | RFSGSGSGTDFTLTISSLQPEDFATYYCQQ |
| | | SGSWPYTFGQGTKLEIK |
| 210 | C3CRB133 Chain A, | GFTFSRYNMN |
| | CD3 Heavy CDR1 AbM | |
| 211 | C3CRB133 Chain A, | SISTSSNYIY |
| | CD3 Heavy CDR2 AbM | |
| 212 | C3CRB133 Chain A, | GWGPFDY |
| | CD3 Heavy CDR3 AbM | |
| 267 | C3CRB133 Chain A, | GFTFSRY |
| | CD3 Heavy CDR1 | |
| | Chothia | |
| 268 | C3CRB133 Chain A, | STSSNY |
| | CD3 Heavy CDR2 | |
| | Chothia | |
| 212 | C3CRB133 Chain A, | GWGPFDY |
| | CD3 Heavy CDR3 | |
| | Chothia | |
| 265 | C3CRB133 Chain A, | RYNMN |
| | CD3 Heavy CDR1 Kabat | |
| 266 | C3CRB133 Chain A, | SISTSSNYIYYADSVKG |
| | CD3 Heavy CDR2 Kabat | |
| 212 | C3CRB133 Chain A, | GWGPFDY |
| | CD3 Heavy CDR3 Kabat | |
| 269 | C3CRB133 Chain A, | GFTFSRYN |
| | CD3 Heavy CDR1 | |
| | IMGT | |
| 270 | C3CRB133 Chain A, | ISTSSNYI |
| | CD3 Heavy CDR2 | |
| | IMGT | |
| 271 | C3CRB133 Chain A, | TRGWGPFDY |
| | CD3 Heavy CDR3 | |
| | IMGT | |
| 213 | C3CRB133 Chain A, | EVOLVESGGGLVKPGGSLRLSCAASGFTF |
| | CD3 VH | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 218 | C3CRB133 Chain A | DIQMTQSPSSLSASVGDRVTITCRARQSIG |
| | Heavy Chain | TAIHWYQQKPGCAPKLLIKYASESISGVPS |
| | | RFSGSGSGTDFTLTISSLQPEDFATYYCQQ |
| | | SGSWPYTFGQGTKLEIKGGGSGGSGGCPP |
| | | CGGSGGEVOLVESGGGLVKPGGSLRLSCA |
| | | ASGFTFSRYNMNWVRQAPGKGLEWVSSI |
| | | STSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |
| 70 | C3CRB133 Chain B, | QASQSLYNNKNLA |
| | CALRB1114 Light | |
| | CDR1 AbM | |
| 71 | C3CRB133 Chain B, | YASALAS |
| | CALRB1114 Light | |
| | CDR2 AbM | |
| 72 | C3CRB133 Chain B, | QGEFNCNSADCNS |
| | CALRB1114 Light | |
| | CDR3 AbM | |
| 70 | C3CRB133 Chain B, | QASQSLYNNKNLA |
| | CALRB1114 Light | |
| | CDR1 Chothia | |
| 71 | C3CRB133 Chain B, | YASALAS |
| | CALRB1114 Light | |
| | CDR2 Chothia | |
| 72 | C3CRB133 Chain B, | QGEFNCNSADCNS |
| | CALRB1114 Light | |
| | CDR3 Chothia | |
| 70 | C3CRB133 Chain B, | QASQSLYNNKNLA |
| | CALRB1114 Light | |
| | CDR1 Kabat | |
| 71 | C3CRB133 Chain B, | YASALAS |
| | CALRB1114 Light | |
| | CDR2 Kabat | |
| 72 | C3CRB133 Chain B, | QGEFNCNSADCNS |
| | CALRB1114 Light | |
| | CDR3 Kabat | |
| 283 | C3CRB133 Chain B, | QSLYNNKN |
| | CALRB1114 Light | |
| | CDR1 IMGT | |
| n/a | C3CRB133 Chain B, | YAS |
| | CALRB1114 Light | |
| | CDR2 IMGT | |
| 72 | C3CRB133 Chain B, | QGEFNCNSADCNS |
| | CALRB1114 Light | |
| | CDR3 IMGT | |
| 286 | C3CRB133 Chain B, | DIQMTQSPSTLSASVGDRVTITCQASQSLY |
| | CALRB1114 VL | NNKNLAWYQQKPGCAPKLLIYYASALAS |
| | | GVPSRFSGSGSGTEFTLTISSLQPDDFATYY |
| | | CQGEFNCNSADCNSFGGGTKVEIK |
| 273 | C3CRB133 Chain B, | GFSLSSYAMS |
| | CALRB1114 Heavy | |
| | CDR1 AbM | |
| 274 | C3CRB133 Chain B, | VIDSGGTTY |
| | CALRB1114 Heavy | |
| | CDR2 AbM | |
| 275 | C3CRB133 Chain B, | ESYAGYDYYGHPIPYGMEP |
| | CALRB1114 Heavy | |
| | CDR3 AbM | |
| 276 | C3CRB133 Chain B, | GFSLSSY |
| | CALRB1114 Heavy | |
| | CDR1 Chothia | |
| 277 | C3CRB133 Chain B, | DSGGT |
| | CALRB1114 Heavy | |
| | CDR2 Chothia | |
| 275 | C3CRB133 Chain B, | ESYAGYDYYGHPIPYGMEP |
| | CALRB1114 Heavy | |
| | CDR3 Chothia | |
| 66 | C3CRB133 Chain B, | SYAMS |
| | CALRB1114 Heavy | |
| | CDR1 Kabat | |
| 278 | C3CRB133 Chain B, | VIDSGGTTYYAQKFQG |
| | CALRB1114 Heavy | |
| | CDR2 Kabat | |
| 275 | C3CRB133 Chain B, | ESYAGYDYYGHPIPYGMEP |
| | CALRB1114 Heavy | |
| | CDR3 Kabat | |
| 279 | C3CRB133 Chain B, | GFSLSSYA |
| | CALRB1114 Heavy | |
| | CDR1 IMGT | |
| 280 | C3CRB133 Chain B, | IDSGGTT |
| | CALRB1114 Heavy | |
| | CDR2 IMGT | |
| 281 | C3CRB133 Chain B, | ARESYAGYDYYGHPIPYGMEP |
| | CALRB1114 Heavy | |
| | CDR3 IMGT | |
| 287 | C3CRB133 Chain B, | QVQLVQSGAEVKKPGASVKVSCKASGFS |
| | CALRB1114 VH | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGCGTLVTVSS |
| 200 | C3CRB133 Chain B, | GIDLSNNAIS |
| | CALRB421 Heavy | |
| | CDR1 AbM | |
| 201 | C3CRB133 Chain B, | VIGNTGDTY |
| | CALRB421 Heavy | |
| | CDR2 AbM | |
| 202 | C3CRB133 Chain B, | GPPSYSSSVKNI |
| | CALRB421 Heavy | |
| | CDR3 AbM | |
| 260 | C3CRB133 Chain B, | GIDLSNN |
| | CALRB421 Heavy | |
| | CDR1 Chothia | |
| 261 | C3CRB133 Chain B, | GNTGD |
| | CALRB421 Heavy | |
| | CDR2 Chothia | |
| 202 | C3CRB133 Chain B, | GPPSYSSSVKNI |
| | CALRB421 Heavy | |
| | CDR3 Chothia | |
| 81 | C3CRB133 Chain B, | NNAIS |
| | CALRB421 Heavy | |
| | CDR1 Kabat | |
| 259 | C3CRB133 Chain B, | VIGNTGDTYYADSAKG |
| | CALRB421 Heavy | |
| | CDR2 Kabat | |
| 202 | C3CRB133 Chain B, | GPPSYSSSVKNI |
| | CALRB421 Heavy | |
| | CDR3 Kabat | |
| 262 | C3CRB133 Chain B, | GIDLSNNA |
| | CALRB421 Heavy | |
| | CDR1 IMGT | |
| 263 | C3CRB133 Chain B, | IGNTGDT |
| | CALRB421 Heavy | |
| | CDR2 IMGT | |
| 264 | C3CRB133 Chain B, | VRGPPSYSSSVKNI |
| | CALRB421 Heavy | |
| | CDR3 IMGT | |
| 203 | C3CRB133 Chain B, | EVQLLESGGGLVQPGGSLRLSCAVSGIDLS |
| | CALRB421 VH | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSS |
| 293 | C3CRB133 Chain B | DIQMTQSPSTLSASVGDRVTITCQASQSLY |
| | Heavy Chain | NNKNLAWYQQKPGCAPKLLIYYASALAS |
| | | GVPSRFSGSGSGTEFTLTISSLQPDDFATYY |
| | | CQGEFNCNSADCNSFGGGTKVEIKGGGSG |
| | | GSGGCPPCGGSGGQVQLVQSGAEVKKPG |
| | | ASVKVSCKASGFSLSSYAMSWVRQAPGQ |
| | | GLEWMGVIDSGGTTYYAQKFQGRVTMTR |
| | | DTSTSTVYMELSSLRSEDTAVYYCARESY |
| | | AGYDYYGHPIPYGMEPWGCGTLVTVSSG |
| | | GGGSGGGGSEVQLLESGGGLVQPGGSLRL |
| | | SCAVSGIDLSNNAISWVRQAPGKGLEYVG |
| | | VIGNTGDTYYADSAKGRFTISRDSSKTTLY |
| | | LQMNSLRAEDTAVYFCVRGPPSYSSSVKN |
| | | IWGQGTLVTVSSASTKGPSVFPLAPSSKST |
| | | SGGTAALGCLVKDYFPEPVTVSWNSGALT |
| | | SGVHTFPAVLQSSGLYSLSSVVTVPSSSLG |
| | | TQTYICNVNHKPSNTKVDKKVEPKSCDKT |
| | | HTCPPCPAPEAAGGPSVFLFPPKPKDTLMI |
| | | SRTPEVTCVVVSVSHEDPEVKFNWYVDG |
| | | VEVHNAKTKPREEQYNSTYRVVSVLTVL |
| | | HQDWLNGKEYKCKVSNKALPAPIEKTISK |
| | | AKGQPREPQVYTLPPSREEMTKNQVSLSC |
| | | AVKGFYPSDIAVEWESNGQPENNYKTTPP |
| | | VLDSDGSFFLVSKLTVDKSRWQQGNVFSC |
| | | SVMHEALHNRFTQKSLSLSPGK |
| 33 | C3CRB133 Chain B, | QSSQSVYNNNWLS |
| | CALRB421 Light CDR1 | |
| | AbM | |
| 23 | C3CRB133 Chain B, | DASKLES |
| | CALRB421 Light CDR2 | |
| | AbM | |
| 34 | C3CRB133 Chain B, | AGGFTGNVYT |
| | CALRB421 Light CDR3 | |
| | AbM | |
| 33 | C3CRB133 Chain B, | QSSQSVYNNNWLS |
| | CALRB421 Light CDR1 | |
| | Chothia | |
| 23 | C3CRB133 Chain B, | DASKLES |
| | CALRB421 Light CDR2 | |
| | Chothia | |
| 34 | C3CRB133 Chain B, | AGGFTGNVYT |
| | CALRB421 Light CDR3 | |
| | Chothia | |
| 33 | C3CRB133 Chain B, | QSSQSVYNNNWLS |
| | CALRB421 Light CDR1 | |
| | Kabat | |
| 23 | C3CRB133 Chain B, | DASKLES |
| | CALRB421 Light CDR2 | |
| | Kabat | |
| 34 | C3CRB133 Chain B, | AGGFTGNVYT |
| | CALRB421 Light CDR3 | |
| | Kabat | |
| 256 | C3CRB133 Chain B, | QSVYNNNW |
| | CALRB421 Light CDR1 | |
| | IMGT | |
| n/a | C3CRB133 Chain B, | DAS |
| | CALRB421 Light CDR2 | |
| | IMGT | |
| 34 | C3CRB133 Chain B, | AGGFTGNVYT |
| | CALRB421 Light CDR3 | |
| | IMGT | |
| 205 | C3CRB133 Chain B, | DIQMTQSPSSVSASVGDRVTITCQSSQSVY |
| | CALRB421 VL | NNNWLSWLQQKPGKAPKRLIYDASKLES |
| | | GVPSRFSGSGSGTDFTLTISSVQPEDAATY |
| | | YCAGGFTGNVYTFGGGTKVEIK |
| 206 | C3CRB133 Chain B | DIQMTQSPSSVSASVGDRVTITCQSSQSVY |
| | Light Chain | NNNWLSWLQQKPGKAPKRLIYDASKLES |
| | | GVPSRFSGSGSGTDFTLTISSVQPEDAATY |
| | | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |

TABLE 26
Sequences for C3CRB134 CALRmut x CD3 Bispecific Antibody

| SEQ ID NO | Description | Sequence |
|---|---|---|
| 214 | C3CRB134 Chain A, | RARQSIGTAIH |
| | CD3 Light CDR1 AbM | |
| 215 | C3CRB134 Chain A, | YASESIS |
| | CD3 Light CDR2 AbM | |
| 216 | C3CRB134 Chain A, | QQSGSWPYT |
| | CD3 Light CDR3 AbM | |
| 214 | C3CRB134 Chain A, | RARQSIGTAIH |
| | CD3 Light CDR1 | |
| | Chothia | |
| 215 | C3CRB134 Chain A, | YASESIS |
| | CD3 Light CDR2 | |
| | Chothia | |
| 216 | C3CRB134 Chain A, | QQSGSWPYT |
| | CD3 Light CDR3 | |
| | Chothia | |
| 214 | C3CRB134 Chain A, | RARQSIGTAIH |
| | CD3 Light CDR1 Kabat | |
| 215 | C3CRB134 Chain A, | YASESIS |
| | CD3 Light CDR2 Kabat | |
| 216 | C3CRB134 Chain A, | QQSGSWPYT |
| | CD3 Light CDR3 Kabat | |
| 272 | C3CRB134 Chain A, | QSIGTA |
| | CD3 Light CDR1 IMGT | |
| n/a | C3CRB134 Chain A, | YAS |
| | CD3 Light CDR2 IMGT | |
| 216 | C3CRB134 Chain A, | QQSGSWPYT |
| | CD3 Light CDR3 IMGT | |
| 217 | C3CRB134 Chain A, | DIQMTQSPSSLSASVGDRVTITCRARQSIG |
| | CD3 VL | TAIHWYQQKPGCAPKLLIKYASESISGVPS |
| | | RFSGSGSGTDFTLTISSLOPEDFATYYCQQ |
| | | SGSWPYTFGQGTKLEIK |
| 210 | C3CRB134 Chain A, | GFTFSRYNMN |
| | CD3 Heavy CDR1 AbM | |
| 211 | C3CRB134 Chain A, | SISTSSNYIY |
| | CD3 Heavy CDR2 AbM | |
| 212 | C3CRB134 Chain A, | GWGPFDY |
| | CD3 Heavy CDR3 AbM | |
| 267 | C3CRB134 Chain A, | GFTFSRY |
| | CD3 Heavy CDR1 | |
| | Chothia | |
| 268 | C3CRB134 Chain A, | STSSNY |
| | CD3 Heavy CDR2 | |
| | Chothia | |
| 212 | C3CRB134 Chain A, | GWGPFDY |
| | CD3 Heavy CDR3 | |
| | Chothia | |
| 265 | C3CRB134 Chain A, | RYNMN |
| | CD3 Heavy CDR1 Kabat | |
| 266 | C3CRB134 Chain A, | SISTSSNYIYYADSVKG |
| | CD3 Heavy CDR2 Kabat | |
| 212 | C3CRB134 Chain A, | GWGPFDY |
| | CD3 Heavy CDR3 Kabat | |
| 269 | C3CRB134 Chain A, | GFTFSRYN |
| | CD3 Heavy CDR1 | |
| | IMGT | |
| 270 | C3CRB134 Chain A, | ISTSSNYI |
| | CD3 Heavy CDR2 | |
| | IMGT | |
| 271 | C3CRB134 Chain A, | TRGWGPFDY |
| | CD3 Heavy CDR3 | |
| | IMGT | |
| 213 | C3CRB134 Chain A, | EVOLVESGGGLVKPGGSLRLSCAASGFTF |
| | CD3 VH | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 218 | C3CRB134 Chain A | DIQMTQSPSSLSASVGDRVTITCRARQSIG |
| | Heavy Chain | TAIHWYQQKPGCAPKLLIKYASESISGVPS |
| | | RFSGSGSGTDFTLTISSLOPEDFATYYCQQ |
| | | SGSWPYTFGQGTKLEIKGGGSGGSGGCPP |
| | | CGGSGGEVOLVESGGGLVKPGGSLRLSCA |
| | | ASGFTFSRYNMNWVRQAPGKGLEWVSSI |
| | | STSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |
| 200 | C3CRB134 Chain B, | GIDLSNNAIS |
| | CALRB421 Heavy | |
| | CDR1 AbM | |
| 201 | C3CRB134 Chain B, | VIGNTGDTY |
| | CALRB421 Heavy | |
| | CDR2 AbM | |
| 202 | C3CRB134 Chain B, | GPPSYSSSVKNI |
| | CALRB421 Heavy | |
| | CDR3 AbM | |
| 260 | C3CRB134 Chain B, | GIDLSNN |
| | CALRB421 Heavy | |
| | CDR1 Chothia | |
| 261 | C3CRB134 Chain B, | GNTGD |
| | CALRB421 Heavy | |
| | CDR2 Chothia | |
| 202 | C3CRB134 Chain B, | GPPSYSSSVKNI |
| | CALRB421 Heavy | |
| | CDR3 Chothia | |
| 81 | C3CRB134 Chain B, | NNAIS |
| | CALRB421 Heavy | |
| | CDR1 Kabat | |
| 259 | C3CRB134 Chain B, | VIGNTGDTYYADSAKG |
| | CALRB421 Heavy | |
| | CDR2 Kabat | |
| 202 | C3CRB134 Chain B, | GPPSYSSSVKNI |
| | CALRB421 Heavy | |
| | CDR3 Kabat | |
| 262 | C3CRB134 Chain B, | GIDLSNNA |
| | CALRB421 Heavy | |
| | CDR1 IMGT | |
| 263 | C3CRB134 Chain B, | IGNTGDT |
| | CALRB421 Heavy | |
| | CDR2 IMGT | |
| 264 | C3CRB134 Chain B, | VRGPPSYSSSVKNI |
| | CALRB421 Heavy | |
| | CDR3 IMGT | |
| 203 | C3CRB134 Chain B, | EVQLLESGGGLVQPGGSLRLSCAVSGIDLS |
| | CALRB421 VH | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSS |
| 70 | C3CRB134 Chain B, | QASQSLYNNKNLA |
| | CALRB1114 Light | |
| | CDR1 AbM | |
| 71 | C3CRB134 Chain B, | YASALAS |
| | CALRB1114 Light | |
| | CDR2 AbM | |
| 72 | C3CRB134 Chain B, | QGEFNCNSADCNS |
| | CALRB1114 Light | |
| | CDR3 AbM | |
| 70 | C3CRB134 Chain B, | QASQSLYNNKNLA |
| | CALRB1114 Light | |
| | CDR1 Chothia | |
| 71 | C3CRB134 Chain B, | YASALAS |
| | CALRB1114 Light | |
| | CDR2 Chothia | |
| 72 | C3CRB134 Chain B, | QGEFNCNSADCNS |
| | CALRB1114 Light | |
| | CDR3 Chothia | |
| 70 | C3CRB134 Chain B, | QASQSLYNNKNLA |
| | CALRB1114 Light | |
| | CDR1 Kabat | |
| 71 | C3CRB134 Chain B, | YASALAS |
| | CALRB1114 Light | |
| | CDR2 Kabat | |
| 72 | C3CRB134 Chain B, | QGEFNCNSADCNS |
| | CALRB1114 Light | |
| | CDR3 Kabat | |
| 283 | C3CRB134 Chain B, | QSLYNNKN |
| | CALRB1114 Light | |
| | CDR1 IMGT | |
| n/a | C3CRB134 Chain B, | YAS |
| | CALRB1114 Light | |
| | CDR2 IMGT | |
| 72 | C3CRB134 Chain B, | QGEFNCNSADCNS |
| | CALRB1114 Light | |
| | CDR3 IMGT | |
| 286 | C3CRB134 Chain B, | DIQMTQSPSTLSASVGDRVTITCQASQSLY |
| | CALRB1114 VL | NNKNLAWYQQKPGCAPKLLIYYASALAS |
| | | GVPSRFSGSGSGTEFTLTISSLQPDDFATYY |
| | | CQGEFNCNSADCNSFGGGTKVEIK |
| 273 | C3CRB134 Chain B, | GFSLSSYAMS |
| | CALRB1114 Heavy | |
| | CDR1 AbM | |
| 274 | C3CRB134 Chain B, | VIDSGGTTY |
| | CALRB1114 Heavy | |
| | CDR2 AbM | |
| 275 | C3CRB134 Chain B, | ESYAGYDYYGHPIPYGMEP |
| | CALRB1114 Heavy | |
| | CDR3 AbM | |
| 276 | C3CRB134 Chain B, | GFSLSSY |
| | CALRB1114 Heavy | |
| | CDR1 Chothia | |
| 277 | C3CRB134 Chain B, | DSGGT |
| | CALRB1114 Heavy | |
| | CDR2 Chothia | |
| 275 | C3CRB134 Chain B, | ESYAGYDYYGHPIPYGMEP |
| | CALRB1114 Heavy | |
| | CDR3 Chothia | |
| 66 | C3CRB134 Chain B, | SYAMS |
| | CALRB1114 Heavy | |
| | CDR1 Kabat | |
| 278 | C3CRB134 Chain B, | VIDSGGTTYYAQKFQG |
| | CALRB1114 Heavy | |
| | CDR2 Kabat | |
| 275 | C3CRB134 Chain B, | ESYAGYDYYGHPIPYGMEP |
| | CALRB1114 Heavy | |
| | CDR3 Kabat | |
| 279 | C3CRB134 Chain B, | GFSLSSYA |
| | CALRB1114 Heavy | |
| | CDR1 IMGT | |
| 280 | C3CRB134 Chain B, | IDSGGTT |
| | CALRB1114 Heavy | |
| | CDR2 IMGT | |
| 281 | C3CRB134 Chain B, | ARESYAGYDYYGHPIPYGMEP |
| | CALRB1114 Heavy | |
| | CDR3 IMGT | |
| 287 | C3CRB134 Chain B, | QVQLVQSGAEVKKPGASVKVSCKASGFS |
| | | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGCGTLVTVSS |
| 294 | CALRB1114 VH | EVQLLESGGGLVQPGGSLRLSCAVSGIDLS |
| | C3CRB134 Chain B | NNAISWVRQAPGKGLEYVGVIGNTGDTY |
| | Heavy Chain | YADSAKGRFTISRDSSKTTLYLQMNSLRA |
| | | EDTAVYFCVRGPPSYSSSVKNIWGQGTLV |
| | | TVSSASTKGPSVFPLAPSSKSTSGGTAALG |
| | | CLVKDYFPEPVTVSWNSGALTSGVHTFPA |
| | | VLQSSGLYSLSSVVTVPSSSLGTQTYICNV |
| | | NHKPSNTKVDKKVEPKSCGGGGSGGGGS |
| | | DIQMTQSPSTLSASVGDRVTITCQASQSLY |
| | | NNKNLAWYQQKPGCAPKLLIYYASALAS |
| | | GVPSRFSGSGSGTEFTLTISSLQPDDFATYY |
| | | CQGEFNCNSADCNSFGGGTKVEIKGGGSG |
| | | GSGGCPPCGGSGGQVQLVQSGAEVKKPG |
| | | ASVKVSCKASGFSLSSYAMSWVRQAPGQ |
| | | GLEWMGVIDSGGTTYYAQKFQGRVTMTR |
| | | DTSTSTVYMELSSLRSEDTAVYYCARESY |
| | | AGYDYYGHPIPYGMEPWGCGTLVTVSSEP |
| | | KSSDKTHTCPPCPAPEAAGGPSVFLFPPKP |
| | | KDTLMISRTPEVTCVVVSVSHEDPEVKFN |
| | | WYVDGVEVHNAKTKPREEQYNSTYRVVS |
| | | VLTVLHQDWLNGKEYKCKVSNKALPAPI |
| | | EKTISKAKGQPREPQVYTLPPSREEMTKN |
| | | QVSLSCAVKGFYPSDIAVEWESNGQPENN |
| | | YKTTPPVLDSDGSFFLVSKLTVDKSRWQQ |
| | | GNVFSCSVMHEALHNRFTQKSLSLSPGK |
| 33 | C3CRB134 Chain B, | QSSQSVYNNNWLS |
| | CALRB421 Light CDR1 | |
| | AbM | |
| 23 | C3CRB134 Chain B, | DASKLES |
| | CALRB421 Light CDR2 | |
| | AbM | |
| 34 | C3CRB134 Chain B, | AGGFTGNVYT |
| | CALRB421 Light CDR3 | |
| | AbM | |
| 33 | C3CRB134 Chain B, | QSSQSVYNNNWLS |
| | CALRB421 Light CDR1 | |
| | Chothia | |
| 23 | C3CRB134 Chain B, | DASKLES |
| | CALRB421 Light CDR2 | |
| | Chothia | |
| 34 | C3CRB134 Chain B, | AGGFTGNVYT |
| | CALRB421 Light CDR3 | |
| | Chothia | |
| 33 | C3CRB134 Chain B, | QSSQSVYNNNWLS |
| | CALRB421 Light CDR1 | |
| | Kabat | |
| 23 | C3CRB134 Chain B, | DASKLES |
| | CALRB421 Light CDR2 | |
| | Kabat | |
| 34 | C3CRB134 Chain B, | AGGFTGNVYT |
| | CALRB421 Light CDR3 | |
| | Kabat | |
| 256 | C3CRB134 Chain B, | QSVYNNNW |
| | CALRB421 Light CDR1 | |
| | IMGT | |
| n/a | C3CRB134 Chain B, | DAS |
| | CALRB421 Light CDR2 | |
| | IMGT | |
| 34 | C3CRB134 Chain B, | AGGFTGNVYT |
| | CALRB421 Light CDR3 | |
| | IMGT | |
| 205 | C3CRB134 Chain B, | DIQMTQSPSSVSASVGDRVTITCQSSQSVY |
| | CALRB421 VL | NNNWLSWLQQKPGKAPKRLIYDASKLES |
| | | GVPSRFSGSGSGTDFTLTISSVQPEDAATY |
| | | YCAGGFTGNVYTFGGGTKVEIK |
| 206 | C3CRB134 Chain B | DIQMTQSPSSVSASVGDRVTITCQSSQSVY |
| | Light Chain | NNNWLSWLQQKPGKAPKRLIYDASKLES |
| | | GVPSRFSGSGSGTDFTLTISSVQPEDAATY |
| | | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |

TABLE 27
Sequences for C3CRB135 CALRmut x CD3 Bispecific Antibody

| SEQ ID NO | Description | Sequence |
|---|---|---|
| 200 | C3CRB135 Chain A, | GIDLSNNAIS |
| | CALRB421 Heavy | |
| | CDR1 AbM | |
| 201 | C3CRB135 Chain A, | VIGNTGDTY |
| | CALRB421 Heavy | |
| | CDR2 AbM | |
| 202 | C3CRB135 Chain A, | GPPSYSSSVKNI |
| | CALRB421 Heavy | |
| | CDR3 AbM | |
| 260 | C3CRB135 Chain A, | GIDLSNN |
| | CALRB421 Heavy | |
| | CDR1 Chothia | |
| 261 | C3CRB135 Chain A, | GNTGD |
| | CALRB421 Heavy | |
| | CDR2 Chothia | |
| 202 | C3CRB135 Chain A, | GPPSYSSSVKNI |
| | CALRB421 Heavy | |
| | CDR3 Chothia | |
| 81 | C3CRB135 Chain A, | NNAIS |
| | CALRB421 Heavy | |
| | CDR1 Kabat | |
| 259 | C3CRB135 Chain A, | VIGNTGDTYYADSAKG |
| | CALRB421 Heavy | |
| | CDR2 Kabat | |
| 202 | C3CRB135 Chain A, | GPPSYSSSVKNI |
| | CALRB421 Heavy | |
| | CDR3 Kabat | |
| 262 | C3CRB135 Chain A, | GIDLSNNA |
| | CALRB421 Heavy | |
| | CDR1 IMGT | |
| 263 | C3CRB135 Chain A, | IGNTGDT |
| | CALRB421 Heavy | |
| | CDR2 IMGT | |
| 264 | C3CRB135 Chain A, | VRGPPSYSSSVKNI |
| | CALRB421 Heavy | |
| | CDR3 IMGT | |
| 203 | C3CRB135 Chain A, | EVQLLESGGGLVQPGGSLRLSCAVSGIDLS |
| | CALRB421 VH | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSS |
| 70 | C3CRB135 Chain A, | QASQSLYNNKNLA |
| | CALRB1114 Light | |
| | CDR1 AbM | |
| 71 | C3CRB135 Chain A, | YASALAS |
| | CALRB1114 Light | |
| | CDR2 AbM | |
| 72 | C3CRB135 Chain A, | QGEFNCNSADCNS |
| | CALRB1114 Light | |
| | CDR3 AbM | |
| 70 | C3CRB135 Chain A, | QASQSLYNNKNLA |
| | CALRB1114 Light | |
| | CDR1 Chothia | |
| 71 | C3CRB135 Chain A, | YASALAS |
| | CALRB1114 Light | |
| | CDR2 Chothia | |
| 72 | C3CRB135 Chain A, | QGEFNCNSADCNS |
| | CALRB1114 Light | |
| | CDR3 Chothia | |
| 70 | C3CRB135 Chain A, | QASQSLYNNKNLA |
| | CALRB1114 Light | |
| | CDR1 Kabat | |
| 71 | C3CRB135 Chain A, | YASALAS |
| | CALRB1114 Light | |
| | CDR2 Kabat | |
| 72 | C3CRB135 Chain A, | QGEFNCNSADCNS |
| | CALRB1114 Light | |
| | CDR3 Kabat | |
| 283 | C3CRB135 Chain A, | QSLYNNKN |
| | CALRB1114 Light | |
| | CDR1 IMGT | |
| n/a | C3CRB135 Chain A, | YAS |
| | CALRB1114 Light | |
| | CDR2 IMGT | |
| 72 | C3CRB135 Chain A, | QGEFNCNSADCNS |
| | CALRB1114 Light | |
| | CDR3 IMGT | |
| 286 | C3CRB135 Chain A, | DIQMTQSPSTLSASVGDRVTITCQASQSLY |
| | CALRB1114 VL | NNKNLAWYQQKPGCAPKLLIYYASALAS |
| | | GVPSRFSGSGSGTEFTLTISSLQPDDFATYY |
| | | CQGEFNCNSADCNSFGGGTKVEIK |
| 273 | C3CRB135 Chain A, | GFSLSSYAMS |
| | CALRB1114 Heavy | |
| | CDR1 AbM | |
| 274 | C3CRB135 Chain A, | VIDSGGTTY |
| | CALRB1114 Heavy | |
| | CDR2 AbM | |
| 275 | C3CRB135 Chain A, | ESYAGYDYYGHPIPYGMEP |
| | CALRB1114 Heavy | |
| | CDR3 AbM | |
| 276 | C3CRB135 Chain A, | GFSLSSY |
| | CALRB1114 Heavy | |
| | CDR1 Chothia | |
| 277 | C3CRB135 Chain A, | DSGGT |
| | CALRB1114 Heavy | |
| | CDR2 Chothia | |
| 275 | C3CRB135 Chain A, | ESYAGYDYYGHPIPYGMEP |
| | CALRB1114 Heavy | |
| | CDR3 Chothia | |
| 66 | C3CRB135 Chain A, | SYAMS |
| | CALRB1114 Heavy | |
| | CDR1 Kabat | |
| 278 | C3CRB135 Chain A, | VIDSGGTTYYAQKFQG |
| | CALRB1114 Heavy | |
| | CDR2 Kabat | |
| 275 | C3CRB135 Chain A, | ESYAGYDYYGHPIPYGMEP |
| | CALRB1114 Heavy | |
| | CDR3 Kabat | |
| 279 | C3CRB135 Chain A, | GFSLSSYA |
| | CALRB1114 Heavy | |
| | CDR1 IMGT | |
| 280 | C3CRB135 Chain A, | IDSGGTT |
| | CALRB1114 Heavy | |
| | CDR2 IMGT | |
| 281 | C3CRB135 Chain A, | ARESYAGYDYYGHPIPYGMEP |
| | CALRB1114 Heavy | |
| | CDR3 IMGT | |
| 288 | C3CRB135 Chain A, | QVQLVQSGAEVKKPGASVKVSCKASGFS |
| | | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGCGTLVTVSSH |
| 295 | CALRB1114 VH | EVQLLESGGGLVQPGGSLRLSCAVSGIDLS |
| | C3CRB135 Chain A | NNAISWVRQAPGKGLEYVGVIGNTGDTY |
| | Heavy Chain | YADSAKGRFTISRDSSKTTLYLQMNSLRA |
| | | EDTAVYFCVRGPPSYSSSVKNIWGQGTLV |
| | | TVSSASTKGPSVFPLAPSSKSTSGGTAALG |
| | | CLVKDYFPEPVTVSWNSGALTSGVHTFPA |
| | | VLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTC |
| | | VVVSVSHEDPEVKFNWYVDGVEVHNAKT |
| | | KPREEQYNSTYRVVSVLTVLHQDWLNGK |
| | | EYKCKVSNKALPAPIEKTISKAKGQPREPQ |
| | | VYTLPPSREEMTKNQVSLSCAVKGFYPSDI |
| | | AVEWESNGQPENNYKTTPPVLDSDGSFFL |
| | | VSKLTVDKSRWQQGNVFSCSVMHEALHN |
| | | RFTQKSLSLSPGKGGGGSGGGGSDIQMTQ |
| | | SPSTLSASVGDRVTITCQASQSLYNNKNLA |
| | | WYQQKPGCAPKLLIYYASALASGVPSRFS |
| | | GSGSGTEFTLTISSLQPDDFATYYCQGEFN |
| | | CNSADCNSFGGGTKVEIKGGGSGGSGGCP |
| | | PCGGSGGQVQLVQSGAEVKKPGASVKVS |
| | | CKASGFSLSSYAMSWVRQAPGQGLEWM |
| | | GVIDSGGTTYYAQKFQGRVTMTRDTSTST |
| | | VYMELSSLRSEDTAVYYCARESYAGYDY |
| | | YGHPIPYGMEPWGCGTLVTVSSH |
| 33 | C3CRB135 Chain A, | QSSQSVYNNNWLS |
| | CALRB421 Light CDR1 | |
| | AbM | |
| 23 | C3CRB135 Chain A, | DASKLES |
| | CALRB421 Light CDR2 | |
| | AbM | |
| 34 | C3CRB135 Chain A, | AGGFTGNVYT |
| | CALRB421 Light CDR3 | |
| | AbM | |
| 33 | C3CRB135 Chain A, | QSSQSVYNNNWLS |
| | CALRB421 Light CDR1 | |
| | Chothia | |
| 23 | C3CRB135 Chain A, | DASKLES |
| | CALRB421 Light CDR2 | |
| | Chothia | |
| 34 | C3CRB135 Chain A, | AGGFTGNVYT |
| | CALRB421 Light CDR3 | |
| | Chothia | |
| 33 | C3CRB135 Chain A, | QSSQSVYNNNWLS |
| | CALRB421 Light CDR1 | |
| | Kabat | |
| 23 | C3CRB135 Chain A, | DASKLES |
| | CALRB421 Light CDR2 | |
| | Kabat | |
| 34 | C3CRB135 Chain A, | AGGFTGNVYT |
| | CALRB421 Light CDR3 | |
| | Kabat | |
| 256 | C3CRB135 Chain A, | QSVYNNNW |
| | CALRB421 Light CDR1 | |
| | IMGT | |
| n/a | C3CRB135 Chain A, | DAS |
| | CALRB421 Light CDR2 | |
| | IMGT | |
| 34 | C3CRB 135 Chain A, | AGGFTGNVYT |
| | CALRB421 Light CDR3 | |
| | IMGT | |
| 205 | C3CRB135 Chain A, | DIQMTQSPSSVSASVGDRVTITCQSSQSVY |
| | CALRB421 VL | NNNWLSWLQQKPGKAPKRLIYDASKLES |
| | | GVPSRFSGSGSGTDFTLTISSVQPEDAATY |
| | | YCAGGFTGNVYTFGGGTKVEIK |
| 206 | C3CRB135 Chain A | DIQMTQSPSSVSASVGDRVTITCQSSQSVY |
| | Light Chain | NNNWLSWLQQKPGKAPKRLIYDASKLES |
| | | GVPSRFSGSGSGTDFTLTISSVQPEDAATY |
| | | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |
| 214 | C3CRB135 Chain B, | RARQSIGTAIH |
| | CD3 Light CDR1 AbM | |
| 215 | C3CRB135 Chain B, | YASESIS |
| | CD3 Light CDR2 AbM | |
| 216 | C3CRB135 Chain B, | QQSGSWPYT |
| | CD3 Light CDR3 AbM | |
| 214 | C3CRB135 Chain B, | RARQSIGTAIH |
| | CD3 Light CDR1 | |
| | Chothia | |
| 215 | C3CRB135 Chain B, | YASESIS |
| | CD3 Light CDR2 | |
| | Chothia | |
| 216 | C3CRB135 Chain B, | QQSGSWPYT |
| | CD3 Light CDR3 | |
| | Chothia | |
| 214 | C3CRB135 Chain B, | RARQSIGTAIH |
| | CD3 Light CDR1 Kabat | |
| 215 | C3CRB135 Chain B, | YASESIS |
| | CD3 Light CDR2 Kabat | |
| 216 | C3CRB135 Chain B, | QQSGSWPYT |
| | CD3 Light CDR3 Kabat | |
| 272 | C3CRB135 Chain B, | QSIGTA |
| | CD3 Light CDR1 IMGT | |
| n/a | C3CRB135 Chain B, | YAS |
| | CD3 Light CDR2 IMGT | |
| 216 | C3CRB135 Chain B, | QQSGSWPYT |
| | CD3 Light CDR3 IMGT | |
| 217 | C3CRB135 Chain B, | DIQMTQSPSSLSASVGDRVTITCRARQSIG |
| | CD3 VL | TAIHWYQQKPGCAPKLLIKYASESISGVPS |
| | | RFSGSGSGTDFTLTISSLQPEDFATYYCQQ |
| | | SGSWPYTFGQGTKLEIK |
| 210 | C3CRB135 Chain B, | GFTFSRYNMN |
| | CD3 Heavy CDR1 AbM | |
| 211 | C3CRB135 Chain B, | SISTSSNYIY |
| | CD3 Heavy CDR2 AbM | |
| 212 | C3CRB135 Chain B, | GWGPFDY |
| | CD3 Heavy CDR3 AbM | |
| 267 | C3CRB135 Chain B, | GFTFSRY |
| | CD3 Heavy CDR1 | |
| | Chothia | |
| 268 | C3CRB135 Chain B, | STSSNY |
| | CD3 Heavy CDR2 | |
| | Chothia | |
| 212 | C3CRB135 Chain B, | GWGPFDY |
| | CD3 Heavy CDR3 | |
| | Chothia | |
| 265 | C3CRB135 Chain B, | RYNMN |
| | CD3 Heavy CDR1 Kabat | |
| 266 | C3CRB135 Chain B, | SISTSSNYIYYADSVKG |
| | CD3 Heavy CDR2 Kabat | |
| 212 | C3CRB135 Chain B, | GWGPFDY |
| | CD3 Heavy CDR3 Kabat | |
| 269 | C3CRB135 Chain B, | GFTFSRYN |
| | CD3 Heavy CDR1 | |
| | IMGT | |
| 270 | C3CRB135 Chain B, | ISTSSNYI |
| | CD3 Heavy CDR2 | |
| | IMGT | |
| 271 | C3CRB135 Chain B, | TRGWGPFDY |
| | CD3 Heavy CDR3 | |
| | IMGT | |
| 213 | C3CRB135 Chain B, | EVOLVESGGGLVKPGGSLRLSCAASGFTF |
| | CD3 VH | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 218 | C3CRB135 Chain B | DIQMTQSPSSLSASVGDRVTITCRARQSIG |
| | Heavy Chain | TAIHWYQQKPGCAPKLLIKYASESISGVPS |
| | | RFSGSGSGTDFTLTISSLQPEDFATYYCQQ |
| | | SGSWPYTFGQGTKLEIKGGGSGGSGGCPP |
| | | CGGSGGEVQLVESGGGLVKPGGSLRLSCA |
| | | ASGFTFSRYNMNWVRQAPGKGLEWVSSI |
| | | STSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |

TABLE 28
Sequences for C3CRB136 CALRmut x CD3 Bispecific Antibody

| SEQ ID NO | Description | Sequence |
|---|---|---|
| 214 | C3CRB136 Chain A, | RARQSIGTAIH |
| | CD3 Light CDR1 AbM | |
| 215 | C3CRB136 Chain A, | YASESIS |
| | CD3 Light CDR2 AbM | |
| 216 | C3CRB136 Chain A, | QQSGSWPYT |
| | CD3 Light CDR3 AbM | |
| 214 | C3CRB136 Chain A, | RARQSIGTAIH |
| | CD3 Light CDR1 | |
| | Chothia | |
| 215 | C3CRB136 Chain A, | YASESIS |
| | CD3 Light CDR2 | |
| | Chothia | |
| 216 | C3CRB136 Chain A, | QQSGSWPYT |
| | CD3 Light CDR3 | |
| | Chothia | |
| 214 | C3CRB 136 Chain A, | RARQSIGTAIH |
| | CD3 Light CDR1 Kabat | |
| 215 | C3CRB136 Chain A, | YASESIS |
| | CD3 Light CDR2 Kabat | |
| 216 | C3CRB136 Chain A, | QQSGSWPYT |
| | CD3 Light CDR3 Kabat | |
| 272 | C3CRB136 Chain A, | QSIGTA |
| | CD3 Light CDR1 IMGT | |
| n/a | C3CRB136 Chain A, | YAS |
| | CD3 Light CDR2 IMGT | |
| 216 | C3CRB136 Chain A, | QQSGSWPYT |
| | CD3 Light CDR3 IMGT | |
| 217 | C3CRB 136 Chain A, | DIQMTQSPSSLSASVGDRVTITCRARQSIG |
| | CD3 VL | TAIHWYQQKPGCAPKLLIKYASESISGVPS |
| | | RFSGSGSGTDFTLTISSLOPEDFATYYCQQ |
| | | SGSWPYTFGQGTKLEIK |
| 210 | C3CRB136 Chain A, | GFTFSRYNMN |
| | CD3 Heavy CDR1 AbM | |
| 211 | C3CRB136 Chain A, | SISTSSNYIY |
| | CD3 Heavy CDR2 AbM | |
| 212 | C3CRB136 Chain A, | GWGPFDY |
| | CD3 Heavy CDR3 AbM | |
| 267 | C3CRB136 Chain A, | GFTFSRY |
| | CD3 Heavy CDR1 | |
| | Chothia | |
| 268 | C3CRB136 Chain A, | STSSNY |
| | CD3 Heavy CDR2 | |
| | Chothia | |
| 212 | C3CRB136 Chain A, | GWGPFDY |
| | CD3 Heavy CDR3 | |
| | Chothia | |
| 265 | C3CRB136 Chain A, | RYNMN |
| | CD3 Heavy CDR1 Kabat | |
| 266 | C3CRB136 Chain A, | SISTSSNYIYYADSVKG |
| | CD3 Heavy CDR2 Kabat | |
| 212 | C3CRB136 Chain A, | GWGPFDY |
| | CD3 Heavy CDR3 Kabat | |
| 269 | C3CRB136 Chain A, | GFTFSRYN |
| | CD3 Heavy CDR1 | |
| | IMGT | |
| 270 | C3CRB136 Chain A, | ISTSSNYI |
| | CD3 Heavy CDR2 | |
| | IMGT | |
| 271 | C3CRB136 Chain A, | TRGWGPFDY |
| | CD3 Heavy CDR3 | |
| | IMGT | |
| 213 | C3CRB136 Chain A, | EVOLVESGGGLVKPGGSLRLSCAASGFTF |
| | CD3 VH | SRYNMNWVRQAPGKGLEWVSSISTSSNYI |
| | | YYADSVKGRFTFSRDNAKNSLDLQMSGL |
| | | RAEDTAIYYCTRGWGPFDYWGCGTLVTV |
| | | SS |
| 218 | C3CRB136 Chain A | DIQMTQSPSSLSASVGDRVTITCRARQSIG |
| | Heavy Chain | TAIHWYQQKPGCAPKLLIKYASESISGVPS |
| | | RFSGSGSGTDFTLTISSLQPEDFATYYCQQ |
| | | SGSWPYTFGQGTKLEIKGGGSGGSGGCPP |
| | | CGGSGGEVOLVESGGGLVKPGGSLRLSCA |
| | | ASGFTFSRYNMNWVRQAPGKGLEWVSSI |
| | | STSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |
| 273 | C3CRB136 Chain B, | GFSLSSYAMS |
| | CALRB1114 Heavy 1 | |
| | CDR1 AbM | |
| 274 | C3CRB136 Chain B, | VIDSGGTTY |
| | CALRB1114 Heavy 1 | |
| | CDR2 AbM | |
| 275 | C3CRB136 Chain B, | ESYAGYDYYGHPIPYGMEP |
| | CALRB1114 Heavy 1 | |
| | CDR3 AbM | |
| 276 | C3CRB136 Chain B, | GFSLSSY |
| | CALRB1114 Heavy 1 | |
| | CDR1 Chothia | |
| 277 | C3CRB136 Chain B, | DSGGT |
| | CALRB1114 Heavy 1 | |
| | CDR2 Chothia | |
| 275 | C3CRB136 Chain B, | ESYAGYDYYGHPIPYGMEP |
| | CALRB1114 Heavy 1 | |
| | CDR3 Chothia | |
| 66 | C3CRB136 Chain B, | SYAMS |
| | CALRB1114 Heavy 1 | |
| | CDR1 Kabat | |
| 278 | C3CRB136 Chain B, | VIDSGGTTYYAQKFQG |
| | CALRB1114 Heavy 1 | |
| | CDR2 Kabat | |
| 275 | C3CRB136 Chain B, | ESYAGYDYYGHPIPYGMEP |
| | CALRB1114 Heavy 1 | |
| | CDR3 Kabat | |
| 279 | C3CRB136 Chain B, | GFSLSSYA |
| | CALRB1114 Heavy 1 | |
| | CDR1 IMGT | |
| 280 | C3CRB136 Chain B, | IDSGGTT |
| | CALRB1114 Heavy 1 | |
| | CDR2 IMGT | |
| 281 | C3CRB136 Chain B, | ARESYAGYDYYGHPIPYGMEP |
| | CALRB1114 Heavy 1 | |
| | CDR3 IMGT | |
| 282 | C3CRB136 Chain B, | QVQLVQSGAEVKKPGASVKVSCKASGFS |
| | CALRB1114 VH1 | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSS |
| 273 | C3CRB136 Chain B, | GFSLSSYAMS |
| | CALRB1114 Heavy 2 | |
| | CDR1 AbM | |
| 274 | C3CRB136 Chain B, | VIDSGGTTY |
| | CALRB1114 Heavy 2 | |
| | CDR2 AbM | |
| 275 | C3CRB136 Chain B, | ESYAGYDYYGHPIPYGMEP |
| | CALRB1114 Heavy 2 | |
| | CDR3 AbM | |
| 276 | C3CRB136 Chain B, | GFSLSSY |
| | CALRB1114 Heavy 2 | |
| | CDR1 Chothia | |
| 277 | C3CRB136 Chain B, | DSGGT |
| | CALRB1114 Heavy 2 | |
| | CDR2 Chothia | |
| 275 | C3CRB136 Chain B, | ESYAGYDYYGHPIPYGMEP |
| | CALRB1114 Heavy 2 | |
| | CDR3 Chothia | |
| 66 | C3CRB136 Chain B, | SYAMS |
| | CALRB1114 Heavy 2 | |
| | CDR1 Kabat | |
| 278 | C3CRB136 Chain B, | VIDSGGTTYYAQKFQG |
| | CALRB1114 Heavy 2 | |
| | CDR2 Kabat | |
| 275 | C3CRB136 Chain B, | ESYAGYDYYGHPIPYGMEP |
| | CALRB1114 Heavy 2 | |
| | CDR3 Kabat | |
| 279 | C3CRB136 Chain B, | GFSLSSYA |
| | CALRB1114 Heavy 2 | |
| | CDR1 IMGT | |
| 280 | C3CRB136 Chain B, | IDSGGTT |
| | CALRB1114 Heavy 2 | |
| | CDR2 IMGT | |
| 281 | C3CRB136 Chain B, | ARESYAGYDYYGHPIPYGMEP |
| | CALRB1114 Heavy 2 | |
| | CDR3 IMGT | |
| 282 | C3CRB136 Chain B, | QVQLVQSGAEVKKPGASVKVSCKASGFS |
| | CALRB1114 VH2 | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSS |
| 298 | C3CRB136 Chain B | QVQLVQSGAEVKKPGASVKVSCKASGFS |
| | Heavy Chain | LSSYAMSWVRQAPGQGLEWMGVIDSGGT |
| | | TYYAQKFQGRVTMTRDTSTSTVYMELSSL |
| | | RSEDTAVYYCARESYAGYDYYGHPIPYG |
| | | MEPWGQGTLVTVSSASTKGPSVFPLAPSS |
| | | KSTSGGTAALGCLVKDYFPEPVTVSWNSG |
| | | ALTSGVHTFPAVLQSSGLYSLSSVVTVPSS |
| | | SLGTQTYICNVNHKPSNTKVDKKVEPKSC |
| | | GGGGSGGGGSQVQLVQSGAEVKKPGASV |
| | | KVSCKASGFSLSSYAMSWVRQAPGQGLE |
| | | WMGVIDSGGTTYYAQKFQGRVTMTRDTS |
| | | TSTVYMELSSLRSEDTAVYYCARESYAGY |
| | | DYYGHPIPYGMEPWGQGTLVTVSSASTK |
| | | GPSVFPLAPSSKSTSGGTAALGCLVKDYFP |
| | | EPVTVSWNSGALTSGVHTFPAVLQSSGLY |
| | | SLSSVVTVPSSSLGTQTYICNVNHKPSNTK |
| | | VDKKVEPKSCDKTHTCPPCPAPEAAGGPS |
| | | VFLFPPKPKDTLMISRTPEVTCVVVSVSHE |
| | | DPEVKFNWYVDGVEVHNAKTKPREEQYN |
| | | STYRVVSVLTVLHQDWLNGKEYKCKVSN |
| | | KALPAPIEKTISKAKGQPREPQVYTLPPSR |
| | | EEMTKNQVSLSCAVKGFYPSDIAVEWESN |
| | | GQPENNYKTTPPVLDSDGSFFLVSKLTVD |
| | | KSRWQQGNVFSCSVMHEALHNRFTQKSL |
| | | SLSPGK |
| 70 | C3CRB136 Chain B, | QASQSLYNNKNLA |
| | CALRB1114 Light 1 | |
| | CDR1 AbM | |
| 71 | C3CRB136 Chain B, | YASALAS |
| | CALRB1114 Light 1 | |
| | CDR2 AbM | |
| 72 | C3CRB136 Chain B, | QGEFNCNSADCNS |
| | CALRB1114 Light 1 | |
| | CDR3 AbM | |
| 70 | C3CRB136 Chain B, | QASQSLYNNKNLA |
| | CALRB1114 Light 1 | |
| | CDR1 Chothia | |
| 71 | C3CRB136 Chain B, | YASALAS |
| | CALRB1114 Light 1 | |
| | CDR2 Chothia | |
| 72 | C3CRB136 Chain B, | QGEFNCNSADCNS |
| | CALRB1114 Light 1 | |
| | CDR3 Chothia | |
| 70 | C3CRB136 Chain B, | QASQSLYNNKNLA |
| | CALRB1114 Light 1 | |
| | CDR1 Kabat | |
| 71 | C3CRB136 Chain B, | YASALAS |
| | CALRB1114 Light 1 | |
| | CDR2 Kabat | |
| 72 | C3CRB136 Chain B, | QGEFNCNSADCNS |
| | CALRB1114 Light 1 | |
| | CDR3 Kabat | |
| 283 | C3CRB136 Chain B, | QSLYNNKN |
| | CALRB1114 Light 1 | |
| | CDR1 IMGT | |
| n/a | C3CRB136 Chain B, | YAS |
| | CALRB1114 Light 1 | |
| | CDR2 IMGT | |
| 72 | C3CRB136 Chain B, | QGEFNCNSADCNS |
| | CALRB1114 Light 1 | |
| | CDR3 IMGT | |
| 284 | C3CRB136 Chain B, | DIQMTQSPSTLSASVGDRVTITCQASQSLY |
| | CALRB1114 VL 1 | NNKNLAWYQQKPGKAPKLLIYYASALAS |
| | | GVPSRFSGSGSGTEFTLTISSLQPDDFATYY |
| | | CQGEFNCNSADCNSFGGGTKVEIK |
| 291 | C3CRB136 Chain B | DIQMTQSPSTLSASVGDRVTITCQASQSLY |
| | Light Chain 1 | NNKNLAWYQQKPGKAPKLLIYYASALAS |
| | | GVPSRFSGSGSGTEFTLTISSLQPDDFATYY |
| | | CQGEFNCNSADCNSFGGGTKVEIKRTVAA |
| | | PSVFIFPPSDEQLKSGTASVVCLLNNFYPR |
| | | EAKVQWKVDNALQSGNSQESVTEQDSKD |
| | | STYSLSSTLTLSKADYEKHKVYACEVTHQ |
| | | GLSSPVTKSFNRGEC |
| 70 | C3CRB136 Chain B, | QASQSLYNNKNLA |
| | CALRB1114 Light 2 | |
| | CDR1 AbM | |
| 71 | C3CRB136 Chain B, | YASALAS |
| | CALRB1114 Light 2 | |
| | CDR2 AbM | |
| 72 | C3CRB136 Chain B, | QGEFNCNSADCNS |
| | CALRB1114 Light 2 | |
| | CDR3 AbM | |
| 70 | C3CRB136 Chain B, | QASQSLYNNKNLA |
| | CALRB1114 Light 2 | |
| | CDR1 Chothia | |
| 71 | C3CRB136 Chain B, | YASALAS |
| | CALRB1114 Light 2 | |
| | CDR2 Chothia | |
| 72 | C3CRB136 Chain B, | QGEFNCNSADCNS |
| | CALRB1114 Light 2 | |
| | CDR3 Chothia | |
| 70 | C3CRB136 Chain B, | QASQSLYNNKNLA |
| | CALRB1114 Light 2 | |
| | CDR1 Kabat | |
| 71 | C3CRB136 Chain B, | YASALAS |
| | CALRB1114 Light 2 | |
| | CDR2 Kabat | |
| 72 | C3CRB136 Chain B, | QGEFNCNSADCNS |
| | CALRB1114 Light 2 | |
| | CDR3 Kabat | |
| 283 | C3CRB136 Chain B, | QSLYNNKN |
| | CALRB1114 Light 2 | |
| | CDR1 IMGT | |
| n/a | C3CRB136 Chain B, | YAS |
| | CALRB1114 Light 2 | |
| | CDR2 IMGT | |
| 72 | C3CRB136 Chain B, | QGEFNCNSADCNS |
| | CALRB1114 Light 2 | |
| | CDR3 IMGT | |
| 284 | C3CRB136 Chain B, | DIQMTQSPSTLSASVGDRVTITCQASQSLY |
| | CALRB1114 VL 2 | NNKNLAWYQQKPGKAPKLLIYYASALAS |
| | | GVPSRFSGSGSGTEFTLTISSLQPDDFATYY |
| | | CQGEFNCNSADCNSFGGGTKVEIK |
| 291 | C3CRB136 Chain B | DIQMTQSPSTLSASVGDRVTITCQASQSLY |
| | Light Chain 2 | NNKNLAWYQQKPGKAPKLLIYYASALAS |
| | | GVPSRFSGSGSGTEFTLTISSLQPDDFATYY |
| | | CQGEFNCNSADCNSFGGGTKVEIKRTVAA |
| | | PSVFIFPPSDEQLKSGTASVVCLLNNFYPR |
| | | EAKVQWKVDNALQSGNSQESVTEQDSKD |
| | | STYSLSSTLTLSKADYEKHKVYACEVTHQ |
| | | GLSSPVTKSFNRGEC |

TABLE 29
Sequences for C3CRB137 CALRmut x CD3
Bispecific Antibody

| SEQ | | |
|---|---|---|
| ID | Descrip- | |
| NO | tion | Sequence |
| 214 | C3CRB137 | RARQSIGTAIH |
| | Chain A, | |
| | CD3 Light | |
| | CDR1 AbM | |
| 215 | C3CRB137 | YASESIS |
| | Chain A, | |
| | CD3 Light | |
| | CDR2 AbM | |
| 216 | C3CRB137 | QQSGSWPYT |
| | Chain A, | |
| | CD3 Light | |
| | CDR3 AbM | |
| 214 | C3CRB137 | RARQSIGTAIH |
| | Chain A, | |
| | CD3 Light | |
| | CDR1 | |
| | Chothia | |
| 215 | C3CRB137 | YASESIS |
| | Chain A, | |
| | CD3 Light | |
| | CDR2 | |
| | Chothia | |
| 216 | C3CRB137 | QQSGSWPYT |
| | Chain A, | |
| | CD3 Light | |
| | CDR3 | |
| | Chothia | |
| 214 | C3CRB137 | RARQSIGTAIH |
| | Chain A, | |
| | CD3 Light | |
| | CDR1 | |
| | Kabat | |
| 215 | C3CRB137 | YASESIS |
| | Chain A, | |
| | CD3 Light | |
| | CDR2 | |
| | Kabat | |
| 216 | C3CRB137 | QQSGSWPYT |
| | Chain A, | |
| | CD3 Light | |
| | CDR3 | |
| | Kabat | |
| 272 | C3CRB137 | QSIGTA |
| | Chain A, | |
| | CD3 Light | |
| | CDR1 IMGT | |
| n/a | C3CRB137 | YAS |
| | Chain A, | |
| | CD3 Light | |
| | CDR2 IMGT | |
| 216 | C3CRB137 | QQSGSWPYT |
| | Chain A, | |
| | CD3 Light | |
| | CDR3 IMGT | |
| 217 | C3CRB137 | DIQMTQSPSSLSASVGDRVTITCRARQSIG |
| | Chain A, | TAIHWYQQKPGCAPKLLIKYASESISGVPS |
| | CD3 VL | RFSGSGSGTDFTLTISSLQPEDFATYYCQQ |
| | | SGSWPYTFGQGTKLEIK |
| 210 | C3CRB137 | GFTFSRYNMN |
| | Chain A, | |
| | CD3 Heavy | |
| | CDR1 AbM | |
| 211 | C3CRB137 | SISTSSNYIY |
| | Chain A, | |
| | CD3 Heavy | |
| | CDR2 AbM | |
| 212 | C3CRB137 | GWGPFDY |
| | Chain A, | |
| | CD3 Heavy | |
| | CDR3 AbM | |
| 267 | C3CRB137 | GFTFSRY |
| | Chain A, | |
| | CD3 Heavy | |
| | CDR1 | |
| | Chothia | |
| 268 | C3CRB137 | STSSNY |
| | Chain A, | |
| | CD3 Heavy | |
| | CDR2 | |
| | Chothia | |
| 212 | C3CRB137 | GWGPFDY |
| | Chain A, | |
| | CD3 Heavy | |
| | CDR3 | |
| | Chothia | |
| 265 | C3CRB137 | RYNMN |
| | Chain A, | |
| | CD3 Heavy | |
| | CDR1 | |
| | Kabat | |
| 266 | C3CRB137 | SISTSSNYIYYADSVKG |
| | Chain A, | |
| | CD3 Heavy | |
| | CDR2 | |
| | Kabat | |
| 212 | C3CRB137 | GWGPFDY |
| | Chain A, | |
| | CD3 Heavy | |
| | CDR3 | |
| | Kabat | |
| 269 | C3CRB137 | GFTFSRYN |
| | Chain A, | |
| | CD3 Heavy | |
| | CDR1 | |
| | IMGT | |
| 270 | C3CRB137 | ISTSSNYI |
| | Chain A, | |
| | CD3 Heavy | |
| | CDR2 | |
| | IMGT | |
| 271 | C3CRB137 | TRGWGPFDY |
| | Chain A, | |
| | CD3 Heavy | |
| | CDR3 | |
| | IMGT | |
| 213 | C3CRB137 | EVQLVESGGGLVKPGGSLRLSCAASGFTF |
| | Chain A, | SRYNMNWVRQAPGKGLEWVSSISTSSNYI |
| | CD3 VH | YYADSVKGRFTFSRDNAKNSLDLQMSGL |
| | | RAEDTAIYYCTRGWGPFDYWGCGTLVTV |
| | | SS |
| 218 | C3CRB137 | DIQMTQSPSSLSASVGDRVTITCRARQSIG |
| | Chain A | TAIHWYQQKPGCAPKLLIKYASESISGVPS |
| | Heavy | RFSGSGSGTDFTLTISSLQPEDFATYYCQQ |
| | Chain | SGSWPYTFGQGTKLEIKGGGSGGSGGCPP |
| | | CGGSGGEVQLVESGGGLVKPGGSLRLSCA |
| | | ASGFTFSRYNMNWVRQAPGKGLEWVSSI |
| | | STSSNYIYYADSVKGRFTFSRDNAKNSLD |
| | | LQMSGLRAEDTAIYYCTRGWGPFDYWGC |
| | | GTLVTVSSEPKSSDKTHTCPPCPAPEAAGG |
| | | PSVFLFPPKPKDTLMISRTPEVTCVVVSVS |
| | | HEDPEVKFNWYVDGVEVHNAKTKPREEQ |
| | | YNSTYRVVSVLTVLHQDWLNGKEYKCKV |
| | | SNKALPAPIEKTISKAKGQPREPQVYTLPP |
| | | SREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQ |
| | | KSLSLSPGK |
| 200 | C3CRB137 | GIDLSNNAIS |
| | Chain B, | |
| | CALRB421 | |
| | Heavy 1 | |
| | CDR1 AbM | |
| 201 | C3CRB137 | VIGNTGDTY |
| | Chain B, | |
| | CALRB421 | |
| | Heavy 1 | |
| | CDR2 AbM | |
| 202 | C3CRB137 | GPPSYSSSVKNI |
| | Chain B, | |
| | CALRB421 | |
| | Heavy 1 | |
| | CDR3 AbM | |
| 260 | C3CRB137 | GIDLSNN |
| | Chain B, | |
| | CALRB421 | |
| | Heavy 1 | |
| | CDR1 | |
| | Chothia | |
| 261 | C3CRB137 | GNTGD |
| | Chain B, | |
| | CALRB421 | |
| | Heavy 1 | |
| | CDR2 | |
| | Chothia | |
| 202 | C3CRB137 | GPPSYSSSVKNI |
| | Chain B, | |
| | CALRB421 | |
| | Heavy 1 | |
| | CDR3 | |
| | Chothia | |
| 81 | C3CRB137 | NNAIS |
| | Chain B, | |
| | CALRB421 | |
| | Heavy 1 | |
| | CDR1 | |
| | Kabat | |
| 259 | C3CRB137 | VIGNTGDTYYADSAKG |
| | Chain B, | |
| | CALRB421 | |
| | Heavy 1 | |
| | CDR2 | |
| | Kabat | |
| 202 | C3CRB137 | GPPSYSSSVKNI |
| | Chain B, | |
| | CALRB421 | |
| | Heavy 1 | |
| | CDR3 | |
| | Kabat | |
| 262 | C3CRB137 | GIDLSNNA |
| | Chain B, | |
| | CALRB421 | |
| | Heavy 1 | |
| | CDR1 IMGT | |
| 263 | C3CRB137 | IGNTGDT |
| | Chain B, | |
| | CALRB421 | |
| | Heavy 1 | |
| | CDR2 IMGT | |
| 264 | C3CRB137 | VRGPPSYSSSVKNI |
| | Chain B, | |
| | CALRB421 | |
| | Heavy 1 | |
| | CDR3 IMGT | |
| 203 | C3CRB137 | EVQLLESGGGLVQPGGSLRLSCAVSGIDLS |
| | Chain B, | NNAISWVRQAPGKGLEYVGVIGNTGDTY |
| | CALRB421 | YADSAKGRFTISRDSSKTTLYLQMNSLRA |
| | VH1 | EDTAVYFCVRGPPSYSSSVKNIWGQGTLV |
| | | TVSS |
| 200 | C3CRB137 | GIDLSNNAIS |
| | Chain B, | |
| | CALRB421 | |
| | Heavy 2 | |
| | CDR1 AbM | |
| 201 | C3CRB137 | VIGNTGDTY |
| | Chain B, | |
| | CALRB421 | |
| | Heavy 2 | |
| | CDR2 AbM | |
| 202 | C3CRB137 | GPPSYSSSVKNI |
| | Chain B, | |
| | CALRB421 | |
| | Heavy 2 | |
| | CDR3 AbM | |
| 260 | C3CRB137 | GIDLSNN |
| | Chain B, | |
| | CALRB421 | |
| | Heavy 2 | |
| | CDR1 | |
| | Chothia | |
| 261 | C3CRB137 | GNTGD |
| | Chain B, | |
| | CALRB421 | |
| | Heavy 2 | |
| | CDR2 | |
| | Chothia | |
| 202 | C3CRB137 | GPPSYSSSVKNI |
| | Chain B, | |
| | CALRB421 | |
| | Heavy 2 | |
| | CDR3 | |
| | Chothia | |
| 81 | C3CRB137 | NNAIS |
| | Chain B, | |
| | CALRB421 | |
| | Heavy 2 | |
| | CDR1 | |
| | Kabat | |
| 259 | C3CRB137 | VIGNTGDTYYADSAKG |
| | Chain B, | |
| | CALRB421 | |
| | Heavy 2 | |
| | CDR2 | |
| | Kabat | |
| 202 | C3CRB137 | GPPSYSSSVKNI |
| | Chain B, | |
| | CALRB421 | |
| | Heavy 2 | |
| | CDR3 | |
| | Kabat | |
| 262 | C3CRB137 | GIDLSNNA |
| | Chain B, | |
| | CALRB421 | |
| | Heavy 2 | |
| | CDR1 IMGT | |
| 263 | C3CRB137 | IGNTGDT |
| | Chain B, | |
| | CALRB421 | |
| | Heavy 2 | |
| | CDR2 IMGT | |
| 264 | C3CRB137 | VRGPPSYSSSVKNI |
| | Chain B, | |
| | CALRB421 | |
| | Heavy 2 | |
| | CDR3 IMGT | |
| 203 | C3CRB137 | EVQLLESGGGLVQPGGSLRLSCAVSGIDLS |
| | Chain B, | NNAISWVRQAPGKGLEYVGVIGNTGDTY |
| | CALRB421 | YADSAKGRFTISRDSSKTTLYLQMNSLRA |
| | VH2 | EDTAVYFCVRGPPSYSSSVKNIWGQGTLV |
| | | TVSS |
| 296 | C3CRB137 | EVQLLESGGGLVQPGGSLRLSCAVSGIDLS |
| | Chain B | NNAISWVRQAPGKGLEYVGVIGNTGDTY |
| | Heavy | YADSAKGRFTISRDSSKTTLYLQMNSLRA |
| | Chain | EDTAVYFCVRGPPSYSSSVKNIWGQGTLV |
| | | TVSSASTKGPSVFPLAPSSKSTSGGTAALG |
| | | CLVKDYFPEPVTVSWNSGALTSGVHTFPA |
| | | VLQSSGLYSLSSVVTVPSSSLGTQTYICNV |
| | | NHKPSNTKVDKKVEPKSCGGGGSGGGGS |
| | | EVQLLESGGGLVQPGGSLRLSCAVSGIDLS |
| | | NNAISWVRQAPGKGLEYVGVIGNTGDTY |
| | | YADSAKGRFTISRDSSKTTLYLQMNSLRA |
| | | EDTAVYFCVRGPPSYSSSVKNIWGQGTLV |
| | | TVSSASTKGPSVFPLAPSSKSTSGGTAALG |
| | | CLVKDYFPEPVTVSWNSGALTSGVHTFPA |
| | | VLQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVTC |
| | | VVVSVSHEDPEVKFNWYVDGVEVHNAKT |
| | | KPREEQYNSTYRVVSVLTVLHQDWLNGK |
| | | EYKCKVSNKALPAPIEKTISKAKGQPREPQ |
| | | VYTLPPSREEMTKNQVSLSCAVKGFYPSDI |
| | | AVEWESNGQPENNYKTTPPVLDSDGSFFL |
| | | VSKLTVDKSRWQQGNVFSCSVMHEALHN |
| | | RFTQKSLSLSPGK |
| 33 | C3CRB137 | QSSQSVYNNNWLS |
| | Chain B, | |
| | CALRB421 | |
| | Light 1 | |
| | CDR1 AbM | |
| 23 | C3CRB137 | DASKLES |
| | Chain B, | |
| | CALRB421 | |
| | Light 1 | |
| | CDR2 AbM | |
| 34 | C3CRB137 | AGGFTGNVYT |
| | Chain B, | |
| | CALRB421 | |
| | Light 1 | |
| | CDR3 AbM | |
| 33 | C3CRB137 | QSSQSVYNNNWLS |
| | Chain B, | |
| | CALRB421 | |
| | Light 1 | |
| | CDR1 | |
| | Chothia | |
| 23 | C3CRB137 | DASKLES |
| | Chain B, | |
| | CALRB421 | |
| | Light 1 | |
| | CDR2 | |
| | Chothia | |
| 34 | C3CRB137 | AGGFTGNVYT |
| | Chain B, | |
| | CALRB421 | |
| | Light 1 | |
| | CDR3 | |
| | Chothia | |
| 33 | C3CRB137 | QSSQSVYNNNWLS |
| | Chain B, | |
| | CALRB421 | |
| | Light 1 | |
| | CDR1 | |
| | Kabat | |
| 23 | C3CRB137 | DASKLES |
| | Chain B, | |
| | CALRB421 | |
| | Light 1 | |
| | CDR2 | |
| | Kabat | |
| 34 | C3CRB137 | AGGFTGNVYT |
| | Chain B, | |
| | CALRB421 | |
| | Light 1 | |
| | CDR3 | |
| | Kabat | |
| 256 | C3CRB137 | QSVYNNNW |
| | Chain B, | |
| | CALRB421 | |
| | Light 1 | |
| | CDR1 IMGT | |
| n/a | C3CRB137 | DAS |
| | Chain B, | |
| | CALRB421 | |
| | Light 1 | |
| | CDR2 IMGT | |
| 34 | C3CRB137 | AGGFTGNVYT |
| | Chain B, | |
| | CALRB421 | |
| | Light 1 | |
| | CDR3 IMGT | |
| 205 | C3CRB137 | DIQMTQSPSSVSASVGDRVTITCQSSQSVY |
| | Chain B, | NNNWLSWLQQKPGKAPKRLIYDASKLES |
| | CALRB421 | GVPSRFSGSGSGTDFTLTISSVQPEDAATY |
| | VL 1 | YCAGGFTGNVYTFGGGTKVEIK |
| 206 | C3CRB137 | DIQMTQSPSSVSASVGDRVTITCQSSQSVY |
| | Chain B | NNNWLSWLQQKPGKAPKRLIYDASKLES |
| | Light | GVPSRFSGSGSGTDFTLTISSVQPEDAATY |
| | Chain 1 | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |
| 33 | C3CRB137 | QSSQSVYNNNWLS |
| | Chain B, | |
| | CALRB421 | |
| | Light 2 | |
| | CDR1 AbM | |
| 23 | C3CRB137 | DASKLES |
| | Chain B, | |
| | CALRB421 | |
| | Light 2 | |
| | CDR2 AbM | |
| 34 | C3CRB137 | AGGFTGNVYT |
| | Chain B, | |
| | CALRB421 | |
| | Light 2 | |
| | CDR3 AbM | |
| 33 | C3CRB137 | QSSQSVYNNNWLS |
| | Chain B, | |
| | CALRB421 | |
| | Light 2 | |
| | CDR1 | |
| | Chothia | |
| 23 | C3CRB137 | DASKLES |
| | Chain B, | |
| | CALRB421 | |
| | Light 2 | |
| | CDR2 | |
| | Chothia | |
| 34 | C3CRB137 | AGGFTGNVYT |
| | Chain B, | |
| | CALRB421 | |
| | Light 2 | |
| | CDR3 | |
| | Chothia | |
| 33 | C3CRB137 | QSSQSVYNNNWLS |
| | Chain B, | |
| | CALRB421 | |
| | Light 2 | |
| | CDR1 | |
| | Kabat | |
| 23 | C3CRB137 | DASKLES |
| | Chain B, | |
| | CALRB421 | |
| | Light 2 | |
| | CDR2 | |
| | Kabat | |
| 34 | C3CRB137 | AGGFTGNVYT |
| | Chain B, | |
| | CALRB421 | |
| | Light 2 | |
| | CDR3 | |
| | Kabat | |
| 256 | C3CRB137 | QSVYNNNW |
| | Chain B, | |
| | CALRB421 | |
| | Light 2 | |
| | CDR1 IMGT | |
| n/a | C3CRB137 | DAS |
| | Chain B, | |
| | CALRB421 | |
| | Light 2 | |
| | CDR2 IMGT | |
| 34 | C3CRB137 | AGGFTGNVYT |
| | Chain B, | |
| | CALRB421 | |
| | Light 2 | |
| | CDR3 IMGT | |
| 205 | C3CRB137 | DIQMTQSPSSVSASVGDRVTITCQSSQSVY |
| | Chain B, | NNNWLSWLQQKPGKAPKRLIYDASKLES |
| | CALRB421 | GVPSRFSGSGSGTDFTLTISSVQPEDAATY |
| | VL 2 | YCAGGFTGNVYTFGGGTKVEIK |
| 206 | C3CRB137 | DIQMTQSPSSVSASVGDRVTITCQSSQSVY |
| | Chain B | NNNWLSWLQQKPGKAPKRLIYDASKLES |
| | Light | GVPSRFSGSGSGTDFTLTISSVQPEDAATY |
| | Chain 2 | YCAGGFTGNVYTFGGGTKVEIKRTVAAPS |
| | | VFIFPPSDEQLKSGTASVVCLLNNFYPREA |
| | | KVQWKVDNALQSGNSQESVTEQDSKDST |
| | | YSLSSTLTLSKADYEKHKVYACEVTHQGL |
| | | SSPVTKSFNRGEC |

### B. CALRmutxCD3-Induced T Cell-Mediated Cytotoxicity of CALRmut-Engineered Cancer Cells

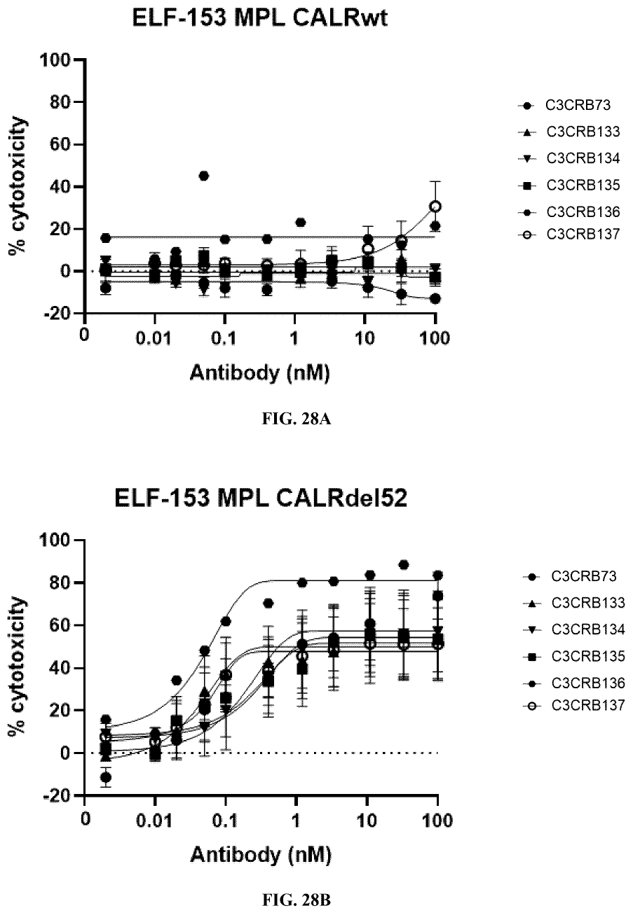

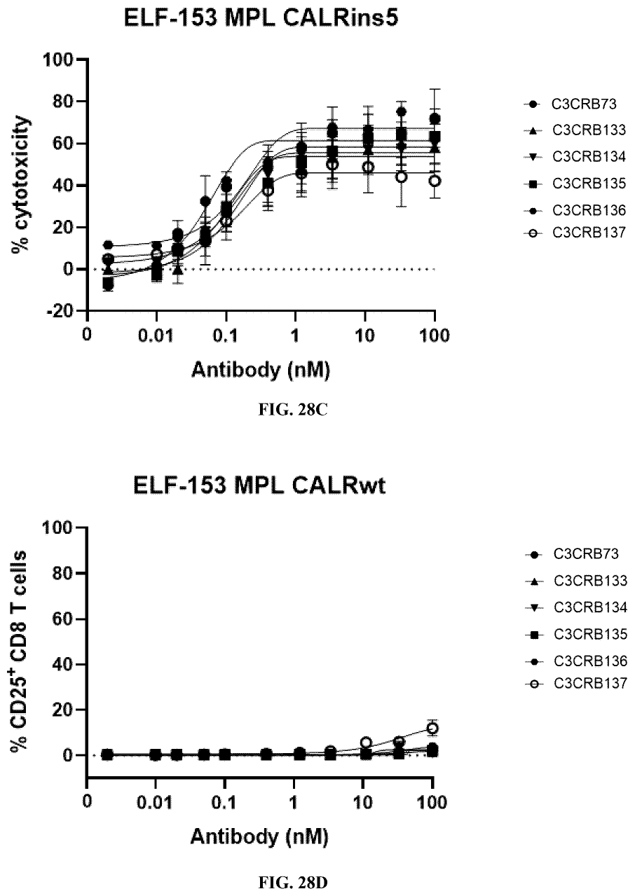

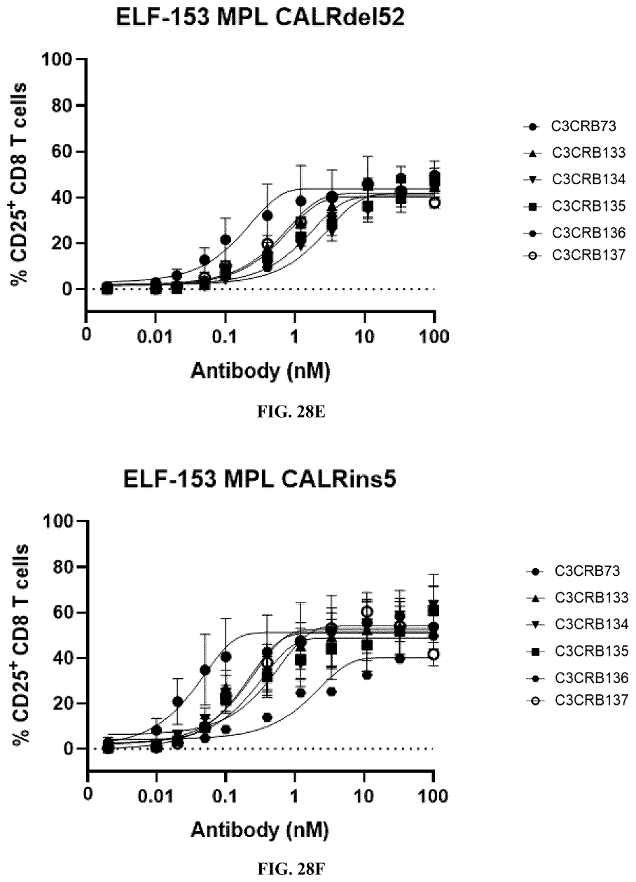

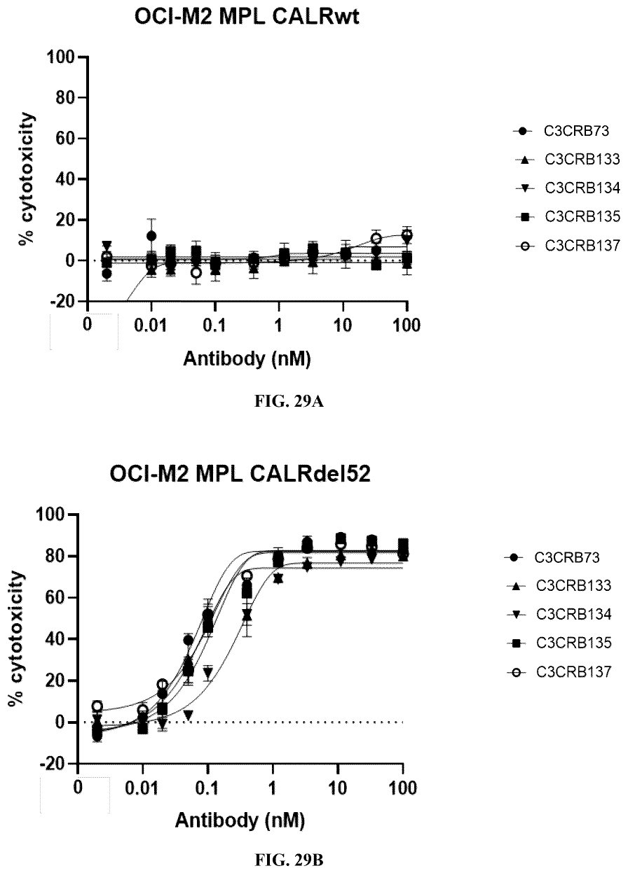

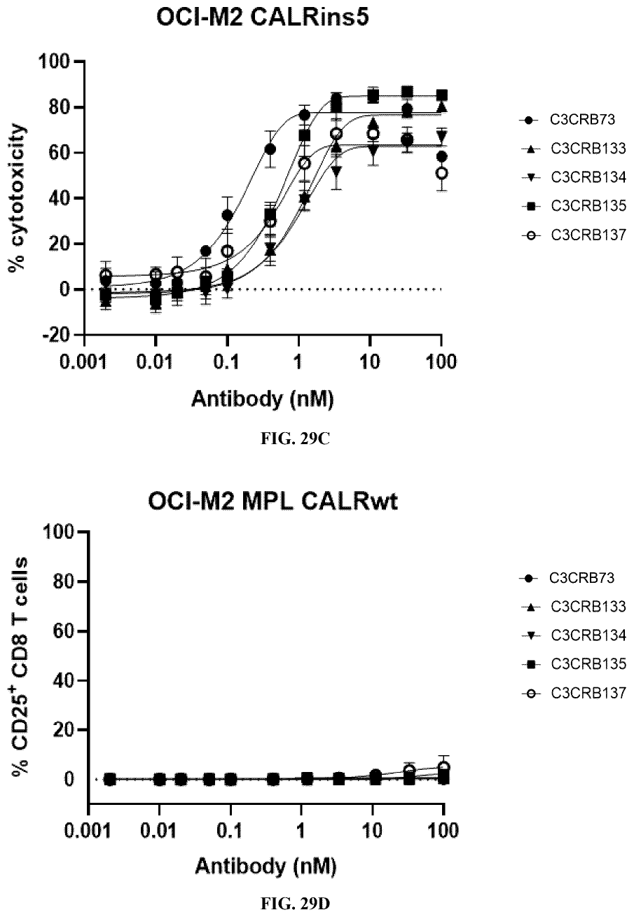

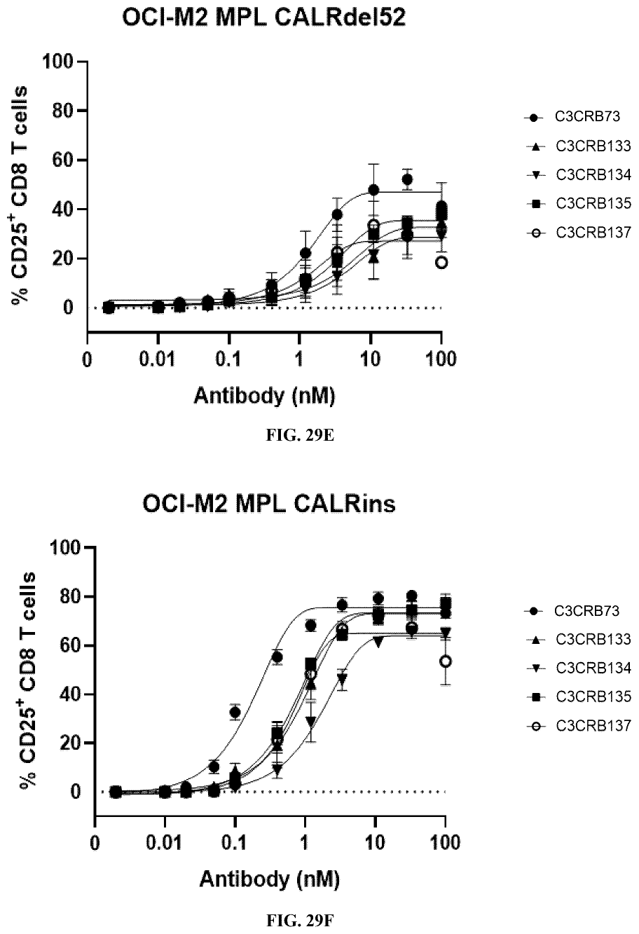

T-cell cytotoxicity assays were performed to evaluate the ability of CALRmutxCD3 to induce CALRmut-selective cytotoxicity of cell lines engineered to express the most common CALRmuts, i.e., CALRdel52 and CALRins5. T cells from healthy donors were used at 5:1 E:T ratios. After 72 hours, CALRmutxCD3 antibodies elicited concentration-dependent cytotoxicity of the cell lines expressing MPL CALRdel52 or MPL CALRins5 (FIGS. 28B, 28C, and FIGS. 29B, 29C). No cytotoxicity was observed for the ELF-153 MPL CALRwt (FIG. 28A) or OCI-M2 MPL CALRwt (FIG. 29A) cell lines. These results demonstrate that all tested CALRmutxCD3 bispecifics can induce selective cytotoxicity of CALRmut cell lines expressing different levels of CALRmut on the cell surface. CD25 expression as a measure of T-cell activation was monitored in the cytotoxicity assays described herein. CALRmutxCD3 elicited concentration-dependent increases in the activation of T cells in the presence of cell lines expressing CALRmut after 72 hours (FIGS. 28E, 28F and FIGS. 29 E, 29F) but did not elicit activation in coculture with target-negative cell lines (FIG. 28D, FIG. 29D).

C. CALRmutxCD3-induced T cell-mediated Cytotoxicity of CALRmut CD34+ Cells Derived from CALRmut MPN Patient Cells in Autologous Setting

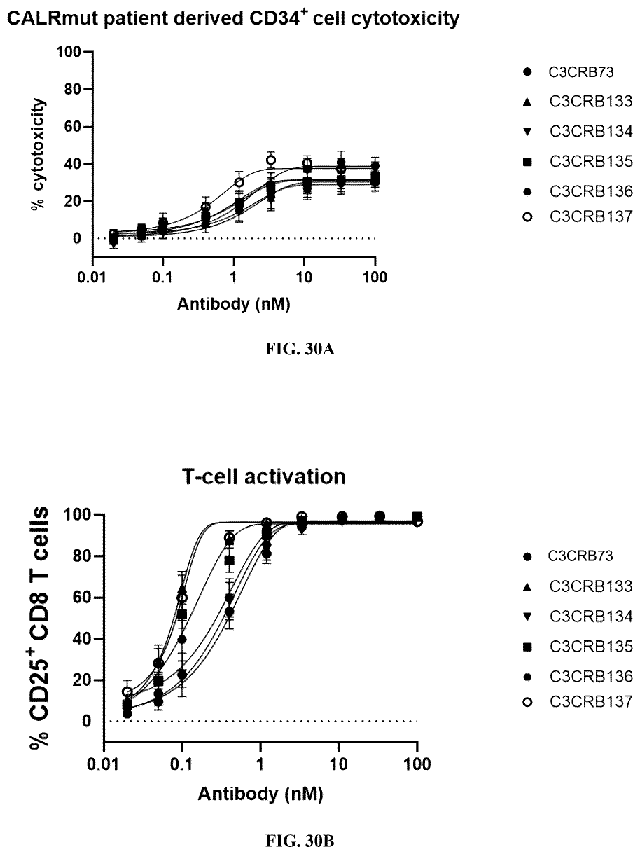

To evaluate the impact of CALRmutxCD3 in a more physiologically relevant setting, T-cell cytotoxicity assays were conducted in an autologous setting using CD34+ cells isolated from CALRmut MPN patients as target cells together with T cells isolated from the same patient. The studies were conducted using an E:T ratio of 5:1. CALRmutxCD3 antibodies, listed in Table 22, elicited concentration-dependent cytotoxicity of patient-derived CALRmut CD34+ cells (FIG. 30A). T-cell activation was assessed in parallel by measuring % CD25-positive cell on the surface of CD8 T cells. CALRmutxCD3 antibodies elicited concentration-dependent T-cell activation in autologous cytotoxicity assays (FIG. 30B).

Although previous studies reported exhaustion of T cells isolated from CALRmnut MPN patients, the current results confirm that T cells in the presence of CALRmutxCD3 antibodies are capable of engaging CALRmut-patient-derived T cells to mediate cytotoxicity to CALRmut-expressing CD34+ cells in an autologous setting.

Sequence Listing

| SEQ ID NO: | Sequence |
|---|---|
| 1 | KMSPARPRTSCREACLQGWTEA |
| 2 | RRKMSPARPRTSCREACLQGWTEA |
| 3 | EPAVYFKEQFLDGDGWTSRWIESKHKSDFGKFVLSSGKFYGDEEKDKGLQTS |
| | QDARFYALSASFEPFSNKGQTLVVQFTVKHEQNIDCGGGYVKLFPNSLDQTD |
| | MHGDSEYNIMFGPDICGPGTKKVHVIFNYKGKNVLINKDIRCKDDEFTHLYTLI |
| | VRPDNTYEVKIDNSQVESGSLEDDWDFLPPKKIKDPDASKPEDWDERAKIDDP |
| | TDSKPEDWDKPEHIPDPDAKKPEDWDEEMDGEWEPPVIQNPEYKGEWKPRQI |
| | DNPDYKGTWIHPEIDNPEYSPDPSIYAYDNFGVLGLDLWQVKSGTIFDNFLITN |
| | DEAYAEEFGNETWGVTKAAEKQMKDKQDEEQRLKEEEEDKKRKEEEEAEDN |
| | CRRMMRTKMRMRRMRRTRRKMRRKMSPARPRTSCREACLQGWTEADYKDD |
| | DDK |
| 4 | EPAVYFKEQFLDGDGWTSRWIESKHKSDFGKFVLSSGKFYGDEEKDKGLQTS |
| | QDARFYALSASFEPFSNKGQTLVVQFTVKHEQNIDCGGGYVKLFPNSLDQTD |
| | MHGDSEYNIMFGPDICGPGTKKVHVIFNYKGKNVLINKDIRCKDDEFTHLYTLI |
| | VRPDNTYEVKIDNSQVESGSLEDDWDFLPPKKIKDPDASKPEDWDERAKIDDP |
| | TDSKPEDWDKPEHIPDPDAKKPEDWDEEMDGEWEPPVIQNPEYKGEWKPRQI |
| | DNPDYKGTWIHPEIDNPEYSPDPSIYAYDNFGVLGLDLWQVKSGTIFDNFLITN |
| | DEAYAEEFGNETWGVTKAAEKQMKDKQDEEQRLKEEEEDKKRKEEEEAEDK |
| | EDDEDKDEDEEDEEDKEEDEEEDVPGQAKDELDYKDDDDK |
| 5 | MRGSHHHHHHGMASMTGGQQMGRDLYDDDDKDRWGSELEAEPAVYFKEQF |
| | LDGDGWTSRWIESKHKSDFGKFVLSSGKFYGDEEKDKGLQTSQDARFYALSA |
| | SFEPFSNKGQTLVVQFTVKHEQNIDCGGGYVKLFPNSLDQTDMHGDSEYNIMF |
| | GPDICGPGTKKVHVIFNYKGKNVLINKDIRCKDDEFTHLYTLIVRPDNTYEVKI |
| | DNSQVESGSLEDDWDFLPPKKIKDPDASKPEDWDERAKIDDPTDSKPEDWDKP |
| | EHIPDPDAKKPEDWDEEMDGEWEPPVIQNPEYKGEWKPRQIDNPDYKGTWIH |
| | PEIDNPEYSPDPSIYAYDNFGVLGLDLWQVKSGTIFDNFLITNDEAYAEEFGNE |
| | TWGVTKAAEKQMKDKQDEEQRTRRMMRTKMRMRRMRRTRRKMRRKMSPA |
| | RPRTSCREACLQGWTEA |
| 6 | MGSHHHHHHGSSGEPAVYFKEQFLDGDGWTSRWIESKHKSDFGKFVLSSGKF |
| | YGDEEKDKGLQTSQDARFYALSASFEPFSNKGQTLVVQFTVKHEQNIDCGGG |
| | YVKLFPNSLDQTDMHGDSEYNIMFGPDICGPGTKKVHVIFNYKGKNVLINKDI |
| | RSKDDEFTHLYTLIVRPDNTYEVKIDNSQVESGSLEDDWDFLPPKKIKDPDASK |
| | PEDWDERAKIDDPTDSKPEDWDKPEHIPDPDAKKPEDWDEEMDGEWEPPVIQ |
| | NPEYKGEWKPRQIDNPDYKGTWIHPEIDNPEYSPDPSIYAYDNFGVLGLDLWQ |
| | VKSGTIFDNFLITNDEAYAEEFGNETWGVTKAAEKQMKDKQDEEQRTRRMM |
| | RTKMRMRRMRRTRRKMRRKMSPARPRTSCREACLQGWTEA |
| 10 | IYGMS |
| 11 | IIYIEGITYYANWAKGRFT |
| 12 | GVGRI |
| 13 | QEQLKESGGRLVTPGTPLTLTCAVSGFSLDIYGMSWVRQAPGKGLEWIGIIYIE |
| | GITYYANWAKGRFTISKTSSTTLELKMTSLTTEDTAMYFCVAGVGRIWGPGTL |
| | VTVSS |
| 14 | QASQSLYNNNNLA |
| 15 | PASTLAS |
| 16 | LGEFSCSSADCYA |
| 17 | YVMMTQTPSSVSAAVGGTVTINCQASQSLYNNNNLAWFQQKPGQPPKRLIYP |
| | ASTLASGVSSRFKGSGSGTQFTLTISDVQRDDAATYFCLGEFSCSSADCYAFGG |
| | GTEVVVK |
| 19 | VIGNTGTTYYASWAKGRFT |
| 20 | GPPSYSNSVKNI |
| 21 | QSVEESGGRLVTPGTPLTLTCTVSGIDLSNNAISWVRQAPGKGLEYIGVIGNTG |
| | TTYYASWAKGRFTISKTSSTTVDLKMTSLTASDTATYFCVRGPPSYSNSVKNI |
| | WGPGTLVTVSS |
| 22 | QSSQSVYDKNWLS |
| 23 | DASKLES |
| 24 | AGGFTGNVYA |
| 25 | DPMLTQTPSSVSAAVGGTVTIGCQSSQSVYDKNWLSWYQQKPGQPPSRLIYDA |
| | SKLESGVPSRFSGSGSGTQFTLTIGGVQCDDAATYYCAGGFTGNVYAFGGGTK |
| | LEIK |
| 26 | NNAVS |
| 27 | VIGNTGSTYYANWAKGRFT |
| 28 | VRGPPSYSNSVKNI |
| 29 | QEQLKESGGRLVAPGTPLRLTCRVSGIDLSNNAVSWVRQAPGKGLEYIGVIGN |
| | TGSTYYANWAKGRFTISKTSSTTVDLKMTSLTADDTATYFCVRGPPSYSNSVK |
| | NIWGPGTLVTVSS |
| 30 | DPMLTQTPSSVSAAVGGTVTIGCQSSQSVYDKNWLSWYQQKPGQPPSRLIYDA |
| | SKLESGVPSRFSGSGSGTQFTLTISGVQCDDAATYYCAGGFTGNVYAFGGGTE |
| | VVVK |
| 31 | VIGNTGDTYYASWAKGRFT |
| 32 | QEQLEESGGRLVTPGTPLTLTCKVSGIDLSNNAISWVRQAPGKGLEYIGVIGNT |
| | GDTYYASWAKGRFTISKTSSTTVDLKMTSLTTEDTATYFCVRGPPSYSNSVKNI |
| | WGPGTLVTISS |
| 33 | QSSQSVYNNNWLS |
| 34 | AGGFTGNVYT |
| 35 | DPMLTQTPSSVSAAVGGTVTINCQSSQSVYNNNWLSWLQQKPGQPPNRLIYD |
| | ASKLESGVPSRFSGSGSGTQFTLTISGVQRDDAATYYCAGGFTGNVYTFGGGT |
| | EVVVK |
| 37 | QEQLKESGGRLVAPGTPLRLTCTVSGIDLSNNAVSWVRQAPGKGLEYIGVIGN |
| | TGTTYYASWAKGRFTISKTTSTTVDLKMTSLTTDDTATYFCVRGPPSYSNSVK |
| | NIWGPGTLVTVSS |
| 38 | AGGFTGNVYG |
| 39 | DPVLTQTPSSVSAAVGGTVTIGCQSSQSVYDKNWLSWYQQKPGQPPSRLIYDA |
| | SKLESGVPSRFSGSGSGTQFTLTISGVQCDDAATYYCAGGFTGNVYGFGGGTE |
| | VVVK |
| 39 | VIGNTGTTYYANWAKGRFT |
| 40 | GPPSYSNSVRNI |
| 41 | QQLEESGGRLVAPGTPLRLTCTVSGIDLSNNAVSWVRQAPGKGLEYIGVIGNT |
| | GTTYYANWAKGRFTISKTSSTTVDLKMTSLTTDDTATYFCVRGPPSYSNSVRN |
| | IWGPGTLVTVSS |
| 42 | QSSQSVYDNNWLS |
| 43 | DASKLAS |
| 44 | AGGYTGNVYS |
| 45 | DPMLTQTPSSVSAAVGGTVTIGCQSSQSVYDNNWLSWYQQKPGQPPNRLIYD |
| | ASKLASGVPSRFSGSGSGTQFTLTISGVKCDDAATYYCAGGYTGNVYSFGGGT |
| | MVEIK |
| 46 | TVSGIDLSNNAISW |
| 47 | QSVEESGGRLVTPGTPLTLTCTVSGIDLSNNAISWVRQAPGKGLEYIGVIGNTG |
| | DTYYASWAKGRFTISKTSSTTVDLKMTSLTTEDTATYFCVRGPPSYSNSVKNI |
| | WGPGTLVTISS |
| 48 | DPMLTQTPSSVSAAVGGTVTIGCQSSQSVYDKNWLSWYQQKPGQPPSRLIYDA |
| | SKLESGVPSRFSGSGSGTQFTLTISGVQCDDVATYYCAGGFTGNVYAFGGGTE |
| | VVVK |
| 49 | TVSGIDLSNNAVS |
| 50 | QEQLKESGGRLVAPGTPLRLTCTVSGIDLSNNAVSWVRQAPGKGLEYIGVIGN |
| | TGTTYYANWAKGRFTISKTSSTTVDLKMTSLTTDDTATYFCVRGPPSYSNSVK |
| | NIWGPGTLVTVSS |
| 51 | QASQSVYNNNWLT |
| 52 | AAGWTGNVYS |
| 53 | DPMLTQTPSPVSAAVGGTVTINCQASQSVYNNNWLTWYQQKPGQPPNRLIYD |
| | ASKLASGVPSRFSGSGFGTQFTLTISGVQCDDAATYYCAAGWTGNVYSFGGGT |
| | EVVVK |
| 54 | QQLEESGGRLVTPGTPLTLTCTVSGIDLSNNAISWVRQAPGKGLEYIGVIGNTG |
| | TTYYASWAKGRFTISKTSSTTVDLKMTSLTASDTATYFCVRGPPSYSNSVKNI |
| | WGPGTLVTISS |
| 55 | DVVMTQTPSSVSAAVGGTVTINCQSSQSVYNNNWLSWLQQKPGQPPNRLIYD |
| | ASKLESGVPSRFSGSGSGTQFTLTISGVQCDDAATYYCAGGFTGNVYTFGGGT |
| | EVVVK |
| 56 | NYYMS |
| 57 | GISSRAGTWYASWAKGRFT |
| 58 | GLKI |
| 59 | QEQLKESGGRLVTPGTPLTLTCTASGFSLSNYYMSWVRQAPGKGLEWIGGISS |
| | RAGTWYASWAKGRFTISGTSTTMDLKITSPTTEDTATYFCARGLKIWGPGTLV |
| | TVSS |
| 60 | ASQSVYDDNNLA |
| 61 | QGEFSCTSADCVA |
| 62 | DPVLTQTESPVSAAVGGTVTINCQASQSVYDDNNLAWYQQKPGQPPKRLIYPA |
| | STLASGVSSRFSGSGSGSQFTLTISDVQCDDAATYYCQGEFSCTSADCVAFGGG |
| | TEVVVK |
| 63 | NNAMS |
| 64 | QEQLEESGGRLVTPGTPLTLTCTVSGIDLSNNAMSWVRQAPGKGLEYIGVIGN |
| | TGTTYYANWAKGRFTISKTSSATVDLKMTSLTTDDTATYFCVRGPPSYSNSVK |
| | NIWGPGTLVTVSS |
| 65 | DVMLTQTPSSVSAAVGGTVTINCQSSQSVYDKNWLSWLQQKPGQPPSRLIYD |
| | ASKLESGVPSRFSGSGSGTQFTLTISGVQCDDAATYYCAGGFTGNVYTFGGGT |
| | KVVVK |
| 66 | SYAMS |
| 67 | VIDSGGTTYYATWAKS |
| 68 | ESYAGYDGYGHPIPYGMDP |
| 69 | QSVEESGGRLVTPGTPLTITCTVSGFSLSSYAMSWVRQAPGKGLEWIGVIDSGG |
| | TTYYATWAKSRFTISKTQTKVALKITSPTTEDTATYFCARESYAGYDGYGHPIP |
| | YGMDPWGPGTLVTVSS |
| 70 | QASQSLYNNKNLA |
| 71 | YASALAS |
| 72 | QGEFNCNSADCNS |
| 73 | DVVMTQTPSSVSAAVGGTVTINCQASQSLYNNKNLAWYQQKPGQPPKLLIYY |
| | ASALASGVSSRFKGSGSGTQFTLTISGVQCDDAATYYCQGEFNCNSADCNSFG |
| | GGTEVVVK |
| 75 | QSLEESGGRLVTPGTPLTLTCTVSGIDLSNNAISWVRQAPGKGLEYIGVIGNTG |
| | DTYYASWAKGRFTISKTSSTTVDLKMTSLTTEDTATYFCVRGPPSYSNSVKNI |
| | WGPGTLVTISS |
| 76 | DPMLTQTPSSVSAAVGGTVTIGCQSSQSVYDKNWLSWYQQKPGQPPSRLIYDA |
| | SKLESGVPSRFSGSGSGTQFTLTISGVQCDDAATYYCAGGFTGNVYAFGGGTK |
| | LEIK |
| 77 | QEQLKESGGRLVAPGTPLRLTCTVSGIDLSNNAVSWVRQAPGKGLEYIGVIGN |
| | TGTTYYANWAKGRFTISKTSSTTVDLKMTSLTTDDTATYFCVRGPPSYSNSVK |
| | NIWGPGTLVTISS |
| 78 | DVVMTQTPSSVSAAVGGTVTIGCQSSQSVYDKNWLSWYQQKPGQPPSRLIYD |
| | ASKLESGVPSRFSGSGSGTQFTLTISGVQCDDAATYYCAGGFTGNVYAFGGGT |
| | MVEIK |
| 79 | QQLEESGGRLVTPGTPLTLTCTVSGIDLSNNAMSWVRQAPGKGLEYIGVIGNT |
| | GTTYYASWAKGRFTISKTSSTTVDLKMTSLTTEDTATYFCVRGPPSYSNSVKNI |
| | WGPGTLVTIS |
| 80 | DPVLTQTPSSVSAAVGGTVTINCQSSQSVYNNNWLSWLQQKPGQPPNRLIYDA |
| | SKLESGVPSRFSGSGSGTQFTLTISGVQCDDAATYYCAGGFTGNVYTFGGGTE |
| | VVVK |
| 81 | NNAIS |
| 100 | YVMMTQTPSSVSAAVGGTVTINCQASQSLYNNNNLAWFQQKPGQPPKRLIYP |
| | ASTLASGVSSRFKGSGSGTQFTLTISDVQSDDAATYFCLGEFSCSSADCYAFGG |
| | GTEVVVKQEQLKESGGRLVTPGTPLTLTCAVSGFSLDIYGMSWVRQAPGKGL |
| | EWIGIIYIEGITYYANWAKGRFTISKTSSTTLELKMTSLTTEDTAMYFCVAGVG |
| | RIWGPGTLVTVSS |
| 101 | DPMLTQTPSSVSAAVGGTVTINCQSSQSVYNNNWLSWLQQKPGQPPNRLIYD |
| | ASKLESGVPSRFSGSGSGTQFTLTISGVQSDDAATYYCAGGFTGNVYTFGGGT |
| | EVVVKQEQLEESGGRLVTPGTPLTLTCKVSGIDLSNNAISWVRQAPGKGLEYIG |
| | VIGNTGDTYYASWAKGRFTISKTSSTTVDLKMTSLTTEDTATYFCVRGPPSYSN |
| | SVKNIWGPGTLVTISS |
| 102 | DPMLTQTPSSVSAAVGGTVTIGCQSSQSVYDNNWLSWYQQKPGQPPNRLIYD |
| | ASKLASGVPSRFSGSGSGTQFTLTISGVKSDDAATYYCAGGYTGNVYSFGGGT |
| | MVEIKQQLEESGGRLVAPGTPLRLTCTVSGIDLSNNAVSWVRQAPGKGLEYIG |
| | VIGNTGTTYYANWAKGRFTISKTSSTTVDLKMTSLTTDDTATYFCVRGPPSYS |
| | NSVRNIWGPGTLVTVSS |
| 103 | DPMLTQTPSSVSAAVGGTVTIGCQSSQSVYDKNWLSWYQQKPGQPPSRLIYDA |
| | SKLESGVPSRFSGSGSGTQFTLTISGVQSDDVATYYCAGGFTGNVYAFGGGTE |
| | VVVKQSVEESGGRLVTPGTPLTLTCTVSGIDLSNNAISWVRQAPGKGLEYIGVI |
| | GNTGDTYYASWAKGRFTISKTSSTTVDLKMTSLTTEDTATYFCVRGPPSYSNS |
| | VKNIWGPGTLVTISS |
| 104 | DPVLTQTESPVSAAVGGTVTINCQASQSVYDDNNLAWYQQKPGQPPKRLIYPA |
| | STLASGVSSRESGSGSGSQFTLTISDVQSDDAATYYCQGEFSCTSADCVAFGGG |
| | TEVVVKQEQLKESGGRLVTPGTPLTLTCTASGFSLSNYYMSWVRQAPGKGLE |
| | WIGGISSRAGTWYASWAKGRFTISGTSSTTMDLKITSPTTEDTATYFCARGLKI |
| | WGPGTLVTVSS |
| 105 | DPVLTQTESPVSAAVGGTVTINCQASQSVYDDNNLAWYQQKPGQPPKRLIYPA |
| | STLASGVSSRFSGSGSGSQFTLTISDVQSDDAATYYCQGEFSCTSADCVAFGGG |
| | TEVVVKQEQLKESGGRLVTPGTPLTLTCTASGFSLSNYYMSWVRQAPGKGLE |
| | WIGGISSRAGTWYASWAKGRFTISGTSTTMDLKITSPTTEDTATYFCARGLKIW |
| | GPGTLVTVSS |
| 106 | DVVMTQTPSSVSAAVGGTVTINCQASQSLYNNKNLAWYQQKPGQPPKLLIYY |
| | ASALASGVSSRFKGSGSGTQFTLTISGVQSDDAATYYCQGEFNCNSADCNSFG |
| | GGTEVVVKQSVEESGGRLVTPGTPLTITCTVSGFSLSSYAMSWVRQAPGKGLE |
| | WIGVIDSGGTTYYATWAKSRFTISKTSSTKVALKITSPTTEDTATYFCARESYA |
| | GYDGYGHPIPYGMDPWGPGTLVTVSS |
| 107 | DVVMTQTPSSVSAAVGGTVTINCQASQSLYNNKNLAWYQQKPGQPPKLLIYY |
| | ASALASGVSSRFKGSGSGTQFTLTISGVQSDDAATYYCQGEFNCNSADCNSFG |
| | GGTEVVVKQSVEESGGRLVTPGTPLTITCTVSGFSLSSYAMSWVRQAPGKGLE |
| | WIGVIDSGGTTYYATWAKSRFTISKTTKVALKITSPTTEDTATYFCARESYAGY |
| | DGYGHPIPYGMDPWGPGTLVTVSS |
| 200 | GIDLSNNAIS |
| 201 | VIGNTGDTY |
| 202 | GPPSYSSSVKNI |
| 203 | EVQLLESGGGLVQPGGSLRLSCAVSGIDLSNNAISWVRQAPGKGLEYVGVIGN |
| | TGDTYYADSAKGRFTISRDSSKTTLYLQMNSLRAEDTAVYFCVRGPPSYSSSV |
| | KNIWGQGTLVTVSS |
| 204 | EVQLLESGGGLVQPGGSLRLSCAVSGIDLSNNAISWVRQAPGKGLEYVGVIGN |
| | TGDTYYADSAKGRFTISRDSSKTTLYLQMNSLRAEDTAVYFCVRGPPSYSSSV |
| | KNIWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCDKTHTCPPCPAPEAAGGPSVFLFPPKPKDTLMISRTPEVTCVVVS |
| | VSHEDPEVKFNWYVDGVEVHNAKTKPREEQYNSTYRVVSVLTVLHQDWLNG |
| | KEYKCKVSNKALPAPIEKTISKAKGQPREPQVYTLPPSREEMTKNQVSLSCAV |
| | KGFYPSDIAVEWESNGQPENNYKTTPPVLDSDGSFFLVSKLTVDKSRWQQGN |
| | VFSCSVMHEALHNRFTQKSLSLSPGK |
| 205 | DIQMTQSPSSVSASVGDRVTITCQSSQSVYNNNWLSWLQQKPGKAPKRLIYDA |
| | SKLESGVPSRFSGSGSGTDFTLTISSVQPEDAATYYCAGGFTGNVYTFGGGTKV |
| | EIK |
| 206 | DIQMTQSPSSVSASVGDRVTITCQSSQSVYNNNWLSWLQQKPGKAPKRLIYDA |
| | SKLESGVPSRFSGSGSGTDFTLTISSVQPEDAATYYCAGGFTGNVYTFGGGTKV |
| 207 | EIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFN |
| | RGEC |
| | ATGGCCAGGAAGTCCGCTCTGCTCGCTCTGGCACTTCTGCTTCTGGGATTTG |
| | GACCTGCTTGGGCTGAGGTTCAGCTGCTGGAATCTGGCGGAGGATTGGTTC |
| | AGCCTGGCGGCTCTCTGAGACTGTCTTGTGCTGTGTCTGGCATCGACCTGTC |
| | CAACAACGCCATCTCCTGGGTCCGACAGGCTCCTGGCAAAGGCCTGGAATA |
| | TGTGGGCGTGATCGGCAACACCGGCGACACCTACTACGCCGATTCTGCCAA |
| | GGGCAGATTCACCATCTCTCGGGACTCCTCCAAGACCACACTGTACCTGCA |
| | GATGAACTCCCTGAGAGCCGAGGATACCGCCGTGTACTTTTGTGTGCGGGG |
| | ACCTCCTAGCTACTCCTCCAGCGTGAAGAACATCTGGGGCCAGGGCACACT |
| | GGTCACCGTTTCTTCTGCCTCCACCAAGGGCCCATCGGTCTTCCCCCTGGCA |
| | CCCTCCTCCAAGAGCACCTCTGGGGGCACAGCGGCCCTGGGCTGCCTGGTC |
| | AAGGACTACTTCCCCGAACCGGTGACGGTGTCGTGGAACTCAGGCGCCCTG |
| | ACCAGCGGCGTGCACACCTTCCCGGCTGTCCTACAGTCCTCAGGACTCTAC |
| | TCCCTCAGCAGCGTGGTGACCGTGCCCTCCAGCAGCTTGGGCACCCAGACC |
| | TACATCTGCAACGTGAATCACAAGCCCAGCAACACCAAGGTGGACAAGAA |
| | AGTTGAGCCCAAATCTTGTGACAAAACTCACACATGCCCACCGTGCCCAGC |
| | ACCTGAAGCCGCCGGGGGACCGTCAGTCTTCCTCTTCCCCCCAAAACCCAA |
| | GGACACCCTCATGATCTCCCGGACCCCTGAGGTCACATGCGTGGTGGTGAG |
| | CGTGAGCCACGAAGACCCTGAGGTCAAGTTCAACTGGTACGTGGACGGCG |
| | TGGAGGTGCATAATGCCAAGACAAAGCCGCGGGAGGAGCAGTACAACAGC |
| | ACGTACCGTGTGGTCAGCGTCCTCACCGTCCTGCACCAGGACTGGCTGAAT |
| | GGCAAGGAGTACAAGTGCAAGGTGTCGAACAAAGCCCTCCCAGCCCCCAT |
| | CGAGAAAACCATCTCCAAAGCCAAAGGGCAGCCCCGAGAACCACAGGTGT |
| | ACACCCTGCCCCCATCCCGGGAGGAGATGACCAAGAACCAGGTCAGCCTG |
| | TCCTGCGCCGTCAAAGGCTTCTATCCCAGCGACATCGCCGTGGAGTGGGAG |
| | AGCAATGGGCAGCCGGAGAACAACTACAAGACCACGCCTCCCGTGCTGGA |
| | CTCCGACGGCTCCTTCTTCCTCGTGAGCAAGCTCACCGTGGACAAGAGCAG |
| | ATGGCAGCAGGGGAACGTCTTCTCATGCTCCGTGATGCATGAGGCTCTGCA |
| | CAACCGGTTCACGCAGAAGTCTCTCTCCCTGTCTCCGGGAAAA |
| 208 | ATGGCCAGGAAGTCCGCTCTGCTCGCTCTGGCACTTCTGCTTCTGGGATTTG |
| | GACCTGCTTGGGCTGACATCCAGATGACCCAGTCTCCATCCTCCGTGTCTGC |
| | CTCTGTGGGCGACAGAGTGACCATCACCTGTCAGTCCAGCCAATCCGTGTA |
| | CAACAACAACTGGCTGTCCTGGCTGCAGCAGAAGCCTGGCAAGGCTCCCA |
| | AGAGACTGATCTACGACGCCTCCAAGCTGGAATCCGGCGTGCCCTCCAGAT |
| | TTTCCGGCTCTGGCTCTGGCACCGACTTTACCCTGACCATCTCCTCCGTGCA |
| | GCCTGAGGATGCCGCCACCTATTATTGTGCTGGAGGCTTCACCGGCAACGT |
| | GTACACATTTGGCGGAGGCACCAAGGTGGAAATCAAGCGTACTGTGGCTG |
| | CACCATCTGTCTTCATCTTCCCGCCATCTGATGAGCAGTTGAAATCTGGAAC |
| | TGCCTCTGTTGTGTGCCTGCTGAATAACTTCTATCCCAGAGAGGCCAAAGT |
| | ACAGTGGAAGGTGGATAACGCCCTCCAATCGGGTAACTCCCAGGAGAGTG |
| | TCACAGAGCAGGACAGCAAGGACAGCACCTACAGCCTCAGCAGCACCCTG |
| | ACGCTGAGCAAAGCAGACTACGAGAAACACAAAGTCTACGCCTGCGAAGT |
| | CACCCATCAGGGCCTGAGCTCGCCCGTCACAAAGAGCTTCAACAGGGGAG |
| | AGTGT |
| 210 | GFTFSRYNMN |
| 211 | SISTSSNYIY |
| 212 | GWGPFDY |
| 213 | EVQLVESGGGLVKPGGSLRLSCAASGFTFSRYNMNWVRQAPGKGLEWVSSIS |
| | TSSNYIYYADSVKGRFTFSRDNAKNSLDLQMSGLRAEDTAIYYCTRGWGPFDY |
| | WGCGTLVTVSS |
| 214 | RARQSIGTAIH |
| 215 | YASESIS |
| 216 | QQSGSWPYT |
| 217 | DIQMTQSPSSLSASVGDRVTITCRARQSIGTAIHWYQQKPGCAPKLLIKYASESI |
| | SGVPSRFSGSGSGTDFTLTISSLQPEDFATYYCQQSGSWPYTFGQGTKLEIK |
| 218 | DIQMTQSPSSLSASVGDRVTITCRARQSIGTAIHWYQQKPGCAPKLLIKYASESI |
| | SGVPSRFSGSGSGTDFTLTISSLQPEDFATYYCQQSGSWPYTFGQGTKLEIKGG |
| | GSGGSGGCPPCGGSGGEVQLVESGGGLVKPGGSLRLSCAASGFTFSRYNMNW |
| | VRQAPGKGLEWVSSISTSSNYIYYADSVKGRFTFSRDNAKNSLDLQMSGLRAE |
| | DTAIYYCTRGWGPFDYWGCGTLVTVSSEPKSSDKTHTCPPCPAPEAAGGPSVF |
| | LFPPKPKDTLMISRTPEVTCVVVSVSHEDPEVKFNWYVDGVEVHNAKTKPREE |
| | QYNSTYRVVSVLTVLHQDWLNGKEYKCKVSNKALPAPIEKTISKAKGQPREP |
| | QVYTLPPSREEMTKNQVSLWCLVKGFYPSDIAVEWESNGQPENNYKTTPPVL |
| | DSDGSFFLYSKLTVDKSRWQQGNVFSCSVMHEALHNHYTQKSLSLSPGK |
| 219 | ATGGCCAGGAAGTCCGCTCTGCTCGCTCTGGCACTTCTGCTTCTGGGATTTG |
| | GACCTGCTTGGGCTGACATCCAGATGACCCAGTCTCCATCCTCTCTGTCCGC |
| | CTCTGTGGGCGACAGAGTGACCATTACCTGCCGGGCCAGACAGTCTATCGG |
| | CACCGCTATCCACTGGTATCAGCAGAAGCCTGGCTGTGCCCCTAAGCTGCT |
| | GATTAAGTACGCCTCCGAGTCCATCTCCGGCGTGCCCTCCAGATTTTCTGGC |
| | TCTGGATCTGGCACCGACTTTACCCTGACAATCTCCAGCCTGCAGCCTGAG |
| | GACTTCGCCACCTACTACTGTCAGCAGTCCGGCTCTTGGCCTTACACCTTTG |
| | GTCAGGGCACCAAGCTGGAAATCAAGGGCGGAGGTTCTGGTGGATCTGGC |
| | GGATGTCCTCCTTGTGGTGGAAGCGGCGGAGAGGTGCAGCTGGTTGAATCT |
| | GGCGGAGGACTGGTTAAGCCTGGCGGCTCTCTGAGACTGTCTTGTGCTGCT |
| | TCTGGCTTCACCTTCAGCCGGTACAACATGAACTGGGTCCGACAGGCTCCT |
| | GGCAAAGGCCTGGAATGGGTGTCCTCCATCTCCACCTCCAGCAACTACATC |
| | TACTACGCCGACTCCGTGAAGGGCAGATTCACCTTCTCCAGAGACAACGCC |
| | AAGAACTCCCTGGACCTGCAGATGTCTGGCCTGAGAGCTGAGGACACCGCT |
| | ATCTACTACTGCACCAGAGGCTGGGGACCCTTCGATTATTGGGGCTGTGGA |
| | ACCCTGGTCACCGTGTCATCTGAGCCCAAATCTAGCGACAAAACTCACACA |
| | TGCCCACCGTGCCCAGCACCTGAAGCCGCCGGGGGACCGTCAGTCTTCCTC |
| | TTCCCCCCAAAACCCAAGGACACCCTCATGATCTCCCGGACCCCTGAGGTC |
| | ACATGCGTGGTGGTGAGCGTGAGCCACGAAGACCCTGAGGTCAAGTTCAA |
| | CTGGTACGTGGACGGCGTGGAGGTGCATAATGCCAAGACAAAGCCGCGGG |
| | AGGAGCAGTACAACAGCACGTACCGTGTGGTCAGCGTCCTCACCGTCCTGC |
| | ACCAGGACTGGCTGAATGGCAAGGAGTACAAGTGCAAGGTGTCGAACAAA |
| | GCCCTCCCAGCCCCCATCGAGAAAACCATCTCCAAAGCCAAAGGGCAGCC |
| | CCGAGAACCACAGGTGTACACCCTGCCCCCATCCCGGGAGGAGATGACCA |
| | AGAACCAGGTCAGCCTGTGGTGCCTGGTCAAAGGCTTCTATCCCAGCGACA |
| | TCGCCGTGGAGTGGGAGAGCAATGGGCAGCCGGAGAACAACTACAAGACC |
| | ACGCCTCCCGTGCTGGACTCCGACGGCTCCTTCTTCCTCTACAGCAAGCTCA |
| | CCGTGGACAAGAGCAGATGGCAGCAGGGGAACGTCTTCTCATGCTCCGTG |
| | ATGCATGAGGCTCTGCACAACCACTACACGCAGAAGTCTCTCTCCCTGTCT |
| | CCGGGAAAA |
| 220 | TRRMMRTKMRMRRMRRTRRKMRRKMSPARPRTSCREACLQGWTEA |
| 221 | NCRRMMRTKMRMRRMRRTRRKMRRKMSPARPRTSCREACLQGWTEA |
| 222 | QRTRRMMRTKMRMRRMRRTRRKMRRKMSPARPRTSCREACLQGWTEA |
| 223 | RRRQRTRRMMRTKMRMRRMRRTRRKMRRKMSPARPRTSCREACLQGWTEA |
| 224 | GRTRRMMRTKMRMRRMRRTRRKMRRKMSPARPRTSCREACLQGWTEA |
| 225 | RRQRTRRMMRTKMRMRRMRRTRRKMRRKMSPARPRTSCREACLQGWTEA |
| 226 | RRMMRTKMRMRRMRRTRRKMRRKMSPARPRTSCREACLQGWTEA |
| 228 | RQRTRRMMRTKMRMRRMRRTRRKMRRKMSPARPRTSCREACLQGWTEA |
| 229 | MCRRMMRTKMRMRRMRRTRRKMRRKMSPARPRTSCREACLQGWTEA |
| 230 | DQRQRTRRMMRTKMRMRRMRRTRRKMRRKMSPARPRTSCREACLQGWTEA |
| 231 | RRRRQRTRRMMRTKMRMRRMRRTRRKMRRKMSPARPRTSCREACLQGWTE |
| | A |
| 232 | QRRRQRTRRMMRTKMRMRRMRRTRRKMRRKMSPARPRTSCREACLQGWTE |
| | A |
| 234 | RRRERTRRMMRTKMRMRRMRRTRRKMRRKMSPARPRTSCREACLQGWTEA |
| 235 | QRRQRTRRMMRTKMRMRRMRRTRRKMRRKMSPARPRTSCREACLQGWTEA |
| 236 | RRQWTRRMMRTKMRMRRMRRTRRKMRRKMSPARPRTSCREACLQGWTEA |
| 237 | RMMRTKMRMRRMRRTRRKMRRKMSPARPRTSCREACLQGWTEA |
| 239 | GRRQRTRRMMRTKMRMRRMRRTRRKMRRKMSPARPRTSCREACLQGWTEA |
| 240 | AFKRTRRMMRTKMRMRRMRRTRRKMRRKMSPARPRTSCREACLQGWTEA |
| 241 | NAKRRRRQRTRRMMRTKMRMRRMRRTRRKMRRKMSPARPRTSCREACLQG |
| | WTE |
| 242 | CVRRRRQRTRRMMRTKMRMRRMRRTRRKMRRKMSPARPRTSCREACLQGW |
| | TEA |
| 243 | RRQRTRRMMRTKMRMRRMRRTRRKMRRKMSPARPRTSCREACLQGWTEA |
| 244 | RQRTRRMMRTKMRMRRMRRTRRKMRRKMSPARPRTSCREACLQGWTEA |
| 246 | CFAKRRRRQRTRRMMRTKMRMRRMRRTRRKMRRKMSPARPRTSCREACLQ |
| | GWT |
| 248 | PPLCLRRMMRTKMRMRRMRRTRRKMRRKMSPARPRTSCREACLQGWTEA |
| 249 | DHPCRRMMRTKMRMRRMRRTRRKMRRKMSPARPRTSCREACLQGWTEA |
| 250 | GNCRRMMRTKMRMRRMRRTRRKMRRKMSPARPRTSCREACLQGWTEA |
| 251 | CRRMMRTKMRMRRMRRTRRKMRRKMSPARPRTSCREACLQGWTEA |
| 253 | TCRRMMRTKMRMRRMRRTRRKMRRKMSPARPRTSCREACLQGWTEA |
| 254 | ICRRMMRTKMRMRRMRRTRRKMRRKMSPARPRTSCREACLQGWTEA |
| 256 | QSVYNNNW |
| 259 | VIGNTGDTYYADSAKG |
| 260 | GIDLSNN |
| 261 | GNTGD |
| 262 | GIDLSNNA |
| 263 | IGNTGDT |
| 264 | VRGPPSYSSSVKNI |
| 265 | RYNMN |
| 266 | SISTSSNYIYYADSVKG |
| 267 | GFTFSRY |
| 268 | STSSNY |
| 269 | GFTFSRYN |
| 270 | ISTSSNYI |
| 271 | TRGWGPFDY |
| 272 | QSIGTA |
| 273 | GFSLSSYAMS |
| 274 | VIDSGGTTY |
| 275 | ESYAGYDYYGHPIPYGMEP |
| 276 | GFSLSSY |
| 277 | DSGGT |
| 278 | VIDSGGTTYYAQKFQG |
| 279 | GFSLSSYA |
| 280 | IDSGGTT |
| 281 | ARESYAGYDYYGHPIPYGMEP |
| 282 | QVQLVQSGAEVKKPGASVKVSCKASGFSLSSYAMSWVRQAPGQGLEWMGVI |
| | DSGGTTYYAQKFQGRVTMTRDTSTSTVYMELSSLRSEDTAVYYCARESYAGY |
| | DYYGHPIPYGMEPWGQGTLVTVSS |
| 283 | QSLYNNKN |
| 284 | DIQMTQSPSTLSASVGDRVTITCQASQSLYNNKNLAWYQQKPGKAPKLLIYYA |
| | SALASGVPSRFSGSGSGTEFTLTISSLQPDDFATYYCQGEFNCNSADCNSFGGG |
| | TKVEIK |
| 285 | EVQLVESGGGLVKPGGSLRLSCAASGFTFSRYNMNWVRQAPGCGLEWVSSIS |
| | TSSNYIYYADSVKGRFTFSRDNAKNSLDLQMSGLRAEDTAIYYCTRGWGPFDY |
| | WGQGTLVTVSS |
| 286 | DIQMTQSPSTLSASVGDRVTITCQASQSLYNNKNLAWYQQKPGCAPKLLIYYA |
| | SALASGVPSRFSGSGSGTEFTLTISSLQPDDFATYYCQGEFNCNSADCNSFGGG |
| | TKVEIK |
| 287 | QVQLVQSGAEVKKPGASVKVSCKASGFSLSSYAMSWVRQAPGQGLEWMGVI |
| | DSGGTTYYAQKFQGRVTMTRDTSTSTVYMELSSLRSEDTAVYYCARESYAGY |
| | DYYGHPIPYGMEPWGCGTLVTVSS |
| 288 | QVQLVQSGAEVKKPGASVKVSCKASGFSLSSYAMSWVRQAPGQGLEWMGVI |
| | DSGGTTYYAQKFQGRVTMTRDTSTSTVYMELSSLRSEDTAVYYCARESYAGY |
| | DYYGHPIPYGMEPWGCGTLVTVSSH |
| 289 | EVQLLESGGGLVQPGGSLRLSCAVSGIDLSNNAISWVRQAPGKGLEYVGVIGN |
| | TGDTYYADSAKGRFTISRDSSKTTLYLQMNSLRAEDTAVYFCVRGPPSYSSSV |
| | KNIWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCDGGGGSGGGGSDIQMTQSPSSLSASVGDRVTITCRARQSIGTAIH |
| | WYQQKPGCAPKLLIKYASESISGVPSRFSGSGSGTDFTLTISSLQPEDFATYYCQ |
| | QSGSWPYTFGQGTKLEIKGGGSGGSGGCPPCGGSGGEVQLVESGGGLVKPGG |
| | SLRLSCAASGFTFSRYNMNWVRQAPGKGLEWVSSISTSSNYIYYADSVKGRFT |
| | FSRDNAKNSLDLQMSGLRAEDTAIYYCTRGWGPFDYWGCGTLVTVSSEPKSS |
| | DKTHTCPPCPAPEAAGGPSVFLFPPKPKDTLMISRTPEVTCVVVSVSHEDPEVK |
| | FNWYVDGVEVHNAKTKPREEQYNSTYRVVSVLTVLHQDWLNGKEYKCKVS |
| | NKALPAPIEKTISKAKGQPREPQVYTLPPSREEMTKNQVSLWCLVKGFYPSDIA |
| | VEWESNGQPENNYKTTPPVLDSDGSFFLYSKLTVDKSRWQQGNVFSCSVMHE |
| | ALHNHYTQKSLSLSPGK |
| 290 | QVQLVQSGAEVKKPGASVKVSCKASGFSLSSYAMSWVRQAPGQGLEWMGVI |
| | DSGGTTYYAQKFQGRVTMTRDTSTSTVYMELSSLRSEDTAVYYCARESYAGY |
| | DYYGHPIPYGMEPWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYIC |
| | NVNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEMT |
| | KNQVSLSCAVKGFYPSDIAVEWESNGQPENNYKTTPPVLDSDGSFFLVSKLTV |
| | DKSRWQQGNVFSCSVMHEALHNRFTQKSLSLSPGK |
| 291 | DIQMTQSPSTLSASVGDRVTITCQASQSLYNNKNLAWYQQKPGKAPKLLIYYA |
| | SALASGVPSRFSGSGSGTEFTLTISSLQPDDFATYYCQGEFNCNSADCNSFGGG |
| | TKVEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPVTK |
| | SFNRGEC |
| 292 | QVQLVQSGAEVKKPGASVKVSCKASGFSLSSYAMSWVRQAPGQGLEWMGVI |
| | DSGGTTYYAQKFQGRVTMTRDTSTSTVYMELSSLRSEDTAVYYCARESYAGY |
| | DYYGHPIPYGMEPWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYIC |
| | NVNHKPSNTKVDKKVEPKSCGGGGSGGGGSDIQMTQSPSSLSASVGDRVTITC |
| | RARQSIGTAIHWYQQKPGCAPKLLIKYASESISGVPSRFSGSGSGTDFTLTISSLQ |
| | PEDFATYYCQQSGSWPYTFGQGTKLEIKGGGSGGSGGCPPCGGSGGEVQLVES |
| | GGGLVKPGGSLRLSCAASGFTFSRYNMNWVRQAPGKGLEWVSSISTSSNYIYY |
| | ADSVKGRFTFSRDNAKNSLDLQMSGLRAEDTAIYYCTRGWGPFDYWGCGTL |
| | VTVSSEPKSSDKTHTCPPCPAPEAAGGPSVFLFPPKPKDTLMISRTPEVTCVVVS |
| | VSHEDPEVKFNWYVDGVEVHNAKTKPREEQYNSTYRVVSVLTVLHQDWLNG |
| | KEYKCKVSNKALPAPIEKTISKAKGQPREPQVYTLPPSREEMTKNQVSLWCLV |
| | KGFYPSDIAVEWESNGQPENNYKTTPPVLDSDGSFFLYSKLTVDKSRWQQGN |
| | VFSCSVMHEALHNHYTQKSLSLSPGK |
| 293 | DIQMTQSPSTLSASVGDRVTITCQASQSLYNNKNLAWYQQKPGCAPKLLIYYA |
| | SALASGVPSRFSGSGSGTEFTLTISSLQPDDFATYYCQGEFNCNSADCNSFGGG |
| | TKVEIKGGGSGGSGGCPPCGGSGGQVQLVQSGAEVKKPGASVKVSCKASGFS |
| | LSSYAMSWVRQAPGQGLEWMGVIDSGGTTYYAQKFQGRVTMTRDTSTSTVY |
| | MELSSLRSEDTAVYYCARESYAGYDYYGHPIPYGMEPWGCGTLVTVSSGGGG |
| | SGGGGSEVQLLESGGGLVQPGGSLRLSCAVSGIDLSNNAISWVRQAPGKGLEY |
| | VGVIGNTGDTYYADSAKGRFTISRDSSKTTLYLQMNSLRAEDTAVYFCVRGPP |
| | SYSSSVKNIWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNHK |
| | PSNTKVDKKVEPKSCDKTHTCPPCPAPEAAGGPSVFLFPPKPKDTLMISRTPEV |
| | TCVVVSVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQV |
| | SLSCAVKGFYPSDIAVEWESNGQPENNYKTTPPVLDSDGSFFLVSKLTVDKSR |
| | WQQGNVFSCSVMHEALHNRFTQKSLSLSPGK |
| 294 | EVQLLESGGGLVQPGGSLRLSCAVSGIDLSNNAISWVRQAPGKGLEYVGVIGN |
| | TGDTYYADSAKGRFTISRDSSKTTLYLQMNSLRAEDTAVYFCVRGPPSYSSSV |
| | KNIWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCGGGGSGGGGSDIQMTQSPSTLSASVGDRVTITCQASQSLYNNKN |
| | LAWYQQKPGCAPKLLIYYASALASGVPSRFSGSGSGTEFTLTISSLQPDDFATY |
| | YCQGEFNCNSADCNSFGGGTKVEIKGGGSGGSGGCPPCGGSGGQVQLVQSGA |
| | EVKKPGASVKVSCKASGFSLSSYAMSWVRQAPGQGLEWMGVIDSGGTTYYA |
| | QKFQGRVTMTRDTSTSTVYMELSSLRSEDTAVYYCARESYAGYDYYGHPIPY |
| | GMEPWGCGTLVTVSSEPKSSDKTHTCPPCPAPEAAGGPSVFLFPPKPKDTLMIS |
| | RTPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEMT |
| | KNQVSLSCAVKGFYPSDIAVEWESNGQPENNYKTTPPVLDSDGSFFLVSKLTV |
| | DKSRWQQGNVFSCSVMHEALHNRFTQKSLSLSPGK |
| 295 | EVQLLESGGGLVQPGGSLRLSCAVSGIDLSNNAISWVRQAPGKGLEYVGVIGN |
| | TGDTYYADSAKGRFTISRDSSKTTLYLQMNSLRAEDTAVYFCVRGPPSYSSSV |
| | KNIWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCDKTHTCPPCPAPEAAGGPSVFLFPPKPKDTLMISRTPEVTCVVVS |
| | VSHEDPEVKFNWYVDGVEVHNAKTKPREEQYNSTYRVVSVLTVLHQDWLNG |
| | KEYKCKVSNKALPAPIEKTISKAKGQPREPQVYTLPPSREEMTKNQVSLSCAV |
| | KGFYPSDIAVEWESNGQPENNYKTTPPVLDSDGSFFLVSKLTVDKSRWQQGN |
| | VFSCSVMHEALHNRFTQKSLSLSPGKGGGGSGGGGSDIQMTQSPSTLSASVGD |
| | RVTITCQASQSLYNNKNLAWYQQKPGCAPKLLIYYASALASGVPSRFSGSGSG |
| | TEFTLTISSLQPDDFATYYCQGEFNCNSADCNSFGGGTKVEIKGGGSGGSGGCP |
| | PCGGSGGQVQLVQSGAEVKKPGASVKVSCKASGFSLSSYAMSWVRQAPGQG |
| | LEWMGVIDSGGTTYYAQKFQGRVTMTRDTSTSTVYMELSSLRSEDTAVYYCA |
| | RESYAGYDYYGHPIPYGMEPWGCGTLVTVSSH |
| 296 | EVQLLESGGGLVQPGGSLRLSCAVSGIDLSNNAISWVRQAPGKGLEYVGVIGN |
| | TGDTYYADSAKGRFTISRDSSKTTLYLQMNSLRAEDTAVYFCVRGPPSYSSSV |
| | KNIWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCGGGGSGGGGSEVQLLESGGGLVQPGGSLRLSCAVSGIDLSNNAI |
| | SWVRQAPGKGLEYVGVIGNTGDTYYADSAKGRFTISRDSSKTTLYLQMNSLR |
| | AEDTAVYFCVRGPPSYSSSVKNIWGQGTLVTVSSASTKGPSVFPLAPSSKSTSG |
| | GTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPEAAGGPSVFLFP |
| | PKPKDTLMISRTPEVTCVVVSVSHEDPEVKFNWYVDGVEVHNAKTKPREEQY |
| | NSTYRVVSVLTVLHQDWLNGKEYKCKVSNKALPAPIEKTISKAKGQPREPQV |
| | YTLPPSREEMTKNQVSLSCAVKGFYPSDIAVEWESNGQPENNYKTTPPVLDSD |
| | GSFFLVSKLTVDKSRWQQGNVFSCSVMHEALHNRFTQKSLSLSPGK |
| 297 | QVQLVQSGAEVKKPGASVKVSCKASGFSLSSYAMSWVRQAPGQGLEWMGVI |
| | DSGGTTYYAQKFQGRVTMTRDTSTSTVYMELSSLRSEDTAVYYCARESYAGY |
| | DYYGHPIPYGMEPWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYIC |
| | NVNHKPSNTKVDKKVEPKSCGGGGSGGGGSEVQLVESGGGLVKPGGSLRLSC |
| | AASGFTFSRYNMNWVRQAPGCGLEWVSSISTSSNYIYYADSVKGRFTFSRDNA |
| | KNSLDLQMSGLRAEDTAIYYCTRGWGPFDYWGQGTLVTVSSGGGSGGSGGCP |
| | PCGGSGGDIQMTQSPSSLSASVGDRVTITCRARQSIGTAIHWYQQKPGKAPKLL |
| | IKYASESISGVPSRFSGSGSGTDFTLTISSLQPEDFATYYCQQSGSWPYTFGCGT |
| | KLEIKEPKSSDKTHTCPPCPAPEAAGGPSVFLFPPKPKDTLMISRTPEVTCVVVS |
| | VSHEDPEVKFNWYVDGVEVHNAKTKPREEQYNSTYRVVSVLTVLHQDWLNG |
| | KEYKCKVSNKALPAPIEKTISKAKGQPREPQVYTLPPSREEMTKNQVSLWCLV |
| | KGFYPSDIAVEWESNGQPENNYKTTPPVLDSDGSFFLYSKLTVDKSRWQQGN |
| | VFSCSVMHEALHNHYTQKSLSLSPGK |
| 298 | QVQLVQSGAEVKKPGASVKVSCKASGFSLSSYAMSWVRQAPGQGLEWMGVI |
| | DSGGTTYYAQKFQGRVTMTRDTSTSTVYMELSSLRSEDTAVYYCARESYAGY |
| | DYYGHPIPYGMEPWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYIC |
| | NVNHKPSNTKVDKKVEPKSCGGGGSGGGGSQVQLVQSGAEVKKPGASVKVS |
| | CKASGFSLSSYAMSWVRQAPGQGLEWMGVIDSGGTTYYAQKFQGRVTMTRD |
| | TSTSTVYMELSSLRSEDTAVYYCARESYAGYDYYGHPIPYGMEPWGQGTLVT |
| | VSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDK |
| | THTCPPCPAPEAAGGPSVFLFPPKPKDTLMISRTPEVTCVVVSVSHEDPEVKFN |
| | WYVDGVEVHNAKTKPREEQYNSTYRVVSVLTVLHQDWLNGKEYKCKVSNK |
| | ALPAPIEKTISKAKGQPREPQVYTLPPSREEMTKNQVSLSCAVKGFYPSDIAVE |
| | WESNGQPENNYKTTPPVLDSDGSFFLVSKLTVDKSRWQQGNVFSCSVMHEAL |
| | HNRFTQKSLSLSPGK |

1. An antibody, or an antigen-binding fragment thereof, comprising:
a heavy chain comprising a heavy chain complementarity determining region (CDR) 1 (CDR1) comprising the amino acid sequence of SEQ ID NO: 81, a heavy chain CDR2 comprising the amino acid sequence of SEQ ID NO: 259, and a heavy chain CDR3 comprising the amino acid sequence of SEQ ID NO: 202; and
a light chain comprising a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34.

2.-33. (canceled)

34. A bispecific antibody comprising a heavy chain, a light chain, and a stapled single chain fragment variable (spFv) chain,
wherein the heavy chain associates with the light chain to form an antigen binding site that immunospecifically binds to a mutant calreticulin (CALRmut);
wherein the spFv chain immunospecifically binds to CD3;
wherein the heavy chain comprises a heavy chain CDR1 comprising the amino acid sequence of SEQ ID NO: 81, a heavy chain CDR2 comprising the amino acid sequence of SEQ ID NO: 259, and a heavy chain CDR3 comprising the amino acid sequence of SEQ ID NO: 202;
wherein the light chain comprises a light chain CDR1 comprising the amino acid sequence of SEQ ID NO: 33, a light chain CDR2 comprising the amino acid sequence of SEQ ID NO: 23, and a light chain CDR3 comprising the amino acid sequence of SEQ ID NO: 34; and
wherein the spFv chain comprises a light chain CDR1 comprising the amino acid sequence of SEQ ID NO: 214, a light chain CDR2 comprising the amino acid sequence of SEQ ID NO: 215, a light chain CDR3 comprising the amino acid sequence of SEQ ID NO: 216, a heavy chain CDR2 comprising the amino acid sequence of SEQ ID NO: 265, a heavy chain CDR2 comprising the amino acid sequence of SEQ ID NO: 266, and a heavy chain CDR3 comprising the amino acid sequence of SEQ ID NO: 212.

35. (canceled)

36. The bispecific antibody of claim 34, wherein the VH comprises the amino acid sequence of SEQ ID NO: 203.

37. (canceled)

38. The bispecific antibody of claim 34, wherein the VL comprises the amino acid sequence of SEQ ID NO: 205.

39. The bispecific antibody of claim 34, comprising a heavy chain variable region (VH) comprising the amino acid sequence of SEQ ID NO: 203, and a light chain variable region (VL) comprising the amino acid sequence of SEQ ID NO: 205.

40. The bispecific antibody of claim 34, wherein the bispecific antibody is an IgG.

41. The bispecific antibody of claim 34, wherein the bispecific antibody comprises an IgG1 isotype Fc region.

42. The bispecific antibody of claim 41, further comprising L234A, L235A, and D265S substitutions in the Fc region.

43. The bispecific antibody of claim 41, further comprising knob-into-hole (KiH) substitutions.

44. The bispecific antibody of claim 41, further comprising H435R and Y436F substitutions in the Fc region.

45. (canceled)

46. The bispecific antibody of claim 34, wherein the heavy chain comprises the amino acid sequence of SEQ ID NO: 204.

47. (canceled)

48. The bispecific antibody of claim 34, wherein the light chain comprises the amino acid sequence of SEQ ID NO: 206.

49. (canceled)

50. The bispecific antibody of claim 34, wherein the spFv chain comprises the amino acid sequence of SEQ ID NO: 218.

51.-57. (canceled)

58. A pharmaceutical composition comprising the bispecific antibody of claim 34 and a pharmaceutically acceptable carrier.

59.-60. (canceled)

61. A method of inhibiting the growth or proliferation of a myeloproliferative neoplasm (MPN), the method comprising administering to a subject in need thereof a therapeutically effective amount of the bispecific antibody claim 34 to inhibit the growth or proliferation of the MPN.

62. (canceled)

63. The method of claim 61, wherein the MPN is characterized by the presence of a mutant calreticulin.

64. The method of claim 61, wherein the MPN is selected from the group consisting of chronic myelogenous leukemia, polycythemia vera, primary myelofibrosis, essential thrombocythemia, chronic neutrophilic leukemia, and chronic eosinophilic leukemia.

65.-72. (canceled)

73. A bispecific antibody comprising a Chain A and a Chain B,
wherein Chain A comprises a stapled single chain fragment variable (spFv) region that immunospecifically binds to CD3,
wherein Chain B comprises a heavy chain and a light chain, wherein the heavy chain associates with the light chain to form a mutant calreticulin (CALRmut) antigen binding site that immunospecifically binds to CALRmut,
wherein the Chain A spFv region comprises a light complementarity determining region (CDR) 1 (CDR1) comprising the amino acid sequence of SEQ ID NO: 214, a light CDR2 comprising the amino acid sequence of SEQ ID NO: 215, a light CDR3 comprising the amino acid sequence of SEQ ID NO: 216, a heavy CDR1 comprising the amino acid sequence of SEQ ID NO: 265, a heavy CDR2 comprising the amino acid sequence of SEQ ID NO: 266, and a heavy CDR3 comprising the amino acid sequence of SEQ ID NO: 212,
wherein the Chain B heavy chain comprises a heavy CDR1 comprising the amino acid sequence of SEQ ID NO: 66, a heavy CDR2 comprising the amino acid sequence of SEQ ID NO: 278, and a heavy CDR3 comprising the amino acid sequence of SEQ ID NO: 275; and
wherein the Chain B light chain comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.

74.-112. (canceled)

113. The bispecific antibody of claim 34, wherein the spFv comprises a heavy chain variable region (VH) comprising an amino acid sequence of SEQ ID NO: 213.

114. The bispecific antibody of claim 34, wherein the spFv comprises a light chain variable region (VL) comprising an amino acid sequence of SEQ ID NO: 217.

115. The bispecific antibody of claim 34, wherein the spFv chain further comprises a heavy chain comprising a mutant calreticulin (CALRmut) heavy binding fragment and a light chain comprising a CALRmut light binding fragment, and wherein the CALRmut heavy binding fragment is associated with a CALRmut light binding fragment to form a CALRmut antigen binding site,
wherein the CALRmut antigen binding site of the spFv chain immunospecifically binds to CALRmut, and
wherein the CALRmut heavy binding fragment comprises a heavy complementarity determining region (CDR) 1 (CDR1) comprising the amino acid sequence of SEQ ID NO: 81, a heavy CDR2 comprising the amino acid sequence of SEQ ID NO: 259, and a heavy CDR3 comprising the amino acid sequence of SEQ ID NO: 202; and
wherein, the CALRmut light binding fragment comprises a light CDR1 comprising the amino acid sequence of SEQ ID NO: 33, a CDR2 comprising the amino acid sequence of SEQ ID NO: 23, and a CDR3 comprising the amino acid sequence of SEQ ID NO: 34.

116. The bispecific antibody of claim 34, wherein the heavy chain further comprises a stapled single chain fragment variable (spFv) region,
wherein the spFv region immunospecifically binds to CALRmut, and
wherein the spFv region comprises a light CDR1 comprising the amino acid sequence of SEQ ID NO: 70, a light CDR2 comprising the amino acid sequence of SEQ ID NO: 71, a light CDR3 comprising the amino acid sequence of SEQ ID NO: 72, a heavy CDR1 comprising the amino acid sequence of SEQ ID NO: 66, a heavy CDR2 comprising the amino acid sequence of SEQ ID NO: 278, and a heavy CDR3 comprising the amino acid sequence of SEQ ID NO: 275.

117. The bispecific antibody of claim 34, wherein the heavy chain further comprises a second CALRmut heavy binding fragment, and wherein the light chain further comprises a second CALRmut light binding fragment, wherein the second CALRmut heavy binding fragment is associated with the second CALRmut light binding fragment to form a second CALRmut antigen binding site, and
wherein the second CALRmut heavy binding fragment comprises a heavy CDR1 comprising the amino acid sequence of SEQ ID NO: 81, a heavy CDR2 comprising the amino acid sequence of SEQ ID NO: 259, and a heavy CDR3 comprising the amino acid sequence of SEQ ID NO: 202, and
wherein the second CALRmut light binding fragment comprises a light CDR1 comprising the amino acid sequence of SEQ ID NO: 33, a light CDR2 comprising the amino acid sequence of SEQ ID NO: 23, and a light CDR3 comprising the amino acid sequence of SEQ ID NO: 34.

118. The bispecific antibody of claim 43, wherein the KiH substitutions comprise a T336W substitution in the Fc domain of the spFv chain, and T366S, L368A and Y407V substitutions in the Fc domain of the heavy chain.

119. The bispecific antibody of claim 43, wherein the KiH substitutions comprise a T336W substitution in the Fc domain of the heavy chain, and T366S, L368A and Y407V substitutions in the Fc domain of the spFv chain.

120. The bispecific antibody of claim 73, wherein Chain A further comprises a heavy chain comprising a mutant calreticulin (CALRmut) heavy binding fragment and a light chain comprising a CALRmut light binding fragment, wherein the CALRmut heavy binding fragment is associated with the CALRmut light binding fragment to form a CALRmut antigen binding site,
wherein the CALRmut antigen binding site of Chain A immunospecifically binds to CALRmut,
wherein the Chain A CALRmut heavy binding fragment comprises a heavy complementarity determining region (CDR) 1 (CDR1) comprising the amino acid sequence of SEQ ID NO: 66, a heavy CDR2 comprising the amino acid sequence of SEQ ID NO: 278, and a heavy CDR3 comprising the amino acid sequence of SEQ ID NO: 275; and
wherein the Chain A CALRmut light binding fragment comprises a light CDR1 comprising the amino acid sequence of SEQ ID NO: 70, a CDR2 comprising the amino acid sequence of SEQ ID NO: 71, and a CDR3 comprising the amino acid sequence of SEQ ID NO: 72.

121. The bispecific antibody of claim 73, wherein Chain B further comprises a second CALRmut heavy binding fragment, and a second light chain comprising a second CALRmut light binding fragment,
wherein the second CALRmut heavy binding fragment is associated with the second CALRmut light binding fragment to form a second CALRmut antigen binding site,
wherein the Chain B second CALRmut heavy binding fragment comprises a heavy CDR1 comprising the amino acid sequence of SEQ ID NO: 66, a heavy CDR2 comprising the amino acid sequence of SEQ ID NO: 278, and a heavy CDR3 comprising the amino acid sequence of SEQ ID NO: 275, and
wherein the Chain B second CALRmut light binding fragment comprises a light CDR1 comprising the amino acid sequence of SEQ ID NO: 70, a heavy CDR2 comprising the amino acid sequence of SEQ ID NO: 71, and a heavy CDR3 comprising the amino acid sequence of SEQ ID NO: 72.

122. A bispecific antibody comprising a heavy chain comprising the amino acid sequence of SEQ ID NO: 204 and a light chain comprising the amino acid sequence of SEQ ID NO: 206, wherein the heavy chain associates with the light chain to form an antigen binding site that immunospecifically binds to a mutant calreticulin (CALRmut).

123. The bispecific antibody of claim 122 further comprising a spFv chain comprising the amino acid sequence of SEQ ID NO: 218, wherein the spFv chain immunospecifically binds to CD3.
